SEQUENCE LISTING (69 sequences)

SEQ ID NO: 1 - protein, 5 aa, Streptomyces xanthochromogenus
TVPGV

SEQ ID NO: 2 - DNA, 52589 bp, Artificial Sequence
  Synthetic Sequence - pKU508acmCW
tcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacggcctcctcacccggtcacgtttcgtcgtttctcctccacgcgctcggcttcggggccgcacctgcattcgtatgcggtcgcccggttacaggtgcggcacggcctgatggaggccgcatgtgagaggagaattgagcttgcagtgggcttacatggcgatagctagactgggcggttttatggacagcaagcgaaccttcatgagctcagccaatcgactggcgagcggcatcgcattcttcgcatcccgccctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcattttccgctgcataaccctgcttcggggtcattatagcgattttttcggtatatccatcctttttcgcacgatatacaggattttgccaaagggttcgtgtagactttccttggtgtatccaacgggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccagaattcgaggtcttcgccccctaagcgtgtccgtcgccgacgccgaccggcacgggcacttcagcgccccattccttgcccccgaaggagtggggcgctggccgtttagggtggccgtatgaagctcatttggcagggcgaagacatgcccccaggcgaagccgccggaacccctgacgccgtcctcgaagcgctcacagacgccgtcctagcggcctacgccgaccacacgcggacgacccgcgcgcacctgatgttgaacgtcgtcgccccgctccgcgaacggctcatcaggtacggcgtgccgacggtcgaggcgggcggcgtatgggccaaccaggaagggccggtaagcgtcacggcacgcgccgactgagagacgtttccgcaggtcaaccccgttccagcccaacagtgttagtctttgctcttacccagttgggcgggatagcctgcccggcatgagcgtgaaggttgaaggcatggtcattctggcaggcggctacgaccgacagtcggcggaacgggagaacagttcgaccgcttcaccggccacccagcgcgccgcgaaccgggggaaggctgaggcgctggcgaaggagtacgcgcgcgacggcgtcgaggtgaagtggctgggtcacttcagcgaagcgcccggcacgtcggcattcacgggcgtcgaccggccggagttcaaccggattttggacatgtgccggaaccgggaaatgaacatgatcattgttcattacatttcgcgcctcagccgcgaagagccgctggacattattccggtcgtcacggaattgctccggctgggcgtgaccattgtcagcgtgaacgaaggcacattccgccccggcgaaatgatggaccttattcacctgatcatgcgccttcaggcttcgcatgatgagtcgaagaacaagagcgtcgccgtgtcgaacgctaaggaattggcgaagcggctgggcggacacacggggtcgacgccgtacggattcgacacggtcgaggaaatggttccgaacccggaagacggcggaaagctggttgccattcgccgactggtgcccagcgcgcacacctgggaaggcgcacacggcagcgaaggggcggtaatccgctgggcgtggcaggagatcaagacgcaccgcgatacgcccttcaagggtggcggagccgggtcgtttcaccctgggtcgctgaacgggctttgtgagcggctgtaccgcgacaaggtgcctacgcgcggcacgctggtcggtaagaagcgcgccggttccgattgggaccccggcgttttgaagcgcgtactcagcgacccgcgcattgccgggtatcaagctgacatcgcatacaaggtgcgcgccgacggttcgcggggcggcttcagccattacaagatcaggcgcgacccggtcaccatggagccgctgaccctgcccggcttcgagccgtacattcccccggcggaatggtgggaacttcaggagtggcttcagggtcgaggacgcgggaagggtcagtaccgggggcaatcgctcctgtcggcaatggacgtcctttactgctacggctccggccagctcgacccggagacgggttacagcaacgggtcgaccatggcgggcaacgtccgcgaaggtgatcaagctcacaagtcgtcgtacgcgtgcaagtgcccccgccgggttcatgacgggtcgtcatgctcgatcacgatgcacaaccttgacccgtacatcgtcggcgcgatcttcgcgcgcatcacggccttcgaccctgccgaccctgacgacctcgaaggcgacacggcagcgctcatgtacgaagccgcacggcgctggggagcgacgcacgaacgcccggagttgaagggtcagcgctccgaactgatggcacagcgcgcggacgccgtgaaggcgctcgaagagctttacgaagacaagcggaacggcggctaccggtccgccatgggacggcgcgcgtttctcgaagaggaagccgcgctgacgctccgcatggaaggggccgaagaacggcttcgtcagctcgacgccgccgactcccccgtgctgccgatcggcgaatggctgggcgaccggggcagcgacccgacgggaccgggttcgtggtgggcgttggcgccccttgaagaccgtcgggcgttcgtccggctcttcgtggaccggatcgaggtgatcaagttgccgaagggcgttcagcggcccggacgggttcccccgatcgccgaccgtgtgcgtatccactgggcgaagccgaaggtcgaggaagagacggagccggagacgctgaacgggttcacagcggcggcgtgacggcggcaccagcgcaacgggaaggggcttcggccccttttctcgtgcccggcgtcggttcgttgccctaagcaactgttcctagcgtcacgtcagcgccggaccggcaggcttcccacctgggcaaagagacggaattcccggggatcagctttatgcttgtaaaccgttttgtgaaaaaatttttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgccggatccacgtgatcgatgcatacgcgttcgaaagcttcgcgacgtacgacgtccgcggagctctcgagcatgctagcggccgcgacatcgacgacgaccaagccatcctcgtcctgaccgccaccgacccggatcccgaggcctgacccgacccggaccccgaccccgggcagaccccagcaccgccacgaaaccggcggatggcgacgacagccgcgcccgggtgcctgtcccaggcatcctgggcaatgagggcatcgatgttccggcccccagcagtacggccgtgtcccatgggtacgatcgcatcccgttcaagggcgggacgacatccacgcacggggagttcgcatggccaccatccatcgcaccaccatgacgccgacgaagctggaactgctcgccggctggctgccgaagcagagctggtacgtcggcgatgcggcaaagccggaactggtcaaggcaggtgggttccggttggacgatccgaagggtgcggtcgggatcgaattcatggtcgtggtggacaccaccgcgcaggagccggtggcctacttggtgccgatggcctaccgggatgctgcactggagggcgtccccgacgaggcgctgatcggcacgtccgagcacggcgtgctcggcactcgctggatctacgacggcgtccacgacccggtggtggtggcgcagttgaacgcgctgctgcgcggcgaggcgaccccgcagcgccagaacgagagcgacacccccgacccgaccgtcaccgtgcacggaacgggtaccgccgacggccgcgacgttcggctccaccgcgtcctgcggtcgggggacacagctcgcccgtcgtccgtacaccttgtcgccgggtggacctggccggacggaacggctgcgcggggcgtaatcgcggggagttgagaccgcaggccgggcttggagggtggggtaagccggagagctcgtcacctctccggcgggcggccgagccgcgagcggcgatggtcggggaagcgttccgcgcgcggatcgaggagatcgatcaggtccatgagggcgtgctgcaacgcgatcagccggtacggcacttccgccgcccgcgccaccacctcgacactgtcactcagcgggccgaaccactcggtgaactccgcgtcctcgtcgagtcggcggcagaactccacgcacccaagacaggagatgccgtccgcggccggcatgagctcgccgatcgcccgctgatcgctgcggaagacgatgaagtgcccatcgaggaagcgtgtgttgccgccaccatcgttgttcagcacgttgctgatcgccgtgaagtgacccacgatccgccggttgagcgtgctgtccccgagatcgaggaactggacaccgcggcgcaggatttccacccggcccaggtactcgcccagtacatagagcgtgctgcgacgggcgtacgcgcgttcccggtcggagccgtcgcggaaatacacccgcaggaatccacggtccacgatgttgtagaggcgggactgcagatcgaaggcggcccacaagagcggatcacggtggcggcccatcaggtcctgctgcgtggtccggctcttcaactcgtgtgcgagcagcgcttgtcggcggctggagcgggccgagaaccaggcgccgccgagcgccaccacacctgacagggacgcgacgaccaattcggtattcacaggcccgggagcgtagcgggcacgacagccacctgcactggtttcgccgcctccggatgcgctgggaggcatgcggtgacacacacccgccgagaccttcctcaccctgccgcgtaggcctgctcgctctcccggaaacgctcgatcaggagcggatccatcagggtggaatccggcccgtagaacttgccgaacttcgccctgtattgctggaaccagtgcggacgttcggagaggaagaagatgtcgtggagggcgatggagcggatcgcgagcaagggtacgggtgcccggctcaccgcgaagcgggagttgcgtgccgctgttacccggcagttctcgtggaactggccgatcatgaggccgtgcgccacgaagtcgtccttcacacgggcttgggcctggtcgagcagtcccgtgtcctcgctgcgcagatcgggcaggacgatcaccatggcctgcagcatggggttccggccctgccaggtcgtcagctcgtactcccgcaggccgctgcgtgcgatctcctcgaccagttcgaggctcggggcatgcccgacaaggcgtatgcggacctccatggtccggtttttgcgggagggtgacacgaacgggcagatcggcccggtgcggccgatctccggatggctcgccgatatgtactcgctcagccacgagtcgaccgactccagcgcggactcgaagtcgacgtcggatgtcttgggcgtcatggcgtacccgtcaggccggcaacgcgatcgacggcgatgcgggcgagccgatggatctcggacccgtccggaatacgggtgttgatgaagttgaggatgccgtccttcggcacgatcacccacgcggtctcgatgcggaagtggtcgacggcggacagccagcgcacgacgaagccgccgtcccccaactccaccgcccccaggtccactttcgtgatgtcgagttcgacgccgtcgttcagccccatccggtagtgctcgcagtcgcgcaccgcttcgaagagctcctggtacacctgccggcaagccccgttgccgaactgtgcgacgtggtggaacaccacggaacccacgatgttcgtgaacagggcggaggcgtgatgcgtcgtctccgggtgcgtgccgtgcgtgagcatgttagtcaggtcgacgatggcccgccccgactcgtcgccggagacgaacaccggatcgtaggcggcgctcggctcctccccgtagaacgactcctccaagtccttttcgttcagcagtagttgtgcgaaagcggcgtcgtccatcgcgttctcctatcggcgcacgaggctcagcggtcgcatgtcggtccagtgctcctcgacgtaggtgaggcagctctggcggtcggtctcggcgagcgcgacggcccagcccgcgggcacctcggcgaacgcgggccacagcgaatgctggccttcctcgttgacgagcacgaggaaggtgccgtcggaattctcgaaggggttgctcatcattgctccctgcgtagacagttgcggtgggggagggcggggcccgtcactcctcgtgcagtaccgccgtcacgtacttgtggatgccgttgatcgaggtgaacagcgtcatgtcctcgaactgcgggtcgatgacgatgccgtgccgctcctccagctcgtgctggagaaggaccagggtgaacgagtcgatcaccagctcggcatcgtccggcagctcctcgggcggaccgagacccgcgttctcggccaggatcgctctgatgtcgtctttcgagatcaccggttcccctgtctgcgccatgtggtctgccctgtcgtcatgcctggggcgatgcttccgcggtgatgcgtccgtgcatcagtgcacggtcgcgtacgagtttgcccatcgagttccgcggcagctgccgcgtgacgaagtaccgcttgggattcttgaccgggctcagccgctcgcggcaccagtcggtgagctcgtccgaggtgggcgcccggtcggttccggtgcccacgaacgcctcgatgaactgctcatgggtcaccaccgcctcggtcacccccggatgggagaggaggacggtctccacttccgtgaggtcgaccttcagcccgcccaccacggccagtgaatcggccctgccgagcatgctgaggactccggtcgccggatcctggtggacgcggtcgtaggtccgcagccaaccgtccgagaagcgatcggcatgctcgctgaacaggtacggcgaacgttccatccggacgaagagctcctcgtcgagcaccttcacctcgaccccccgcaccggaggccccaattggggcggtggggagccgccggacaggtcgcccgcgatcaacccgatctcggtcacgccgtagaccggactgatcggcaggccgaaccgggcgtggaaacgttcataggtctcctgcggcacgcgttccccgccggacaccgcgagccgaagggcgggcagttcgggccgccctccggtccgggaaaggagatcgtagtgaacgggcgttccgtacacggcagttgcctcggcccgcgccatcaggcgcaccacttcggccgggcggagactgggcgggacgatcagggtcgccccgaccgcgagggcgtgcaggacgccgctgaccaggcccatgttgtgcatgacggagttcagcagcatcacccgctcacccctgccggggattccgggcaacgcgctgtgccggtcgagttcggccaggacggagtcggccgggcggccgatcaccttgggtcggcccgtcgagcccgagctgaactgcaccaggcggatgccgtcgacggccggtaggccgtcgggaagccgctgcaccgagaacccggactcctggcggaatccggcgaccggcccgtccgccccggtcgccacgacgaggtactgcggtcgcaccaggtcggccagcggttcgtattcggtgggcgtcagccggaagtcgaccagaaccacctgtgcgccgcggctccacaaggcgagcagtacttggagataggtgaagctcggcttcattcgcagaagaaccgagctgccgtctgtaatgccttgtttccgaaaacgctcaccgaaatcattgactgctgcacgtaactgcccgcgcgtaactggcgcactggaaattgcccacagttcgtcgtccgcgccccggctcagcagatggctaccccaccatgtatttgccatacacgcttccttttgcgaaaacaggtccgcgaaacgttcgatcaggcatttagtgaaggtaaagctagcaccgccgagaaacctcgggcaagcactcggacgtcaatccgttccagccccggcaaagacgtgcagccccagtcggatgccctgacggttggtgcggaccaagcctcgccgtgggcctccacgcttgcacgtgaagaacttggcgggtcccctgcctgcttcgatcgctgtgccggtgtgctccttcggtccccgccgaccgatttcttgtcccgccggcggtacaaccggcggcacatgcgcatggcgtaggcctttgatctccttgaccccctggggctcccgagttaggttgcccttgattgcgtttccgaggcgttgcgctcgattccggtgcgtactgcgagatttctcgcccagttggcccgacgatcgccttacatccattacccggtcgcatttccgacgtgtcttcatcgggccgtcgtgagcttttcactcgtgtctctccggccgtccgtgcgggggaaacttcggaggtggagacttgttcgccagtcctggcaccacgctgccgctcacggccgcgcagcgcgagatatggatcgccgagcagcgtcttggggaggggaatcgggttttccgggtgggtgaatacctggagatccacggtcaggtggacccggcgctgttcgaacgggcgctggtgcgcgtcgtcggtgaagcggacgccttgcacgtcaggttcgtcgaggaacgcggcgaggtccggcaggtcgtccacgagctcggcgactggtccccttccgtggtcgatctcagcgcggagccggaccccgaacgggccgcccgcgactggctggacgaagcggtcgcccggccgatggacctggccggtgaccggctgttcgagcacgcactcctgcggctgggcggcgaccgctgctactggtaccagggctaccaccacgccgtgatggacgccttcgggtcgctgctgatcacccgccgggtcgcggacgtctacacctcgctcgcccagggcaggccggtgtcaccgagcccgttcggctccctgtccgacctgctggccgccgaaggggagtaccgcgattcgagacgattcaccgaagaccgcgagtactggacggagctcttcgccgaccgcccagcgccggccgggatcgtcggtcgaccctccaccacccccgaccgctaccttcggcacaccgcaggccttgaccatgccgggctgagcgagctgcgcgaagcggcccgccgcgcacgcgtcccctggtcgtacctggtcatcgccgccacagccgtccatctgcaccggatgaccggcgcgtcgaccgtggtgctcggtctgccagtgacggcccgcctgaaccaggtccagcgccgaacgcccggcacggcggccaacgtcctgccgctcaggctcgcgctccgatcggagatgacgcatcgtcaactcctcgatcagatcggggaacggatacgcgaactcggcacccaccagcgctatcgcgccgaggacctccagcgcgacctcgaactgccgggccacgcggggacctggtacgcaccggtcgtcaacatcatgtccttcgactacgcggtgaccttcgccggcctgtcgaccacggcccacaacctctcctcgggactggtgggcgacctcacgttcgccgtgtgggatcgacgcgacggcaccggactgacgctcgacctcaacgcccaccccgagatctgcgcggaccatgaattggccgcacaccagcgccgtttgatcgccgcgctgcgcgccgtcacggcggcggacccctccttgccgattggccggatcgacctgctcaccccggaggagcgggccgccttcctggccgtccccgacgacgttccgccgctgacggaagccacactgacgacgcggttcgaggcgcaggccgacaagacacccgacgcggcggccgtcacctgcggcgaggcgtccctgacataccgggacctgaatgagcgggccaatcggctggcccatcgactggtggcccggggcatgggtccggaacacgtcgtcgcgctggccctgccccgctccgccgacctcgtcgtggccatcctggcgaccctcaaggcgggcgccgcctacctgcccctcgaccccgaatacccgtcggcccgtctcgcccacatggtcaccgacgcctgccccggcctcctcctcacgacaacggcggcccggtccaacctccctgccacacacggcactccggtcctccttctggatgccccggacagcttcaccgacctcgcgcacaaccccgctccggacctggtacccgaccaccccgcctacgtcatctacacctccggctcgaccggacggcccaaaggcgtcgtcaacacccaccggaacgttgtccggctgttcgacgcctcccgcaagtggttcggcttcgggccgcaggacgtctggacgctgttccactcgtatgccttcgacttctccgtctgggagctgtggggtgctctgctgcacggcggccgtcttgtcgtggtgccctacgaggtgagtcgggcgccgggcgccttcctcgacctgctggcggacgagggtgtcaccgtcctcaaccaaaccccctcagccttctaccagttggcgcaggccgaagccgaaggcgaatccggagccgaggcagtaggaggaccggaggccggaacgaacgagggtgccgaggcggtggatgcctccaggccgactcgccgactcgcgctgcggaccgtggtcttcggcggtgaggcgctgcagccgtcccgcctcgccgactggtacgagcggcatcccgacgacgcgccgacgctggtcaacatgtacggcatcaccgagaccacggtgcatgtgacgtaccagccgctgaaccgccggcgggccacgcccacggcggccagtgcgatcggcaccggcctcccggatctgcggacccatgtgctcgacggcgggatgcagttggtgccccccggggtggtgggcgagctgtatgtcgcgggaggcggcgtcgctcgcggctacctgggccgaccgggcctcacggccgaacgcttcgtcgccgacccgtacgcgtccgaacccggtgcccgcatgtaccgcaccggagacctggtgcgctggaatccggatggcgagctggagttcatgggccgcgccgaccatcaggtcaagatccgcggcttccgcatcgaaccgggcgagatcgaggcggtggtgagcagccatccgagcattgccgaggcggctgtggtggtccgccaggagcagtccggtgacgggcgcctggtggcctatgcggtcgcgcgcgatgcactccgggccgaagacgtgcgggagttcgcgcgggagcggctgccggagtacatggttccggccgtcttcctgctggaccgcatgccgctgacggccaacggcaagctggacaaggcggcgctcccggagccggactactcggtgtcggctgcggggcgggaggcgcgtacgccgcaggagcagatcgtgtgcgatctgttcgcgcaggtcctcgggctgccacgggtcgggggcgacgaccatttcttcgagctcggtgggcactcgctcctcgccacccgcctgatcgcccaggtccgggccgtcttcggcgtcgagctggaactgcgcctgctgttcgaggggccgaccccggccgcggtggccgcccgcctcgacaccgccgggcccgggcgcctagcgctcgcgccgcgggagcggccgacggccgttccgctctcctcggcccagcggcggctctggttcctggcccggatggaggggccgagcgccacgtacaacatcccgctcgtcgtgcgactgtccggcgagttggaccacgacgcgctgcgctctgcactcggagacgtggtggcccggcacgaaagtctacggacggtgtttccggagaccgaggacgtgccgtgccagcgggtgctggcccccgaggcggccgtgccgagtctgaccgtcgtgccgacctccgagaccgaactgccggcagccttggaagcgggcgcacggtacaccttcgacctggcggccgaacctccgctgcgggccgggctgttcacgctgtcaccgcgagagcacgtgcttctcgtcgtggtgcaccacatcgccggtgacggctggtcgatgggcccgctgtcgcgggagctgacggaggcgtacacggcacgtgcgcggggccaggcccccgaatggccggcgctggcggtccagtacgccgactacacgctgtggcagaacgaactcctgggcgaccaggacgaccccggcagcctcctcgccacccaactcgactactggacccgcgctctcgccgaccttccggaccaactcaccctgcccacgacccgccctcgcccggccgtcatgacctaccgcggcgactacctcaccgtcgagatcgacgccgcactgcaccaacgcctcgccgacctcgcacggtcgaccggcgccagcctgttcatggtcctccaagcgggactcgcggccctcctcacccgcctcggtgcgggggaggacattccactcggcagccccatcgcgggacgcaccgaccaaaatctggaccacctcatcggcttcttcgtcaacaccctcgtcctgcgcaccgacaccgcaggcaatcccacgttcacccagctcgtcgcccgcgtccgcgagacctgcctcgccgcgtacacccaccaggacgtgcccttcgagtacctcgtcgaacatctcagtcccacgcgcaccctcgcccaccaccctctcttccagatcatgctcgccctccagaacgcccccgagggcgagttcgagctccctggtctgcaagcggctgtcgagctgggcaggacgggcacggcgaagttcgacctgttcttcagcctcaccgagcagcgcggcgaaagcggcaccccccagggcatcaccggtgccgtcgagtactccagcgacctgtacgacgccgacgacgtccgggtcctcttcgagcgctggatccgcctgctggaggcggccacggccaacccgaaccgcccgctcagccaccttgccatcctctcggacgaggaacaccacgacgccgtggtcgagttcaacgacacggccgtgcctttgcccgagttgtctcttgcggagctcttcgtgcggcaggtggccaggactccggatgctcctgctgtgatggatggggagagctctctcacgtatgcggagcttgatgtccgtgcggaccggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggcagagcgcatccggcacgtcctcaccgacgccggggtctcactcgtcgtgaccgacgaagtctcgcggggtgaactcgggtctgtggcaacagagttgttggtcatcggccgcgagtttcgggaaggcgagcactcgccgccggacgtggctgttgcccctgatgccgcggcgtatgtgatgtatacgtctggttcgtcgggtgtgccgaagggggttgtggtcagtcatcggaatgtggctggtctggcgttggatgctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtctgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtcccccgcgacgatctgacgccggaaggccttcgcgagttcgcgcggcgaagcctgccgcagtacatggtgccgtcggcgttcgtcacgctggaccacttgccgctgacggtcaacggcaagctcgacagcgccgcgctgcccgcccccggcccggccgtgcgggcggacgtggagagccgtgagccgcgcaccgcgcgcgagcggctgctcgcggagttgttcgcgcaggctctgggaatggaccgggtcgggtgcgacgacgacttcttcgtgctcggcggggacagcatcgtgtcgatccgtttggtcagcctggcgcgttcggcgggcatcggattcacggtccgcgacgtcttcgagcagcggacggtcgccggactcgccgaagcggccactgaactggccctttctacaacggagttcgacgactcgggcatcggagcggtggaaccgaccccgatcatgcgttggttcgacgagcgcggcggcggaatcgaccagttccatcaggcgatgctgctggaggtgccggccgatctgggcgcggagcgcctcaccctggccgtccaggcactcgtggaccaccatgacgcgctgcgtctgacccggacgccgggtggcacggatggcaggtcgtggtcactggcggtcgcgtcggtgggttcggtgacggccgccgagctggtgcacacggtggacctcgacggggcggaccttgacgaggcgctcgtccgggaccatgcggaggcggcggccacccggctgtcggccgagcggggggcgctgctccaggcggtctggttcgatgccggaccggtccgccacgggaggctgctgctcctcgtcaaccacctggtggtggacggcgtgtcctggcgcatcctgcttccggacctcatcgccgcctgggaggcggtcgcgctcggccgcccgccccggctcgacccggtgggcacctcgctgcgccgctggtcggcgctgctggccgcggaggcccgacacccggcccgagccgcggaggcgaccctgtgggccgatctcctcgccgccgccgacccaccgctgacggccggccggctcgatcccgttcgggacaccgtgggccgggccagtgcgctcacgctctcccttccgcccgacgtcaccaccccgctcctgaccacggtgcccacggccttccacgccggagtgaacgacgtcctgctgaccgctctgggcctggcggtggcccagtggcgccgggggcacgcccgcgggaggcacacggcactgctggtcgacgtggaggggcacggccgggaggagatcgtcgagggcgtggacctgtcgcgcacggtgggttggttcacctcgctgtatcccgtccgtgtcgacccgggaccactggtctgggaggaggtcgtggaaggcgggcccgctctcggacaggcgctgaaacgggtcaaggaacagctgcgggcgctgcccgaccgaggcatgggcttcggtctgctgcgccacctcaacaccgggaccgggccggagctggccgggctcgccgtgccccaactcggcttcaactacctcggccgcttcccgtccgcgggcactccggtggtgcccggccatccgggatggaccgtggcgtcggaggccggcctgctgagcggcgccgatccggcgaccgccctggcgcacggcatcgaggtcaactccatggtccgtgacaccccggacgggccgtggttggaggccgtctggacctgggctcctgatctctggcccgaaccacacgtccgcgccctcgcggagcagtggttcctggcgatccgtggcctggtccggcacgtcgaccggtccggtgcgggcggccacacaccctcggacttccccctgaccgaactcagtcagcacgacatcgagcagctcgaagccgcgtggaggattcagaaatgacgtcgtcgggcctggaagacatccttcccctctcgcccatgcaggaggggctcctgttccactcccgctacgagcaggacggcgcggacgtctatgcggtgcagcacgtcttcgacatcgaaggcaccctcgacgcggcgcgcctgcggacggccgtccgcgccctcgtccagcggcaccctaaccttcgggccggcttccggcaggtcgaatccggagggaccgttcagctgatacctcgtcaagtggatgttccctgggccgagtacgacctcgcgtcgttgcccgccgccgaagcggaggcggaaatcctccgactcgccgccaaggaacacggcatccgcttcgacctggcacagccgccgctgctgcgcttcaccctggtgcggctggccggggcacaccatcggctggtcatgaccacccatcacatcctgctcgacggctggtcgatgcccatcctcgtccacgagctgctcacgctgtacggcagccacggcgacgccgccgggcttccccggacgaccccgtaccggcagtatctgggctggctcgcgcagcaggaccggccggcggcggaggccgcctggcgcgaggcgctcgtcggccttgagcagccgaccctggtcgccgcggtggatccggcgcggcccgcactcatgccggagcggatcaccgtcgagctggacgaggaccgcacgacggcactgacggactgggcgcgtcggcagggcgtgaccacgaacacggttctccaggccgcctgggctctggtgctgagccggcacacggggcacgacgacgtggtgttcggggccgtcgtggcggggcgcgacccgcaactgcccggtgtcgaggccatggtggggctgctcatcaccatggtgcccgtccgggtcgtgctggacccggcgcagtcgctgacggagaccgtcatgcggctccaggacgagcagacgcggctgaccgcgcaccaccatctcgggctgccgcgcatccagcaactggccggccccggcgacctcttcgacacggccatggtgttcgagaactacccctgggacgaatccaccgagctgccggacaccgggctgcgcgtcaccccggacctcggcagcgggcgggacgcgacccactatccgctcaccctggtcgcggccccggggcggcgcctctatctccgcctcgactaccgggacgacctcgtcgacagggcctcggccgccgggttcctcgaccggctgatccaggtgctcgacctcgtctccaccgccaccagcactgattccgataccggcaccggacccgccgcaaccgagctgcccgtcgggcggatcgacctgctcagcgatgaggagcgcgccactctgtcggccgcccccgacgacgtcgcgcgcacgacaggggcgaccctgccggggccgttcgaggcgtgggccgtcgggaccccggacaccccggcggtggtctgcggcgagacggccctcacctaccgggaactcgacgagcgggcgaaccggctggcgcatgtgctgacgacccgtggcatcggcccggaggacatcgtcgccctcgcgctgccccgctcgaccgacctcgtcctcgccgtcctcgccgtcctcaaagcaggcgccgcctacctgcccctcgaccccgaatacccccccgcccggctgacccacatggtcaccgacgcccggcccgcccttctcctcaccaccacggcgatccggtccagcctccccaccacgcacgacaccccggtcctcctcctcgacgccccggacaccgccaccgacctcgccgggcgccccacgcacagccccacccccgccctcgcacccgatcacccggcgtacgtcatctacacctccggctccaccggacgacccaagggggtcgtcatgcccgcgggcgtcctgctgaatctcctggagtggcaccaccgcgagatcggcggccaaccgggcaccaggaccgcgcagttcacggcgatcagcttcgacgtgtccgcccaggaggtcctttcggccctgctcttcggcaagacattggtgatacccgccgaggacgtccggcgcgacgcggcccgcttcgccgagtggctcgaagaccagcgggtggaggagttgttcgcgccgaacctggtccttgaggccctagccgaggccgcagtcgagcagggccgaacgctgccgcggctgcttaccgtcgcccaggccggtgaagcgctgacaccgagccgcctgtcccgtgagttccaccggtcggtgccgggccgggtactgcacaaccactacggccccaccgagacccatgtggtcacggcccacaccttggggcacgacccggacgactggccgttgtccgtgccgatcggccgtccgatcgtcaacacccgtgcgtatgtgctgggttccgggctgcagctggcggctccgggcgtggtgggggagctgtacatcgcgggtgctggcgtggcccgcgggtatctgcaccgccccgctctcaccgccgagcgtttcgtggccgacccgtacggcagcgagcccggtgcccgcatgtatcgcaccggggacctggtacgccgcaacgccgacggcaacctcgaattcgtcggccgcgccgaccagcaggtcaagatccgggggttccgtatcgaacccggcgagatcgaggccgtgttgaccgatcatcccgacatcacccaggtcgccgtggtcgtgcgtgaggacgagcccggcaggaccctcctggtcgcctacgtgacggcgcgggaggccctgcggcccgacgcagtacgggagttcacccgagaccggttgcccgaacacatggtgccgtcggccgtcgtactcgtggagcaccttcccctgacggcgaacggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccacctgggcggtcattcgctcctggccactcgcctcatcgcccgcatccgggccgccttcggcgtcgaggtcggcctgcgcacgctcttcgaggcccggaccgccggcgcggtggccgcccgcctcgacaccgccgggcccgcgcggctggccctgaccaaacagcaactgcccgacctcgtaccgctgtccttcgcccagcgtcgactgtggttcctgcacaagatggagggccccagcgccacctacaacattccgctcgtcctgcggctgacgggcgagctggaccgggaggcgctgcgcgcggccgtggaggacgtggtggcccggcacgacagcctgcgcaccgtgttccccgaagcccaggacactccgtatcagcacgtcctggactccgtcacggtcgagctgtccgccatcgacatctccgaggcggaactgccggacgccctcacgtcggcgtcccgtcatcccttcgacctggcggccgaacccccgctgcgcgtcggcctgttctgcctcgcacccgaccggcacgtcctcctgctcgtgctgcatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcgtctggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctggtttgcagattgatgtggcgccggggcggacgcatacggcgaagttcgatctgttcttcagtcttgccgagcggcgtggtgggcgtggtgagccgcagggcatcgtgggggcggtcgagtactccagtgacctgtatgacgcatctaccgtgcggtcgctgttcgagcgttgggtcaggctcctcgacgaggcgaccgcccatcccgagcagccactcgacaccatcgacctcctcaccaccgaagagcaacaacagattctcggcacctggctggacaccgaggcggaagtcggtgcggacctgttgccggcgtgctttgccaggcaggcgcgcaccacggcggaggccgtcgccctgatcaccggggacacctccctgacatacggggagctgaacagccgcgccaatcgcctggcgcacgccctgctgcgccaaggcgccggacctggcacggtgatcgccgtcgccctgccgcgctcggccgagctggtggtcgccctgctggcggtgctcaagaccggcgccgcctacctgccgctggacccggaccatccggccgggcgcaccgcgtacgtcctggcggacgcggaacccaccctgctgctcaccaccttggcgacggacgcgcggctgccgggcgacggctcgccacggcggctgatgctggactcaccggacgtacaggcgctggtggccgactgccccgacaccgacccgtccgacgccgaccgcaccgccccgctgcgcgccgcggacgccgcatatgtgatctacacgtccggctcgaccggccgccccaagggtgtcgtggtgccgcacggcgcgctgaccaacttcctggaagccatgcggcagaacgtgccgctccggccggacgagcggctgctcgcggtcacgacggtcgccttcgacatcgcggccctggagttgtaccacccgctgctctcgggcgccgccgtcgttctcgcgcccaaggaggccgtcccccagccgtccgtcgtgctcgacctgatcgcccggcacggtgtcacggtcatgcagggaacgccgtccctgtggcaactgctgctcgaacacgagccggaggccttgcgcggactacgcgttctggtcggcggagaggcgctgcccgccgccctggcccagtcgctgcgggccctcacggacgacctcaccaacctgtacgggccgaccgaaaccaccatctggtcgaccgccgccgacctctccggcgccacggcagccccgcccatcggcaggccgatcgccaacacccgggtgtacgtcctggacggccggctgcagttggtggctccgggtgtggtgggggagctgtacatcgcgggcgccggcgtggcccgcggctacctgggccgccccgccctgaccgcggaacgcttcccggccgacccgtacgggagtgagcccggcgcccgcatgtaccgcaccggggacctggtgcgctggaatccgggcggcgagctggagttcgtgggccgggccgaccatcaggtcaagatccgggggttccgcatcgaacccggcgagatcgaggccgtgctggccgatcaccccgccgtcgcccaggccgccgtcgtcgtccgcgaggacgagcccggggacgcccggctggccgcctacgtcgtcgccgacacctcgggacgcgcgtacgacgagaacgccgagctgaaccagctgagcgagtggcaggacctgtacgactccgtctacacgagcgcacccaggacggcgttcggcgagaacttcgccagctggaacagcagttacgacggacagcccatcccgctgcccgacatgcgggagtggcgcgacacgaccgtcgaacgcatcagggccctcagtcctcgccgcatcctcgaaataggcgtgggcaccggactgctcctcgccggcctcgcccccgagtgcgaggagtactggggaaccgacttctcgcccaccgtcatcaaggacctgcagcgccacgtcgatgccgatcccgccctcgcctcccgggtccacctgcacacccggccggcccacgacttcgacggccttcccacgggccacttcgacacgatcgtcctcaactccgtcgcccagtacttcccgagcgccggctacctggagcaggtcgtcgccaacgccctccgcgcgctggtccccggcggagccctcttcatcggcgacatccgcaacccgcggctgctgcgcaccttcgccgccgcggtcagtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcaccgacacccccacccacccctggccccggcacaccgaccccggcgccaccctcaccgatctgagccgtcacctcgaagccgagcgccccgccgggctccgcgtcaccggcgtcccgaaccctcgcctcgcgcacgagctggccgttcagcacaccctcttcgacgacacacctgcgtccgcggacagcggccggccgcacgtcacgctggaggccttccacgctttgggcgacgagcacggctactggaccggcgtcacctggagcactcacgaccatgacgcggtagacgtcctgttcgtcgaccggaacctcatggacgaaggcatccccgtaggcacctacgcccctgccggtacgggcacctcggccatgccgctgtccacctggacgaccaacccggccgcccgccgcggcaccggcgccctcgtcaccgaactccgcgaacacagccgtcagcatctgcccgactacatggtccccgcgttcatcgtccccctcgaccggctccccctgacggcgaatggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccagctgggcgggcactcgctcctggccactcgcctcatcgcccaggcccgggcggtcttcggcgtcgagctggagctccgctcgctcttcgagggtccgacaccggccgaggtcgccgcactcctggacaccgctgggcccggccggctggcgctcacacggcgcgagcggcccgagaccatgccgctgtccttcgctcagcgtcgactgtggttcatccacaagatggagggccccagcgccacctacaacattccgctggccctgcgtctgacgggcgagctcgaccgcgacgcgctgcgggccgcactcgctgacgtggttgcccggcacgagagcctgcgcacggtattcccggaggccgagggagttccgttccagcaggtgctgcccgtcgaaacggccgtaccgcatctcaccgtcacaccgacgaccgaggcggagctgccggacctcctcacgtcggcgtcccgtcatcccttcgatctggcggccgaaccgccgctgcgcgcggccctgttcgagctgtcggcgcaggaatacgtgctgctgatagtggtccatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcggttggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctgggttgcagattgatgtggcgccggggcggacgcatacggcgaagttcgatctgttcttcagtcttgccgagcggcgtggtgggcgtggtgagccgcagggcatcgtgggggcggtcgagtactccagcgacctgtatgacgcgtccactgtgcggtcgctgttcgagcgttgggtcaggctcctcgacgaggcgaccgcccatcccgagcagccactcgacaccatcgacctcctcaccaccgacgaacaccggcacgccgtcgtcgagttcaacgacacggccgtgcctttgcccgagttgtctctcgcggagctcttcgtgcggcaggtggccgggactccggatgctcctgctgtgatggatggggagagctctctcacgtatgcggagctcgatgtccgtgcggatcggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggaagagcgcatccggcacgtccttactgaaacaggtgcgcgactgctggtcaccgacgacccggcggagttccaaccgggcgcggaggactgtgagttgatcggagcggcctcgatgggagtggccgggacggaacctggcggtcctgttgaggtggccgttgcccctgatgctgcggcgtatgtgatgtatacgtctggttcgtcgggtgtgccgaagggggttgtggtcagtcatcggaatgtggctggtctggcgttggatcctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtttgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtgccggacgtggacgggacaacccccggcgaggaagcggaacaagggcagatcgaggagtggaaggatctctacaactcgctctattccgagcccggttcggagttcggcgaggacttctccggatggaacagcagttacgacggtgagcccatcccgctgaccgagatgcgcgagtggcgtgccgccacgaccgcgaggatcaaggcgcttcgaccgcgccgggtcctggagatcggcgtgggtaccggactgctcctcgccggcctcgcacccgaatgtgaggagtactggggaaccgacttctcacccaccgtcgtagaagcgctgcgacgccatgtcgacgccgaccccgaactcgcccggcgcgtcaccctgcgcgtgcaggccgcgcacgagaacggcgagctgccgcaaggccacttcgacacgatcgtcctcaactccgtcgcccagtacttcccgagcgccgactatctggagcaggtcatcgaccaggccttccgcctgctcactcccggcggagccgtgttcatcggcgacatccgcaacccgcggctgctgcgcaccttcgccgccgcggtcggtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcgacgacacccccacccacccctggtcgggggatctcacggacctcgctgagcagttgggcacgggacgggccgatcggctgcgcgtgaccggcgtcccgaactcacgcattgcccacgatctcgccgtgcggcaggcgatcgagggcgggacggcggctcccggaccgctcgtggccggtgtcgatccggaggaactgcaccggctgggggagcgccacggttactggaccgcgatcagctggagcacgcacgatccggccgccgtcgacgtgacgtacgttcggcccggactcctggcagagcgcgtcgccgtaggtacgtacgcctccgcgggcagcgccgcttccggtatgccgctgtcctcctggaccacgagcccgggcgccggccgcgacacgggtgctcttctcatcgccctgcgcgagcacgcccgtcggctactgcccgactacatgcgccctgccgccatcgtgcctctcgaccggctgccgctcaccgccaacggcaaactcgaccgcgccgcgctgcccgccttcgcgccggagcaggccgacatcggacgggcaccggccacaccacaggagcaggtcgtgtgcgagctgttcgcggaggtgctcgggcggccggtcgtcggagtggacgaggacttcttcgccctgggagggcactccctgctggccacccggttgatggcgcggctgcgggccgccttcggcgtcgagttggggctgcggagcctgttcgaggcgtcgaccccgggcgggatcgcggcccggctcgaagccgacgactcggacggctcctacgaggtgctgttgccgctgcgcccccggggcacacgggcgccgctgttcttcgtccatcccggcggcggcatcagctggtcgtacagcgcgctgatcaaacacctgggcccgcagtacccgctgtacggaatccaggcacgcagtctggcccggccggaggccaggccgggcagcatcgaggacatggcgaccgactacgccgaccagatccagaccgtgcagccgcacggcccctaccacttggcgggctggtccttcggcgggctgtgcgcgcacgcactggcagccgagttccggcggcgcggtgaaccggtggcgctggtcgcggtgctcgacgtgatccccaactggcaggggctgacccacgcggacgtacctgcgcccgacgaccgggtgatgctgctgtaccacgtgggtctcgtcgacgacggcacccaacggcaggacgacgaggagatgaccttcgccagggcgcgggagatcctgcgtcgccagggcagtgcactggccagcctggacgaggaccggctcgcggcgatcaccgagatctcggcgaacaacacccacctgaccatcgactaccggcctggcgcgatcgacggcgatctgctgctgatcgcctgctcggagcagcaggacccgccggtcgacgctgcggcctggcagccgtacgtccgtggcaccgtcgaagcccatgtagtgcccggcgagcacgggtccatgctgacccggccggacacgctggccgagatcggtcggatcctgtcggccaagctccacgaactgaccggcgacgagtgacgttcccagcccgccttccaggctcgccccgtccgccttccgcgtggaccgatcccgaggactgacaacgatgctgaccgacgtgctctccgtcgacttggccaaggcgctagaggccgccgacggctggctgagcgactacatccgccgaccacacagcgaactgggacgtaccggcccggtgtgccccttcgtggaacccgctcagcgcgccggcgcgctggagatccgggtcagactggtgggtcccacccccagccagaccctcatcgacgagatcgtccgctgtgggctggacgagttcagcgaggtcgactggaaggccggcaacccgaatctgcgctcgctgctgctcgtcctgccggacctgccgcccgagcatctgcacctgttggacgaggcgcacgccgcgctcaagcccgagagcgtgcaccgcggcctgatgatcgcccagttccacgagaagtgccgggagaaggccgcccgcaacccggagttcgaggtgagctacgcccccgtgccgatgatggcggttcggtccatggcgatccatgacgtgctgttcctcgcggaccggcgcgagtggttcgaggagtacgcgagccgcttcggggcgcgttaccggaactcggcgcacggcatcgatccgctgctgaccgagatgtacgaacgggcccgtgccaagcatgggctgatgaccccgtgatgcacggcggattacggcccgggggtgcacgatgacggacaccgccgtgtaccggggcatggatcaggccacgctcgaccggcagtactcgccgagttcccgggtgccgagcctcggggcctacctgcgggagtacgcacggcggagcacggccgcccgccggtgccacccggtgcgcaccgggctcgcctacggcggacatccggccgaggtgctggactactttcccggccgcccggggcccgggcggccccccttgctggtgttcgtgcacggcgggaactggcaggcgctcggccgtgccgaatcggccttcccggtaccgccgttgctcgccgccggtgcggcggtcgcggtggtcgagtacgggctggcgccggacgtcggactcgacgcgatggcggggatggtgcggcgcgcgatcgactggctgctgcaccacgcggaccggctcgggttcgcctccgaccggctccacctgtgcggcacttcggccggcgcgcacttggccgcgatggccctgttgcccggtccgccgcaggggccggacgtctccggccggatcgccggggcggtgctgctcagcggcatgtacgaccttgagccggtccgtctgtcgtacgtcaacgaggcgctgcgccttgacgaagcgggggcgcgccgcaacagcccgctcggcctgctgccgccgcggttgcctcaggtggtggtcgcccgcgggggcaacgagaccgaggagtacgtccgccagcacgaccggatggtcgcggcgctgcgtccccgggccgcggtgtgcgaggtcgtcgccgagcggcgcgaccacttcgacctcccctacgacctgggggtgaggggcaccggactgggcgacgcggtgctcgcgcagatggaactgggaggagcggccacatgaccaccgaacagcccgcgcagggctccgggcagagcgccggatgcccgctgtccgcagccgtcccgccgggcaccaccccgtacgcgtactacgcgcgcatggacgagctgctggcgctgcagcatccgctcagcccggcggacaccgagccggggttcatcatcatgagccaggtcaaggagctcctcttcaagctgctgcacaccgaactgaccaccgcccgcgatcagttggacaaggaccggatcgacgccgcgctgtggaccctgcgcaggtcggcgcgggtgcagcaggtcctcctcgtgtcctgggagacgttctccgtgctttcgcccgccgagttcgccgagttccgggacgtcctcggatcggcgtccggcttccagtcggccggctaccggcgactggagttcctgctcggcaacaagaacccggcgatggcgcagccgcaccgggacacggccggtcacgacgcggtgctgaaccaactgcacgagccgagcctgtacgacgcggccctggccctgctggcccgccgggggctgctcacgccggacgactccgcggcgcgggacgccaccctgccgtaccggtccggcgaggacgtcgaggaagcctggcgggcgatctaccaggaccggcccggccaccacgatctgcatgcgctggccgaggcgctgatggacacggcggagcagttcgcgcgctggcgctacacgcatctcctcaccgtgcagcggatgttgggagccaagccgggcaccggcgggaccgatggcgtcgcctggctcgcccggatcagcgaacaccgattcttccccgagctgtggtcggtgcgctcgacgctctgacccaccggacaggtgccaggagcaccagggacacgacagatgccgacggactgaaggtgaggagtgccgatggagggcggaatcccgcgcgcggtggccgaggagctggacgcggccgatccgctggccgcgttccggaagcggttcgtcatcaccgatccggagttggtctacctggacggcaactcgctgggccgactgcccgcggccacgtcggacctgttgcgcgaggtggtcgagaagggctggggcggcggcctcgtacggtcgtggcaggaatggatcgactggggcgcccggctcggcgaccgtctcgccgcccacgtgctcggcgcccggcccggcgaggtggtgatctcggactcgacgtcggtcaacctgtacaagctggccggcgccgcgctggacgcggcgcgcggacggaacaccgtcctcatggacgccgaggactttcccaccaaccggtacgtcctgcaggggctcgccgaccagcgcgggctgcggctgcgacggctcccgtccgatctcgacggcggcctggatctcggtatgctgcgctccgccctcgaccgggacgtcgccctggtggtgctgtcgctggtgtcctaccgcagcggcgcgctcctggacatggcgaaggtgaacgaactggcccgcgcggctggcgccagggtgctgtgggacttgtcgcacgcggccggagccgtccccgtcgatctcgccgcggccggtgcggaactcgccgtggggtgcacctacaagtacctgaacggcggccctggttcacccgccttcctctatgtgcgcagtgatctgcacgccgtgctgcgccagccggtgtggggctggtacgggcagcgggagcagttccggatggggcccgactacgatccggtgccggggatcgagcgcttcctggtgagcaccccgcccctgctgtcgctggcggcgatcgatccggccctcgccctcgtggaagaggccggcgtggaccggatacgtacgaaggggctgctgctcggccggctggccgacgatctggccgccgcctggctcaccccgctgggcttccggtcggcttcgccggggacgccggagcagcgaggctcgcatgtgtcgctgcaccacccggacgcctggcggatctgtcagacgctcgccggtgaagcgaaggtcgtctgcgactaccgggtacccgaccggttgaggatcggtctgtcgcccctctacacccggttcacggacgtctgggacgcactggcacgcctccgcgaggtcaccgccgggcgcacctacgcgtcccgccccgccgacctggcccgggtgacctgaccgtcggcctcggggccaccgccttgcccgcacctcaccacccacccgatacggagtgcccatgaccgacacatcgccgctcaccaccgacggcctggtacggatcctgttcggctcctcggccttccagatgctcaacgcgggccgcaacctgggtctgttcgcgctgctcagtcggcagtccgggctgaccgctcaggagatcggacgtgaactcggcctggcggaacgcccggtgcagatcctgctcctgggtactacagctttggggctgacggtccgtcagggcgagggctacctcaatgccgctgtcctgaacaacacgtttgaggacggcacttgggagatcatcgaggatctgatcgagtacgaggagcggatcgtccgccccgccgaggtggacttcacggagtcgctgcgccaaaacaccaacgtcgggctgcgccggatcgacgggaccggcaccgacctctaccaccggctgtccgcgaaccccgagcttgagcagttgttctaccgctgcatgcggtcctggtcacggctgtcgaatcccgtcctgatcgagcaggccgacctgaccggggtgcgccgggtcctcgacgtcgggggcggcgacggcgtgaacgccatcgccctcgcccaggccaaccccggcgtcgagttcaccgtcctcgacctccccggcaccgtggagatcgcgcgacgcaagatcgccgagcacggcttggccgaacggatctccgtccgggcggcggacatcttcgccgacgactacccggcggggcacgactgtgtgctgttcgccaaccagttggtgatctggtcaccggaggagaacgtgcgcctgctgcgcaaggcccacgcggcgctgcccgacggcgggcgcgtgctggtgttcaacgccatgtccgacgacagcggcgacggccccctgtacgcggccctggacaacgtgtatttcgcgacgctgccggccgcgagcagcaccatctaccgatggggccagtacgaggagtggttcgccgcggccgggttcgtgaagcccgagcggctgccgggcggccggtggacgccgcacggcgtgatcagcgcggtcaagtgacgccccagcgagaaccggagtcggccatgtccctcaagtcccacgacgccccgccgaccggtggggccgcggcgtgccccgccggtccgcacatgatggatccggatctgctccgggaccctttcggcggctacggccggctgcgcgaacaggacccggtggtgcacggcaggttcgtcgacggcaccccggtgtggttcgtgacccgctacgacgacgtccgcgcggcgctgcgcgacccgcggttcgtcaacaccccctcccacgtgccgggcgagaagggcgcggacccgcgcgagggcatgatggaactcctcaaggtccccgagcatctgcgcggctatctgctcggctccatcctggacagcgacccgccggaccacccgaggctgcgccgcctggtgacccgggcgttcgcggcccgccgggtcctggatctgcgccaggacatcgagcggatcgccgaccggctgctggccgagctgccgcaccgggaggaggacgggacggtcgatctcctggagcacttcgcgtatccgctgtcgatcacggtgatctgcgagctcgtcggcatcccggcggccgacctcggccggtggcgggagtggggcggcgacctggtgtcgatgcggcccgaacgactccagcactccttccccgtaatgatcgattactgccacgaactgatcgagcggcgccgcgccgcgctcaaggacgatctgctgagcgagctgatccgggcccaggacgacgacggcggacggctcagcgacgtcgagatggtcaccatgatcctcacgctggtcctggccggccacgagacgagcgcgcatctgatcggcaacggcaccgccgcgctgctcacccaccccgaccagctggcattgctgcgcaaggacccggcactgctgccgcgcgcggtgcacgagttgatgcgctggtgcgggtcggtgcacgtggcccgactgcgctacgccaccgaggacctggagctcgcgggcacagcgatcgcccggggtgacgcggtccaactggtcctggtgtcggccaacttcgacccgcgccactacagcgacccggaccgcctggacatcacgcgccaacaggaggggcaggccgagaaccacgtcggcttcggacacggcatccactactgcctgggtgcgacgctggcccggcaggaaggcgaggcagccttcgtgaaactgctggagcactatccggacctggcgctcgccgatggggccgccgagctggaccgggcccggctgccgggcagttggcggctgaactcgcttcgccttcggctccggccctgacctcggtcgcggacaggggccgcccggcatcgggcggcccctgtcggccggcgtctcgccttggcggcgcacggacgggtgctcagccgggtggtccgtctcccagggtgatggcttgcaccgggcatgccagcgccgcttcgtgcacctggtgcggcaccgcggtcgcggcgccttcgggcaccaactccgagtatccgtcgtcgtcctgagtgaatacgtccggggccgtcagaacgcactgcccggaccctatacacgtgtgatgatcgatactgatctgcatggaagaccgtctccgatggtcgggcggacaggggccactcagtccccgagtgcgtgcgcggcggcgcggtcgagggcgacgagtatccactcctcgaagttcccttccaccttcgccgcggcctcgcgccagcggtccacgcgatcgacggacacgctgatcctgggcagaccgacctgtgcgagcggggccgcccgactgccctgacggctctcccgcagctggagccggagccggttcctcgaccagccctgctcctccgcgcggtcgagccaggtgtccgcctgctcgggcgggagcgaggcgacctcggcatggtggccgaagctcaggttctcgcgccgtcgcccgagctcgaagtgccgtgccacccatgcgtagttgcgcagagtctgatagtcgagtccagcggcctgtacgccggtccggtagcggtcggagtaccgctcctggccgtacacgagccagtcgcccaggcaccaggcggatgagtcggcgatctggaagatgtgcagtccggccttctcccaccggtcgtacgacaggatggcgggaatcttcaggccgacccgtgtggtcaggatccgcccttgatcgttgctcccgcggcgagccgccgcctcgcccggtctgtggccgactgctaaatgactgtcacgctggtggtacttggcgatttgctgcacgggttttccccctggaattgagccattgccaaatctcgtcccagtcgtcagcgaccggggaccatgaaaggaccccggcccaaaaaccgtagattactgaacgatgtatcgtcaatgatcaattccggacggatgtgaccggttggagcgcaaaggagcccggggatgacagcctcatgtgtccgtgatctctaaaacccgtgtgcaatccctgcgcccggacccttacggcgcggacggggtcgctcgggccgggctgccgaggccgggccgttcggccgcggcgatcccgtcgaggatcagttccaggccgacccggaaggcctccttggcctcccgctccacatggggtgtcgcgtcggccggctccttccccgcgtcgtcggccgcggcagcggtggcgtgcgactccagcgccgccagagccgggaagcgctccgcgaagtccggcgccacctcggtgagcagagcggtccgctccatccaccactcctcgtcggacacccccgtctccggcgcggcctgccggcgctcggcggccacccgggccatgccccggacgaactggaacagcacggcgatgacccggcggactctcaagggctccaggcccaccccgcccaggatgcgcaccaccgtctccagttgcgcgtactcgcccggcccgagcaccggccgggcctgggagacctgcaggacccaggggtgccgcagatagaaggaccaggtgtcctcggcccactccgcgatcgccgcgcgccagcccgcggcggtgtcgtactcggtgggcagctcggccagcacccggtcgtgcatgagatcgaccagttcgctcttgctcggcacataggtgtacagcgccatggccgtccgtcccagccaggaccccaccgcccgcatcgacagggctgacattccctgctcctcggccaccgcgatgcccgccgccacgatcgtgtcgacgtcgagcgcgggcttgggcccgggtgcggatctggtgcccttcgggccggccgagcgccagagcagagccattgagcgacgggcgtcgccctgtccggcaaaaatgaccaccttgcaactccttactgcataaagtaacctcgccccgagcatctccttacggtgtaaggatatcaggtggacgtggggtcccgcctcgggatgcccggcactcttggaaggtttttgatggcagagctccctgtttcattcatccaggtcaccgacatcaagcacgtcacgccgcgcatggtccgagtgacgttcgacgcggaccacatcgacgactccgtcggcggcacgccggaccaacaggtcaagctctgcttcccccgatccggccagcgcgtgcccgtgctgccggaacagggtgacgacgcgaccagctggtaccaggcgtttctggccattccggaggacgagcggccctggatgcgcagcttcaccatccggcggcgccggccgggcaccaacaccctcgaaatcgacttcgtcctgcacggggacaccggccccgccacccgctgggccggttcggcccggcccggcagtcagctcggcatggtcggaccttcgacgctctactccgcacccgtctccctgacggactcgatcgcgaactccgactggctcctgctcgccggtgacgagaccgcgctgccggccatcggcaccctgctggaggcgctgcccgagggggcacgtgccctggccttcgtcgaagtcgccgacgccgaagagcggcaggacttcgagaccgccgggaacgtcagcgtcaactggctctaccgagacgggacttcgcccggccagagcgacaagctggtcgacgccgtgcgcgaggccgagttcccggccggccggccgttcgcctggctggccggcgaatcgggggccgtgcgggccctgcgccgtcatctggtcaacgaccgggagctggacaagaggtcgatcgacttcagcggctactggcggttcaagctcacccaggacgatgcgccgaccacggaggacatggccgaggcgcaggagcggctcgccgagttccaggccgccgccgacgcccgcccggaatgaaccgcacggaggggccgggcttcggctgggcccctccgcgcccgaccgagaagcggtacgcgccgacaagcggtacgcgcgaaagaaggctttcatgatcacagtgcgaggtctcgcccggcagttcaccctccgtggacggaccgtcgatgcggtgaagggcatcgacttcgatgtggcggccggggaactggtcggtttcctcggccccaacggggcgggaaagaccacgaccctgcgcatgctgaccacgttactgcgccccacccggggttcgggcaccgtggccggctgcgacctcctggccgacccgcagggggtccggcgcaagatcggttacgtggcgcagggcgggtccacctggccggagtccaaggtcggggaggagatcgagctgcaggcccgcctctacggcctgtcgcgggccgaggcccggcggaggggagcgctcctggcggaacgactcgacctcgccggcctggagcagcgtcccgcgaagacgctctccggcggccagcggcgccggctcgacatcgtgctgggccttgtgcacgagccccaactggtgttcctggacgaacccacctccggactcgacccgcagagccgggccaacctgtgggaccacgtccgccggatgcgcgccgaacagggcatgaccgtcttcctcaccacccactacctcgacgaggccgacgccctctgcgaccggatcctggtgacggacgacggcaagatcgtcgccgaaggaaccccggacgaactcaaggcaggcgtcgcaggtgatgcgatcaccgtcgacgtcgcggaccgggccgccgaggccgccgaagtggtgcgccgactgcccggcgcccacgacgtcaccgtcgtcgccgagaccgtacggctcagggttccccggggtgactccgtgctgccggagctgctgcgcgccctggacagccatcggatcacgatgacctcggtcaaggtgaaccggcccaccctcgacgacgtcttcctcagcctgacggggcgttccctgcgcgacggcgaggcggcgagcccggccatggaggccactcatgctgcgtgacacctggctgatctttctgcgggacatgaagctgtgtctgcgcaatcccacctggctgatcatcggcatcatgcagccgctgctgtacctgttcctcttcgggccgctcatggtgacggtggtggcccatacgcccggcttcccgtccggcaacgcctgggccgtgctcacgcccgcgctcatcgtgcagacggcgctgttcagcggttcgttcgccggagtcgggctgctcaccgagtaccgggtcggcgtggtggaccgcttccgcggaacaccggccagccggtccgcgctcctgttcggcaaggtcctggcccaggccgtccaggccaccgcgcagtcggtgctgatcgtcctggtgacgctgctcgtcttcgacctggacgcgccggcgtccggcatgctcctgagcctcgccatggcggcggtactcgccatcacgctgtcgtccggatcgtacgcactggcgatggtcctcaagagcgaggccgcgttcccctcgctgatgaacgccgtgctgctgccgctgctcctgctgtccggcgtgctgctgccggtcaccgcgcaactggctcccggctggctctacaacctggcccgcatcaacccgctgacccatgtggtcgacgccgaacgggccgcgttccgcggggacttgggctccacagggctgctgaccgggtccctcgtcctgttggcgatggccgccctggcgctcgtctggggcacgcggacgttccagaaggagaacgcctgacggcgacatcccgtgaggcgacagcctctctgcccgaccacgaccggggctctccggggagccccacccgtaccacccgaggagcacatgtgagtaccgacgccatcaaggtgaccggtgcgcgggagaacaacctcaagaacatctcggtggagatcccgaaacggagtctgacggtcttcaccggcgtctccggctcgggcaagtcctccctggtgttcgacaccatcgccgccgaggcccagcgccaactcaacgagaccttcaccgccttcgtccgcaacttcctgcccgaccacggccgtccggccatggacgccatcgagaacatctccgccgccgtcgtcatcgaccagaaacggctgggcggcaacccacgctccacggtcggcaccatcacggacatcaacccgctgctgcgactgctgttctcacgggcgggcgaccccgtgggcctgccgcccagcgcgttctcgttcaacgacccccaaggcatgtgcccgacgtgcgaggggctgggcagggtcgtggaactcgacctcgacgcgttcgtcgaccgcgccaaatccctcaaccagggcgcgctgctgcaccccgacttctccacggacagctggtactggctgagttacgtggagtccggcttcttcgacaacgacaagccgctcgccgacttcaccgaacgggagttggggctgctgctgcacggcgagatgccacagaagctccgcctggtctggcagggcaacacactgaacgtcaagtacgagggtctggtggacaagttcaccaggctctacatcaagaaggaggacatgtccgagcgcagccgccaggtgttccagcggttcgtacgctcggctccctgccgggactgcgcgggcacccgcctcgcgcccgccgcccgtggctgccggatcgccggccaccacatcgcggatctcacctccatggaggtcgagcgcctggccgcggtcctggcggggctggacctgccccacgtgaaaccggtcctgaaggacctggtctcccggcttcggcacctggtcgacatcgggctcggctatctgagcctggaccgcgagacccgaacgctgtcgggcggcgagtcgcaacggatcaagatggtccgccatctgtcgagcagtctgaccgacatgctgtacgtcttcgacgaaccgagcgtcggactgcacgcccgcgacgtgcaccggctcaaggaactcctgctcgaactgcgggacaagggcaacacggtgctcgtggtggagcacgacccggacgtcatggccatcgcggaccacgtggtcgacatgggaccgaaggccgggacgctgggtggcgaagtggtgttcgagggcagcttcaccggtctcgtcgaagccgacaccctcaccggccgccaccttcgccaccgcctgcccgtgaaggccgcaccccggcgcgcgaccggcgcgctcagcgtcaccggcgcgaacagtcacaacctcaccgatgtcacggtcgacttccccacgggcgtcctcaccgttgtcaccggagtcgccggatcgggcaagagttctctcgtcaacgacgacttcctgagccaacatccggatgcgatcgtgatcgaccagtccgcggtgacggggagccggcggtccaacaccgccacgtacaccggactcctcgacccgatccgcaagctgttcgccaaggccaacggggtcagtgcgtcgctgttcagcgccaattccaagggcgcctgccccaactgccaaggactgggcgtcctttacaccgacctggcgttcatggagacgctgaagtccgtgtgcgaggtctgccagggccgtcgtttcgccgatgacgtcctcggctaccgactgcgcggccggtcgatcagcgacgtactggccatgacggcggccgaggcggcggacttcttcaccgagccgaagctccgccggacgctgcacgcgctgaacgaggtgggcctggactacgtggggctcggccagccgctcagtaccctgtccggcggcgagtgccagcgcctcaagctcgccgccgacctgcaccgcgtcggcagcgtctacgtgatggacgagcccaccaccggccttcacctgtccgacacccaccgcctcctcggcatcgtcgagcgcatcgtcgaccagggcaacacggtgatcgtcatcgagcacaacctcgacatcgtcaagaacgccgactggatcatcgacctcggccccgaaggcggcagcggcggcgggaaggtcctgttcgagggaaccccggcggaactcctgaaggccgccggctcgtacaccgccgagtacctccgccgggacctttcgggcgcctgacgcgtaggtgcccgtccaccgccggggcgtggccggtgtcgtcggggccggatcgcggcggatcggacggaaggcgcctgacctgggcagaacctcaggtcaggcgctttcttcggctggggaggacgttcagcgtcctcgccaccgccgtcgtccggtcggcgggacgaggccagtcgagctgcctgggcccgtcccgcgccggtacaccgctgcgggcgaaggcccgcagctcgccgtcgcagccgacgacgtgaagctgcgggggaccctcgctggaccacgccggttctgctccgcccaagctggcgcggcgtcggccaggtgtcgttcctcggcccgcgactgacagccgtgcggatggtgtggttggctggcctgcgcgattgcggcgtgacgggcaagggaggcgaggcatgggcggagtggactatcccggcggggtacgggaggacgggctgccggcggcgctctccgatccggccagactggcggcgctggaggccagcgggatgatcggcagcgccccggagcagatgttcgacgacctgacgcgactggccgtgtcggtgacgggctcagccatctccgcgatcacgttcgtggggaagacccgcacctactggaagtcggtgccgcacctgccctacggcaccgtggacgagtggcagaacgcggtgggcgacagcttctgctacttcgccgtcggggcggagggcccgttcatcgtcgaggacgccgagaaggacccgcgcaccgccggacaccaggccatcgggccctggggcgttggcgcctgggccggattcccgatcgtcaccagcgacggtcaggcgatcggcaccatgtgcgtcatcgacgcgagcccgcgccagtggaagacccaggacctggaaaccctcgccgtgctggcccgcgcggcatccaacgagatcaacctgcgcatctccctgggcgcgacacaggccgcgctgagcaccgcgcaggtggcgctgcaccaggcacagggggcgctggcgaactcccgcgaactggcccgctccctccaggacagccttctgccgccggcgctgcgcccggtacccggcatcgacgcctcggcgctctacctgcctgccagtggcgacgcggatgtggtgggcgacttctacgacctcttccactcccgcgggccgtggtggacggccgtgctgggcgacgtgtgcggcaagggcaccgaagcggccaaggtcaccgcgttggcccgctacaccctgcgcgccgacgccggggaccacctgtcgcccgcgacggtcctcggccggctgaacgccgccatgctcgcccagcgctccccgcgcttcctgaccgccgtccaggccacgctccgcaccacacgggccggcgtggccggacgcctgtgcctggcgggccacccgccggccctcgtccgccgcgccacggggcgtgtgcagcaggtcggcaccagcggcactctcctcggcgtcctggaatccgtccgcctcaccgacgtccgcttccggctcgccccgggcgacctgctgttcctctacaccgacggcgcctgcgaagcccgcccggacccccaggggcccgcacccgaccggtcgatcttcgacgaaggagccctcgcccaggccctggcggacacccacggcctcgacgcggccgccctatagtgagtcgtattacgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataaagctgatccccgggaatt

SEQ ID NO: 3 - DNA, 41219 bp, Streptomyces xanthochromogenes
gcggccgcgacatcgacgacgaccaagccatcctcgtcctgaccgccaccgacccggatcccgaggcctgacccgacccggaccccgaccccgggcagaccccagcaccgccacgaaaccggcggatggcgacgacagccgcgcccgggtgcctgtcccaggcatcctgggcaatgagggcatcgatgttccggcccccagcagtacggccgtgtcccatgggtacgatcgcatcccgttcaagggcgggacgacatccacgcacggggagttcgcatggccaccatccatcgcaccaccatgacgccgacgaagctggaactgctcgccggctggctgccgaagcagagctggtacgtcggcgatgcggcaaagccggaactggtcaaggcaggtgggttccggttggacgatccgaagggtgcggtcgggatcgaattcatggtcgtggtggacaccaccgcgcaggagccggtggcctacttggtgccgatggcctaccgggatgctgcactggagggcgtccccgacgaggcgctgatcggcacgtccgagcacggcgtgctcggcactcgctggatctacgacggcgtccacgacccggtggtggtggcgcagttgaacgcgctgctgcgcggcgaggcgaccccgcagcgccagaacgagagcgacacccccgacccgaccgtcaccgtgcacggaacgggtaccgccgacggccgcgacgttcggctccaccgcgtcctgcggtcgggggacacagctcgcccgtcgtccgtacaccttgtcgccgggtggacctggccggacggaacggctgcgcggggcgtaatcgcggggagttgagaccgcaggccgggcttggagggtggggtaagccggagagctcgtcacctctccggcgggcggccgagccgcgagcggcgatggtcggggaagcgttccgcgcgcggatcgaggagatcgatcaggtccatgagggcgtgctgcaacgcgatcagccggtacggcacttccgccgcccgcgccaccacctcgacactgtcactcagcgggccgaaccactcggtgaactccgcgtcctcgtcgagtcggcggcagaactccacgcacccaagacaggagatgccgtccgcggccggcatgagctcgccgatcgcccgctgatcgctgcggaagacgatgaagtgcccatcgaggaagcgtgtgttgccgccaccatcgttgttcagcacgttgctgatcgccgtgaagtgacccacgatccgccggttgagcgtgctgtccccgagatcgaggaactggacaccgcggcgcaggatttccacccggcccaggtactcgcccagtacatagagcgtgctgcgacgggcgtacgcgcgttcccggtcggagccgtcgcggaaatacacccgcaggaatccacggtccacgatgttgtagaggcgggactgcagatcgaaggcggcccacaagagcggatcacggtggcggcccatcaggtcctgctgcgtggtccggctcttcaactcgtgtgcgagcagcgcttgtcggcggctggagcgggccgagaaccaggcgccgccgagcgccaccacacctgacagggacgcgacgaccaattcggtattcacaggcccgggagcgtagcgggcacgacagccacctgcactggtttcgccgcctccggatgcgctgggaggcatgcggtgacacacacccgccgagaccttcctcaccctgccgcgtaggcctgctcgctctcccggaaacgctcgatcaggagcggatccatcagggtggaatccggcccgtagaacttgccgaacttcgccctgtattgctggaaccagtgcggacgttcggagaggaagaagatgtcgtggagggcgatggagcggatcgcgagcaagggtacgggtgcccggctcaccgcgaagcgggagttgcgtgccgctgttacccggcagttctcgtggaactggccgatcatgaggccgtgcgccacgaagtcgtccttcacacgggcttgggcctggtcgagcagtcccgtgtcctcgctgcgcagatcgggcaggacgatcaccatggcctgcagcatggggttccggccctgccaggtcgtcagctcgtactcccgcaggccgctgcgtgcgatctcctcgaccagttcgaggctcggggcatgcccgacaaggcgtatgcggacctccatggtccggtttttgcgggagggtgacacgaacgggcagatcggcccggtgcggccgatctccggatggctcgccgatatgtactcgctcagccacgagtcgaccgactccagcgcggactcgaagtcgacgtcggatgtcttgggcgtcatggcgtacccgtcaggccggcaacgcgatcgacggcgatgcgggcgagccgatggatctcggacccgtccggaatacgggtgttgatgaagttgaggatgccgtccttcggcacgatcacccacgcggtctcgatgcggaagtggtcgacggcggacagccagcgcacgacgaagccgccgtcccccaactccaccgcccccaggtccactttcgtgatgtcgagttcgacgccgtcgttcagccccatccggtagtgctcgcagtcgcgcaccgcttcgaagagctcctggtacacctgccggcaagccccgttgccgaactgtgcgacgtggtggaacaccacggaacccacgatgttcgtgaacagggcggaggcgtgatgcgtcgtctccgggtgcgtgccgtgcgtgagcatgttagtcaggtcgacgatggcccgccccgactcgtcgccggagacgaacaccggatcgtaggcggcgctcggctcctccccgtagaacgactcctccaagtccttttcgttcagcagtagttgtgcgaaagcggcgtcgtccatcgcgttctcctatcggcgcacgaggctcagcggtcgcatgtcggtccagtgctcctcgacgtaggtgaggcagctctggcggtcggtctcggcgagcgcgacggcccagcccgcgggcacctcggcgaacgcgggccacagcgaatgctggccttcctcgttgacgagcacgaggaaggtgccgtcggaattctcgaaggggttgctcatcattgctccctgcgtagacagttgcggtgggggagggcggggcccgtcactcctcgtgcagtaccgccgtcacgtacttgtggatgccgttgatcgaggtgaacagcgtcatgtcctcgaactgcgggtcgatgacgatgccgtgccgctcctccagctcgtgctggagaaggaccagggtgaacgagtcgatcaccagctcggcatcgtccggcagctcctcgggcggaccgagacccgcgttctcggccaggatcgctctgatgtcgtctttcgagatcaccggttcccctgtctgcgccatgtggtctgccctgtcgtcatgcctggggcgatgcttccgcggtgatgcgtccgtgcatcagtgcacggtcgcgtacgagtttgcccatcgagttccgcggcagctgccgcgtgacgaagtaccgcttgggattcttgaccgggctcagccgctcgcggcaccagtcggtgagctcgtccgaggtgggcgcccggtcggttccggtgcccacgaacgcctcgatgaactgctcatgggtcaccaccgcctcggtcacccccggatgggagaggaggacggtctccacttccgtgaggtcgaccttcagcccgcccaccacggccagtgaatcggccctgccgagcatgctgaggactccggtcgccggatcctggtggacgcggtcgtaggtccgcagccaaccgtccgagaagcgatcggcatgctcgctgaacaggtacggcgaacgttccatccggacgaagagctcctcgtcgagcaccttcacctcgaccccccgcaccggaggccccaattggggcggtggggagccgccggacaggtcgcccgcgatcaacccgatctcggtcacgccgtagaccggactgatcggcaggccgaaccgggcgtggaaacgttcataggtctcctgcggcacgcgttccccgccggacaccgcgagccgaagggcgggcagttcgggccgccctccggtccgggaaaggagatcgtagtgaacgggcgttccgtacacggcagttgcctcggcccgcgccatcaggcgcaccacttcggccgggcggagactgggcgggacgatcagggtcgccccgaccgcgagggcgtgcaggacgccgctgaccaggcccatgttgtgcatgacggagttcagcagcatcacccgctcacccctgccggggattccgggcaacgcgctgtgccggtcgagttcggccaggacggagtcggccgggcggccgatcaccttgggtcggcccgtcgagcccgagctgaactgcaccaggcggatgccgtcgacggccggtaggccgtcgggaagccgctgcaccgagaacccggactcctggcggaatccggcgaccggcccgtccgccccggtcgccacgacgaggtactgcggtcgcaccaggtcggccagcggttcgtattcggtgggcgtcagccggaagtcgaccagaaccacctgtgcgccgcggctccacaaggcgagcagtacttggagataggtgaagctcggcttcattcgcagaagaaccgagctgccgtctgtaatgccttgtttccgaaaacgctcaccgaaatcattgactgctgcacgtaactgcccgcgcgtaactggcgcactggaaattgcccacagttcgtcgtccgcgccccggctcagcagatggctaccccaccatgtatttgccatacacgcttccttttgcgaaaacaggtccgcgaaacgttcgatcaggcatttagtgaaggtaaagctagcaccgccgagaaacctcgggcaagcactcggacgtcaatccgttccagccccggcaaagacgtgcagccccagtcggatgccctgacggttggtgcggaccaagcctcgccgtgggcctccacgcttgcacgtgaagaacttggcgggtcccctgcctgcttcgatcgctgtgccggtgtgctccttcggtccccgccgaccgatttcttgtcccgccggcggtacaaccggcggcacatgcgcatggcgtaggcctttgatctccttgaccccctggggctcccgagttaggttgcccttgattgcgtttccgaggcgttgcgctcgattccggtgcgtactgcgagatttctcgcccagttggcccgacgatcgccttacatccattacccggtcgcatttccgacgtgtcttcatcgggccgtcgtgagcttttcactcgtgtctctccggccgtccgtgcgggggaaacttcggaggtggagacttgttcgccagtcctggcaccacgctgccgctcacggccgcgcagcgcgagatatggatcgccgagcagcgtcttggggaggggaatcgggttttccgggtgggtgaatacctggagatccacggtcaggtggacccggcgctgttcgaacgggcgctggtgcgcgtcgtcggtgaagcggacgccttgcacgtcaggttcgtcgaggaacgcggcgaggtccggcaggtcgtccacgagctcggcgactggtccccttccgtggtcgatctcagcgcggagccggaccccgaacgggccgcccgcgactggctggacgaagcggtcgcccggccgatggacctggccggtgaccggctgttcgagcacgcactcctgcggctgggcggcgaccgctgctactggtaccagggctaccaccacgccgtgatggacgccttcgggtcgctgctgatcacccgccgggtcgcggacgtctacacctcgctcgcccagggcaggccggtgtcaccgagcccgttcggctccctgtccgacctgctggccgccgaaggggagtaccgcgattcgagacgattcaccgaagaccgcgagtactggacggagctcttcgccgaccgcccagcgccggccgggatcgtcggtcgaccctccaccacccccgaccgctaccttcggcacaccgcaggccttgaccatgccgggctgagcgagctgcgcgaagcggcccgccgcgcacgcgtcccctggtcgtacctggtcatcgccgccacagccgtccatctgcaccggatgaccggcgcgtcgaccgtggtgctcggtctgccagtgacggcccgcctgaaccaggtccagcgccgaacgcccggcacggcggccaacgtcctgccgctcaggctcgcgctccgatcggagatgacgcatcgtcaactcctcgatcagatcggggaacggatacgcgaactcggcacccaccagcgctatcgcgccgaggacctccagcgcgacctcgaactgccgggccacgcggggacctggtacgcaccggtcgtcaacatcatgtccttcgactacgcggtgaccttcgccggcctgtcgaccacggcccacaacctctcctcgggactggtgggcgacctcacgttcgccgtgtgggatcgacgcgacggcaccggactgacgctcgacctcaacgcccaccccgagatctgcgcggaccatgaattggccgcacaccagcgccgtttgatcgccgcgctgcgcgccgtcacggcggcggacccctccttgccgattggccggatcgacctgctcaccccggaggagcgggccgccttcctggccgtccccgacgacgttccgccgctgacggaagccacactgacgacgcggttcgaggcgcaggccgacaagacacccgacgcggcggccgtcacctgcggcgaggcgtccctgacataccgggacctgaatgagcgggccaatcggctggcccatcgactggtggcccggggcatgggtccggaacacgtcgtcgcgctggccctgccccgctccgccgacctcgtcgtggccatcctggcgaccctcaaggcgggcgccgcctacctgcccctcgaccccgaatacccgtcggcccgtctcgcccacatggtcaccgacgcctgccccggcctcctcctcacgacaacggcggcccggtccaacctccctgccacacacggcactccggtcctccttctggatgccccggacagcttcaccgacctcgcgcacaaccccgctccggacctggtacccgaccaccccgcctacgtcatctacacctccggctcgaccggacggcccaaaggcgtcgtcaacacccaccggaacgttgtccggctgttcgacgcctcccgcaagtggttcggcttcgggccgcaggacgtctggacgctgttccactcgtatgccttcgacttctccgtctgggagctgtggggtgctctgctgcacggcggccgtcttgtcgtggtgccctacgaggtgagtcgggcgccgggcgccttcctcgacctgctggcggacgagggtgtcaccgtcctcaaccaaaccccctcagccttctaccagttggcgcaggccgaagccgaaggcgaatccggagccgaggcagtaggaggaccggaggccggaacgaacgagggtgccgaggcggtggatgcctccaggccgactcgccgactcgcgctgcggaccgtggtcttcggcggtgaggcgctgcagccgtcccgcctcgccgactggtacgagcggcatcccgacgacgcgccgacgctggtcaacatgtacggcatcaccgagaccacggtgcatgtgacgtaccagccgctgaaccgccggcgggccacgcccacggcggccagtgcgatcggcaccggcctcccggatctgcggacccatgtgctcgacggcgggatgcagttggtgccccccggggtggtgggcgagctgtatgtcgcgggaggcggcgtcgctcgcggctacctgggccgaccgggcctcacggccgaacgcttcgtcgccgacccgtacgcgtccgaacccggtgcccgcatgtaccgcaccggagacctggtgcgctggaatccggatggcgagctggagttcatgggccgcgccgaccatcaggtcaagatccgcggcttccgcatcgaaccgggcgagatcgaggcggtggtgagcagccatccgagcattgccgaggcggctgtggtggtccgccaggagcagtccggtgacgggcgcctggtggcctatgcggtcgcgcgcgatgcactccgggccgaagacgtgcgggagttcgcgcgggagcggctgccggagtacatggttccggccgtcttcctgctggaccgcatgccgctgacggccaacggcaagctggacaaggcggcgctcccggagccggactactcggtgtcggctgcggggcgggaggcgcgtacgccgcaggagcagatcgtgtgcgatctgttcgcgcaggtcctcgggctgccacgggtcgggggcgacgaccatttcttcgagctcggtgggcactcgctcctcgccacccgcctgatcgcccaggtccgggccgtcttcggcgtcgagctggaactgcgcctgctgttcgaggggccgaccccggccgcggtggccgcccgcctcgacaccgccgggcccgggcgcctagcgctcgcgccgcgggagcggccgacggccgttccgctctcctcggcccagcggcggctctggttcctggcccggatggaggggccgagcgccacgtacaacatcccgctcgtcgtgcgactgtccggcgagttggaccacgacgcgctgcgctctgcactcggagacgtggtggcccggcacgaaagtctacggacggtgtttccggagaccgaggacgtgccgtgccagcgggtgctggcccccgaggcggccgtgccgagtctgaccgtcgtgccgacctccgagaccgaactgccggcagccttggaagcgggcgcacggtacaccttcgacctggcggccgaacctccgctgcgggccgggctgttcacgctgtcaccgcgagagcacgtgcttctcgtcgtggtgcaccacatcgccggtgacggctggtcgatgggcccgctgtcgcgggagctgacggaggcgtacacggcacgtgcgcggggccaggcccccgaatggccggcgctggcggtccagtacgccgactacacgctgtggcagaacgaactcctgggcgaccaggacgaccccggcagcctcctcgccacccaactcgactactggacccgcgctctcgccgaccttccggaccaactcaccctgcccacgacccgccctcgcccggccgtcatgacctaccgcggcgactacctcaccgtcgagatcgacgccgcactgcaccaacgcctcgccgacctcgcacggtcgaccggcgccagcctgttcatggtcctccaagcgggactcgcggccctcctcacccgcctcggtgcgggggaggacattccactcggcagccccatcgcgggacgcaccgaccaaaatctggaccacctcatcggcttcttcgtcaacaccctcgtcctgcgcaccgacaccgcaggcaatcccacgttcacccagctcgtcgcccgcgtccgcgagacctgcctcgccgcgtacacccaccaggacgtgcccttcgagtacctcgtcgaacatctcagtcccacgcgcaccctcgcccaccaccctctcttccagatcatgctcgccctccagaacgcccccgagggcgagttcgagctccctggtctgcaagcggctgtcgagctgggcaggacgggcacggcgaagttcgacctgttcttcagcctcaccgagcagcgcggcgaaagcggcaccccccagggcatcaccggtgccgtcgagtactccagcgacctgtacgacgccgacgacgtccgggtcctcttcgagcgctggatccgcctgctggaggcggccacggccaacccgaaccgcccgctcagccaccttgccatcctctcggacgaggaacaccacgacgccgtggtcgagttcaacgacacggccgtgcctttgcccgagttgtctcttgcggagctcttcgtgcggcaggtggccaggactccggatgctcctgctgtgatggatggggagagctctctcacgtatgcggagcttgatgtccgtgcggaccggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggcagagcgcatccggcacgtcctcaccgacgccggggtctcactcgtcgtgaccgacgaagtctcgcggggtgaactcgggtctgtggcaacagagttgttggtcatcggccgcgagtttcgggaaggcgagcactcgccgccggacgtggctgttgcccctgatgccgcggcgtatgtgatgtatacgtctggttcgtcgggtgtgccgaagggggttgtggtcagtcatcggaatgtggctggtctggcgttggatgctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtctgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtcccccgcgacgatctgacgccggaaggccttcgcgagttcgcgcggcgaagcctgccgcagtacatggtgccgtcggcgttcgtcacgctggaccacttgccgctgacggtcaacggcaagctcgacagcgccgcgctgcccgcccccggcccggccgtgcgggcggacgtggagagccgtgagccgcgcaccgcgcgcgagcggctgctcgcggagttgttcgcgcaggctctgggaatggaccgggtcgggtgcgacgacgacttcttcgtgctcggcggggacagcatcgtgtcgatccgtttggtcagcctggcgcgttcggcgggcatcggattcacggtccgcgacgtcttcgagcagcggacggtcgccggactcgccgaagcggccactgaactggccctttctacaacggagttcgacgactcgggcatcggagcggtggaaccgaccccgatcatgcgttggttcgacgagcgcggcggcggaatcgaccagttccatcaggcgatgctgctggaggtgccggccgatctgggcgcggagcgcctcaccctggccgtccaggcactcgtggaccaccatgacgcgctgcgtctgacccggacgccgggtggcacggatggcaggtcgtggtcactggcggtcgcgtcggtgggttcggtgacggccgccgagctggtgcacacggtggacctcgacggggcggaccttgacgaggcgctcgtccgggaccatgcggaggcggcggccacccggctgtcggccgagcggggggcgctgctccaggcggtctggttcgatgccggaccggtccgccacgggaggctgctgctcctcgtcaaccacctggtggtggacggcgtgtcctggcgcatcctgcttccggacctcatcgccgcctgggaggcggtcgcgctcggccgcccgccccggctcgacccggtgggcacctcgctgcgccgctggtcggcgctgctggccgcggaggcccgacacccggcccgagccgcggaggcgaccctgtgggccgatctcctcgccgccgccgacccaccgctgacggccggccggctcgatcccgttcgggacaccgtgggccgggccagtgcgctcacgctctcccttccgcccgacgtcaccaccccgctcctgaccacggtgcccacggccttccacgccggagtgaacgacgtcctgctgaccgctctgggcctggcggtggcccagtggcgccgggggcacgcccgcgggaggcacacggcactgctggtcgacgtggaggggcacggccgggaggagatcgtcgagggcgtggacctgtcgcgcacggtgggttggttcacctcgctgtatcccgtccgtgtcgacccgggaccactggtctgggaggaggtcgtggaaggcgggcccgctctcggacaggcgctgaaacgggtcaaggaacagctgcgggcgctgcccgaccgaggcatgggcttcggtctgctgcgccacctcaacaccgggaccgggccggagctggccgggctcgccgtgccccaactcggcttcaactacctcggccgcttcccgtccgcgggcactccggtggtgcccggccatccgggatggaccgtggcgtcggaggccggcctgctgagcggcgccgatccggcgaccgccctggcgcacggcatcgaggtcaactccatggtccgtgacaccccggacgggccgtggttggaggccgtctggacctgggctcctgatctctggcccgaaccacacgtccgcgccctcgcggagcagtggttcctggcgatccgtggcctggtccggcacgtcgaccggtccggtgcgggcggccacacaccctcggacttccccctgaccgaactcagtcagcacgacatcgagcagctcgaagccgcgtggaggattcagaaatgacgtcgtcgggcctggaagacatccttcccctctcgcccatgcaggaggggctcctgttccactcccgctacgagcaggacggcgcggacgtctatgcggtgcagcacgtcttcgacatcgaaggcaccctcgacgcggcgcgcctgcggacggccgtccgcgccctcgtccagcggcaccctaaccttcgggccggcttccggcaggtcgaatccggagggaccgttcagctgatacctcgtcaagtggatgttccctgggccgagtacgacctcgcgtcgttgcccgccgccgaagcggaggcggaaatcctccgactcgccgccaaggaacacggcatccgcttcgacctggcacagccgccgctgctgcgcttcaccctggtgcggctggccggggcacaccatcggctggtcatgaccacccatcacatcctgctcgacggctggtcgatgcccatcctcgtccacgagctgctcacgctgtacggcagccacggcgacgccgccgggcttccccggacgaccccgtaccggcagtatctgggctggctcgcgcagcaggaccggccggcggcggaggccgcctggcgcgaggcgctcgtcggccttgagcagccgaccctggtcgccgcggtggatccggcgcggcccgcactcatgccggagcggatcaccgtcgagctggacgaggaccgcacgacggcactgacggactgggcgcgtcggcagggcgtgaccacgaacacggttctccaggccgcctgggctctggtgctgagccggcacacggggcacgacgacgtggtgttcggggccgtcgtggcggggcgcgacccgcaactgcccggtgtcgaggccatggtggggctgctcatcaccatggtgcccgtccgggtcgtgctggacccggcgcagtcgctgacggagaccgtcatgcggctccaggacgagcagacgcggctgaccgcgcaccaccatctcgggctgccgcgcatccagcaactggccggccccggcgacctcttcgacacggccatggtgttcgagaactacccctgggacgaatccaccgagctgccggacaccgggctgcgcgtcaccccggacctcggcagcgggcgggacgcgacccactatccgctcaccctggtcgcggccccggggcggcgcctctatctccgcctcgactaccgggacgacctcgtcgacagggcctcggccgccgggttcctcgaccggctgatccaggtgctcgacctcgtctccaccgccaccagcactgattccgataccggcaccggacccgccgcaaccgagctgcccgtcgggcggatcgacctgctcagcgatgaggagcgcgccactctgtcggccgcccccgacgacgtcgcgcgcacgacaggggcgaccctgccggggccgttcgaggcgtgggccgtcgggaccccggacaccccggcggtggtctgcggcgagacggccctcacctaccgggaactcgacgagcgggcgaaccggctggcgcatgtgctgacgacccgtggcatcggcccggaggacatcgtcgccctcgcgctgccccgctcgaccgacctcgtcctcgccgtcctcgccgtcctcaaagcaggcgccgcctacctgcccctcgaccccgaatacccccccgcccggctgacccacatggtcaccgacgcccggcccgcccttctcctcaccaccacggcgatccggtccagcctccccaccacgcacgacaccccggtcctcctcctcgacgccccggacaccgccaccgacctcgccgggcgccccacgcacagccccacccccgccctcgcacccgatcacccggcgtacgtcatctacacctccggctccaccggacgacccaagggggtcgtcatgcccgcgggcgtcctgctgaatctcctggagtggcaccaccgcgagatcggcggccaaccgggcaccaggaccgcgcagttcacggcgatcagcttcgacgtgtccgcccaggaggtcctttcggccctgctcttcggcaagacattggtgatacccgccgaggacgtccggcgcgacgcggcccgcttcgccgagtggctcgaagaccagcgggtggaggagttgttcgcgccgaacctggtccttgaggccctagccgaggccgcagtcgagcagggccgaacgctgccgcggctgcttaccgtcgcccaggccggtgaagcgctgacaccgagccgcctgtcccgtgagttccaccggtcggtgccgggccgggtactgcacaaccactacggccccaccgagacccatgtggtcacggcccacaccttggggcacgacccggacgactggccgttgtccgtgccgatcggccgtccgatcgtcaacacccgtgcgtatgtgctgggttccgggctgcagctggcggctccgggcgtggtgggggagctgtacatcgcgggtgctggcgtggcccgcgggtatctgcaccgccccgctctcaccgccgagcgtttcgtggccgacccgtacggcagcgagcccggtgcccgcatgtatcgcaccggggacctggtacgccgcaacgccgacggcaacctcgaattcgtcggccgcgccgaccagcaggtcaagatccgggggttccgtatcgaacccggcgagatcgaggccgtgttgaccgatcatcccgacatcacccaggtcgccgtggtcgtgcgtgaggacgagcccggcaggaccctcctggtcgcctacgtgacggcgcgggaggccctgcggcccgacgcagtacgggagttcacccgagaccggttgcccgaacacatggtgccgtcggccgtcgtactcgtggagcaccttcccctgacggcgaacggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccacctgggcggtcattcgctcctggccactcgcctcatcgcccgcatccgggccgccttcggcgtcgaggtcggcctgcgcacgctcttcgaggcccggaccgccggcgcggtggccgcccgcctcgacaccgccgggcccgcgcggctggccctgaccaaacagcaactgcccgacctcgtaccgctgtccttcgcccagcgtcgactgtggttcctgcacaagatggagggccccagcgccacctacaacattccgctcgtcctgcggctgacgggcgagctggaccgggaggcgctgcgcgcggccgtggaggacgtggtggcccggcacgacagcctgcgcaccgtgttccccgaagcccaggacactccgtatcagcacgtcctggactccgtcacggtcgagctgtccgccatcgacatctccgaggcggaactgccggacgccctcacgtcggcgtcccgtcatcccttcgacctggcggccgaacccccgctgcgcgtcggcctgttctgcctcgcacccgaccggcacgtcctcctgctcgtgctgcatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcgtctggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctggtttgcagattgatgtggcgccggggcggacgcatacggcgaagttcgatctgttcttcagtcttgccgagcggcgtggtgggcgtggtgagccgcagggcatcgtgggggcggtcgagtactccagtgacctgtatgacgcatctaccgtgcggtcgctgttcgagcgttgggtcaggctcctcgacgaggcgaccgcccatcccgagcagccactcgacaccatcgacctcctcaccaccgaagagcaacaacagattctcggcacctggctggacaccgaggcggaagtcggtgcggacctgttgccggcgtgctttgccaggcaggcgcgcaccacggcggaggccgtcgccctgatcaccggggacacctccctgacatacggggagctgaacagccgcgccaatcgcctggcgcacgccctgctgcgccaaggcgccggacctggcacggtgatcgccgtcgccctgccgcgctcggccgagctggtggtcgccctgctggcggtgctcaagaccggcgccgcctacctgccgctggacccggaccatccggccgggcgcaccgcgtacgtcctggcggacgcggaacccaccctgctgctcaccaccttggcgacggacgcgcggctgccgggcgacggctcgccacggcggctgatgctggactcaccggacgtacaggcgctggtggccgactgccccgacaccgacccgtccgacgccgaccgcaccgccccgctgcgcgccgcggacgccgcatatgtgatctacacgtccggctcgaccggccgccccaagggtgtcgtggtgccgcacggcgcgctgaccaacttcctggaagccatgcggcagaacgtgccgctccggccggacgagcggctgctcgcggtcacgacggtcgccttcgacatcgcggccctggagttgtaccacccgctgctctcgggcgccgccgtcgttctcgcgcccaaggaggccgtcccccagccgtccgtcgtgctcgacctgatcgcccggcacggtgtcacggtcatgcagggaacgccgtccctgtggcaactgctgctcgaacacgagccggaggccttgcgcggactacgcgttctggtcggcggagaggcgctgcccgccgccctggcccagtcgctgcgggccctcacggacgacctcaccaacctgtacgggccgaccgaaaccaccatctggtcgaccgccgccgacctctccggcgccacggcagccccgcccatcggcaggccgatcgccaacacccgggtgtacgtcctggacggccggctgcagttggtggctccgggtgtggtgggggagctgtacatcgcgggcgccggcgtggcccgcggctacctgggccgccccgccctgaccgcggaacgcttcccggccgacccgtacgggagtgagcccggcgcccgcatgtaccgcaccggggacctggtgcgctggaatccgggcggcgagctggagttcgtgggccgggccgaccatcaggtcaagatccgggggttccgcatcgaacccggcgagatcgaggccgtgctggccgatcaccccgccgtcgcccaggccgccgtcgtcgtccgcgaggacgagcccggggacgcccggctggccgcctacgtcgtcgccgacacctcgggacgcgcgtacgacgagaacgccgagctgaaccagctgagcgagtggcaggacctgtacgactccgtctacacgagcgcacccaggacggcgttcggcgagaacttcgccagctggaacagcagttacgacggacagcccatcccgctgcccgacatgcgggagtggcgcgacacgaccgtcgaacgcatcagggccctcagtcctcgccgcatcctcgaaataggcgtgggcaccggactgctcctcgccggcctcgcccccgagtgcgaggagtactggggaaccgacttctcgcccaccgtcatcaaggacctgcagcgccacgtcgatgccgatcccgccctcgcctcccgggtccacctgcacacccggccggcccacgacttcgacggccttcccacgggccacttcgacacgatcgtcctcaactccgtcgcccagtacttcccgagcgccggctacctggagcaggtcgtcgccaacgccctccgcgcgctggtccccggcggagccctcttcatcggcgacatccgcaacccgcggctgctgcgcaccttcgccgccgcggtcagtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcaccgacacccccacccacccctggccccggcacaccgaccccggcgccaccctcaccgatctgagccgtcacctcgaagccgagcgccccgccgggctccgcgtcaccggcgtcccgaaccctcgcctcgcgcacgagctggccgttcagcacaccctcttcgacgacacacctgcgtccgcggacagcggccggccgcacgtcacgctggaggccttccacgctttgggcgacgagcacggctactggaccggcgtcacctggagcactcacgaccatgacgcggtagacgtcctgttcgtcgaccggaacctcatggacgaaggcatccccgtaggcacctacgcccctgccggtacgggcacctcggccatgccgctgtccacctggacgaccaacccggccgcccgccgcggcaccggcgccctcgtcaccgaactccgcgaacacagccgtcagcatctgcccgactacatggtccccgcgttcatcgtccccctcgaccggctccccctgacggcgaatggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccagctgggcgggcactcgctcctggccactcgcctcatcgcccaggcccgggcggtcttcggcgtcgagctggagctccgctcgctcttcgagggtccgacaccggccgaggtcgccgcactcctggacaccgctgggcccggccggctggcgctcacacggcgcgagcggcccgagaccatgccgctgtccttcgctcagcgtcgactgtggttcatccacaagatggagggccccagcgccacctacaacattccgctggccctgcgtctgacgggcgagctcgaccgcgacgcgctgcgggccgcactcgctgacgtggttgcccggcacgagagcctgcgcacggtattcccggaggccgagggagttccgttccagcaggtgctgcccgtcgaaacggccgtaccgcatctcaccgtcacaccgacgaccgaggcggagctgccggacctcctcacgtcggcgtcccgtcatcccttcgatctggcggccgaaccgccgctgcgcgcggccctgttcgagctgtcggcgcaggaatacgtgctgctgatagtggtccatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcggttggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctgggttgcagattgatgtggcgccggggcggacgcatacggcgaagttcgatctgttcttcagtcttgccgagcggcgtggtgggcgtggtgagccgcagggcatcgtgggggcggtcgagtactccagcgacctgtatgacgcgtccactgtgcggtcgctgttcgagcgttgggtcaggctcctcgacgaggcgaccgcccatcccgagcagccactcgacaccatcgacctcctcaccaccgacgaacaccggcacgccgtcgtcgagttcaacgacacggccgtgcctttgcccgagttgtctctcgcggagctcttcgtgcggcaggtggccgggactccggatgctcctgctgtgatggatggggagagctctctcacgtatgcggagctcgatgtccgtgcggatcggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggaagagcgcatccggcacgtccttactgaaacaggtgcgcgactgctggtcaccgacgacccggcggagttccaaccgggcgcggaggactgtgagttgatcggagcggcctcgatgggagtggccgggacggaacctggcggtcctgttgaggtggccgttgcccctgatgctgcggcgtatgtgatgtatacgtctggttcgtcgggtgtgccgaagggggttgtggtcagtcatcggaatgtggctggtctggcgttggatcctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtttgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtgccggacgtggacgggacaacccccggcgaggaagcggaacaagggcagatcgaggagtggaaggatctctacaactcgctctattccgagcccggttcggagttcggcgaggacttctccggatggaacagcagttacgacggtgagcccatcccgctgaccgagatgcgcgagtggcgtgccgccacgaccgcgaggatcaaggcgcttcgaccgcgccgggtcctggagatcggcgtgggtaccggactgctcctcgccggcctcgcacccgaatgtgaggagtactggggaaccgacttctcacccaccgtcgtagaagcgctgcgacgccatgtcgacgccgaccccgaactcgcccggcgcgtcaccctgcgcgtgcaggccgcgcacgagaacggcgagctgccgcaaggccacttcgacacgatcgtcctcaactccgtcgcccagtacttcccgagcgccgactatctggagcaggtcatcgaccaggccttccgcctgctcactcccggcggagccgtgttcatcggcgacatccgcaacccgcggctgctgcgcaccttcgccgccgcggtcggtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcgacgacacccccacccacccctggtcgggggatctcacggacctcgctgagcagttgggcacgggacgggccgatcggctgcgcgtgaccggcgtcccgaactcacgcattgcccacgatctcgccgtgcggcaggcgatcgagggcgggacggcggctcccggaccgctcgtggccggtgtcgatccggaggaactgcaccggctgggggagcgccacggttactggaccgcgatcagctggagcacgcacgatccggccgccgtcgacgtgacgtacgttcggcccggactcctggcagagcgcgtcgccgtaggtacgtacgcctccgcgggcagcgccgcttccggtatgccgctgtcctcctggaccacgagcccgggcgccggccgcgacacgggtgctcttctcatcgccctgcgcgagcacgcccgtcggctactgcccgactacatgcgccctgccgccatcgtgcctctcgaccggctgccgctcaccgccaacggcaaactcgaccgcgccgcgctgcccgccttcgcgccggagcaggccgacatcggacgggcaccggccacaccacaggagcaggtcgtgtgcgagctgttcgcggaggtgctcgggcggccggtcgtcggagtggacgaggacttcttcgccctgggagggcactccctgctggccacccggttgatggcgcggctgcgggccgccttcggcgtcgagttggggctgcggagcctgttcgaggcgtcgaccccgggcgggatcgcggcccggctcgaagccgacgactcggacggctcctacgaggtgctgttgccgctgcgcccccggggcacacgggcgccgctgttcttcgtccatcccggcggcggcatcagctggtcgtacagcgcgctgatcaaacacctgggcccgcagtacccgctgtacggaatccaggcacgcagtctggcccggccggaggccaggccgggcagcatcgaggacatggcgaccgactacgccgaccagatccagaccgtgcagccgcacggcccctaccacttggcgggctggtccttcggcgggctgtgcgcgcacgcactggcagccgagttccggcggcgcggtgaaccggtggcgctggtcgcggtgctcgacgtgatccccaactggcaggggctgacccacgcggacgtacctgcgcccgacgaccgggtgatgctgctgtaccacgtgggtctcgtcgacgacggcacccaacggcaggacgacgaggagatgaccttcgccagggcgcgggagatcctgcgtcgccagggcagtgcactggccagcctggacgaggaccggctcgcggcgatcaccgagatctcggcgaacaacacccacctgaccatcgactaccggcctggcgcgatcgacggcgatctgctgctgatcgcctgctcggagcagcaggacccgccggtcgacgctgcggcctggcagccgtacgtccgtggcaccgtcgaagcccatgtagtgcccggcgagcacgggtccatgctgacccggccggacacgctggccgagatcggtcggatcctgtcggccaagctccacgaactgaccggcgacgagtgacgttcccagcccgccttccaggctcgccccgtccgccttccgcgtggaccgatcccgaggactgacaacgatgctgaccgacgtgctctccgtcgacttggccaaggcgctagaggccgccgacggctggctgagcgactacatccgccgaccacacagcgaactgggacgtaccggcccggtgtgccccttcgtggaacccgctcagcgcgccggcgcgctggagatccgggtcagactggtgggtcccacccccagccagaccctcatcgacgagatcgtccgctgtgggctggacgagttcagcgaggtcgactggaaggccggcaacccgaatctgcgctcgctgctgctcgtcctgccggacctgccgcccgagcatctgcacctgttggacgaggcgcacgccgcgctcaagcccgagagcgtgcaccgcggcctgatgatcgcccagttccacgagaagtgccgggagaaggccgcccgcaacccggagttcgaggtgagctacgcccccgtgccgatgatggcggttcggtccatggcgatccatgacgtgctgttcctcgcggaccggcgcgagtggttcgaggagtacgcgagccgcttcggggcgcgttaccggaactcggcgcacggcatcgatccgctgctgaccgagatgtacgaacgggcccgtgccaagcatgggctgatgaccccgtgatgcacggcggattacggcccgggggtgcacgatgacggacaccgccgtgtaccggggcatggatcaggccacgctcgaccggcagtactcgccgagttcccgggtgccgagcctcggggcctacctgcgggagtacgcacggcggagcacggccgcccgccggtgccacccggtgcgcaccgggctcgcctacggcggacatccggccgaggtgctggactactttcccggccgcccggggcccgggcggccccccttgctggtgttcgtgcacggcgggaactggcaggcgctcggccgtgccgaatcggccttcccggtaccgccgttgctcgccgccggtgcggcggtcgcggtggtcgagtacgggctggcgccggacgtcggactcgacgcgatggcggggatggtgcggcgcgcgatcgactggctgctgcaccacgcggaccggctcgggttcgcctccgaccggctccacctgtgcggcacttcggccggcgcgcacttggccgcgatggccctgttgcccggtccgccgcaggggccggacgtctccggccggatcgccggggcggtgctgctcagcggcatgtacgaccttgagccggtccgtctgtcgtacgtcaacgaggcgctgcgccttgacgaagcgggggcgcgccgcaacagcccgctcggcctgctgccgccgcggttgcctcaggtggtggtcgcccgcgggggcaacgagaccgaggagtacgtccgccagcacgaccggatggtcgcggcgctgcgtccccgggccgcggtgtgcgaggtcgtcgccgagcggcgcgaccacttcgacctcccctacgacctgggggtgaggggcaccggactgggcgacgcggtgctcgcgcagatggaactgggaggagcggccacatgaccaccgaacagcccgcgcagggctccgggcagagcgccggatgcccgctgtccgcagccgtcccgccgggcaccaccccgtacgcgtactacgcgcgcatggacgagctgctggcgctgcagcatccgctcagcccggcggacaccgagccggggttcatcatcatgagccaggtcaaggagctcctcttcaagctgctgcacaccgaactgaccaccgcccgcgatcagttggacaaggaccggatcgacgccgcgctgtggaccctgcgcaggtcggcgcgggtgcagcaggtcctcctcgtgtcctgggagacgttctccgtgctttcgcccgccgagttcgccgagttccgggacgtcctcggatcggcgtccggcttccagtcggccggctaccggcgactggagttcctgctcggcaacaagaacccggcgatggcgcagccgcaccgggacacggccggtcacgacgcggtgctgaaccaactgcacgagccgagcctgtacgacgcggccctggccctgctggcccgccgggggctgctcacgccggacgactccgcggcgcgggacgccaccctgccgtaccggtccggcgaggacgtcgaggaagcctggcgggcgatctaccaggaccggcccggccaccacgatctgcatgcgctggccgaggcgctgatggacacggcggagcagttcgcgcgctggcgctacacgcatctcctcaccgtgcagcggatgttgggagccaagccgggcaccggcgggaccgatggcgtcgcctggctcgcccggatcagcgaacaccgattcttccccgagctgtggtcggtgcgctcgacgctctgacccaccggacaggtgccaggagcaccagggacacgacagatgccgacggactgaaggtgaggagtgccgatggagggcggaatcccgcgcgcggtggccgaggagctggacgcggccgatccgctggccgcgttccggaagcggttcgtcatcaccgatccggagttggtctacctggacggcaactcgctgggccgactgcccgcggccacgtcggacctgttgcgcgaggtggtcgagaagggctggggcggcggcctcgtacggtcgtggcaggaatggatcgactggggcgcccggctcggcgaccgtctcgccgcccacgtgctcggcgcccggcccggcgaggtggtgatctcggactcgacgtcggtcaacctgtacaagctggccggcgccgcgctggacgcggcgcgcggacggaacaccgtcctcatggacgccgaggactttcccaccaaccggtacgtcctgcaggggctcgccgaccagcgcgggctgcggctgcgacggctcccgtccgatctcgacggcggcctggatctcggtatgctgcgctccgccctcgaccgggacgtcgccctggtggtgctgtcgctggtgtcctaccgcagcggcgcgctcctggacatggcgaaggtgaacgaactggcccgcgcggctggcgccagggtgctgtgggacttgtcgcacgcggccggagccgtccccgtcgatctcgccgcggccggtgcggaactcgccgtggggtgcacctacaagtacctgaacggcggccctggttcacccgccttcctctatgtgcgcagtgatctgcacgccgtgctgcgccagccggtgtggggctggtacgggcagcgggagcagttccggatggggcccgactacgatccggtgccggggatcgagcgcttcctggtgagcaccccgcccctgctgtcgctggcggcgatcgatccggccctcgccctcgtggaagaggccggcgtggaccggatacgtacgaaggggctgctgctcggccggctggccgacgatctggccgccgcctggctcaccccgctgggcttccggtcggcttcgccggggacgccggagcagcgaggctcgcatgtgtcgctgcaccacccggacgcctggcggatctgtcagacgctcgccggtgaagcgaaggtcgtctgcgactaccgggtacccgaccggttgaggatcggtctgtcgcccctctacacccggttcacggacgtctgggacgcactggcacgcctccgcgaggtcaccgccgggcgcacctacgcgtcccgccccgccgacctggcccgggtgacctgaccgtcggcctcggggccaccgccttgcccgcacctcaccacccacccgatacggagtgcccatgaccgacacatcgccgctcaccaccgacggcctggtacggatcctgttcggctcctcggccttccagatgctcaacgcgggccgcaacctgggtctgttcgcgctgctcagtcggcagtccgggctgaccgctcaggagatcggacgtgaactcggcctggcggaacgcccggtgcagatcctgctcctgggtactacagctttggggctgacggtccgtcagggcgagggctacctcaatgccgctgtcctgaacaacacgtttgaggacggcacttgggagatcatcgaggatctgatcgagtacgaggagcggatcgtccgccccgccgaggtggacttcacggagtcgctgcgccaaaacaccaacgtcgggctgcgccggatcgacgggaccggcaccgacctctaccaccggctgtccgcgaaccccgagcttgagcagttgttctaccgctgcatgcggtcctggtcacggctgtcgaatcccgtcctgatcgagcaggccgacctgaccggggtgcgccgggtcctcgacgtcgggggcggcgacggcgtgaacgccatcgccctcgcccaggccaaccccggcgtcgagttcaccgtcctcgacctccccggcaccgtggagatcgcgcgacgcaagatcgccgagcacggcttggccgaacggatctccgtccgggcggcggacatcttcgccgacgactacccggcggggcacgactgtgtgctgttcgccaaccagttggtgatctggtcaccggaggagaacgtgcgcctgctgcgcaaggcccacgcggcgctgcccgacggcgggcgcgtgctggtgttcaacgccatgtccgacgacagcggcgacggccccctgtacgcggccctggacaacgtgtatttcgcgacgctgccggccgcgagcagcaccatctaccgatggggccagtacgaggagtggttcgccgcggccgggttcgtgaagcccgagcggctgccgggcggccggtggacgccgcacggcgtgatcagcgcggtcaagtgacgccccagcgagaaccggagtcggccatgtccctcaagtcccacgacgccccgccgaccggtggggccgcggcgtgccccgccggtccgcacatgatggatccggatctgctccgggaccctttcggcggctacggccggctgcgcgaacaggacccggtggtgcacggcaggttcgtcgacggcaccccggtgtggttcgtgacccgctacgacgacgtccgcgcggcgctgcgcgacccgcggttcgtcaacaccccctcccacgtgccgggcgagaagggcgcggacccgcgcgagggcatgatggaactcctcaaggtccccgagcatctgcgcggctatctgctcggctccatcctggacagcgacccgccggaccacccgaggctgcgccgcctggtgacccgggcgttcgcggcccgccgggtcctggatctgcgccaggacatcgagcggatcgccgaccggctgctggccgagctgccgcaccgggaggaggacgggacggtcgatctcctggagcacttcgcgtatccgctgtcgatcacggtgatctgcgagctcgtcggcatcccggcggccgacctcggccggtggcgggagtggggcggcgacctggtgtcgatgcggcccgaacgactccagcactccttccccgtaatgatcgattactgccacgaactgatcgagcggcgccgcgccgcgctcaaggacgatctgctgagcgagctgatccgggcccaggacgacgacggcggacggctcagcgacgtcgagatggtcaccatgatcctcacgctggtcctggccggccacgagacgagcgcgcatctgatcggcaacggcaccgccgcgctgctcacccaccccgaccagctggcattgctgcgcaaggacccggcactgctgccgcgcgcggtgcacgagttgatgcgctggtgcgggtcggtgcacgtggcccgactgcgctacgccaccgaggacctggagctcgcgggcacagcgatcgcccggggtgacgcggtccaactggtcctggtgtcggccaacttcgacccgcgccactacagcgacccggaccgcctggacatcacgcgccaacaggaggggcaggccgagaaccacgtcggcttcggacacggcatccactactgcctgggtgcgacgctggcccggcaggaaggcgaggcagccttcgtgaaactgctggagcactatccggacctggcgctcgccgatggggccgccgagctggaccgggcccggctgccgggcagttggcggctgaactcgcttcgccttcggctccggccctgacctcggtcgcggacaggggccgcccggcatcgggcggcccctgtcggccggcgtctcgccttggcggcgcacggacgggtgctcagccgggtggtccgtctcccagggtgatggcttgcaccgggcatgccagcgccgcttcgtgcacctggtgcggcaccgcggtcgcggcgccttcgggcaccaactccgagtatccgtcgtcgtcctgagtgaatacgtccggggccgtcagaacgcactgcccggaccctatacacgtgtgatgatcgatactgatctgcatggaagaccgtctccgatggtcgggcggacaggggccactcagtccccgagtgcgtgcgcggcggcgcggtcgagggcgacgagtatccactcctcgaagttcccttccaccttcgccgcggcctcgcgccagcggtccacgcgatcgacggacacgctgatcctgggcagaccgacctgtgcgagcggggccgcccgactgccctgacggctctcccgcagctggagccggagccggttcctcgaccagccctgctcctccgcgcggtcgagccaggtgtccgcctgctcgggcgggagcgaggcgacctcggcatggtggccgaagctcaggttctcgcgccgtcgcccgagctcgaagtgccgtgccacccatgcgtagttgcgcagagtctgatagtcgagtccagcggcctgtacgccggtccggtagcggtcggagtaccgctcctggccgtacacgagccagtcgcccaggcaccaggcggatgagtcggcgatctggaagatgtgcagtccggccttctcccaccggtcgtacgacaggatggcgggaatcttcaggccgacccgtgtggtcaggatccgcccttgatcgttgctcccgcggcgagccgccgcctcgcccggtctgtggccgactgctaaatgactgtcacgctggtggtacttggcgatttgctgcacgggttttccccctggaattgagccattgccaaatctcgtcccagtcgtcagcgaccggggaccatgaaaggaccccggcccaaaaaccgtagattactgaacgatgtatcgtcaatgatcaattccggacggatgtgaccggttggagcgcaaaggagcccggggatgacagcctcatgtgtccgtgatctctaaaacccgtgtgcaatccctgcgcccggacccttacggcgcggacggggtcgctcgggccgggctgccgaggccgggccgttcggccgcggcgatcccgtcgaggatcagttccaggccgacccggaaggcctccttggcctcccgctccacatggggtgtcgcgtcggccggctccttccccgcgtcgtcggccgcggcagcggtggcgtgcgactccagcgccgccagagccgggaagcgctccgcgaagtccggcgccacctcggtgagcagagcggtccgctccatccaccactcctcgtcggacacccccgtctccggcgcggcctgccggcgctcggcggccacccgggccatgccccggacgaactggaacagcacggcgatgacccggcggactctcaagggctccaggcccaccccgcccaggatgcgcaccaccgtctccagttgcgcgtactcgcccggcccgagcaccggccgggcctgggagacctgcaggacccaggggtgccgcagatagaaggaccaggtgtcctcggcccactccgcgatcgccgcgcgccagcccgcggcggtgtcgtactcggtgggcagctcggccagcacccggtcgtgcatgagatcgaccagttcgctcttgctcggcacataggtgtacagcgccatggccgtccgtcccagccaggaccccaccgcccgcatcgacagggctgacattccctgctcctcggccaccgcgatgcccgccgccacgatcgtgtcgacgtcgagcgcgggcttgggcccgggtgcggatctggtgcccttcgggccggccgagcgccagagcagagccattgagcgacgggcgtcgccctgtccggcaaaaatgaccaccttgcaactccttactgcataaagtaacctcgccccgagcatctccttacggtgtaaggatatcaggtggacgtggggtcccgcctcgggatgcccggcactcttggaaggtttttgatggcagagctccctgtttcattcatccaggtcaccgacatcaagcacgtcacgccgcgcatggtccgagtgacgttcgacgcggaccacatcgacgactccgtcggcggcacgccggaccaacaggtcaagctctgcttcccccgatccggccagcgcgtgcccgtgctgccggaacagggtgacgacgcgaccagctggtaccaggcgtttctggccattccggaggacgagcggccctggatgcgcagcttcaccatccggcggcgccggccgggcaccaacaccctcgaaatcgacttcgtcctgcacggggacaccggccccgccacccgctgggccggttcggcccggcccggcagtcagctcggcatggtcggaccttcgacgctctactccgcacccgtctccctgacggactcgatcgcgaactccgactggctcctgctcgccggtgacgagaccgcgctgccggccatcggcaccctgctggaggcgctgcccgagggggcacgtgccctggccttcgtcgaagtcgccgacgccgaagagcggcaggacttcgagaccgccgggaacgtcagcgtcaactggctctaccgagacgggacttcgcccggccagagcgacaagctggtcgacgccgtgcgcgaggccgagttcccggccggccggccgttcgcctggctggccggcgaatcgggggccgtgcgggccctgcgccgtcatctggtcaacgaccgggagctggacaagaggtcgatcgacttcagcggctactggcggttcaagctcacccaggacgatgcgccgaccacggaggacatggccgaggcgcaggagcggctcgccgagttccaggccgccgccgacgcccgcccggaatgaaccgcacggaggggccgggcttcggctgggcccctccgcgcccgaccgagaagcggtacgcgccgacaagcggtacgcgcgaaagaaggctttcatgatcacagtgcgaggtctcgcccggcagttcaccctccgtggacggaccgtcgatgcggtgaagggcatcgacttcgatgtggcggccggggaactggtcggtttcctcggccccaacggggcgggaaagaccacgaccctgcgcatgctgaccacgttactgcgccccacccggggttcgggcaccgtggccggctgcgacctcctggccgacccgcagggggtccggcgcaagatcggttacgtggcgcagggcgggtccacctggccggagtccaaggtcggggaggagatcgagctgcaggcccgcctctacggcctgtcgcgggccgaggcccggcggaggggagcgctcctggcggaacgactcgacctcgccggcctggagcagcgtcccgcgaagacgctctccggcggccagcggcgccggctcgacatcgtgctgggccttgtgcacgagccccaactggtgttcctggacgaacccacctccggactcgacccgcagagccgggccaacctgtgggaccacgtccgccggatgcgcgccgaacagggcatgaccgtcttcctcaccacccactacctcgacgaggccgacgccctctgcgaccggatcctggtgacggacgacggcaagatcgtcgccgaaggaaccccggacgaactcaaggcaggcgtcgcaggtgatgcgatcaccgtcgacgtcgcggaccgggccgccgaggccgccgaagtggtgcgccgactgcccggcgcccacgacgtcaccgtcgtcgccgagaccgtacggctcagggttccccggggtgactccgtgctgccggagctgctgcgcgccctggacagccatcggatcacgatgacctcggtcaaggtgaaccggcccaccctcgacgacgtcttcctcagcctgacggggcgttccctgcgcgacggcgaggcggcgagcccggccatggaggccactcatgctgcgtgacacctggctgatctttctgcgggacatgaagctgtgtctgcgcaatcccacctggctgatcatcggcatcatgcagccgctgctgtacctgttcctcttcgggccgctcatggtgacggtggtggcccatacgcccggcttcccgtccggcaacgcctgggccgtgctcacgcccgcgctcatcgtgcagacggcgctgttcagcggttcgttcgccggagtcgggctgctcaccgagtaccgggtcggcgtggtggaccgcttccgcggaacaccggccagccggtccgcgctcctgttcggcaaggtcctggcccaggccgtccaggccaccgcgcagtcggtgctgatcgtcctggtgacgctgctcgtcttcgacctggacgcgccggcgtccggcatgctcctgagcctcgccatggcggcggtactcgccatcacgctgtcgtccggatcgtacgcactggcgatggtcctcaagagcgaggccgcgttcccctcgctgatgaacgccgtgctgctgccgctgctcctgctgtccggcgtgctgctgccggtcaccgcgcaactggctcccggctggctctacaacctggcccgcatcaacccgctgacccatgtggtcgacgccgaacgggccgcgttccgcggggacttgggctccacagggctgctgaccgggtccctcgtcctgttggcgatggccgccctggcgctcgtctggggcacgcggacgttccagaaggagaacgcctgacggcgacatcccgtgaggcgacagcctctctgcccgaccacgaccggggctctccggggagccccacccgtaccacccgaggagcacatgtgagtaccgacgccatcaaggtgaccggtgcgcgggagaacaacctcaagaacatctcggtggagatcccgaaacggagtctgacggtcttcaccggcgtctccggctcgggcaagtcctccctggtgttcgacaccatcgccgccgaggcccagcgccaactcaacgagaccttcaccgccttcgtccgcaacttcctgcccgaccacggccgtccggccatggacgccatcgagaacatctccgccgccgtcgtcatcgaccagaaacggctgggcggcaacccacgctccacggtcggcaccatcacggacatcaacccgctgctgcgactgctgttctcacgggcgggcgaccccgtgggcctgccgcccagcgcgttctcgttcaacgacccccaaggcatgtgcccgacgtgcgaggggctgggcagggtcgtggaactcgacctcgacgcgttcgtcgaccgcgccaaatccctcaaccagggcgcgctgctgcaccccgacttctccacggacagctggtactggctgagttacgtggagtccggcttcttcgacaacgacaagccgctcgccgacttcaccgaacgggagttggggctgctgctgcacggcgagatgccacagaagctccgcctggtctggcagggcaacacactgaacgtcaagtacgagggtctggtggacaagttcaccaggctctacatcaagaaggaggacatgtccgagcgcagccgccaggtgttccagcggttcgtacgctcggctccctgccgggactgcgcgggcacccgcctcgcgcccgccgcccgtggctgccggatcgccggccaccacatcgcggatctcacctccatggaggtcgagcgcctggccgcggtcctggcggggctggacctgccccacgtgaaaccggtcctgaaggacctggtctcccggcttcggcacctggtcgacatcgggctcggctatctgagcctggaccgcgagacccgaacgctgtcgggcggcgagtcgcaacggatcaagatggtccgccatctgtcgagcagtctgaccgacatgctgtacgtcttcgacgaaccgagcgtcggactgcacgcccgcgacgtgcaccggctcaaggaactcctgctcgaactgcgggacaagggcaacacggtgctcgtggtggagcacgacccggacgtcatggccatcgcggaccacgtggtcgacatgggaccgaaggccgggacgctgggtggcgaagtggtgttcgagggcagcttcaccggtctcgtcgaagccgacaccctcaccggccgccaccttcgccaccgcctgcccgtgaaggccgcaccccggcgcgcgaccggcgcgctcagcgtcaccggcgcgaacagtcacaacctcaccgatgtcacggtcgacttccccacgggcgtcctcaccgttgtcaccggagtcgccggatcgggcaagagttctctcgtcaacgacgacttcctgagccaacatccggatgcgatcgtgatcgaccagtccgcggtgacggggagccggcggtccaacaccgccacgtacaccggactcctcgacccgatccgcaagctgttcgccaaggccaacggggtcagtgcgtcgctgttcagcgccaattccaagggcgcctgccccaactgccaaggactgggcgtcctttacaccgacctggcgttcatggagacgctgaagtccgtgtgcgaggtctgccagggccgtcgtttcgccgatgacgtcctcggctaccgactgcgcggccggtcgatcagcgacgtactggccatgacggcggccgaggcggcggacttcttcaccgagccgaagctccgccggacgctgcacgcgctgaacgaggtgggcctggactacgtggggctcggccagccgctcagtaccctgtccggcggcgagtgccagcgcctcaagctcgccgccgacctgcaccgcgtcggcagcgtctacgtgatggacgagcccaccaccggccttcacctgtccgacacccaccgcctcctcggcatcgtcgagcgcatcgtcgaccagggcaacacggtgatcgtcatcgagcacaacctcgacatcgtcaagaacgccgactggatcatcgacctcggccccgaaggcggcagcggcggcgggaaggtcctgttcgagggaaccccggcggaactcctgaaggccgccggctcgtacaccgccgagtacctccgccgggacctttcgggcgcctgacgcgtaggtgcccgtccaccgccggggcgtggccggtgtcgtcggggccggatcgcggcggatcggacggaaggcgcctgacctgggcagaacctcaggtcaggcgctttcttcggctggggaggacgttcagcgtcctcgccaccgccgtcgtccggtcggcgggacgaggccagtcgagctgcctgggcccgtcccgcgccggtacaccgctgcgggcgaaggcccgcagctcgccgtcgcagccgacgacgtgaagctgcgggggaccctcgctggaccacgccggttctgctccgcccaagctggcgcggcgtcggccaggtgtcgttcctcggcccgcgactgacagccgtgcggatggtgtggttggctggcctgcgcgattgcggcgtgacgggcaagggaggcgaggcatgggcggagtggactatcccggcggggtacgggaggacgggctgccggcggcgctctccgatccggccagactggcggcgctggaggccagcgggatgatcggcagcgccccggagcagatgttcgacgacctgacgcgactggccgtgtcggtgacgggctcagccatctccgcgatcacgttcgtggggaagacccgcacctactggaagtcggtgccgcacctgccctacggcaccgtggacgagtggcagaacgcggtgggcgacagcttctgctacttcgccgtcggggcggagggcccgttcatcgtcgaggacgccgagaaggacccgcgcaccgccggacaccaggccatcgggccctggggcgttggcgcctgggccggattcccgatcgtcaccagcgacggtcaggcgatcggcaccatgtgcgtcatcgacgcgagcccgcgccagtggaagacccaggacctggaaaccctcgccgtgctggcccgcgcggcatccaacgagatcaacctgcgcatctccctgggcgcgacacaggccgcgctgagcaccgcgcaggtggcgctgcaccaggcacagggggcgctggcgaactcccgcgaactggcccgctccctccaggacagccttctgccgccggcgctgcgcccggtacccggcatcgacgcctcggcgctctacctgcctgccagtggcgacgcggatgtggtgggcgacttctacgacctcttccactcccgcgggccgtggtggacggccgtgctgggcgacgtgtgcggcaagggcaccgaagcggccaaggtcaccgcgttggcccgctacaccctgcgcgccgacgccggggaccacctgtcgcccgcgacggtcctcggccggctgaacgccgccatgctcgcccagcgctccccgcgcttcctgaccgccgtccaggccacgctccgcaccacacgggccggcgtggccggacgcctgtgcctggcgggccacccgccggccctcgtccgccgcgccacggggcgtgtgcagcaggtcggcaccagcggcactctcctcggcgtcctggaatccgtccgcctcaccgacgtccgcttccggctcgccccgggcgacctgctgttcctctacaccgacggcgcctgcgaagcccgcccggacccccaggggcccgcacccgaccggtcgatcttcgacgaaggagccctcgcccaggccctggcggacacccacggcctcgacgcggccgc

SEQ ID NO: 4 - DNA, 20 bp, Streptomyces xanthochromogenes
acctcaccacccacccgata

SEQ ID NO: 5 - DNA, 20 bp, Streptomyces xanthochromogenes
gcggcccctgtccgcgaccg

SEQ ID NO: 6 - DNA, 20 bp, Streptomyces xanthochromogenes
ttctaatacgactcactata

SEQ ID NO: 7 - DNA, 20 bp, Streptomyces xanthochromogenes
acctcaccacccacccgata

SEQ ID NO: 8 - DNA, 20 bp, Streptomyces xanthochromogenes
gcggcccctgtccgcgaccg

SEQ ID NO: 9 - DNA, 14 bp, Streptomyces xanthochromogenes
gttttagagctaga

SEQ ID NO: 10 - DNA, 55 bp, Artificial Sequence
  Synthetic Sequence - Fw primer
ttctaatacgactcactatagacctcaccacccacccgatagttttagagctaga

SEQ ID NO: 11 - DNA, 55 bp, Artificial Sequence
  Synthetic Sequence - Rv primer
ttctaatacgactcactataggcggcccctgtccgcgaccggttttagagctaga

SEQ ID NO: 12 - DNA, 51 bp, Artificial Sequence
  Synthetic Sequence - primer 1
ctcggggccaccgccttgcccgcacctcaccacccacccgatacggagtgc

SEQ ID NO: 13 - DNA, 72 bp, Artificial Sequence
  Synthetic Sequence - primer 2
tcagggccggagccgaaggcgaagcgagttcagccgccaactgcccggatcgatcattacggggaaggagtg

SEQ ID NO: 14 - DNA, 74 bp, Artificial Sequence
  Synthetic Sequence - primer 3
ctcggggccaccgccttgcccgcacctcaccacccacccgatacggagtgcccatgaccgacacatcgccgctc

SEQ ID NO: 15 - DNA, 59 bp, Artificial Sequence
  Synthetic Sequence - primer 4
acaggggccgcccgatgccgggcggcccctgtccgcgatcagggccggagccgaaggcg

SEQ ID NO: 16 - DNA, 1853 bp, Artificial Sequence
  Synthetic Sequence - polynucleotide for modifying actinomycin
ctcggggccaccgccttgcccgcacctcaccacccacccgatacggagtgcccatgaccgacacatcgccgctcaccaccgacggcctggtacggatcctgttcggctcctcggccttccagatgctcaacgcgggccgcaacctgggtctgttcgcgctgctcagtcggcagtccgggctgaccgctcaggagatcggacgtgaactcggcctggcggaacgcccggtgcagatcctgctcctgggtactacagctttggggctgacggtccgtcagggcgagggctacctcaatgccgctgtcctgaacaacacgtttgaggacggcacttgggagatcatcgaggatctgatcgagtacgaggagcggatcgtccgccccgccgaggtggacttcacggagtcgctgcgccaaaacaccaacgtcgggctgcgccggatcgacgggaccggcaccgacctctaccaccggctgtccgcgaaccccgagcttgagcagttgttctaccgctgcatgcggtcctggtcacggctgtcgaatcccgtcctgatcgagcaggccgacctgaccggggtgcgccgggtcctcgacgtcgggggcggcgacggcgtgaacgccatcgccctcgcccaggccaaccccggcgtcgagttcaccgtcctcgacctccccggcaccgtggagatcgcgcgacgcaagatcgccgagcacggcttggccgaacggatctccgtccgggcggcggacatcttcgccgacgactacccggcggggcacgactgtgtgctgttcgccaaccagttggtgatctggtcaccggaggagaacgtgcgcctgctgcgcaaggcccacgcggcgctgcccgacggcgggcgcgtgctggtgttcaacgccatgtccgacgacagcggcgacggccccctgtacgcggccctggacaacgtgtatttcgcgacgctgccggccgcgagcagcaccatctaccgatggggccagtacgaggagtggttcgccgcggccgggttcgtgaagcccgagcggctgccgggcggccggtggacgccgcacggcgtgatcagcgcggtcaagtgacgccccagcgagaaccggagtcggccatgtccctcaagtcccacgacgccccgccgaccggtggggccgcggcgtgccccgccggtccgcacatgatggatccggatctgctccgggaccctttcggcggctacggccggctgcgcgaacaggacccggtggtgcacggcaggttcgtcgacggcaccccggtgtggttcgtgacccgctacgacgacgtccgcgcggcgctgcgcgacccgcggttcgtcaacaccccctcccacgtgccgggcgagaagggcgcggacccgcgcgagggcatgatggaactcctcaaggtccccgagcatctgcgcggctatctgctcggctccatcctggacagcgacccgccggaccacccgaggctgcgccgcctggtgacccgggcgttcgcggcccgccgggtcctggatctgcgccaggacatcgagcggatcgccgaccggctgctggccgagctgccgcaccgggaggaggacgggacggtcgatctcctggagcacttcgcgtatccgctgtcgatcacggtgatctgcgagctcgtcggcatcccggcggccgacctcggccggtggcgggagtggggcggcgacctggtgtcgatgcggcccgaacgactccagcactccttccccgtaatgatcgatccgggcagttggcggctgaactcgcttcgccttcggctccggccctgatcgcggacaggggccgcccggcatcgggcggcccctgtcg

SEQ ID NO: 17 - DNA, 24 bp, Artificial Sequence
  Synthetic Sequence - Fw primer
gatcggtctgtcgcccctctacac

SEQ ID NO: 18 - DNA, 24 bp, Artificial Sequence
  Synthetic Sequence - Rv primer
gatactcggagttggtgcccgaag

SEQ ID NO: 19 - DNA, 51983 bp, Artificial Sequence
  Synthetic Sequence - pKU508acmCWacmM
tcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacggcctcctcacccggtcacgtttcgtcgtttctcctccacgcgctcggcttcggggccgcacctgcattcgtatgcggtcgcccggttacaggtgcggcacggcctgatggaggccgcatgtgagaggagaattgagcttgcagtgggcttacatggcgatagctagactgggcggttttatggacagcaagcgaaccttcatgagctcagccaatcgactggcgagcggcatcgcattcttcgcatcccgccctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcattttccgctgcataaccctgcttcggggtcattatagcgattttttcggtatatccatcctttttcgcacgatatacaggattttgccaaagggttcgtgtagactttccttggtgtatccaacgggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccagaattcgaggtcttcgccccctaagcgtgtccgtcgccgacgccgaccggcacgggcacttcagcgccccattccttgcccccgaaggagtggggcgctggccgtttagggtggccgtatgaagctcatttggcagggcgaagacatgcccccaggcgaagccgccggaacccctgacgccgtcctcgaagcgctcacagacgccgtcctagcggcctacgccgaccacacgcggacgacccgcgcgcacctgatgttgaacgtcgtcgccccgctccgcgaacggctcatcaggtacggcgtgccgacggtcgaggcgggcggcgtatgggccaaccaggaagggccggtaagcgtcacggcacgcgccgactgagagacgtttccgcaggtcaaccccgttccagcccaacagtgttagtctttgctcttacccagttgggcgggatagcctgcccggcatgagcgtgaaggttgaaggcatggtcattctggcaggcggctacgaccgacagtcggcggaacgggagaacagttcgaccgcttcaccggccacccagcgcgccgcgaaccgggggaaggctgaggcgctggcgaaggagtacgcgcgcgacggcgtcgaggtgaagtggctgggtcacttcagcgaagcgcccggcacgtcggcattcacgggcgtcgaccggccggagttcaaccggattttggacatgtgccggaaccgggaaatgaacatgatcattgttcattacatttcgcgcctcagccgcgaagagccgctggacattattccggtcgtcacggaattgctccggctgggcgtgaccattgtcagcgtgaacgaaggcacattccgccccggcgaaatgatggaccttattcacctgatcatgcgccttcaggcttcgcatgatgagtcgaagaacaagagcgtcgccgtgtcgaacgctaaggaattggcgaagcggctgggcggacacacggggtcgacgccgtacggattcgacacggtcgaggaaatggttccgaacccggaagacggcggaaagctggttgccattcgccgactggtgcccagcgcgcacacctgggaaggcgcacacggcagcgaaggggcggtaatccgctgggcgtggcaggagatcaagacgcaccgcgatacgcccttcaagggtggcggagccgggtcgtttcaccctgggtcgctgaacgggctttgtgagcggctgtaccgcgacaaggtgcctacgcgcggcacgctggtcggtaagaagcgcgccggttccgattgggaccccggcgttttgaagcgcgtactcagcgacccgcgcattgccgggtatcaagctgacatcgcatacaaggtgcgcgccgacggttcgcggggcggcttcagccattacaagatcaggcgcgacccggtcaccatggagccgctgaccctgcccggcttcgagccgtacattcccccggcggaatggtgggaacttcaggagtggcttcagggtcgaggacgcgggaagggtcagtaccgggggcaatcgctcctgtcggcaatggacgtcctttactgctacggctccggccagctcgacccggagacgggttacagcaacgggtcgaccatggcgggcaacgtccgcgaaggtgatcaagctcacaagtcgtcgtacgcgtgcaagtgcccccgccgggttcatgacgggtcgtcatgctcgatcacgatgcacaaccttgacccgtacatcgtcggcgcgatcttcgcgcgcatcacggccttcgaccctgccgaccctgacgacctcgaaggcgacacggcagcgctcatgtacgaagccgcacggcgctggggagcgacgcacgaacgcccggagttgaagggtcagcgctccgaactgatggcacagcgcgcggacgccgtgaaggcgctcgaagagctttacgaagacaagcggaacggcggctaccggtccgccatgggacggcgcgcgtttctcgaagaggaagccgcgctgacgctccgcatggaaggggccgaagaacggcttcgtcagctcgacgccgccgactcccccgtgctgccgatcggcgaatggctgggcgaccggggcagcgacccgacgggaccgggttcgtggtgggcgttggcgccccttgaagaccgtcgggcgttcgtccggctcttcgtggaccggatcgaggtgatcaagttgccgaagggcgttcagcggcccggacgggttcccccgatcgccgaccgtgtgcgtatccactgggcgaagccgaaggtcgaggaagagacggagccggagacgctgaacgggttcacagcggcggcgtgacggcggcaccagcgcaacgggaaggggcttcggccccttttctcgtgcccggcgtcggttcgttgccctaagcaactgttcctagcgtcacgtcagcgccggaccggcaggcttcccacctgggcaaagagacggaattcccggggatcagctttatgcttgtaaaccgttttgtgaaaaaatttttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgccggatccacgtgatcgatgcatacgcgttcgaaagcttcgcgacgtacgacgtccgcggagctctcgagcatgctagcggccgcgacatcgacgacgaccaagccatcctcgtcctgaccgccaccgacccggatcccgaggcctgacccgacccggaccccgaccccgggcagaccccagcaccgccacgaaaccggcggatggcgacgacagccgcgcccgggtgcctgtcccaggcatcctgggcaatgagggcatcgatgttccggcccccagcagtacggccgtgtcccatgggtacgatcgcatcccgttcaagggcgggacgacatccacgcacggggagttcgcatggccaccatccatcgcaccaccatgacgccgacgaagctggaactgctcgccggctggctgccgaagcagagctggtacgtcggcgatgcggcaaagccggaactggtcaaggcaggtgggttccggttggacgatccgaagggtgcggtcgggatcgaattcatggtcgtggtggacaccaccgcgcaggagccggtggcctacttggtgccgatggcctaccgggatgctgcactggagggcgtccccgacgaggcgctgatcggcacgtccgagcacggcgtgctcggcactcgctggatctacgacggcgtccacgacccggtggtggtggcgcagttgaacgcgctgctgcgcggcgaggcgaccccgcagcgccagaacgagagcgacacccccgacccgaccgtcaccgtgcacggaacgggtaccgccgacggccgcgacgttcggctccaccgcgtcctgcggtcgggggacacagctcgcccgtcgtccgtacaccttgtcgccgggtggacctggccggacggaacggctgcgcggggcgtaatcgcggggagttgagaccgcaggccgggcttggagggtggggtaagccggagagctcgtcacctctccggcgggcggccgagccgcgagcggcgatggtcggggaagcgttccgcgcgcggatcgaggagatcgatcaggtccatgagggcgtgctgcaacgcgatcagccggtacggcacttccgccgcccgcgccaccacctcgacactgtcactcagcgggccgaaccactcggtgaactccgcgtcctcgtcgagtcggcggcagaactccacgcacccaagacaggagatgccgtccgcggccggcatgagctcgccgatcgcccgctgatcgctgcggaagacgatgaagtgcccatcgaggaagcgtgtgttgccgccaccatcgttgttcagcacgttgctgatcgccgtgaagtgacccacgatccgccggttgagcgtgctgtccccgagatcgaggaactggacaccgcggcgcaggatttccacccggcccaggtactcgcccagtacatagagcgtgctgcgacgggcgtacgcgcgttcccggtcggagccgtcgcggaaatacacccgcaggaatccacggtccacgatgttgtagaggcgggactgcagatcgaaggcggcccacaagagcggatcacggtggcggcccatcaggtcctgctgcgtggtccggctcttcaactcgtgtgcgagcagcgcttgtcggcggctggagcgggccgagaaccaggcgccgccgagcgccaccacacctgacagggacgcgacgaccaattcggtattcacaggcccgggagcgtagcgggcacgacagccacctgcactggtttcgccgcctccggatgcgctgggaggcatgcggtgacacacacccgccgagaccttcctcaccctgccgcgtaggcctgctcgctctcccggaaacgctcgatcaggagcggatccatcagggtggaatccggcccgtagaacttgccgaacttcgccctgtattgctggaaccagtgcggacgttcggagaggaagaagatgtcgtggagggcgatggagcggatcgcgagcaagggtacgggtgcccggctcaccgcgaagcgggagttgcgtgccgctgttacccggcagttctcgtggaactggccgatcatgaggccgtgcgccacgaagtcgtccttcacacgggcttgggcctggtcgagcagtcccgtgtcctcgctgcgcagatcgggcaggacgatcaccatggcctgcagcatggggttccggccctgccaggtcgtcagctcgtactcccgcaggccgctgcgtgcgatctcctcgaccagttcgaggctcggggcatgcccgacaaggcgtatgcggacctccatggtccggtttttgcgggagggtgacacgaacgggcagatcggcccggtgcggccgatctccggatggctcgccgatatgtactcgctcagccacgagtcgaccgactccagcgcggactcgaagtcgacgtcggatgtcttgggcgtcatggcgtacccgtcaggccggcaacgcgatcgacggcgatgcgggcgagccgatggatctcggacccgtccggaatacgggtgttgatgaagttgaggatgccgtccttcggcacgatcacccacgcggtctcgatgcggaagtggtcgacggcggacagccagcgcacgacgaagccgccgtcccccaactccaccgcccccaggtccactttcgtgatgtcgagttcgacgccgtcgttcagccccatccggtagtgctcgcagtcgcgcaccgcttcgaagagctcctggtacacctgccggcaagccccgttgccgaactgtgcgacgtggtggaacaccacggaacccacgatgttcgtgaacagggcggaggcgtgatgcgtcgtctccgggtgcgtgccgtgcgtgagcatgttagtcaggtcgacgatggcccgccccgactcgtcgccggagacgaacaccggatcgtaggcggcgctcggctcctccccgtagaacgactcctccaagtccttttcgttcagcagtagttgtgcgaaagcggcgtcgtccatcgcgttctcctatcggcgcacgaggctcagcggtcgcatgtcggtccagtgctcctcgacgtaggtgaggcagctctggcggtcggtctcggcgagcgcgacggcccagcccgcgggcacctcggcgaacgcgggccacagcgaatgctggccttcctcgttgacgagcacgaggaaggtgccgtcggaattctcgaaggggttgctcatcattgctccctgcgtagacagttgcggtgggggagggcggggcccgtcactcctcgtgcagtaccgccgtcacgtacttgtggatgccgttgatcgaggtgaacagcgtcatgtcctcgaactgcgggtcgatgacgatgccgtgccgctcctccagctcgtgctggagaaggaccagggtgaacgagtcgatcaccagctcggcatcgtccggcagctcctcgggcggaccgagacccgcgttctcggccaggatcgctctgatgtcgtctttcgagatcaccggttcccctgtctgcgccatgtggtctgccctgtcgtcatgcctggggcgatgcttccgcggtgatgcgtccgtgcatcagtgcacggtcgcgtacgagtttgcccatcgagttccgcggcagctgccgcgtgacgaagtaccgcttgggattcttgaccgggctcagccgctcgcggcaccagtcggtgagctcgtccgaggtgggcgcccggtcggttccggtgcccacgaacgcctcgatgaactgctcatgggtcaccaccgcctcggtcacccccggatgggagaggaggacggtctccacttccgtgaggtcgaccttcagcccgcccaccacggccagtgaatcggccctgccgagcatgctgaggactccggtcgccggatcctggtggacgcggtcgtaggtccgcagccaaccgtccgagaagcgatcggcatgctcgctgaacaggtacggcgaacgttccatccggacgaagagctcctcgtcgagcaccttcacctcgaccccccgcaccggaggccccaattggggcggtggggagccgccggacaggtcgcccgcgatcaacccgatctcggtcacgccgtagaccggactgatcggcaggccgaaccgggcgtggaaacgttcataggtctcctgcggcacgcgttccccgccggacaccgcgagccgaagggcgggcagttcgggccgccctccggtccgggaaaggagatcgtagtgaacgggcgttccgtacacggcagttgcctcggcccgcgccatcaggcgcaccacttcggccgggcggagactgggcgggacgatcagggtcgccccgaccgcgagggcgtgcaggacgccgctgaccaggcccatgttgtgcatgacggagttcagcagcatcacccgctcacccctgccggggattccgggcaacgcgctgtgccggtcgagttcggccaggacggagtcggccgggcggccgatcaccttgggtcggcccgtcgagcccgagctgaactgcaccaggcggatgccgtcgacggccggtaggccgtcgggaagccgctgcaccgagaacccggactcctggcggaatccggcgaccggcccgtccgccccggtcgccacgacgaggtactgcggtcgcaccaggtcggccagcggttcgtattcggtgggcgtcagccggaagtcgaccagaaccacctgtgcgccgcggctccacaaggcgagcagtacttggagataggtgaagctcggcttcattcgcagaagaaccgagctgccgtctgtaatgccttgtttccgaaaacgctcaccgaaatcattgactgctgcacgtaactgcccgcgcgtaactggcgcactggaaattgcccacagttcgtcgtccgcgccccggctcagcagatggctaccccaccatgtatttgccatacacgcttccttttgcgaaaacaggtccgcgaaacgttcgatcaggcatttagtgaaggtaaagctagcaccgccgagaaacctcgggcaagcactcggacgtcaatccgttccagccccggcaaagacgtgcagccccagtcggatgccctgacggttggtgcggaccaagcctcgccgtgggcctccacgcttgcacgtgaagaacttggcgggtcccctgcctgcttcgatcgctgtgccggtgtgctccttcggtccccgccgaccgatttcttgtcccgccggcggtacaaccggcggcacatgcgcatggcgtaggcctttgatctccttgaccccctggggctcccgagttaggttgcccttgattgcgtttccgaggcgttgcgctcgattccggtgcgtactgcgagatttctcgcccagttggcccgacgatcgccttacatccattacccggtcgcatttccgacgtgtcttcatcgggccgtcgtgagcttttcactcgtgtctctccggccgtccgtgcgggggaaacttcggaggtggagacttgttcgccagtcctggcaccacgctgccgctcacggccgcgcagcgcgagatatggatcgccgagcagcgtcttggggaggggaatcgggttttccgggtgggtgaatacctggagatccacggtcaggtggacccggcgctgttcgaacgggcgctggtgcgcgtcgtcggtgaagcggacgccttgcacgtcaggttcgtcgaggaacgcggcgaggtccggcaggtcgtccacgagctcggcgactggtccccttccgtggtcgatctcagcgcggagccggaccccgaacgggccgcccgcgactggctggacgaagcggtcgcccggccgatggacctggccggtgaccggctgttcgagcacgcactcctgcggctgggcggcgaccgctgctactggtaccagggctaccaccacgccgtgatggacgccttcgggtcgctgctgatcacccgccgggtcgcggacgtctacacctcgctcgcccagggcaggccggtgtcaccgagcccgttcggctccctgtccgacctgctggccgccgaaggggagtaccgcgattcgagacgattcaccgaagaccgcgagtactggacggagctcttcgccgaccgcccagcgccggccgggatcgtcggtcgaccctccaccacccccgaccgctaccttcggcacaccgcaggccttgaccatgccgggctgagcgagctgcgcgaagcggcccgccgcgcacgcgtcccctggtcgtacctggtcatcgccgccacagccgtccatctgcaccggatgaccggcgcgtcgaccgtggtgctcggtctgccagtgacggcccgcctgaaccaggtccagcgccgaacgcccggcacggcggccaacgtcctgccgctcaggctcgcgctccgatcggagatgacgcatcgtcaactcctcgatcagatcggggaacggatacgcgaactcggcacccaccagcgctatcgcgccgaggacctccagcgcgacctcgaactgccgggccacgcggggacctggtacgcaccggtcgtcaacatcatgtccttcgactacgcggtgaccttcgccggcctgtcgaccacggcccacaacctctcctcgggactggtgggcgacctcacgttcgccgtgtgggatcgacgcgacggcaccggactgacgctcgacctcaacgcccaccccgagatctgcgcggaccatgaattggccgcacaccagcgccgtttgatcgccgcgctgcgcgccgtcacggcggcggacccctccttgccgattggccggatcgacctgctcaccccggaggagcgggccgccttcctggccgtccccgacgacgttccgccgctgacggaagccacactgacgacgcggttcgaggcgcaggccgacaagacacccgacgcggcggccgtcacctgcggcgaggcgtccctgacataccgggacctgaatgagcgggccaatcggctggcccatcgactggtggcccggggcatgggtccggaacacgtcgtcgcgctggccctgccccgctccgccgacctcgtcgtggccatcctggcgaccctcaaggcgggcgccgcctacctgcccctcgaccccgaatacccgtcggcccgtctcgcccacatggtcaccgacgcctgccccggcctcctcctcacgacaacggcggcccggtccaacctccctgccacacacggcactccggtcctccttctggatgccccggacagcttcaccgacctcgcgcacaaccccgctccggacctggtacccgaccaccccgcctacgtcatctacacctccggctcgaccggacggcccaaaggcgtcgtcaacacccaccggaacgttgtccggctgttcgacgcctcccgcaagtggttcggcttcgggccgcaggacgtctggacgctgttccactcgtatgccttcgacttctccgtctgggagctgtggggtgctctgctgcacggcggccgtcttgtcgtggtgccctacgaggtgagtcgggcgccgggcgccttcctcgacctgctggcggacgagggtgtcaccgtcctcaaccaaaccccctcagccttctaccagttggcgcaggccgaagccgaaggcgaatccggagccgaggcagtaggaggaccggaggccggaacgaacgagggtgccgaggcggtggatgcctccaggccgactcgccgactcgcgctgcggaccgtggtcttcggcggtgaggcgctgcagccgtcccgcctcgccgactggtacgagcggcatcccgacgacgcgccgacgctggtcaacatgtacggcatcaccgagaccacggtgcatgtgacgtaccagccgctgaaccgccggcgggccacgcccacggcggccagtgcgatcggcaccggcctcccggatctgcggacccatgtgctcgacggcgggatgcagttggtgccccccggggtggtgggcgagctgtatgtcgcgggaggcggcgtcgctcgcggctacctgggccgaccgggcctcacggccgaacgcttcgtcgccgacccgtacgcgtccgaacccggtgcccgcatgtaccgcaccggagacctggtgcgctggaatccggatggcgagctggagttcatgggccgcgccgaccatcaggtcaagatccgcggcttccgcatcgaaccgggcgagatcgaggcggtggtgagcagccatccgagcattgccgaggcggctgtggtggtccgccaggagcagtccggtgacgggcgcctggtggcctatgcggtcgcgcgcgatgcactccgggccgaagacgtgcgggagttcgcgcgggagcggctgccggagtacatggttccggccgtcttcctgctggaccgcatgccgctgacggccaacggcaagctggacaaggcggcgctcccggagccggactactcggtgtcggctgcggggcgggaggcgcgtacgccgcaggagcagatcgtgtgcgatctgttcgcgcaggtcctcgggctgccacgggtcgggggcgacgaccatttcttcgagctcggtgggcactcgctcctcgccacccgcctgatcgcccaggtccgggccgtcttcggcgtcgagctggaactgcgcctgctgttcgaggggccgaccccggccgcggtggccgcccgcctcgacaccgccgggcccgggcgcctagcgctcgcgccgcgggagcggccgacggccgttccgctctcctcggcccagcggcggctctggttcctggcccggatggaggggccgagcgccacgtacaacatcccgctcgtcgtgcgactgtccggcgagttggaccacgacgcgctgcgctctgcactcggagacgtggtggcccggcacgaaagtctacggacggtgtttccggagaccgaggacgtgccgtgccagcgggtgctggcccccgaggcggccgtgccgagtctgaccgtcgtgccgacctccgagaccgaactgccggcagccttggaagcgggcgcacggtacaccttcgacctggcggccgaacctccgctgcgggccgggctgttcacgctgtcaccgcgagagcacgtgcttctcgtcgtggtgcaccacatcgccggtgacggctggtcgatgggcccgctgtcgcgggagctgacggaggcgtacacggcacgtgcgcggggccaggcccccgaatggccggcgctggcggtccagtacgccgactacacgctgtggcagaacgaactcctgggcgaccaggacgaccccggcagcctcctcgccacccaactcgactactggacccgcgctctcgccgaccttccggaccaactcaccctgcccacgacccgccctcgcccggccgtcatgacctaccgcggcgactacctcaccgtcgagatcgacgccgcactgcaccaacgcctcgccgacctcgcacggtcgaccggcgccagcctgttcatggtcctccaagcgggactcgcggccctcctcacccgcctcggtgcgggggaggacattccactcggcagccccatcgcgggacgcaccgaccaaaatctggaccacctcatcggcttcttcgtcaacaccctcgtcctgcgcaccgacaccgcaggcaatcccacgttcacccagctcgtcgcccgcgtccgcgagacctgcctcgccgcgtacacccaccaggacgtgcccttcgagtacctcgtcgaacatctcagtcccacgcgcaccctcgcccaccaccctctcttccagatcatgctcgccctccagaacgcccccgagggcgagttcgagctccctggtctgcaagcggctgtcgagctgggcaggacgggcacggcgaagttcgacctgttcttcagcctcaccgagcagcgcggcgaaagcggcaccccccagggcatcaccggtgccgtcgagtactccagcgacctgtacgacgccgacgacgtccgggtcctcttcgagcgctggatccgcctgctggaggcggccacggccaacccgaaccgcccgctcagccaccttgccatcctctcggacgaggaacaccacgacgccgtggtcgagttcaacgacacggccgtgcctttgcccgagttgtctcttgcggagctcttcgtgcggcaggtggccaggactccggatgctcctgctgtgatggatggggagagctctctcacgtatgcggagcttgatgtccgtgcggaccggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggcagagcgcatccggcacgtcctcaccgacgccggggtctcactcgtcgtgaccgacgaagtctcgcggggtgaactcgggtctgtggcaacagagttgttggtcatcggccgcgagtttcgggaaggcgagcactcgccgccggacgtggctgttgcccctgatgccgcggcgtatgtgatgtatacgtctggttcgtcgggtgtgccgaagggggttgtggtcagtcatcggaatgtggctggtctggcgttggatgctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtctgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtcccccgcgacgatctgacgccggaaggccttcgcgagttcgcgcggcgaagcctgccgcagtacatggtgccgtcggcgttcgtcacgctggaccacttgccgctgacggtcaacggcaagctcgacagcgccgcgctgcccgcccccggcccggccgtgcgggcggacgtggagagccgtgagccgcgcaccgcgcgcgagcggctgctcgcggagttgttcgcgcaggctctgggaatggaccgggtcgggtgcgacgacgacttcttcgtgctcggcggggacagcatcgtgtcgatccgtttggtcagcctggcgcgttcggcgggcatcggattcacggtccgcgacgtcttcgagcagcggacggtcgccggactcgccgaagcggccactgaactggccctttctacaacggagttcgacgactcgggcatcggagcggtggaaccgaccccgatcatgcgttggttcgacgagcgcggcggcggaatcgaccagttccatcaggcgatgctgctggaggtgccggccgatctgggcgcggagcgcctcaccctggccgtccaggcactcgtggaccaccatgacgcgctgcgtctgacccggacgccgggtggcacggatggcaggtcgtggtcactggcggtcgcgtcggtgggttcggtgacggccgccgagctggtgcacacggtggacctcgacggggcggaccttgacgaggcgctcgtccgggaccatgcggaggcggcggccacccggctgtcggccgagcggggggcgctgctccaggcggtctggttcgatgccggaccggtccgccacgggaggctgctgctcctcgtcaaccacctggtggtggacggcgtgtcctggcgcatcctgcttccggacctcatcgccgcctgggaggcggtcgcgctcggccgcccgccccggctcgacccggtgggcacctcgctgcgccgctggtcggcgctgctggccgcggaggcccgacacccggcccgagccgcggaggcgaccctgtgggccgatctcctcgccgccgccgacccaccgctgacggccggccggctcgatcccgttcgggacaccgtgggccgggccagtgcgctcacgctctcccttccgcccgacgtcaccaccccgctcctgaccacggtgcccacggccttccacgccggagtgaacgacgtcctgctgaccgctctgggcctggcggtggcccagtggcgccgggggcacgcccgcgggaggcacacggcactgctggtcgacgtggaggggcacggccgggaggagatcgtcgagggcgtggacctgtcgcgcacggtgggttggttcacctcgctgtatcccgtccgtgtcgacccgggaccactggtctgggaggaggtcgtggaaggcgggcccgctctcggacaggcgctgaaacgggtcaaggaacagctgcgggcgctgcccgaccgaggcatgggcttcggtctgctgcgccacctcaacaccgggaccgggccggagctggccgggctcgccgtgccccaactcggcttcaactacctcggccgcttcccgtccgcgggcactccggtggtgcccggccatccgggatggaccgtggcgtcggaggccggcctgctgagcggcgccgatccggcgaccgccctggcgcacggcatcgaggtcaactccatggtccgtgacaccccggacgggccgtggttggaggccgtctggacctgggctcctgatctctggcccgaaccacacgtccgcgccctcgcggagcagtggttcctggcgatccgtggcctggtccggcacgtcgaccggtccggtgcgggcggccacacaccctcggacttccccctgaccgaactcagtcagcacgacatcgagcagctcgaagccgcgtggaggattcagaaatgacgtcgtcgggcctggaagacatccttcccctctcgcccatgcaggaggggctcctgttccactcccgctacgagcaggacggcgcggacgtctatgcggtgcagcacgtcttcgacatcgaaggcaccctcgacgcggcgcgcctgcggacggccgtccgcgccctcgtccagcggcaccctaaccttcgggccggcttccggcaggtcgaatccggagggaccgttcagctgatacctcgtcaagtggatgttccctgggccgagtacgacctcgcgtcgttgcccgccgccgaagcggaggcggaaatcctccgactcgccgccaaggaacacggcatccgcttcgacctggcacagccgccgctgctgcgcttcaccctggtgcggctggccggggcacaccatcggctggtcatgaccacccatcacatcctgctcgacggctggtcgatgcccatcctcgtccacgagctgctcacgctgtacggcagccacggcgacgccgccgggcttccccggacgaccccgtaccggcagtatctgggctggctcgcgcagcaggaccggccggcggcggaggccgcctggcgcgaggcgctcgtcggccttgagcagccgaccctggtcgccgcggtggatccggcgcggcccgcactcatgccggagcggatcaccgtcgagctggacgaggaccgcacgacggcactgacggactgggcgcgtcggcagggcgtgaccacgaacacggttctccaggccgcctgggctctggtgctgagccggcacacggggcacgacgacgtggtgttcggggccgtcgtggcggggcgcgacccgcaactgcccggtgtcgaggccatggtggggctgctcatcaccatggtgcccgtccgggtcgtgctggacccggcgcagtcgctgacggagaccgtcatgcggctccaggacgagcagacgcggctgaccgcgcaccaccatctcgggctgccgcgcatccagcaactggccggccccggcgacctcttcgacacggccatggtgttcgagaactacccctgggacgaatccaccgagctgccggacaccgggctgcgcgtcaccccggacctcggcagcgggcgggacgcgacccactatccgctcaccctggtcgcggccccggggcggcgcctctatctccgcctcgactaccgggacgacctcgtcgacagggcctcggccgccgggttcctcgaccggctgatccaggtgctcgacctcgtctccaccgccaccagcactgattccgataccggcaccggacccgccgcaaccgagctgcccgtcgggcggatcgacctgctcagcgatgaggagcgcgccactctgtcggccgcccccgacgacgtcgcgcgcacgacaggggcgaccctgccggggccgttcgaggcgtgggccgtcgggaccccggacaccccggcggtggtctgcggcgagacggccctcacctaccgggaactcgacgagcgggcgaaccggctggcgcatgtgctgacgacccgtggcatcggcccggaggacatcgtcgccctcgcgctgccccgctcgaccgacctcgtcctcgccgtcctcgccgtcctcaaagcaggcgccgcctacctgcccctcgaccccgaatacccccccgcccggctgacccacatggtcaccgacgcccggcccgcccttctcctcaccaccacggcgatccggtccagcctccccaccacgcacgacaccccggtcctcctcctcgacgccccggacaccgccaccgacctcgccgggcgccccacgcacagccccacccccgccctcgcacccgatcacccggcgtacgtcatctacacctccggctccaccggacgacccaagggggtcgtcatgcccgcgggcgtcctgctgaatctcctggagtggcaccaccgcgagatcggcggccaaccgggcaccaggaccgcgcagttcacggcgatcagcttcgacgtgtccgcccaggaggtcctttcggccctgctcttcggcaagacattggtgatacccgccgaggacgtccggcgcgacgcggcccgcttcgccgagtggctcgaagaccagcgggtggaggagttgttcgcgccgaacctggtccttgaggccctagccgaggccgcagtcgagcagggccgaacgctgccgcggctgcttaccgtcgcccaggccggtgaagcgctgacaccgagccgcctgtcccgtgagttccaccggtcggtgccgggccgggtactgcacaaccactacggccccaccgagacccatgtggtcacggcccacaccttggggcacgacccggacgactggccgttgtccgtgccgatcggccgtccgatcgtcaacacccgtgcgtatgtgctgggttccgggctgcagctggcggctccgggcgtggtgggggagctgtacatcgcgggtgctggcgtggcccgcgggtatctgcaccgccccgctctcaccgccgagcgtttcgtggccgacccgtacggcagcgagcccggtgcccgcatgtatcgcaccggggacctggtacgccgcaacgccgacggcaacctcgaattcgtcggccgcgccgaccagcaggtcaagatccgggggttccgtatcgaacccggcgagatcgaggccgtgttgaccgatcatcccgacatcacccaggtcgccgtggtcgtgcgtgaggacgagcccggcaggaccctcctggtcgcctacgtgacggcgcgggaggccctgcggcccgacgcagtacgggagttcacccgagaccggttgcccgaacacatggtgccgtcggccgtcgtactcgtggagcaccttcccctgacggcgaacggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccacctgggcggtcattcgctcctggccactcgcctcatcgcccgcatccgggccgccttcggcgtcgaggtcggcctgcgcacgctcttcgaggcccggaccgccggcgcggtggccgcccgcctcgacaccgccgggcccgcgcggctggccctgaccaaacagcaactgcccgacctcgtaccgctgtccttcgcccagcgtcgactgtggttcctgcacaagatggagggccccagcgccacctacaacattccgctcgtcctgcggctgacgggcgagctggaccgggaggcgctgcgcgcggccgtggaggacgtggtggcccggcacgacagcctgcgcaccgtgttccccgaagcccaggacactccgtatcagcacgtcctggactccgtcacggtcgagctgtccgccatcgacatctccgaggcggaactgccggacgccctcacgtcggcgtcccgtcatcccttcgacctggcggccgaacccccgctgcgcgtcggcctgttctgcctcgcacccgaccggcacgtcctcctgctcgtgctgcatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcgtctggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctggtttgcagattgatgtggcgccggggcggacgcatacggcgaagttcgatctgttcttcagtcttgccgagcggcgtggtgggcgtggtgagccgcagggcatcgtgggggcggtcgagtactccagtgacctgtatgacgcatctaccgtgcggtcgctgttcgagcgttgggtcaggctcctcgacgaggcgaccgcccatcccgagcagccactcgacaccatcgacctcctcaccaccgaagagcaacaacagattctcggcacctggctggacaccgaggcggaagtcggtgcggacctgttgccggcgtgctttgccaggcaggcgcgcaccacggcggaggccgtcgccctgatcaccggggacacctccctgacatacggggagctgaacagccgcgccaatcgcctggcgcacgccctgctgcgccaaggcgccggacctggcacggtgatcgccgtcgccctgccgcgctcggccgagctggtggtcgccctgctggcggtgctcaagaccggcgccgcctacctgccgctggacccggaccatccggccgggcgcaccgcgtacgtcctggcggacgcggaacccaccctgctgctcaccaccttggcgacggacgcgcggctgccgggcgacggctcgccacggcggctgatgctggactcaccggacgtacaggcgctggtggccgactgccccgacaccgacccgtccgacgccgaccgcaccgccccgctgcgcgccgcggacgccgcatatgtgatctacacgtccggctcgaccggccgccccaagggtgtcgtggtgccgcacggcgcgctgaccaacttcctggaagccatgcggcagaacgtgccgctccggccggacgagcggctgctcgcggtcacgacggtcgccttcgacatcgcggccctggagttgtaccacccgctgctctcgggcgccgccgtcgttctcgcgcccaaggaggccgtcccccagccgtccgtcgtgctcgacctgatcgcccggcacggtgtcacggtcatgcagggaacgccgtccctgtggcaactgctgctcgaacacgagccggaggccttgcgcggactacgcgttctggtcggcggagaggcgctgcccgccgccctggcccagtcgctgcgggccctcacggacgacctcaccaacctgtacgggccgaccgaaaccaccatctggtcgaccgccgccgacctctccggcgccacggcagccccgcccatcggcaggccgatcgccaacacccgggtgtacgtcctggacggccggctgcagttggtggctccgggtgtggtgggggagctgtacatcgcgggcgccggcgtggcccgcggctacctgggccgccccgccctgaccgcggaacgcttcccggccgacccgtacgggagtgagcccggcgcccgcatgtaccgcaccggggacctggtgcgctggaatccgggcggcgagctggagttcgtgggccgggccgaccatcaggtcaagatccgggggttccgcatcgaacccggcgagatcgaggccgtgctggccgatcaccccgccgtcgcccaggccgccgtcgtcgtccgcgaggacgagcccggggacgcccggctggccgcctacgtcgtcgccgacacctcgggacgcgcgtacgacgagaacgccgagctgaaccagctgagcgagtggcaggacctgtacgactccgtctacacgagcgcacccaggacggcgttcggcgagaacttcgccagctggaacagcagttacgacggacagcccatcccgctgcccgacatgcgggagtggcgcgacacgaccgtcgaacgcatcagggccctcagtcctcgccgcatcctcgaaataggcgtgggcaccggactgctcctcgccggcctcgcccccgagtgcgaggagtactggggaaccgacttctcgcccaccgtcatcaaggacctgcagcgccacgtcgatgccgatcccgccctcgcctcccgggtccacctgcacacccggccggcccacgacttcgacggccttcccacgggccacttcgacacgatcgtcctcaactccgtcgcccagtacttcccgagcgccggctacctggagcaggtcgtcgccaacgccctccgcgcgctggtccccggcggagccctcttcatcggcgacatccgcaacccgcggctgctgcgcaccttcgccgccgcggtcagtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcaccgacacccccacccacccctggccccggcacaccgaccccggcgccaccctcaccgatctgagccgtcacctcgaagccgagcgccccgccgggctccgcgtcaccggcgtcccgaaccctcgcctcgcgcacgagctggccgttcagcacaccctcttcgacgacacacctgcgtccgcggacagcggccggccgcacgtcacgctggaggccttccacgctttgggcgacgagcacggctactggaccggcgtcacctggagcactcacgaccatgacgcggtagacgtcctgttcgtcgaccggaacctcatggacgaaggcatccccgtaggcacctacgcccctgccggtacgggcacctcggccatgccgctgtccacctggacgaccaacccggccgcccgccgcggcaccggcgccctcgtcaccgaactccgcgaacacagccgtcagcatctgcccgactacatggtccccgcgttcatcgtccccctcgaccggctccccctgacggcgaatggcaagttggaccgggcggcgttgccggcgccggagttcacgtcgggcgggtcggggcgggaggcgcggacgccgcaggagcagatcgtgtgcgatctgttcgcgcaggtgctcgggctgccgcgggtgggtgtggacgacgacttcttccagctgggcgggcactcgctcctggccactcgcctcatcgcccaggcccgggcggtcttcggcgtcgagctggagctccgctcgctcttcgagggtccgacaccggccgaggtcgccgcactcctggacaccgctgggcccggccggctggcgctcacacggcgcgagcggcccgagaccatgccgctgtccttcgctcagcgtcgactgtggttcatccacaagatggagggccccagcgccacctacaacattccgctggccctgcgtctgacgggcgagctcgaccgcgacgcgctgcgggccgcactcgctgacgtggttgcccggcacgagagcctgcgcacggtattcccggaggccgagggagttccgttccagcaggtgctgcccgtcgaaacggccgtaccgcatctcaccgtcacaccgacgaccgaggcggagctgccggacctcctcacgtcggcgtcccgtcatcccttcgatctggcggccgaaccgccgctgcgcgcggccctgttcgagctgtcggcgcaggaatacgtgctgctgatagtggtccatcacattgcgggtgatgggtggtcgttgggtccgttggcttcggatttggcgggtgcgtatggggcgagggtgcggggggaggtgccgggttggtcggagcttggggtgcagtatgcggattacacgttgtggcagaacgagttgttgggtgatcaggatgatccggacagtttgttcgtgacgcagatcgagtactggacgcgggtgttggcggggttgccggatcagttgtcgttgccggtggatcgtccgcgtccggcggtgatgtcgtatcggggtgactatgtcacggtcgatattggtgcggagttgcatcagcggttggcggatgtggcgcggtcgtcgggtgcgagtctgttcatggtgttgcaggcgggtctggctgctttgttgtcgcgtctgggtgcgggcagtgatgttccgttggggagtccgattgcggggcgtacggatcagggtctggatgatctggtcgggttcttcgtgaatacgttggtgttgcgtacggatacgtcggggggtccgacgttgggggagttggtttcgcgggttcgtgaggtgtcgttggcggcgtatgcgcatcaggatgtgccgttcgagtatttggtggagcatctgaatccggtgcgtacgttggctcatcatccgttgtttcaggtgatgttggcgttgcagaatgcgccggaggcggtgtttcggcttcctgggttgcagattgatgtggcgccggggcggacgcatacggcgaagttcgatctgttcttcagtcttgccgagcggcgtggtgggcgtggtgagccgcagggcatcgtgggggcggtcgagtactccagcgacctgtatgacgcgtccactgtgcggtcgctgttcgagcgttgggtcaggctcctcgacgaggcgaccgcccatcccgagcagccactcgacaccatcgacctcctcaccaccgacgaacaccggcacgccgtcgtcgagttcaacgacacggccgtgcctttgcccgagttgtctctcgcggagctcttcgtgcggcaggtggccgggactccggatgctcctgctgtgatggatggggagagctctctcacgtatgcggagctcgatgtccgtgcggatcggttcgcgggtgagttggtcgcgcgtggtgtccggccgggtgatgctgtgggtgtgcttctccggcgttcggtggagatggtcacgacggtgctggggctgatgaaggccggtgctgtctatgtgccgctcgatgtgcgttacccggaagagcgcatccggcacgtccttactgaaacaggtgcgcgactgctggtcaccgacgacccggcggagttccaaccgggcgcggaggactgtgagttgatcggagcggcctcgatgggagtggccgggacggaacctggcggtcctgttgaggtggccgttgcccctgatgctgcggcgtatgtgatgtatacgtctggttcgtcgggtgtgccgaagggggttgtggtcagtcatcggaatgtggctggtctggcgttggatcctcgtttcggtgggtgggctcatgagcgtgtgttgttgcattcgccggcggcgttcgatgcgtcgacgtatgagttgtgggtgcctctgttgaatggtggggctgtggtggtggctcctgcgggggatgttgatgtgccggggcttgggcgtgtgattgctcggcggggtgtgacggcgttgtggttgacgagttcgttgttcgatgtggtggctgagctggcgccggggtgtctggctggggtgcgtgaggtgtggacggggggtgaggcggtttctggttcgtctgtgcggcgtgttcaggaggtgtgtccgtctctggtggtggtggatggttatgggccgacggagacgacgacgttcgcgacgtgttttcgggtgggtgcgggttttgtgggtgggtcggtggttccgatcggtggtccgatggcgaacatgcgggcttatgttctcgatgggggtctgcggccggtggtgccgggtgtggtgggtgagttgtatgtgggtggtgtgggtgtggctcgtggttatgtgggccggcctggtttgacggcggagcggtttgttgcggatccgtatggggtggtgtccggtgcgcgtatgtatcggacgggggatctggtgcgtcggggtgcgggtggggtgctggagtttgtggggcgggtggatgagcaggtcaagattcgtgggttccgtattgagccgggtgaggtggaggctgtgctggccggtcatccgggcatcgcgcagagcgcggtggtggtccgggacgaccatgccggcgacgcccgcctcatcgcctacgtcgtgccggacgtggacgggacaacccccggcgaggaagcggaacaagggcagatcgaggagtggaaggatctctacaactcgctctattccgagcccggttcggagttcggcgaggacttctccggatggaacagcagttacgacggtgagcccatcccgctgaccgagatgcgcgagtggcgtgccgccacgaccgcgaggatcaaggcgcttcgaccgcgccgggtcctggagatcggcgtgggtaccggactgctcctcgccggcctcgcacccgaatgtgaggagtactggggaaccgacttctcacccaccgtcgtagaagcgctgcgacgccatgtcgacgccgaccccgaactcgcccggcgcgtcaccctgcgcgtgcaggccgcgcacgagaacggcgagctgccgcaaggccacttcgacacgatcgtcctcaactccgtcgcccagtacttcccgagcgccgactatctggagcaggtcatcgaccaggccttccgcctgctcactcccggcggagccgtgttcatcggcgacatccgcaacccgcggctgctgcgcaccttcgccgccgcggtcggtgccgcgcgcgccgaagacccgtcggacacggccgccgtccgtcgtgccgtggagcagagtctggtcctggagaaggaactcctggtggacccggagtacttcaccgccctcgcccaccacatccccgacctggcgggcaccgacatccagctcaagcgcggcgcctcccacaacgagctcacccgctaccgctacgacgccaccctctacaagaccggcatcaccccccaccccctcgacgacacccccacccacccctggtcgggggatctcacggacctcgctgagcagttgggcacgggacgggccgatcggctgcgcgtgaccggcgtcccgaactcacgcattgcccacgatctcgccgtgcggcaggcgatcgagggcgggacggcggctcccggaccgctcgtggccggtgtcgatccggaggaactgcaccggctgggggagcgccacggttactggaccgcgatcagctggagcacgcacgatccggccgccgtcgacgtgacgtacgttcggcccggactcctggcagagcgcgtcgccgtaggtacgtacgcctccgcgggcagcgccgcttccggtatgccgctgtcctcctggaccacgagcccgggcgccggccgcgacacgggtgctcttctcatcgccctgcgcgagcacgcccgtcggctactgcccgactacatgcgccctgccgccatcgtgcctctcgaccggctgccgctcaccgccaacggcaaactcgaccgcgccgcgctgcccgccttcgcgccggagcaggccgacatcggacgggcaccggccacaccacaggagcaggtcgtgtgcgagctgttcgcggaggtgctcgggcggccggtcgtcggagtggacgaggacttcttcgccctgggagggcactccctgctggccacccggttgatggcgcggctgcgggccgccttcggcgtcgagttggggctgcggagcctgttcgaggcgtcgaccccgggcgggatcgcggcccggctcgaagccgacgactcggacggctcctacgaggtgctgttgccgctgcgcccccggggcacacgggcgccgctgttcttcgtccatcccggcggcggcatcagctggtcgtacagcgcgctgatcaaacacctgggcccgcagtacccgctgtacggaatccaggcacgcagtctggcccggccggaggccaggccgggcagcatcgaggacatggcgaccgactacgccgaccagatccagaccgtgcagccgcacggcccctaccacttggcgggctggtccttcggcgggctgtgcgcgcacgcactggcagccgagttccggcggcgcggtgaaccggtggcgctggtcgcggtgctcgacgtgatccccaactggcaggggctgacccacgcggacgtacctgcgcccgacgaccgggtgatgctgctgtaccacgtgggtctcgtcgacgacggcacccaacggcaggacgacgaggagatgaccttcgccagggcgcgggagatcctgcgtcgccagggcagtgcactggccagcctggacgaggaccggctcgcggcgatcaccgagatctcggcgaacaacacccacctgaccatcgactaccggcctggcgcgatcgacggcgatctgctgctgatcgcctgctcggagcagcaggacccgccggtcgacgctgcggcctggcagccgtacgtccgtggcaccgtcgaagcccatgtagtgcccggcgagcacgggtccatgctgacccggccggacacgctggccgagatcggtcggatcctgtcggccaagctccacgaactgaccggcgacgagtgacgttcccagcccgccttccaggctcgccccgtccgccttccgcgtggaccgatcccgaggactgacaacgatgctgaccgacgtgctctccgtcgacttggccaaggcgctagaggccgccgacggctggctgagcgactacatccgccgaccacacagcgaactgggacgtaccggcccggtgtgccccttcgtggaacccgctcagcgcgccggcgcgctggagatccgggtcagactggtgggtcccacccccagccagaccctcatcgacgagatcgtccgctgtgggctggacgagttcagcgaggtcgactggaaggccggcaacccgaatctgcgctcgctgctgctcgtcctgccggacctgccgcccgagcatctgcacctgttggacgaggcgcacgccgcgctcaagcccgagagcgtgcaccgcggcctgatgatcgcccagttccacgagaagtgccgggagaaggccgcccgcaacccggagttcgaggtgagctacgcccccgtgccgatgatggcggttcggtccatggcgatccatgacgtgctgttcctcgcggaccggcgcgagtggttcgaggagtacgcgagccgcttcggggcgcgttaccggaactcggcgcacggcatcgatccgctgctgaccgagatgtacgaacgggcccgtgccaagcatgggctgatgaccccgtgatgcacggcggattacggcccgggggtgcacgatgacggacaccgccgtgtaccggggcatggatcaggccacgctcgaccggcagtactcgccgagttcccgggtgccgagcctcggggcctacctgcgggagtacgcacggcggagcacggccgcccgccggtgccacccggtgcgcaccgggctcgcctacggcggacatccggccgaggtgctggactactttcccggccgcccggggcccgggcggccccccttgctggtgttcgtgcacggcgggaactggcaggcgctcggccgtgccgaatcggccttcccggtaccgccgttgctcgccgccggtgcggcggtcgcggtggtcgagtacgggctggcgccggacgtcggactcgacgcgatggcggggatggtgcggcgcgcgatcgactggctgctgcaccacgcggaccggctcgggttcgcctccgaccggctccacctgtgcggcacttcggccggcgcgcacttggccgcgatggccctgttgcccggtccgccgcaggggccggacgtctccggccggatcgccggggcggtgctgctcagcggcatgtacgaccttgagccggtccgtctgtcgtacgtcaacgaggcgctgcgccttgacgaagcgggggcgcgccgcaacagcccgctcggcctgctgccgccgcggttgcctcaggtggtggtcgcccgcgggggcaacgagaccgaggagtacgtccgccagcacgaccggatggtcgcggcgctgcgtccccgggccgcggtgtgcgaggtcgtcgccgagcggcgcgaccacttcgacctcccctacgacctgggggtgaggggcaccggactgggcgacgcggtgctcgcgcagatggaactgggaggagcggccacatgaccaccgaacagcccgcgcagggctccgggcagagcgccggatgcccgctgtccgcagccgtcccgccgggcaccaccccgtacgcgtactacgcgcgcatggacgagctgctggcgctgcagcatccgctcagcccggcggacaccgagccggggttcatcatcatgagccaggtcaaggagctcctcttcaagctgctgcacaccgaactgaccaccgcccgcgatcagttggacaaggaccggatcgacgccgcgctgtggaccctgcgcaggtcggcgcgggtgcagcaggtcctcctcgtgtcctgggagacgttctccgtgctttcgcccgccgagttcgccgagttccgggacgtcctcggatcggcgtccggcttccagtcggccggctaccggcgactggagttcctgctcggcaacaagaacccggcgatggcgcagccgcaccgggacacggccggtcacgacgcggtgctgaaccaactgcacgagccgagcctgtacgacgcggccctggccctgctggcccgccgggggctgctcacgccggacgactccgcggcgcgggacgccaccctgccgtaccggtccggcgaggacgtcgaggaagcctggcgggcgatctaccaggaccggcccggccaccacgatctgcatgcgctggccgaggcgctgatggacacggcggagcagttcgcgcgctggcgctacacgcatctcctcaccgtgcagcggatgttgggagccaagccgggcaccggcgggaccgatggcgtcgcctggctcgcccggatcagcgaacaccgattcttccccgagctgtggtcggtgcgctcgacgctctgacccaccggacaggtgccaggagcaccagggacacgacagatgccgacggactgaaggtgaggagtgccgatggagggcggaatcccgcgcgcggtggccgaggagctggacgcggccgatccgctggccgcgttccggaagcggttcgtcatcaccgatccggagttggtctacctggacggcaactcgctgggccgactgcccgcggccacgtcggacctgttgcgcgaggtggtcgagaagggctggggcggcggcctcgtacggtcgtggcaggaatggatcgactggggcgcccggctcggcgaccgtctcgccgcccacgtgctcggcgcccggcccggcgaggtggtgatctcggactcgacgtcggtcaacctgtacaagctggccggcgccgcgctggacgcggcgcgcggacggaacaccgtcctcatggacgccgaggactttcccaccaaccggtacgtcctgcaggggctcgccgaccagcgcgggctgcggctgcgacggctcccgtccgatctcgacggcggcctggatctcggtatgctgcgctccgccctcgaccgggacgtcgccctggtggtgctgtcgctggtgtcctaccgcagcggcgcgctcctggacatggcgaaggtgaacgaactggcccgcgcggctggcgccagggtgctgtgggacttgtcgcacgcggccggagccgtccccgtcgatctcgccgcggccggtgcggaactcgccgtggggtgcacctacaagtacctgaacggcggccctggttcacccgccttcctctatgtgcgcagtgatctgcacgccgtgctgcgccagccggtgtggggctggtacgggcagcgggagcagttccggatggggcccgactacgatccggtgccggggatcgagcgcttcctggtgagcaccccgcccctgctgtcgctggcggcgatcgatccggccctcgccctcgtggaagaggccggcgtggaccggatacgtacgaaggggctgctgctcggccggctggccgacgatctggccgccgcctggctcaccccgctgggcttccggtcggcttcgccggggacgccggagcagcgaggctcgcatgtgtcgctgcaccacccggacgcctggcggatctgtcagacgctcgccggtgaagcgaaggtcgtctgcgactaccgggtacccgaccggttgaggatcggtctgtcgcccctctacacccggttcacggacgtctgggacgcactggcacgcctccgcgaggtcaccgccgggcgcacctacgcgtcccgccccgccgacctggcccgggtgacctgaccgtcggcctcggggccaccgccttgcccgcacctcaccacccacccgatacggagtgcccatgaccgacacatcgccgctcaccaccgacggcctggtacggatcctgttcggctcctcggccttccagatgctcaacgcgggccgcaacctgggtctgttcgcgctgctcagtcggcagtccgggctgaccgctcaggagatcggacgtgaactcggcctggcggaacgcccggtgcagatcctgctcctgggtactacagctttggggctgacggtccgtcagggcgagggctacctcaatgccgctgtcctgaacaacacgtttgaggacggcacttgggagatcatcgaggatctgatcgagtacgaggagcggatcgtccgccccgccgaggtggacttcacggagtcgctgcgccaaaacaccaacgtcgggctgcgccggatcgacgggaccggcaccgacctctaccaccggctgtccgcgaaccccgagcttgagcagttgttctaccgctgcatgcggtcctggtcacggctgtcgaatcccgtcctgatcgagcaggccgacctgaccggggtgcgccgggtcctcgacgtcgggggcggcgacggcgtgaacgccatcgccctcgcccaggccaaccccggcgtcgagttcaccgtcctcgacctccccggcaccgtggagatcgcgcgacgcaagatcgccgagcacggcttggccgaacggatctccgtccgggcggcggacatcttcgccgacgactacccggcggggcacgactgtgtgctgttcgccaaccagttggtgatctggtcaccggaggagaacgtgcgcctgctgcgcaaggcccacgcggcgctgcccgacggcgggcgcgtgctggtgttcaacgccatgtccgacgacagcggcgacggccccctgtacgcggccctggacaacgtgtatttcgcgacgctgccggccgcgagcagcaccatctaccgatggggccagtacgaggagtggttcgccgcggccgggttcgtgaagcccgagcggctgccgggcggccggtggacgccgcacggcgtgatcagcgcggtcaagtgacgccccagcgagaaccggagtcggccatgtccctcaagtcccacgacgccccgccgaccggtggggccgcggcgtgccccgccggtccgcacatgatggatccggatctgctccgggaccctttcggcggctacggccggctgcgcgaacaggacccggtggtgcacggcaggttcgtcgacggcaccccggtgtggttcgtgacccgctacgacgacgtccgcgcggcgctgcgcgacccgcggttcgtcaacaccccctcccacgtgccgggcgagaagggcgcggacccgcgcgagggcatgatggaactcctcaaggtccccgagcatctgcgcggctatctgctcggctccatcctggacagcgacccgccggaccacccgaggctgcgccgcctggtgacccgggcgttcgcggcccgccgggtcctggatctgcgccaggacatcgagcggatcgccgaccggctgctggccgagctgccgcaccgggaggaggacgggacggtcgatctcctggagcacttcgcgtatccgctgtcgatcacggtgatctgcgagctcgtcggcatcccggcggccgacctcggccggtggcgggagtggggcggcgacctggtgtcgatgcggcccgaacgactccagcactccttccccgtaatgatcgatccgggcagttggcggctgaactcgattcgccttcggctccggccctgatcgcggacaggggccgcccggcatcgggcggcccctgtcggccggcgtctcgccttggcggcgcacggacgggtgctcagccgggtggtccgtctcccagggtgatggcttgcaccgggcatgccagcgccgcttcgtgcacctggtgcggcaccgcggtcgcggcgccttcgggcaccaactccgagtatccgtcgtcgtcctgagtgaatacgtccggggccgtcagaacgcactgcccggaccctatacacgtgtgatgatcgatactgatctgcatggaagaccgtctccgatggtcgggcggacaggggccactcagtccccgagtgcgtgcgcggcggcgcggtcgagggcgacgagtatccactcctcgaagttcccttccaccttcgccgcggcctcgcgccagcggtccacgcgatcgacggacacgctgatcctgggcagaccgacctgtgcgagcggggccgcccgactgccctgacggctctcccgcagctggagccggagccggttcctcgaccagccctgctcctccgcgcggtcgagccaggtgtccgcctgctcgggcgggagcgaggcgacctcggcatggtggccgaagctcaggttctcgcgccgtcgcccgagctcgaagtgccgtgccacccatgcgtagttgcgcagagtctgatagtcgagtccagcggcctgtacgccggtccggtagcggtcggagtaccgctcctggccgtacacgagccagtcgcccaggcaccaggcggatgagtcggcgatctggaagatgtgcagtccggccttctcccaccggtcgtacgacaggatggcgggaatcttcaggccgacccgtgtggtcaggatccgcccttgatcgttgctcccgcggcgagccgccgcctcgcccggtctgtggccgactgctaaatgactgtcacgctggtggtacttggcgatttgctgcacgggttttccccctggaattgagccattgccaaatctcgtcccagtcgtcagcgaccggggaccatgaaaggaccccggcccaaaaaccgtagattactgaacgatgtatcgtcaatgatcaattccggacggatgtgaccggttggagcgcaaaggagcccggggatgacagcctcatgtgtccgtgatctctaaaacccgtgtgcaatccctgcgcccggacccttacggcgcggacggggtcgctcgggccgggctgccgaggccgggccgttcggccgcggcgatcccgtcgaggatcagttccaggccgacccggaaggcctccttggcctcccgctccacatggggtgtcgcgtcggccggctccttccccgcgtcgtcggccgcggcagcggtggcgtgcgactccagcgccgccagagccgggaagcgctccgcgaagtccggcgccacctcggtgagcagagcggtccgctccatccaccactcctcgtcggacacccccgtctccggcgcggcctgccggcgctcggcggccacccgggccatgccccggacgaactggaacagcacggcgatgacccggcggactctcaagggctccaggcccaccccgcccaggatgcgcaccaccgtctccagttgcgcgtactcgcccggcccgagcaccggccgggcctgggagacctgcaggacccaggggtgccgcagatagaaggaccaggtgtcctcggcccactccgcgatcgccgcgcgccagcccgcggcggtgtcgtactcggtgggcagctcggccagcacccggtcgtgcatgagatcgaccagttcgctcttgctcggcacataggtgtacagcgccatggccgtccgtcccagccaggaccccaccgcccgcatcgacagggctgacattccctgctcctcggccaccgcgatgcccgccgccacgatcgtgtcgacgtcgagcgcgggcttgggcccgggtgcggatctggtgcccttcgggccggccgagcgccagagcagagccattgagcgacgggcgtcgccctgtccggcaaaaatgaccaccttgcaactccttactgcataaagtaacctcgccccgagcatctccttacggtgtaaggatatcaggtggacgtggggtcccgcctcgggatgcccggcactcttggaaggtttttgatggcagagctccctgtttcattcatccaggtcaccgacatcaagcacgtcacgccgcgcatggtccgagtgacgttcgacgcggaccacatcgacgactccgtcggcggcacgccggaccaacaggtcaagctctgcttcccccgatccggccagcgcgtgcccgtgctgccggaacagggtgacgacgcgaccagctggtaccaggcgtttctggccattccggaggacgagcggccctggatgcgcagcttcaccatccggcggcgccggccgggcaccaacaccctcgaaatcgacttcgtcctgcacggggacaccggccccgccacccgctgggccggttcggcccggcccggcagtcagctcggcatggtcggaccttcgacgctctactccgcacccgtctccctgacggactcgatcgcgaactccgactggctcctgctcgccggtgacgagaccgcgctgccggccatcggcaccctgctggaggcgctgcccgagggggcacgtgccctggccttcgtcgaagtcgccgacgccgaagagcggcaggacttcgagaccgccgggaacgtcagcgtcaactggctctaccgagacgggacttcgcccggccagagcgacaagctggtcgacgccgtgcgcgaggccgagttcccggccggccggccgttcgcctggctggccggcgaatcgggggccgtgcgggccctgcgccgtcatctggtcaacgaccgggagctggacaagaggtcgatcgacttcagcggctactggcggttcaagctcacccaggacgatgcgccgaccacggaggacatggccgaggcgcaggagcggctcgccgagttccaggccgccgccgacgcccgcccggaatgaaccgcacggaggggccgggcttcggctgggcccctccgcgcccgaccgagaagcggtacgcgccgacaagcggtacgcgcgaaagaaggctttcatgatcacagtgcgaggtctcgcccggcagttcaccctccgtggacggaccgtcgatgcggtgaagggcatcgacttcgatgtggcggccggggaactggtcggtttcctcggccccaacggggcgggaaagaccacgaccctgcgcatgctgaccacgttactgcgccccacccggggttcgggcaccgtggccggctgcgacctcctggccgacccgcagggggtccggcgcaagatcggttacgtggcgcagggcgggtccacctggccggagtccaaggtcggggaggagatcgagctgcaggcccgcctctacggcctgtcgcgggccgaggcccggcggaggggagcgctcctggcggaacgactcgacctcgccggcctggagcagcgtcccgcgaagacgctctccggcggccagcggcgccggctcgacatcgtgctgggccttgtgcacgagccccaactggtgttcctggacgaacccacctccggactcgacccgcagagccgggccaacctgtgggaccacgtccgccggatgcgcgccgaacagggcatgaccgtcttcctcaccacccactacctcgacgaggccgacgccctctgcgaccggatcctggtgacggacgacggcaagatcgtcgccgaaggaaccccggacgaactcaaggcaggcgtcgcaggtgatgcgatcaccgtcgacgtcgcggaccgggccgccgaggccgccgaagtggtgcgccgactgcccggcgcccacgacgtcaccgtcgtcgccgagaccgtacggctcagggttccccggggtgactccgtgctgccggagctgctgcgcgccctggacagccatcggatcacgatgacctcggtcaaggtgaaccggcccaccctcgacgacgtcttcctcagcctgacggggcgttccctgcgcgacggcgaggcggcgagcccggccatggaggccactcatgctgcgtgacacctggctgatctttctgcgggacatgaagctgtgtctgcgcaatcccacctggctgatcatcggcatcatgcagccgctgctgtacctgttcctcttcgggccgctcatggtgacggtggtggcccatacgcccggcttcccgtccggcaacgcctgggccgtgctcacgcccgcgctcatcgtgcagacggcgctgttcagcggttcgttcgccggagtcgggctgctcaccgagtaccgggtcggcgtggtggaccgcttccgcggaacaccggccagccggtccgcgctcctgttcggcaaggtcctggcccaggccgtccaggccaccgcgcagtcggtgctgatcgtcctggtgacgctgctcgtcttcgacctggacgcgccggcgtccggcatgctcctgagcctcgccatggcggcggtactcgccatcacgctgtcgtccggatcgtacgcactggcgatggtcctcaagagcgaggccgcgttcccctcgctgatgaacgccgtgctgctgccgctgctcctgctgtccggcgtgctgctgccggtcaccgcgcaactggctcccggctggctctacaacctggcccgcatcaacccgctgacccatgtggtcgacgccgaacgggccgcgttccgcggggacttgggctccacagggctgctgaccgggtccctcgtcctgttggcgatggccgccctggcgctcgtctggggcacgcggacgttccagaaggagaacgcctgacggcgacatcccgtgaggcgacagcctctctgcccgaccacgaccggggctctccggggagccccacccgtaccacccgaggagcacatgtgagtaccgacgccatcaaggtgaccggtgcgcgggagaacaacctcaagaacatctcggtggagatcccgaaacggagtctgacggtcttcaccggcgtctccggctcgggcaagtcctccctggtgttcgacaccatcgccgccgaggcccagcgccaactcaacgagaccttcaccgccttcgtccgcaacttcctgcccgaccacggccgtccggccatggacgccatcgagaacatctccgccgccgtcgtcatcgaccagaaacggctgggcggcaacccacgctccacggtcggcaccatcacggacatcaacccgctgctgcgactgctgttctcacgggcgggcgaccccgtgggcctgccgcccagcgcgttctcgttcaacgacccccaaggcatgtgcccgacgtgcgaggggctgggcagggtcgtggaactcgacctcgacgcgttcgtcgaccgcgccaaatccctcaaccagggcgcgctgctgcaccccgacttctccacggacagctggtactggctgagttacgtggagtccggcttcttcgacaacgacaagccgctcgccgacttcaccgaacgggagttggggctgctgctgcacggcgagatgccacagaagctccgcctggtctggcagggcaacacactgaacgtcaagtacgagggtctggtggacaagttcaccaggctctacatcaagaaggaggacatgtccgagcgcagccgccaggtgttccagcggttcgtacgctcggctccctgccgggactgcgcgggcacccgcctcgcgcccgccgcccgtggctgccggatcgccggccaccacatcgcggatctcacctccatggaggtcgagcgcctggccgcggtcctggcggggctggacctgccccacgtgaaaccggtcctgaaggacctggtctcccggcttcggcacctggtcgacatcgggctcggctatctgagcctggaccgcgagacccgaacgctgtcgggcggcgagtcgcaacggatcaagatggtccgccatctgtcgagcagtctgaccgacatgctgtacgtcttcgacgaaccgagcgtcggactgcacgcccgcgacgtgcaccggctcaaggaactcctgctcgaactgcgggacaagggcaacacggtgctcgtggtggagcacgacccggacgtcatggccatcgcggaccacgtggtcgacatgggaccgaaggccgggacgctgggtggcgaagtggtgttcgagggcagcttcaccggtctcgtcgaagccgacaccctcaccggccgccaccttcgccaccgcctgcccgtgaaggccgcaccccggcgcgcgaccggcgcgctcagcgtcaccggcgcgaacagtcacaacctcaccgatgtcacggtcgacttccccacgggcgtcctcaccgttgtcaccggagtcgccggatcgggcaagagttctctcgtcaacgacgacttcctgagccaacatccggatgcgatcgtgatcgaccagtccgcggtgacggggagccggcggtccaacaccgccacgtacaccggactcctcgacccgatccgcaagctgttcgccaaggccaacggggtcagtgcgtcgctgttcagcgccaattccaagggcgcctgccccaactgccaaggactgggcgtcctttacaccgacctggcgttcatggagacgctgaagtccgtgtgcgaggtctgccagggccgtcgtttcgccgatgacgtcctcggctaccgactgcgcggccggtcgatcagcgacgtactggccatgacggcggccgaggcggcggacttcttcaccgagccgaagctccgccggacgctgcacgcgctgaacgaggtgggcctggactacgtggggctcggccagccgctcagtaccctgtccggcggcgagtgccagcgcctcaagctcgccgccgacctgcaccgcgtcggcagcgtctacgtgatggacgagcccaccaccggccttcacctgtccgacacccaccgcctcctcggcatcgtcgagcgcatcgtcgaccagggcaacacggtgatcgtcatcgagcacaacctcgacatcgtcaagaacgccgactggatcatcgacctcggccccgaaggcggcagcggcggcgggaaggtcctgttcgagggaaccccggcggaactcctgaaggccgccggctcgtacaccgccgagtacctccgccgggacctttcgggcgcctgacgcgtaggtgcccgtccaccgccggggcgtggccggtgtcgtcggggccggatcgcggcggatcggacggaaggcgcctgacctgggcagaacctcaggtcaggcgctttcttcggctggggaggacgttcagcgtcctcgccaccgccgtcgtccggtcggcgggacgaggccagtcgagctgcctgggcccgtcccgcgccggtacaccgctgcgggcgaaggcccgcagctcgccgtcgcagccgacgacgtgaagctgcgggggaccctcgctggaccacgccggttctgctccgcccaagctggcgcggcgtcggccaggtgtcgttcctcggcccgcgactgacagccgtgcggatggtgtggttggctggcctgcgcgattgcggcgtgacgggcaagggaggcgaggcatgggcggagtggactatcccggcggggtacgggaggacgggctgccggcggcgctctccgatccggccagactggcggcgctggaggccagcgggatgatcggcagcgccccggagcagatgttcgacgacctgacgcgactggccgtgtcggtgacgggctcagccatctccgcgatcacgttcgtggggaagacccgcacctactggaagtcggtgccgcacctgccctacggcaccgtggacgagtggcagaacgcggtgggcgacagcttctgctacttcgccgtcggggcggagggcccgttcatcgtcgaggacgccgagaaggacccgcgcaccgccggacaccaggccatcgggccctggggcgttggcgcctgggccggattcccgatcgtcaccagcgacggtcaggcgatcggcaccatgtgcgtcatcgacgcgagcccgcgccagtggaagacccaggacctggaaaccctcgccgtgctggcccgcgcggcatccaacgagatcaacctgcgcatctccctgggcgcgacacaggccgcgctgagcaccgcgcaggtggcgctgcaccaggcacagggggcgctggcgaactcccgcgaactggcccgctccctccaggacagccttctgccgccggcgctgcgcccggtacccggcatcgacgcctcggcgctctacctgcctgccagtggcgacgcggatgtggtgggcgacttctacgacctcttccactcccgcgggccgtggtggacggccgtgctgggcgacgtgtgcggcaagggcaccgaagcggccaaggtcaccgcgttggcccgctacaccctgcgcgccgacgccggggaccacctgtcgcccgcgacggtcctcggccggctgaacgccgccatgctcgcccagcgctccccgcgcttcctgaccgccgtccaggccacgctccgcaccacacgggccggcgtggccggacgcctgtgcctggcgggccacccgccggccctcgtccgccgcgccacggggcgtgtgcagcaggtcggcaccagcggcactctcctcggcgtcctggaatccgtccgcctcaccgacgtccgcttccggctcgccccgggcgacctgctgttcctctacaccgacggcgcctgcgaagcccgcccggacccccaggggcccgcacccgaccggtcgatcttcgacgaaggagccctcgcccaggccctggcggacacccacggcctcgacgcggccgccctatagtgagtcgtattacgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataaagctgatccccgggaatt

SEQ ID NO: 20 - DNA, 55 bp, Artificial Sequence
  Synthetic Sequence - rap_M7_DH_3′_sgRNA
ttctaatacgactcactatagaggtgcacgctagcggacgagttttagagctaga

SEQ ID NO: 21 - DNA, 55 bp, Artificial Sequence
  Synthetic Sequence - rap_M7_ER-KR_sgRNA
ttctaatacgactcactatagccgttggcgtcgagttgctggttttagagctaga

SEQ ID NO: 22 - DNA, 156647 bp, Artificial Sequence
  Synthetic Sequence - BAC vector inserted with the polynucleotide encoding rapamycin synthesis gene cluster
gatccgtcggcgccgccacagcccgcccggccggcggggcagctccgcgatgaccgaggcgcccaggttcgccgcgatctcccggcgcagcacgggtcggtccgccaccaccgtgccgaccgcggccaccgcgagcccgagcgcgagcccgaggacgagcccgatcgcggcgttggtggcagcggccttcagcggggagtgccgcaccgcgcgcggggcgtccacgatctgcgtaccggcgatgaccttgggcgtgccggtgcgcgcctccgcggcgcgctggtcgaaatcggcgatccgcgagttgagctcggcccggcgggcgaagagcgactcgatgctcgccgacgcttccgggtcgctgcccggcgatcggtccccgatcgccttgttgacctcggtgagttcgtcccgtacgcgatcacgctggtcgagcagggccttggactcggctttcgcggcctcccgcatccgcctcacatggtccgcgacgaacgcgtcggccagcgccttggcccgggccaccgcttcggcgtcgctgtcacccgtcacatcgatctgcagcaggttgttggtcaagccggtgccccggtagtcccccatgaagtcctccggtctttccgtggacttgagggaccgcagggcctccccggcgatccgcgaggtccccagcagcgcgacgtcggtgcggatcagcgttccggtgtcgttcggctggtcctcctgatgcgccaccagcaccttggtcaccgcggtcggcggcgacggcagcaggacggccatcgccgcaccggccagcagccccagcagagccatggagcaccagaggcggcggcgcctgcgcaccgccaccaccagcgattggaggtcaaggagcggagtggcggccgacgactccgcggtctcgctcgtcgtcacttcgagcctcccgtcgcgtggccgcgcacggcgagttccagagtgggctcgtccggagggcggtcggcggtccgcgtcgggcgggcccgcaccgttccggcgacgacgatgccgacgacctcatgcccggcgtccgcacacgcctcggcgatgccggcgagctcctccgcggtccggctgcccgcgctgaggaccaccagggcaccggactcggtgtcgcggtccggcacgatcggccggtccaccgagacctccaccacccgcagcacggggtatcccctgctcgaagaggagggggaaggatcgcccttggcctcggcggcgagctgcccggcggcccggcgggcgatctcgtcgccgtccggtacgaccaccagcagccgccggggagccggcaactggtcccggaggcgggcgcacacccgccggtagcggatctgcctgccggcctcgtcgccggaccgccgcggggtcggcatgtcccaccgggtgtcgatgccgaggagccggcggatccgggcccttgggccgccgccttccggccggtgcgcgcgcccttcaccaggtacgtcgacggtgcccagcagtgtcgagcccagcgccgcggcgatctccggttcggtgcgcagccggcggctcatccgtgcggcggtgagatggccgatgaccgcgagcaggaagaacagcagcgctccggcgacggtgagctgcgtcctagtcggcggtgcctcgccggacggccgggccgccggccccatgacgaccatgttggatttgctggtcaccgggtcggcctcggtcagcttcttcatggcctcctgcagcgcgatgcgcagcttctccagctcggtgcggccctgcacgctctccacggtctgccccggatcggccgcgtcggccagttcggtgatgcggcggctggtctgcaccaccatcttccgcagtgtctcgggcccggccgaggcttcggagtcggtgtcgtcgcccgcgagccgcgcggcgaaggcgacgaattgctgggccacctggtcggagaggtgctgtgcgcgctccggggtgtcggccgtacccgagatcttgatgatgttcccgtcggctgccttggcgctcacccggtcccgcagctcgctgccgctgaccccggtccagccgagcgtggcggccgcgcggtcgaccaccaccgaactggtcgcgatctccgcctgggtgagcagctcgcgctcctcccacgcccccggcagcagtaccgacgccgacgccgtgtagcgcggcggaaacagcaaggaggtgccgtagccgacgagcgcccccaccacggcgaggatggtgagaagccgccaccgccgacggaaaatccgcccgatcgtgaccaggcgtatcgtgtcatcgctcaacggcgcggcctcctccgtgcccggtccgggtcgcccgccgacgcgtaggcggcgagcagcgacgcctgcgagttccgccaggagagcggcccgccgatccgctcctgaccgatcttgcccatccgggcccgccgctccggatcgtcgaggagcagcgcgatgagccccgcgaactcggcctcgtcgttggcgggcgcgtagacggcggcgtcaccggcggagacccgcgcctccttcaggtcgaacgagacgatcggccggcccatcgccatgtactccaggaccttgttcatggtcgacacgtcgttgagcggattgcgcgggtcgggagagaggcacacgtccgcggtggacaggtagcgcaccaggtcggcgtccggaacgcgcccggtgaactgcacctgcccggtgagcccgagccgccgggacagctccaccatcgcgtcgaaggcgtcgccggagccgacgaacaccgcgtgccagtcggtacgtccgagctcgtcgcgcagcttcgcgagggcgcgcaaggcgtagtcgacgccgtcctgcgggcccatgacgccgaggtagcacagcagatgaggcttgccgcgcttcaactccggctcgggcggcaccggttggaaccggtcgatctggggcgcgctgcgcaccacgaagacgtcctcagggcgccggccgccacggcgcagcgcgacgtcccggtagctctcgttcgtggcgagcacgacgtccgcggcccggtaggtgcgccgctccagcgcgcacacggcgcggtagagcagatccttgccgcggtcgaaccgggagaggtacagctcgggcaccaggtcgtgctggtcgaagacgaaccgcgcgccgcgccgcttcagccacagtgccggcaggaacagcagatcgggcgggttgcaggcgtggaccacgtcgaccgggccgaccttccgggccagccggaccgtatgccacagcgcggatccgtactcccgcagatagccggccggtccgccggtggccgcgcgcaacgggtagcggtggatccgcaccccgtcgatcaacgcctccggctccgtgtcccgcttgctcccccggggacagatgacgtgcaccgtccagcccgcgtcgcgcagcgtcgtgcactcctgccacacccgccggtcgaacggcaccgacaggttctccaccaggatcagcgcgcgccggcccgacccgtcaccgctgatcgcgttaccaggcaaggcccatgtaccccggttcggcccggcgcgcctcggcgtcgggaaggcggatgaggtcgacgatcaccgggccgtcgccatggggcagcgccgacagcacgtccggatccctggtcccgaccaggcacacctcggcgtgttcgagcacctggtcgacggagtccgcgagcagctgggagaggtgcggcagccgggtctcgatgtactcgcggttggcgccgagcagccgggagaggctcacgttggcgtcgtagatcttcaggtcgtagcccttgccgaagagtctctccgccagctcgacgagcgggctctcgcggaggtcgtcggtgccgggtttgaacgacagcccgaacaggcccacccggcgcctgccggtgcgctcgaccagctccaccgcgcgccgcagatgttcggagttggagggcagcacatgggcgaggatgggcaccgagacgtcggcccgctgcgccgcgtggaccaggctgcgcaggtccttgggcaggcaggagccgccgaaggcgaagccgggccgcaggtaggcggggctgatgttcagcttgcggtcggccaggaacacgtccatcacctggtgcgagtccaccccgagcgcctggcacaccgcgcccagttcgttcgcgaagccgatcttgaggccgtggaacgcgttgtccgcgtatttgatcgcctcggccgtcgggaccggcacccggaacacctcgccgggcaagccgtcgtacagcgccgtcaccgcgtcgccgctcgccgggtcgagctcgccgatgacggtcttgggcgggtcgaagaagtcccgcacgctcgtgccctcgcgcaggaactccgggttgaccgcgacgccgatgtccaccccggccgtgccgccgacgtacttctccaggatcggcagcagcaggttcaggcaggtgcccgggagcatggtgctgcggaacacgacggtgtgccggcccccctgctcggccccctcggccccctcggccagcgcggcgccgatctgctcggtgacccgctccaagtacgtggtgcacaggctgccgttgggctccgacggcgtgcccacacagaccagtgacacctcgctgcccgtgatcgcctcgcggacgtcgccggtggcgcgcagcgctccggtccgcacgacctcggcgatgagctcgccgatccgctcctcgaccaccggggccctgccgtcgttgaccaggtcgaccttcacctggttcacgtccaccccgatgacctcgtgccccatactggccaggcacgcggccgacacgcagcccacgtagccgagcccgaaaacgctgactctcatgacccgttcctccccccgggcaggcccttccggcccgccctccctgcgccgaccggacgacgcccgcccccgcgcatcagtaggccccctgcccctggagcaccgcacgcagcgtcttccacaagatcactgtgtccagggccagcgaccagtcctccacgtaccgcaggtccagccggaccgcttcctcccacggcaggtcactgcgtccgctgatctgccacagaccggtgagccctggcttgaccagcagccgccgccggatgtccgggccgtacgcggcggactcctccggtaacggaggccgcggaccgacgagcgacatcgatccggtgagcacgttgaagagctgcgggagctcgtcgatcgagtaccggcgcagcaccgcccccacccgggtcacccgcggatcccggcggaccttgaacagcagtcccgcgccctcgttgcggtcggccagctcggcacgtgccctgtcagccccggcgaccatggtgcggaacttgaggatggtgaactcgcggccgtccttgccgaccctgcgctggcggtagaacgccccgccccggctgtccaccagcacgagcagcccgacgaacaccatcagcggcgcgaacagcaccagcaggatcgccgcgcccatccggtcgacgacccctttgaccgcccggcggcccccggtgaaggtgggcatgctgacccgcagcagcgggatcccgagcaccgcgtcgatgtgcagccgcgggccggccacctccatcagcacgggggccacgaccatctcggcgtcgctgccttcgaggttccaggccagccgctgcagccggtccggtgaccagtgcgggtccggtgtgacagcgacgacacggtagccgtcgtggcggacgtgcttggcgacgtccgacagccggccgacgacccgcactccgtccagccggtcaccgtcgagcccgcgaccgtccgtcgtgcacaccgcctccacccgccacccgaggtgcgggaacttacgggtccgggtgatcaggtcgcgtacggtggccgggctcccggcggcgagcaccggtcgcagacaccgtccttccttccgctgtttgtgcagccagaggcgcagcagataccgcgcggacatggtgacgagcgcgatcgcggggatcgcgacgaagatccagagcttgatgttgcgcgaggtgagggcgatcccgccgagcgccagtacgacggtcgccgtgaacagtgagcgtccgagccggcggaattcctcggccccctgaccgagcacggccggagcccacgaccggctcaccgcgagcgcccccagcaccagcagttcggtgccgaatgcgagaattccccacttctcgtgccagttggccgcgtcccgggccccgaagaagttgccgatcgccgccaccacgaaggcggtggccacggtatcgctggtgatcacggtacgtcggtaccgctgctcccagtcggtcgcgggctgtctgattgccccgtccgccagacgcccgcgcgctgacggaaacgggttgagtaatcccccttgccgcacagaaccccccaaggtccccagtggttcgacgtattcgcctcacactgttctcgggaggtccccaccccccgtactgcccctcccctcgggaggcccccaccccccggcactgttccccctcgggggcccccgcccccgcgccgcgccgttcctccccgcgggaggcccccaccccccgcgcctgacatgccgactgcttcagagctgctcgaacaacccaccctggcgactgccgactgtccgtagatccggacccatagatcattatttgtcgccggtgtccaaacatgtgaagcacggtcaatctagaccatcggggggcccatctgaagagggaatgtgtgcaacttgtgcccaagatttgaagctggatccaccgaccggtgaccgcccgcgggccttggtgacgaagacgtgccttgtgacctgtggcaacgggagttttccgggccttcggccaactcactgccggtgccggtgggtccggccgggggtcgcacgcaggtgtgtgcgcgccccctgcggtccggacgggatcagcagttggaccgcaccgccgtcgaacccgtgccgcgcgccgctcatcggcccatcttgcagaaaccgcaagatgacgtgccagttcgggcaagtccggcgagagtggtgtgcgcccctccccgcacaaggagtcccgatgagcgcaacagacgccggtacgtactggaacgacatctacgcggcccggtccgcggccgtctccccccgccccaacgcctgcctcaccgcgaccgtcaccggcctgccgcccggcgacgtcctggacctggggtgcggcgacggcggcgacgccctgtggctggcgggccgcggatggctggtaaccgccgtcgacgtctcctccgtggccgtcgggcgcctcacccgcctcgccgccgagaccggcctcgccgaccggctgacggcacaccaccacgacctgcgcacgtccttccccgcgggcgtcttcgacctggtctgcgcccactacctgcacacccccctcgacctggaccggacggccgtgctgcgcacggcggcccacgccctgcgcccgggcggacgactgctggtggtcgaccacggctcgaccgcgccgtggtcatggacgcaggacccgcacatccggtacccgaccccgcaggaggtcgcggcggacctggccctggcccccgcgacctggacggtcgagcgagccgacgcccaggtgcgcgaggcgaccggaccggacggccgcacggccgaggtcaccgaccacgtcctgatcatccgccgtgacgcctgaaccggcacggccgtcgaaagcagccaccatgaccacctccccgcgcccccaccgccgccgcgacgccccgccgccgcgcaccgggaacaccgagaccgaggtcctgcgcggattcctcgactacctgcgcgactcgatcgccgcgaaggtcgaaggcgctcccgaacccgggatccgtacggccgtggtgccctcgggcacgaacctgctcggcctgctccaccacctcacctgcgtcgagcgggcgctgttcctcggcgcccggatcaccgactggaaggcgaccttccacaccgcccccgcgcagaccgccaccgccgtcgtcgcccgctaccgggagaccgtcacggaggcgaacgaggtcctcgacacctgtaccgacctcggcgcgccactgccccgctcccggccggacacaccggcccccgccgtccgctgggcgctcacccacatgatcgaggagaccggccgccacgccggccacgccgacatcatccgcgaactcctcgacggcgccaccggacgctgacccacccgcccacaggcaggaagccatgacatccgcgtcaccgaaccgcgcaccgcgcacggcccgacgggcggcatccgccgtcctggccctcgtcctgaccaccggcgcggccgcctgcgccccgcaaggccccgacgtcccctcctacgcctccgccacacacgacgacccggagttcaaccgcgcctttggccataagttcgccaccgtcgacggcgtgcggatgcactacgtgaagggcggcaaaggctccccggtcgtcctgctccacggctggccgcagacctggtacggctggtggccgatcatgccggagctggccgaacaccacaccgtctacgccatcgacctcccggggctcggcgacagcagcggctcaccgaccggctacgacaaggccaccctggcccgctatgtgcacaccctcgtcgccgggaagctcggcatgcgacacgcccgggtcgtcggccacgacctcggcgccgcggtggccttccagtacgccgcccggttccccgcggacaccgagcgcctcggctacctcgacctgccgctgcccggacccgccatggacgcgcgcacctaccgctccctgagctggcacatcgccttccactcccagcgccgcgtccccgaggccgtcgtcgcggacgacgtccgtgagtatctggcgctgttctatccgcaggtcgccttcggcggcacggcgttcggcggcacctccacccgatcgcccttcaccgacgccgagatcacggagtacgcccgcacctacggccgccccggcgccctgtccggcggcttcgacctctaccgctccctcgacaaggacgtccgcgacaccacggcggcggccccggtgaacacccccaccctcctcatgacggcccagggccaactcgcccccgtccgcgccaccgtgaccccccgcatgagcaacatcgtgcgcgcggtggacgtaccgaaggcgggccactggctcgtcgaggagaaccccgcgttcgtcaccaccgaactgctccgcttcctcgactgacggccaccacctcgcgtgtggtcacgcgggcctcttcagcgcggtgggcgggaccggtggcagctggtcccggcgcaggtgccagaggcgggggtcggacgtggcggccgcggccgatccgagggccagcgcgagcgccacgtccgcgggggtctccacgaatccggcggcgaccgacggggacatcgcgcgctgtgcccgctcgctcatccaggcgatgatcggggcgggcatgatctccaccaggtccggtgccccgcgcgagatcgcgtccgtggagcggggcagattggaccggtcggtgacgaacaccttcatcatcgtcagcaggcccagcggacggcccttctctatgagggagagccgggtgctgaccacgccgtgcgcgcccatgttcttgatgaactccagcgcgccccggtcctgggccagaccgggactgctgtccacgttcacgaacacggtcttccccgcccggaccagcgccggcaccaccttgggcagctgccccacggtgaacgacgcgaggatgcacaccttggccggtgcggtcaggaactgccgcagcggctgggtgccgatcaccgacgcgacgaccggcacctcggcgaagtccgaggcgagccggggcgcgagcgcggcaccggacgggaacgggcggggggccatggtgaagtcctgcctggggaaggagggcgggcggggctgctgggatcagtccagcggggacgtgccaaagcggctgggaaggcccagtttgccagggttgagaatgcccgccgggtcgagggccttcttcaccgcgacgaacgtcgcgaagcccgccccgagggattcctccaggtaggggccgcgcagcagaccgcagccatggtggtggctgagcgccgccgcatgcttgatcagtacggcgttggccgcgtcccacaccgagcggtaccactcgcggcgcgactcgggcgccacatcgccacgcagcgagaagtagacgcaggcgccgtcggtgtacgcgtgggactggtgggccgaggcggccagggtgccgtccaccgtctggatcgcggcgaccacctcgtcatagatcaccgggaggtcggcccaggacgcggccatctccagggtgtcggcgacgaagccaggacccggcttgaagccgtcggcggacttgccgaccagcatccgctcgtccagccagcgctcgaagaccacatcgctgtccagttcggggccgtaactcccgcagacctccttcgccaccttgatcgaggcgtcgacgacggccgggtcaccctcgtcggcgatgagcaggaggttggtctccgggtggccgaagtgggtgccggactccagcgcgtcgtacaggcgcaggacggccggggtggcgccacgctgcatgatcttgcggcaggcgtccaggccctccgcgaaggtcgtgaagccgtacgcggcggcgctcgcgtactccggcagcgggtggacccgcagccgggccgagacgatgacgccgagggtgccctcggagccgatgaacagctggcgcaggtccgggccgaccgcggcgcgggcgtagtcgccgtacgtggcacgcgtaccgtccgcgtgcacgacgtcgacgccgacgaccatgtcctcgatcttgccgtaacgggtggagagctgaccggcgccgcggcaggcgatccagccgccgacggtggagacggcgaacgcggacggccagtggccagtggtgacgccgtactcctcctgcagttgcttctcgaagaggtccccgaacattccggcctggacgtccacgatgttcgactcggcgtcgaagccggtgatccggttcagcccgcagacgtccaggacgacgccgccgaagacgggcagcgccgcgcctgtgacgttggagcggcccgcggcgggggtgaccgggatgcctgcctggtggcagacgcgcagcacggccgcgacctggtcggcgtcggcggcctcgacgatcacggcgttcggggtggcgggacggccctcggtctcgccgatcatcgacccggcccaccagtcacgggtccggatgacgacctcgtggcgggaggtgtggaccgcgtgcgcggcgtcgcgcagcgcctcgaccacgctctcggggacctcgaccggattcgtctggagggcggcctcgccctccccgatcggggtgttccgcgcccacctggcgaagctgggggcgccgacggtgtagtcgccccggttgaacgggtgggtgatggtctggcggctgatcatgctgctgctccttcgaggagtacggacttttcgtggcggacggctgcgaggtactcggcgaccgagcgctcggcctcggcctccgtgaggttcagttcccggccgaggatcgcgccgacggcctcggcggcgtcggcggaggcgtcccgggcgaacagccgggcgcgcatgcggcgggagagcacgtcgtcgaccgagcgggccagttcggcgcgggccgcgtacacgacctcggccttggtgtacgggagtcccgcgacgatcggctcggccagcgccgggtcgtcctggatcaggtcgccgacgaagcgggcctcggtgccgaagcgctcgcccaggtgcgcggccaggccaccggacgcggcgacggcttcggcgtcgtaaccggcgccgccgagcagcgggaggtgggcggtacggctcttccccctgcggccgaggaccgtcatcaccttgtcgatgacgagctcgcccatgtgccggctggtggtgagcttgcctccggtcaccgtcaccatgccggtgcggccgacggtgatgtggtggtcgcggcgcatatcgagggtggcgccctccttgccgccgaccagcgggcgcagaccgccgatggagccgaccacgtcgtccgcggtgagctttccctcgaaggcggtgttggcgccctccagcaggaagttcatctcctcgcgggtgcagtgcacatcgtccggggggccctggtagtcctcgtcggtggtgcccagcacgacgctgttgccccagcgggtgcaggtcgcgcggcgggtccggccggggaccggcacggtgaccgtgcagttgatccgcaccttgtcccaggggaccacgatgtgaacgcccttggccgggcggacccgcggctggtgaccgtcgtccgcctgtccgtcgagctcgtcggtccacaccccggtggcgttgacgacggcccgcgagcggatgtcgaaggtgtcgccgtccgccgtgacccgggcaccggccaccttgtcccgctgcgtcagcagaccctcggccttggcaccgttgaggatcgtcgcgccgagcgcggcggcggtgcggacgatggtgaggacgagccgggcgtcgtcggtgcgggcgtcgaagtacatcagcccgcccttgaggttctcggcgcgcagcgtcggcgcctgcgcgagcacctcgggaaccgtgagccgctggtggagtttgccgatccgccagccgccgaccaggtcgtacgtccacagcagaccctcgaagcccttggcgaggcgggcgtcgaagacaccctccttctccaggaccgggaagaggaacggcagccggtgcaccaggtgcggggcgttcttccggaagcggtgtcgctccaggagcgagtggcggaccagattgacgttgccctgctcgatatagcgcagaccgccgtgcaccatcttcgaggacttcgacgaggtgccggaggcgaagtcgtccttctcgatcagcgccgcgcgcagcccgcgcgaggcggcgtcgagcacggcgtacgccccggtgatgccgccgccgatgacgagcacgtcatacgtgtcgtcggccagccggcgcttgagggctgcccggtcgagcagcagtggcgttcggcgggtcgccgcggcgctgcgccgcggtttccgggcgggcatggtgatcacaacatcaactcctggtgttccggtacggcggccgcgtggtcggcgggccgccagtgcgacggtcagtggggggtgtgaggccgggacgcggtgcgctcgtcggagagcagcgcggggtcgtcgaggtgggcgatcagcaggtcgcaccaggcggcccgctgctcgacgcgttcggccgtggtgatcgcgggctcgaagaaccggccgtgcggctgtgcttcgacgacctcttcgagcgagggccacagcccccgggcgacacccgccagataggcggtgccgcgcaggctggcggtggcggagtcggagacccgctccagtggcagaccggtcagatcggcctgggtctgcatgagcgggtcgctgccggagactccgccgccgacccggagccgggtgaacggcgtgcccatggtgtcggcgaccccgtcgatcagatcgcacaccgagtgcgcgatcccgtcgagcacggcgcgtgcgagatcggcgcgggtggtggcgagggtgagcccggtgaggaccccggtggcctccgggtgccagaccggcgtacggaccccggcgagggccgggaggaaacgcggggtgcggccgggccggctggggacccggcccgcctcctcgccgagttcctcgggcgatgcgaacagccccaggtcgtcgcagagccagcgcagcgccgaacccgccgtggaggtgtatgcctccaggctgtagtggctgatgccgccctgccgccggccgggctgggcgagcaccccggagatgtccggccggggcagggcgggcgtggtgccggtcgccgcgtcgacgaaggcgccggtcccgtacacgcacatgccctgcccggcggcgagcccgccgagcgcgacgagtgcggcgtgctggtcgcccatggaggcggccagcgggacggcgatcccgagggcggccggatcggtggtgccgaatccggcgtcgtcggagcggagttccggcagcaggtcgagggggaagccgagatggctgatccactcctcgtcccaggcgtgccgctccagcagatagccaccggtggacgcggcgttggtcggcgtggtggcgtggacggcgccgccggtgaggcgccagatcagccaggtgtcgacggtcccgaagagcagccggccctggcggtacgcggcggccacctccgggtgcccggcgagctgccgggcggcccagaggaacggtgcgcgggcgccgaccggccggccctgccgggccagcaggacggcgtcccactccgcttcgtacgcggtgagctcatgcgcgtaccggcggtcctgccacaccaccgcgggcagcaagggcagcccggtgacccggtcccacagcatcgcggtggcccgctgggtggagagcgcgacagcggtgatctcgatgccctgcgcgtgggcccggccgacggcccggcccgccacctcggcggtggcggtccagatctccatggggtcctgctcgaccgagaggtcgtccggatggctgacgccgatcgaccggtaggacacctcgtgggcggcgccggagtcgagtacgactccggccctggtgcccgtggtgccctcgtcgatggacagcacaccgcgccgaggggcggcgccggggaacgtctgcgtcattgcagccctttcatcgcgatctgcgcggagggatacggaagggaaggggccagggggaccatcgtgcgatgcggcaggtccggcggcgggtcagcgcgcggtcgcggagagcgtcgcggtggcgtcgcggtcctgtccggtggcctcgtcgagggaggcggccttcatcggttcccacttgatcgtcaggcaggtgatcaggccgagcaggggtacggcggagagcgccaggaaggtgtcggcgagtccgagtgagtccttgatctggggaaagacatacaggccggtgacgctgccgaagctgccgatcattccggtgacaccggtgccgagcgcgcggacatcgctgctgtaggagagcgcggcgatggacttgccgttggccccggggcccgcggagtggcagaggatgaacaggaccggcagcaggaacgccaccgccagcgggaccttcccgaaggtcagccccatcgccaccagcgtgacgaagactccggcgaacccggcggcggagctgcgccgcagtccgaggcggcggccgaagtacgcggacagcagcccgcccgcgatgccgaaggcgttgaagaccatcgagccgatggtggccttctcgaagctctcgccgaagatcgtcaggctgatcagcgggaggtaccagccgacggcgaagtactgcatcgactggccgagcgagatcaccgaggagaggatcgtacgcggcaggtactcgcccctgaacagccgtccggcctgccggagcccgatgaccggcgcctcagccggggtgcgggtcatctcgtcgggcgtaccggcgacggccttgatcccgtagatcttgtccaggttggtgatcgcctcgcccaggcggcccttgccggccagccaggtggggctctccttgagcatcagccactggcccaggaggagcgcgacggcgacgaccgcggccgagccgaccgaccagcgccagatgtccgcgccgacgtccaggttgaagaagaccagggcgaggacgaggttgctggtggtggcgatgtaccagacggcttgccacaggttgagccgaccgcccagcttcgcgggggtgtactcggcgagcagcgccatggcgaccgcgaagtccatcccgtaggcgacacccaccagcacccggccgagccagacgacggtgaagtcacctgcgaaggcggcgagcagggcacccgcgatcgcgaacagcttggcgatgagcagcggggggacccggccgatgcgggtggcgagccagccgccgagggggttgaagaggatcgccagggccggagcggtggccgtgaggatcgagacctgggtgctggtgagctccatctgggtcgtcatgggcccaagtccggcgctcagcgcggcgttcgagtaggcgtcgaggaacagcccgccgaacacgaggaaccagatgatctgcgcccggccggtgatctttcccagcccatcgatgagcgcaacgacgtcggcgacgcttctgacgctcggacgtggtgccatgaatccgccttcggggaggacccatcggcggcacggggagcagaacaggcatgacgaaaccaccgacaggtggtctatgtctatgcctgtcaaaacactgcggaaagcgccgcagcacgtgaactgctggaagtacgaactacgccgcgagattaggtacgggtcaggagcaacgtcaagggttccggccaggaaacgtgacgggggccttcaccggaggaggtgacgcgagacgagccggccccgtggcatggcaagggacaagccgaccggcgaggtcagccggcgagcacggtgtcgagccagtcgaaggtgcgctggtggaacaggaacatcgcaccctcgtggcagtgctcgcccgcgccttcgtcctcgcggaaggagatcagctccttctggcaggtcagctcgtcgaacagccgctgcggctgccccttgaagaactggtcgttctcggcttcgagcacgagtgtcggacaggtgatgcggtcggcgatacccgccatggtgtacggctcggccgccctgaccagctcgtcgacaccggatacaccgaaggtccaccggccgttgcgcaccacccaccgcaccatggtgttctgcgccatcaacgcctccatgccgcccggcgtcgaagcggcacggccggcggtcgcggcgatgaccgcgtggaagtcgtagacgccgtcgtacagcacgcacgcggctacgcggtgctcgaaggccgccgccctcgcggcgaggtagccgcccaggctcatgcccatcagcacgagctgctcggcgtccacctcgggcagggtgagcgcgaagtcgacgaccggggtgaccaccgcctcccagtccggccggaagtgcaggccctgctcgcggacggtgctgccctggccggggccgtcgaacgcgatgacgttgtagccgcgccgcagcgcgccggcggccagcgccaggtagtcctcctcgagggtggagtcgtacccgccgtggaagagcacggtcgggcgcggggtgcccgaatcgtcgacgaggaacaggtagccgggaagcgtggtgtcctcgtacgggatgcgcagggcgcgggccggagtgtcgagcagcggggccgcgtcggcgaacgtctgctgggaggccttggcgagccgcgccgactcggcgtcggctgcgggattctcgcggcggtagaagtcggcggtccggtagtagttggacgcgcgcagcagcgcctcccgggcgctgacccggtgaccggcggccagcgcgtcccggccgatgcgctcgatgcgcgccgccgtcgcggcccactgggccgaccaggcttcctcgtctccctctgggatctgccggcaggtgaccaggacctcgccgaggtcggcgccgccataggccgcgtagcccgcggcgcgcagtgcctcgaaggagaaggactcgtcgtcgtagaggaatttcatcgtcgggctcctttccgagaatggaacggaacggcaccgttccgccttaccttcgaaggtaacacatggaacgtgaccgttccgttcgttatgctcgggagcatgacagcgcagcagaagaagggccggccggcgaccggcggggcggccctgcgacagcgggtaaccgaggcgatcaccgaggcggccttcgccgaactcgccgacgccgggtacgcgcggatgtcgatggagtcggtggcccggcgggccggcgtgggcaaggccgcgctctaccggcgctggccctcgaagcaggcgatggtcaccgaactgatccgcggcaaggtcaccgacacgctcccgcccacgcccgccaccggcgccctgcgcaccgacctgcgcgaactgctcaccaccttccgcggtcaactggccaatccgctgcttgcccggatcgcggccgggctgctcgccgaggccagtcacgacgacgctctggcggaggggctctacaccggagtgaccgcaccgcggcgggcggcggcgcacgcgatactgcggggcgcgatcgaccgcggcgaactccctcccgggctggatctggacctcggcaccgacctgctgatcgccccgctcgcattccgcgtactggtcatacagggccgaagcgacgatgagtatctggagacgctgaccaacgcgatcgaggcggcgttgcgagcggccgtccgctgaccgtgcggcgcacccgtcttcatcgggcgttgacctgttctgtgagttttcccttgcgcagggtgaggacacggtcggactgggcggccagttgctgggagtgggacacgacgaccacgcatttgtcctgctcgtgagcgagttcgcggaaggtgccgatgatgcccgcggcggtgtcctcgtcgaggtttccggtgggttcgtcggcgaagaggatgtcgacgtcgcaggcgagggcgcgggcgatggcgacgcgctgctgctggcctccggagagctggaggacgttgcgggtggccatcgccttgtcgaggccgatcttctccagcaggtgcagagcccgggccttgcgactgccggtcgtgggtttggtgccggtgatctccatggcgcaggtgacgttctggagggcggtcaggtaggtgaggaggttgtactgctggaagatcgtggcggcgtgctggttgcggtaccggccgaggcccagttcggtgaggtcctggccctcgaaggtgatggtgcccttggtgggggtgtcgagaccgctggcgagggagagcagggtggtcttgccgctgccggaagggcccaggatggtgtaaaaggtgccgcgttcgaaggcgtagtcgatgtccttgaggaccgcggtcctgcggcgctggcccgagtaggtgtggctgacgcccgcgagacgcagaacgggcggggcggcggtggcgctggtcatggcgggtcacttgcccttcgtgaggatggtgcgggggctcagccgcagcacggcaccggccgggacggcggtggcgagcaggccgatgccgaggccgatggcgccgagggtggcgaggtccgccggatcgagtcgtacggtgatcttgtcgatggggtcggcgtcctcgacgggcttgtcgttcgggtcgatgccctcgccgaggccggtactgccggacggcgggccctgccaggagtcgatcttgtgctgggcgctggtggcttccctgccgagcagggcctgcccggcgctctgggtcagccgcggggcgaacagggagctcaggccgatggcgagggcggcgacgacgaggatctccagggcctgttgggcgatgagcttgcgtttcttctcgcccatggccagcaggacgccgtactccttgtggcgctgcttgacggcgaggttgaccagcagcgtcaggacggccgcaccggcgagccccatcagccacatggccagggtggcggtagtgctgatggacttgagcgggccggtcatctgccggatggccttgtcgttggcgtccagcttgaaaccgtcgagcgccgggcccgcgacctttttggcctcgtccttgaacgcaccctgcgtgtcggcgtcctggaggaggaaggtcgccttacctgcctggagcggcccgttccgctcggagctgagcgcggagaggccgcccaccgtgccgtacagcatgttggccggactgatgccgtactcgctgtccggttcggtggcagggctcgggtcgcggtagacgccggccacggtgaagtcggccgtggtcttctcgtcgttgccggtcagggtgatcgtgtcgccgacaccgagcttgttctccttggcgagccgttcctcgatcaggagtcgcttcttgcccttgtcggcggcggtgaggtgctcaccggacagcagcgtgaacttgccgctgcggaagtcgggcagcagcgaggtgtcgagcacgcccatgcccacggtgccgccggggcccatgggctccttcggcccaccgtcggccagttgggggccgcccttgaggaggacgcggtcccagatcgagtaggtgtacttccgcacctggggcagagccccgaccttgtcgacggtcgcggcgtcgatgcgcggagccctcagctcgctcccgccgcccaactggcccaaatccgcctcgaggttgacctcggcgccgacggaacgcctggcgtcttgctcggcctgggccgtggcgtcgttgatcaggacaccggccagcaccatcacggagatgacgaggaaggtggcgagggtgatcagggtgcggcccttgcgggaccacaggctgagtcccgcgcgtttgacgaagttcatgggatcggctttctcagtccgtgccggtgagtggctattcggtgtcggtgaggatggagcggggatgcaggcgcaggatcccgatgcccgggatgacggtggcgaccagggagatgccgagcccgatgccggcgaccttgccgacggtggcgggctcgactcgtacggtcggcggcgcgatctcggcatcgggcacaccggccgccgcggccgccgcggccggcttgccgtcctgccgcccgaggagggtgtcacccgcgggctgtccggcgagtgaaccggccagcgcggcgagcgcgatggcgggcagggccacggccgccacctccacggcgtgctggccgatgagcttccacctcttctcgcccatcgcgaggagcaccccgagctcggcgcgccgctcgcggatctgcagcgccacgatcaagccgaggatcagcgtcccggccagcgcgatcacccagacgatcagtcccgcgaacgcgccgacccgctggatcgggcggacctggtccttgtacgccctgtcgttgacgcggaagtcgaagccgtccccgcccaggagacgctcggcctcggtgtgcagccgctcggcctgctccggcgagccgatcttgtacaccgcctcgctgaccgccacgttgccgggaccgagccgctgcgccgtgcccatcggcacgtacagcgcgttgccgggcagtttgtgcggtgccgtccagcgcgccggatcctgcgtggggttctggaagacgccgaccacctgcagcggaacggtttccttgtcgtccaccgaccgcacatggatcgtgtcgcccaccttgaggtgattcctgtcggccagccgctgctcgatcaccgcggcgttgcggcccgcgtcctcgggggtgatgccgcgccctgccgtgatcttcgtcgagccgtacgagaagggcagcagcatgcccgagtcacggacgccgttcacggccagcgggccctgctccttcttcccggcaccggcacctggaccggcacccgcacccgcacccgcatcgcccggcttcggcgcgtccgacgtcagcggcgtgaagccacgcgccccggcccgcaccggaagctccgggttgtagcggtacaccagatccgaggcaccgagccggtccgcgaggaccgcggtaaggcccttcttcctgacggtgacgtcgacgccgatggtgcgctgcgcgccggcttcctggcgggccgcggcggcctgcagcagaaagccgccgagaagcagggtgcagatgacgacgaagatcgccagcaacgcggcgctcttggactttctggcccccaggctcatcccggcgcgcttgacgaagttcatgccgaagacagaagcagctcgttgtttgcagcgagataagcgcgttacagcggcataagcgtgttacggcggcataagcgttacggcaacataaggacctgctcgcaccgtgccccgcgaccggcgccgcacgaccccgccggctcacggcacgggccgccacatccttatccggctgcaatcgcgatcttgatttgatgggggcatgagagttctggtagccgaggaccaccgtgttctcgcccgcaccatcgccaccggcctgcgccgccaggcgatggccgtcgacgtggcggccgccggcgacgaggccgagcggatgtgcctgctcaccgcctacgacgtgctgatcctcgaccgggatctgccggtcctgagcggcgacgaggtgtgccgacggctgcgcgaactccaggaccctccacggatcctgatgctgaccgcggccggcgagctgacggacaagatctacggcctcacgacactcggcgccgacgactacctggcgaaacccttcgatttcagcgaactggtcgcccgggtacggacgttgagccgtcgcgcaccgaagcagccccctaccgtgctgcgcttcgcgagcatcaccctggacgagacgcggcgcgaggtgtccgtggacggccgcccgctggagctgaccccgaaggagctcgcggtcctgcacctgctgcttgtggccgggggcgcgccggtgtcgcacgacgaactcgtccgcaccgtctgggacgagcacctcgacccgcgcaccagcgcggtccgcgccaccgtcagccggctgcgcggcaaactcggcgaccccggcctcattctcgccgacaagggggagggttaccggctgtgcgactgaacccatgggcgctgctgagccgactgcccttccgcgcccgcctgaccgcggccttctccgccctctttctgatcgccggcatcgcactgctcgccttcgtggtgctactcgcccgccacggaacggagcagcaggcccaggggatatcggtcacgtacggggatgtgccgagcggcagtggggcgcccatggggcccgtgcgccccacccggccgaatctcacacagcgggccccgggcgacgtcgccatgttccagaagatcgaccagaccgtgcgagccgtccaggacaccgcgctgcgccagatggtgctctggtccgccgtcggcctcctcgccatggcgctccttgccggagtgctcggctggtggctcgccggccgagccctgcgacccgtcgcctccatgaccgaaaccgcgcgccgcatcagcgaacagagcctccaccagcgtctcgcgctcaccggccccgacgacgaactccaccgcctcgccgacaccttcgacaccatgctcgacaggctcgagaagtccttcgagagccagcgccgcttcgtcgccaacgcctcccacgaactcaagacccctctcacggtccagcgcaccagcctccaggtcggcctcgccgaccccctccccgagggactcgcagacgtccgcgaggacctgctcaccgccaaccacgaggccgaacagctgatcaacggcctgctgctgctggcgcgcagcgaccgcggcctggagaagacccagaccatggacgtcgcggccatcgtccggctcgtgtccaccggcctcaccccactggcaacgaagaacggcgtgcgcatcgacctcgacgccgataccccgctggccgttcccggtgacccggtcctgctgcgccacctgctcacgaacctggtccgcaatgccatccagtacaaccaccccggtggccacgtccgcatacggctcgatacccccaccgtgacggtgaccaatacgggcccccacgtgtccccggaacagatccccgacctgttcgagcccttccaccgcctggacggggaccgtaccgccaccaccggccacggtctgggactgtccatcgcccactcgatcgccaacgcccaccacgccacgctgacggcacaacccggcaccgaaggcggactcaccctcaccctgcgcttcccttgccgccgttgaagccccgacagggcctcccccaccgggccgtcccggcaccgggacggtcagctctcggttcccgcggcccagagcacggtgtcgccggacttgatcacaacatttccgtcggcctggaacacgaggaccgctcctggatggccattggtccgtgaaaaccagaggtgctgcccctcactgctgtagacgaccaggttgccgtccgtctggaagcgggcggagttgttctcgcccttggtcatcgcagcccagcgtggcttgcggtcctggtcgtggaccaccaggttgccgtcttcctgcatgatcagactcctggccccggccaccacggacttcccggtgggcaactcgaacgtggcctctatgaccttgagtgccgcgcccttctcctggggcttctggggcccctggggcttctgagatccctggggtccctgggtcgaggcggacggcttcttccccgcggtcggcgaggcggccgaaccggatggccgcccctgcgcggacggggaggaatcggccgaggaacgctgcgtaccgaagtggacggcgaccaccaccaccacgaccacgaccgcggccgccgcgatggccgtggtcagcagccagccgggccgacggccccggccgggctcggagggggtggcgaggaagctccaccgtgttgcgaccgcggcgtcgaggaggaaaccgccggtcagcgctggctgcggagaataatgatcagcgtcttcgggtgaatggacagcctgtcgatgccccatggttggggcattcatccggtcagaccgtgaaccggcctccgttgctcagcagcaccgcaccaaccacattggacgcggcaggcgacgccagatagcagatgttgtcggcgagctcctccggcgtcgcgtatcccgacgcgcccgactcggccatcttctggcggacggcgtccggggtacgcgccgccatcggtgtgtccactggccccggagcgacggtgttgacgcggataccgaaagcgatggcctcttttgccacggcctgggacagcgcgtgcacaccggctttggaagcggcgtagtgcggatatcccgtgagcgtgtcgaacgccgcggacgagccaacagtcacgatggcgcccctgccgcgtgggcgcatggctcgcaccgcggctcgcagaacgtggaaagtaccgtcgaggttcacggccatcacccggcgccaggcggcgtcatcgagtcgagtcagcacatccaccggctcgcccgactcctgagcctcgaggattcgcgccttggcgtcgggatcgtccaccccggccgtgtggatgaccacgtcgatacggccttcggattcgaggacggacgacactacggcatcaacggcccgcggatcggtcacatcgacaacgtgcgcaacccctttgatgacggacgcgacggtgcgaacaccctccgcgttgaggtcgcccacgtgcacagccgcacatccttcggcagcgagcaggtgcgccaccgcggccccgattcctgacccgcccccggtaacgagggcgacctgtcccgcgaagcggtgctcaagggtcgttgccattgggccatctcctagatctcgtacagagaccctgccatccccattagcgtccacagcagatcctcatccaggtggttgcgatcagtcgtctcagcgtccggcgagcgcacagcggctccaccacgccacccagcgatccgccgaggggccgggggcgagaggccgggcgtgtgctagccaacgtctatgtgcgcccgacgagcatcgaactcaattgaactcaggtatacatctgatctcgtagctgacctgtcgtggatgactgccgactagcatcaaacctaattgaactcatgtcaatacctggctcccgatgaacagatggcccggcacatccccgaacagcttcggtggcagctccagcgcccgcacccgctggagccgttccgcctccgttccggtccttgcggcggcgggtcaccacacgtcgggcagtttgccctcaggccgtgggatcaccgaactcccgatcgatgaagtcaaggatttcgctggcactcgcactcgagagggcggtagtggaaacctcttcggggctcgaaatccgctgccatttattcagaaccgacgacagccgatccttgatcgcggatgcctgtcgatcgtcgaaggtcacggacgtgaacatcttttctatgcgatcgatctcggcgagcagggtcgtcgactgaaccctcgggttcatgaggtcgcccagccgggcggccaggtctgccggggtcgggtagtcgaacaccattgtggcgggcagccgcagccccgtggccttcgtcagactgttgcgcagctccaccgcggtcagcgagtccacacccaaatccttgaacgcaccggtcgccggaatactgcgggcgtcggcatgtcccagaaccgtcgcggcgctgtcacgcaccaccttcagcagtgccttcgcccgctcctccggcgccaagccgaccagccactgcgccgacgaatcaccggtggaggcggcccgccgggcaaccgggcggtgcaacaaacgcagcaatgccggaacctcgacctcccgcaccggctccatcggcgtggccaccagaaccggttcaccctgacgtgaggcgatgtcgaacagccgcatcccgcgctcggcggtgatggttcgcagcccgctgcgccggatccggtcatggtcggtaccggtcagcttcgcggtcaggccgctggcgtcctcccacaggccccaggccagcgcgagggccggcagaccctcagcacgacgctgctcagccagcgcgtccacgaacgcgttcgccaccgcgtagttaccctgccccgcactaccgagcacaccggcagccgaggagtacatgacgaacgcggcaaggtcgatatcgcgggtgagctcgtgcaggttccacgcaccatccgccttgggccgcagcacggtgtccagccgccgcgccgtcaacgattccacgacgccgtcatccagcacacctgcggtgtgaatcaccgccgtcaatggatggtccggcgacacacccgccagcacccgcgccagcccggcacggtccgacacgtcgcacaccgccgtgtccaccaccgcggcacccaactcggccagctcgctgatcagcgcctcgtccggggcgctgcgggacagcaacagcagatgacgcacaccgcgttcggccaccaggtgccgggcggcgatccccgccagcacacccgaaccaccggtgatcagaaccgtgccatccggatcccagacgccttcaggctcaggctcagcgacgcccgtacgagtcagtcgcggtgcctcgtaccggccgtcgatgacccgcagccgcggctcatcgagcccgacggtggcggccagttggtccggggtgagggtgtcgtcgtcactttccaccaggacgaaccggcccgggtgctccgactgagccgaacgcatcaaccccgacaccgcggctgcggccaaaccagttccggtccgcacaaccagcgtactgtcagtgaaacgctcccccgccagccacacctgcaccgcctgcagaacctcagcggtcagccgacgagtctgtgccagcgggtcaccgctgtcggggagtgcggtgaacacaacgacatcgggcatcggcacctcgccgtccgcgaggtcttcaaacttgcccaccgtcacgccgacctgctgggagaccgggatctccgtccacgtcagggcgaacagatcatcgacagcagaaccgagcgcatcggctgccaccggacgggtcaccagcgagccgatggtggccaccggccgaccgatgtcatcagcgacccgcacaccccagccatccgcgacccgtgtcatcgcgacccgcagcgtggccgagccagtgacatggacctggacgtggttccaggagaacggcaaccgcacggtgtcgcggtcggcatcgggtgtgttcaggatcccggcgtgcagggcggcatccaacaccgcagggtggacggcgaatcgtgccgcgtcctgggtctgctcctcagccagagcaacctcggcgaagacggtgtcaccatcacgccacgcggcctgcaaaccctggaaggcaggtccgtactcgtaacccgccctggtcagctggtcgtagaagcctgccacgtccaccggcttggcctgcgcaggtggccacgcggtgaggtccgacgttggcacagtcgtgtcggacacgctgacggtggcggaaacgtgccggacccaggcatcggcgctgtccgcccgggagaagaccgtcaccgcgcggtgtccggactcgtcggcctcgccgacggatacggacagttgcacaccgccggtctgcggcagcagaagcggggcttcgacgatcagctcgtcaacgacgtcgcagcccacctcatcagccgcgcggacaaccagctccacaaacccggtaccgggcagcaggacactgccccggaccgcgtgatcagccagccacgcgtgggtggccagtgatacccgcccggtcagcatcacaccatccgaacccggcatcgccagcaccgcacccagcaacggatggcccaccgcatccagacccgcccccgacacatcggcagaccggccggcctcagcccaataccgctggtgctggaacgcgtacgtcggaagatccagcacccgtgttgccgggacatcacccagcaccgcgcgccagtcgacagtcacgccgtggacataggcctgtgccagggcggtgagcatccgggtggcgtccccgtcgtcacgacgcagcgtggcaaccgtgacgacatcgtcgtccatcgcctgcaacagcaccgggctggcgctgacctcgacgaacacggtgtcgccctgggcctgcaactggctgacggcggggtggaaaccgaccggctcacgcaggttccggtaccagtactccccgtccagcgggctgtcgacccaggtgccgtccacggtcgacagccacggcacgagcggggtctgcgagctgctgtcgctagtgatgtcgagcagttcgtcgcggatcagctcgacgtgcggggtgtgcgaggcgtagtcgacggtgatccgccgcacccgcaccccttgtgcctcatgagcggtgaggacatggtcgaccgcttccggggtgcccgcgatcacggtggaggcgggaccgttgtgggcggcgatccaggccccgtcgaccagctcgacatcctgtgcgggcagggcgacggatgccatcgcaccacggcccgccaggccccgggcgatcgcctggctgcgcaaggtcacgatccgggcggcatcccgcatcgacaccgcacccgccacacacgccgcggcgatctcaccctgcgaatggccgatcaccgcatccggccgcacaccggcggcctgccacaccgcggccagggaaaccatcaccgcccaggaagcgggctggaccacatcaacccggtccaccaccgccggatcatccagcacggcgaacagatcccagtccacaaactcactcaacgccgccgcgcactcggccatccgctcggcgaacaccaccgacgaatcgcgcagtgcactgcccatccccagccactgccatccctgcccgggaaagacgaacacgatcctggggtcggacaccgcggtgccggtgacggtctcatctccaaggagtacggcgcggtgctcgaacaccgaccgtgtcaccgccagcgtcgatgccacagcccgtatatccactccgggcgacgccgccaggtaggcgcggagccggtcttcgtgttcggtcagggcgggctgggtcttggccgatatcaccagcggcaccagttccgaaaccagtacgggtgtagagccagccgtgtcgcccacagactgtgccggtgcactctcaaggatgacgtgggcgttagtgccactgatcccgaacgacgacacacctgcccgacggggtcggccggtctccggccacggctggttctccgccaccagctcgaccgcacccgccgaccagtccacatgccgcgacggctcatccacatgcaacgtgcgcggcaccatgctgtgctgcagggccatcaccatcttgatgacacccgacacacctgcggcagcctgggtatggccgacgttcgacttcagcgatcccagcagcagcggctccccgcggccctggccgtaagtagcgatcaccgcctgggcctcgatcgggtcacccagcgtcgtacccgtgccgtgcgcctcgaccacatccacctcgtgcggcgccaggccggcgttgctgagagcggcccggatcacccgctgctgcgaaggaccgttcggcgcggacaggccgttcgacgcaccgtcctggttcaccgccgagctacggaccaccgccaatatctggtgaccgttggcctgagcatccgacaaacgctccaccaataggacgccgacgccctcagcccaccccgtgccgtccgcggcgtcagcgaacgccttgcaccggccatcggctgacaagcctcgctgccgcgagaactccacgaaagtctgcggcgtggccatcacggtcacgccgccgaccagggccagcgaacactcaccctgtcgcagggcataccccgcctgatgcaacgccaccagcgacgacgaacacgccgtgtccaccgtgaacgcgggaccttcgagaccgaagaagtacgacacccggcccgacaacacactcgccgcaccagcagttgtcccgaagccgccgaggtcggcaccgatgccgtagccaccggggtaagcacctatgaagacgccggtgtcgctgccgcgtacggaaccgggctcgatcccggcccgctcgaacgcctcccaggacacctcaagcatcaaccgctgctgcggatccatcgccaacgcctcacgcggactgatcccgaagaacgaggcgtcgaaaccggccgcggcatccaggaagccgccctgcacgctgtaggacttcccgggcgcgtccgggtccggatcgtacaggttctcgacgtcccagccgcggtcggttgggaagccggaaatcgcgtccgtaccggactcgaccaagcgccacagatcctccggcgacgagactccaccgggtaggcggcaggccattcccacgatcgccagcggctcgtcctgcgccaccgccgacactggcccgcgcaccggagcggggttctcgccggtgaacaactcgtccaaccgggcagccagggccgtcggggtggggtagtcgaacaccagcgtggcgggcagccgcagctccgtggccttcgccaggctgttgcgcagttccaccgcggtcagcgaatcgatgcccagatccttgaacgccgtggttgccggaatcgtgctggtgtcggcgtgtcccagaaccgtcgcggcgccgtcagacaccagcttcagcagtgccttctcccgctcggccggtgccagggcggccagccaccgcgctccaccggcggcagcggcccgccgagcgaccgggcggtgcaacgaccgcagcaatgccgggacttccgcgtcccgtaccggctccatcggcgcggccaccagaaccggttcactgtggcgtgacgcgctgtcgaacagccccatcccgtgctcggcggagatggcccgcaggccaccgcgccggatccggtcacggtccgtgtcggtcagttgcgcggtcaggccactggcgtcctcccacagaccccaggccaccgccagcgcgggcagaccctcagcgtgacgctgctcagccagcgcgtccaggaacgcattggccgccgcgtagttgccctgtcccccactgccgagcacaccagcggcggaggaatacatgacgaacgccgccaggtcggcatcccgggtgagctcgtgcaggttccacgcaccgtcggccttgggccgcaacaccgcgtccagccgctgcgcggtcagggactccaccacaccgtcatcgagtacgcccgcggtgtgaatcacggccgtcaggggatgctccggtgacacacccgccagcacctgggccagcgcggcacgatcggacacgtcacaggccgctgtctcgacccgggcccccagttcgccgagttggttgatcagtgcctcgtccggggcgctgcgggacagcagaaggagatgacgcacgccgcgttcggtcaccaggtgccggacggcgatccccgccagcacacccgaaccaccggtgatcagaaccgtgccatccggatcccacaccttctctgactcgggttcagcggcatgcgtacgggtcagtcgtggcgcttcgaagcggccgtcgctgatccgcagccgcggctcatccaacccgaccgccgccgccaactgatccagagcgagggcatcgtcatcactctcgaccaggacgaaccggccggggtgttcggactgggccgaccgcatcaaccccgacaccccagcagcggccaaaccagtaccggtccgcaccaccagcgtgctgtcgctgaagcgctccccgccaagccactcctgaactgcctggaggacctgaccggtcagctttcgtgtttgctccagcgggtcgtcgtcgttgtcggggagtgcggtgaagaccgccacctcgggtaccggcacatcgccgtccgacaccagctcctcgaaccggccgaccgtcagaccggtcccctcaggggccgggatctccgcccaggtcaaggcgagcaggtcatcggcagcggaaccgagcgcgtcggccgtcaccgggcgcgtcacgagcgaacccacgctagccaccggccgaccgatctcatcagcgacccgcacactccagccatccgccgcctgcgtcaccgcgacacgcaacatggccgaaccaccagcatggacccgaacaccgttccaggagaacggcagccccacacccacctcggcatcggacgcattcaagggtgcaggcatgcagagccgcatccagtagagccggatgcacggcgggatatcgcgccgcctcccgcacctgctcctcagccagagcaacctcggcgaagacagtgtccccatcacgccacgcggcctgcaaaccctggaacgcgggaccgtactcgtacccttctgccatcagccggtcgtagaagcccgtcacgtccaccggctgtgcctgagctggtggccacgaggcaaactccgacagcgagaccgtcgagtcagaggtactgatcacggcggaaacgtgccggacccaggtatcggcgttgtccgcccgggagaagatcgtcaccacccggtgtccggactcgtcagcctcaccaaccgataccgacacctgcacaccgccggtctgcggcagcagaagcggggcttcgacgatcagctcgtcaacgacgtcgcagcccacctcatcagccgcgcggacaaccagctccacaaacccggtaccgggcagcagcacactgccccgcaccgcgtgatcagccagccacgcatgcgtggccaacgacacccgcccggtcaacagcacaccatccgaacccggcaacgccaccaccgcacccagcaacggatgccccaccgcatccagacccgcccccgacacatcggcagaccggccgacctcagcccaataccgctgctgctggaaggcgtacgtcggaagatccagcacccgggttaccgggacatcacccagcagcgcggtccagtcgacggtgacgccgttgacatacaggtgggccagggcgccgatcgcggcctgggtttcgtggtcgccgtgcagcatcgcgacgccgtcgaccaggcgggccagtgaccggtcggcacccagctcgacgaacacggcgtcctcgtatgaggccacctgctcgccgaaccggaccgtatcccggacctgccgcacccaatactcagccgtcatcacccgatcaccagcggccatcacgacaccaggtgcgtgataggtcaggccttgagcgaccgcccggaactcctccagcatcggctccatacgggcggaatggaacgcgtgactggtcgccagccgcgtccacttccccagcccctccgcggcctgcagcacagcggcctcatcaccggagagaaccaccgacgacgggccgttgaccgcggcgatctccacaccctcacccagcacggcccgagcctcatcctccgagaccgggacagcgaccatcaccccacccgcaggcagagcctgcatcagacgagcccgcgccgacaccaaagtgcaggcatcctccaacgaccacaacccggacacatacgcagccgcaagctcacccaccgaatggccgatcaccgcgtccggccgtacaccccacgattccagcaacccgaacagagccacctgcaaagcgaacaaagccggctgggcgtaaccggtctcattcacctccagatcgggcacatcgagcaggtcccacacctgctgatggatccgcgcgaagacggggaacgcggcggccagttccgcacccataccagcacgctgcgacccctgccccgggaagacgaacaccacccgcggatcggtcgccgcggtcccactaacagtgtcatcacccacgatcaccgcacggtgctcgaacaccgaccgtgtcatcgccagcgtcgatgccacagcccgcatatccaccccgggcgacgccgccagatacgcacgcaaccggccctcgtactcagccagtgccgactgcgtcttggccgaaatcaccaacggcaccaactccgaggccatcaccgacgccaccaccggcgcatccgcaggccgcacatcgtccacggcctgcgtggggggtgcgctctccaggatgacgtgggcattagtgccactgactccgaaggacgacacgcctgcccggcgcggccgaccggcttcgggccaggactggttctccgtcaccagctcaaccgcgccatccgtccagtccacatgccgcgacggctcatccacatgcaacgtacgcggcaccagaccgtgctgcagcgccatcaccatcttgatgacacccgacacacccgcggcagcctgcgtatgaccgacgttcgacttcaccgaacccagcagcacaggctggtcacgatcctgaccgtaggtcgcgatcaccgcctgggcctcgatcgggtcacccaacgtcgtacccgtgccgtgggcctcgaccacatccacctcggccgtggtcaggcccgcgttggccagcgcctgccggatcacccgctgttgcgaggggccattcggcgcggacaggccgttcgacgcgccgtcctggttgaccgccgagctgcggaccactgccaatacctggtggcccttggcctgagcgtctgacaggcgctcgaccagcaggacgccgactccctcagaccatcccgtgccgtccgcgctgtcggcgaacgccttgcaccggccatcggatgccaggccacgctgccgggagaactccacgaaggtatgtggtgtggccatcacggtcacaccgccgatcagggccagcgagcactcaccctgtcgcagggcataccccgcttggtgcagggcgaccagcgacgaggaacacgccgtgtccaccgtgacggccggcccctcaagaccgaagaagtacgacagccggccggacagcacgttgaggccagcggtcgcaccataaccttcgaggtctgcgccgatgccgtaaccgcccgggaaagcgcccatgaagacgccggtgtcgctgccgcgtacggaaccgggctcgatcccagcccgctcgaacgcctcccaggacacctccagcaccagccgctgctgcggatccatcgccaacgcctcacgcggactgatcccgaagaacgaggcatcgaagttggcagcggtcgccaggaagccaccctctacgcagtacgacttcccggccgcatccggatccgagtcgaacaggttctccacgtcccagccacggtcggtggggaaaccggaaatcgcatccgtacccgattccacaaggcgccacagatcctcaggcgacgacaccccaccaggcaggcggcaggccatgcccacgatcgccaacggctcgtcctgccccaccgccgacaccggcgtgcgtacctgaacaggattctcgccggtgaacagctcctccaaccgggcggccagggcacctggggtcgggtagtcgaacaccaacgtggcgggcagccgcagtcccgtggccttcgtcaggctgttccgtagttccaccgccgtcagcgaatccacgcccagatccctgaacgcggcggtcaccggaatactgtcgacgtcggcgtgtcccagaaccgtcgcggcactgtcacacaccaccttcaccagtgccttcgcccgctcctccggcgccagagcggccagccaccgcaccgcaccgccggaggccgcccgccgggcgaccgggcggtgcaacgaccggagcatcgcggggacttcggcttcccgtaccggctccatcgccgcggccaccagaaccgggtcgctgcggcgtgacgcgccgtcgaacagccgcattccatgctcggcggagatggcccgcatgccgctgcgccggatccggtcatggtctgtatcggtcagattggcggtcaggccgctggtgccctcccagaggccccaggcgagtgcgagtgcgggcaggccctcggcacgacgttgctctgcgagagcgtccaggaacgcgttcgccaccgcatagttgccttgccccgcgctaccgaatacaccggcggccgaggagtacatgacgaacgcggccaggtcggcgtcacgggtgagctcgtgcaggtgccaggcaccgtcggccttgggccgcagtaccgtgtccagtcgctgcgcggtcagggactccaccacaccgtcgtccaccacacccgcggtatgaatcaccgccgtcaggggatgctccggtgacacgtccgccagcacccgcaccaacccagggcgatccgatacgtcgcaaaccgctgtggccacctgggcgcccagttcgccaagctcgctgatcagtgccccgtccggtgcgctgcgggacagcagcagcagatgacgcacgccacgctcggccaccaagtgccgggcggcgatccccgccagcacacccgaaccaccggtgatcaggaccgtgccatccggatcccacaccttttctgactcgggttcagcggcatgcgtacgggtcagtcgtggcacttcgaaccggccgtcgctgatccgcagccgcggctcatccaattcgacggtcgccgccagctggtcgggggcgagggtgtcgtcgtcgctttccaccaggacaaaccggcccggatgttccgactgggccgaccgcatcagacccgacaccgcggcagcggccaagccagtgccgatccgcacaatcagtgtgctgtcggtgaagcgttcctcggccagccacgcctggatcttctgcaggacctgggcggtcagcgtacgagtctgggccagcgggttctcgctactgtcggggcgtgcggtgaagaccaccacctcgggcaccggcacatcggcgtccgccaggtcttcgaagcggcctatggtcaggctggtctcctgaggggccgggatctccgtccacgtcagggcgagcaggtcgtcggtggtggagccgagtgtgtcggccgtcaccggccgagtcacgagggagccgatggtggccaccggccggccggtgtcatcagcgacccgcacactccagccgtccgccgcctgtgtcacggcgacgcgcaacatcgccgagccgaccgcgtggacctgaacctggttccaggagaacggcaaccgcacgccctgctcggtgtcgggcgtgttcaggatcccggcgtgcagggcagcatccagcaacgcgggatgcacggcgtatcgggccgcgtcctgcgcttgctcttcggcgagggcgacctcggcgtagacggtgtcgccgtcacgccacgcggcctgcagcccctgaaacgcgggaccgtactcgtagcccaccacggtcaactgatcgtagaagtcggccacgttcaccggctgggcctgggcaggtggccacgcactgaggtctggcagcgagagggtcgtatcagagttgctgaccgtgaccgtggcgtgccgcgtccaggtatcgacgttgtccgcccgggagaagaccgtcaccgcccggcgtccggaatcatcggactcggcgaccgcgacggacagttgcacgcctccggtctgcggcagcaggagcggcgtctcgatcaccagctcgtcgatgacgtcacaaccgacctcgtcggcggcgcggacgaccagctccacaaacccggtaccgggcagcagcacactgccccgcaccgcgtgatcagccagccacgggtgcgtggccaacgacacccgcccggtcaacagcacaccatccgagccgggaaccgccaccgcctcacccagcagcggatggccgcccgcggccatccggtccgtgccctcgagccagtagcgctggtgctggaaggcgtacgtcggaaggtccagcacccgtgttgccggagcatcgcccaggagcgcgggccagtcgacggtgacgccgttgacgtacaggtgggccagggcactgacagcggcctgcgcctcgtggtcgccgtgcagcatcgcgacgccgtcgaccaggcgggccagtgaccggtcggcacccagctcgacgaacacggcgtcctcgtacgaggccacctgctcgccgaaccggaccgtgtcccggacctgccgcacccagtactcagtggtggtcacctgatcaccagcggccatggagacctgcggcgtccggtaggtcaggccttcggcgaccgtccggaactcctccagcatcggttccatacgggcggaatggaacgcgtggctggtcgccagccgcgtccacttccccagcccctccgcggcctgcagcacggcggcctcatcaccggagagaaccaccgacgacggcccgttgaccgcggcgatctccacaccctcacccagcacggcccgggcctcatcctccgagaccgggacagcgaccatcaccccacccgcaggcagagcctgcatcagacgagcccgcgccgacaccaaagtgcaggcatcctccaacgaccacaacccggagacgtatccggcggcgagctcaccgacagagtgaccgaccaccgcatccggccgtacaccccacgattccagcaacccgaacagagccacctgcaaagcgaacagggccggctgggcatacccggtctcgttcacctccagatcgggcacatcgagcaggtcccacacctgctgatggatccgcgcgaagacggggaacgcggcggccagttcctcacccataccagcacgctgcgacccctgacccgggaagacgaacaccacccgcggatcggtcgccgcggtaccggtgacggtgtcatctcccagcagcacggcccggtgctcgaacaccgaccgtgtcatcgccagcgtcgatgccacagcccgcatatccaccccgggcgacgccgccagatacgcacgcaaccggccctcgtgctcagtcaaagccgactgggtcctggccgaaatcaccatcggcagccactccggtgcccgctcgatcaccgcgttgtccgcaggctgagtggggggtgcgctttccaggatgacgtgggcgttggtgccactgatcccgaaggacgacacgcctgcccggcgcgctcggcccatatccggccacggctggttctccgtcaccagcgcaaccgcgcccgccgaccagtccacatgccgcgacggctcatccacatgcaacgtacgcggcacgaaaccgcgctgcagcgccatcaccatcttgatgacacccgacacacccgcagcagcctgcgtatgacccagattcgacttcaccgaacccagcagcaacggctgctcacgatcctggccataggtcgccagcaacgcctgggcctcgatcgggtcacccagcgtcgtacccgtgccgtgcgcctcgaccacatccacctcgtgtgccgcaaggccggcgttgctgagggcagcctggatcacccgctgctgggaagggccattcggcgcggtcagaccgttcgacgcaccgtcctggttgaccgccgaactgcgcagcatcgccagcacctggtggcccttggcctgagcgtccgacaaacgctccaccaacaacacaccgacaccttcggagaagccggcgccgtccgcactgtccgcgaacgccttactgcgaccgtcaccggccaggccaccctgacgagcgaactccacgaaagtctgcggcgtggccatcacggtgacaccaccgaccagggccagcgaacactcaccctgccgcagggcataccccgcctggtgcaacgccaccaacgacgacgaacacgccgtgtccaccgtgatggcagggccttcgaggccgaagaagtacgacacccggccggacaggacactgaccgcgctcgcggtggcgccgaagccgccgaggtcggcaccgatgccgtagccaccggggtacgcacccatgaaaaccccggtatcagttccgcgtacggaaccgggctcgatcccggcccgctcaaacgcctcccaggacacctccagcaccaaccgctgctgcggatccatcgccaacgcctcacgcggactgatcccgaagaacgaggcatcgaaaccagccgcggcatccaggaaaccaccctgcaagcgataggagtcgccagctgcggggccgaacagattctccacatcccagccacgatcggtggggaatccggagaccgcgtccgtacccgactccagaagacgccacagatcctcaggcgacgacaccccacccggcaagcgacacgccatgccgacaatcgccaacggctcgtcctgcgccaccaccgacaccgacgtacgtaccggaacaggagtttcaccgacgaacaactcccccagccgggctgccaaggccgtcggggtggggtagtcgaacaccagcgtggcgggcaaccgcagccccgtggccttcgtcaggctgttccgcagttccaccgcggtcagcgaatcgatgcccagatccttgaacgccgcggtcgccggaatcgtgctggcgtcggcatgtcccagaaccgtcgcggcgccatcacacaccagcttcagcagcgccttctcccgctccaccggcgccaaccgggccagccactgcaccgacgagccgccggtagaagcagcccgccgggcaaccggacgatgcaacaaacgcagcagcgccggaacctcggcctcacgcacaggctccatcgccgcagcgagcagcaccggctcaccatgacgtgacgcggcatcgagcaagcccatcccatgccccgcggagatcgcccgcaggccaccacgccggatccggtcacggtccgtgtcagtcatttcggcggtcaggccgctggcgtcctcccacagaccccacgccaccgccagcgcgggcagcccctcaccacgacgctgctcagccaaggcatccaggaacgcgttggccgccgcgtagttgccctgtcccccgctgccgagcacaccggcggcggaggagtacatgacgaacgccgccaggtcggcgtcccgggtgagctcgtgcaggtgccaggcgccgtcggccttgggccgcagtaccgtctccagctgctgcgcggtcagggactccacgacgccgtcgtcaagtgcgccggcggtgtgaatcaccgcggtcaacggatgctctggtgatacaccggcgagtatccgtgccagcccggcacgatcggacacgtcgcagatcgccgtgtcgacccgggcgcccagctcaccgagctcgttgatcagcgcatcgtccggggtactgcgggacagcagcaggagatgacgcacaccctgctcggccaccagatgccgggccgcgatccccgccaggacacctgaaccaccggtgaccagaaccgtgccgttggcgtcgagttgctgagggacggtgaggacgatcttgccgatgtggcgggcacggctcatccagctgaacgcctcacgggcctgacggatgtcccaggcccgcaccggcaggggcaatagcacaccctgcgcgaacagggcgagcaaatcggcgatgatctcccgcagccggtccggaccggcgtccatcagatcgaacgcctggtagctggtacccggccggtcggcggtgacctggtgcgggtcacggatgtcggtcttgcccatctcgacgaaccggccaccacgaggcagcagatcaagcgaagcatcgacaaagtcaccgctgagggagttcaggacgacatcgacaccccggctgtcagtggtgtccagaaccatttcccggaagccagtggtacgcgagtccccgatccgggcatcctccagacccgcctcgcgcaggacgtgctgcttaccggtgctggccgtcgcgtagatccgcgcgccgaggtggcgggcgatctgggtggctgccatgccgacaccacccgcggcggcgtggatcagcacagactcacccgcactcagcccggccagatccaccagcccgtaatacgcggtggcgaacacaaccggcacggacgccgccgtagtgaacgaccacccgtcaggaatcacacccagcattcggcgatcagcgatcgcgaccgccccgaacccaccgcccacaagaccgaacactcggtctcccggggccagatcctggacctccggaccgacctcgagcaccacgcccgcagcctcagccccgatcacagcctcaccggggtaagtgccgagcgcgatgagaacatcgcggaagttcaggcctgcggcgcgtacgtctactcgtacttcaccggattgcaggggccgttcggcggtttcggcgggtaccagcgcgaggtcccgcaaggttccgctgcgggactgctccagcagccacgcacgatccccgggaatcgtgagcgggctgctttccggcgtattcgcccgcgccagccgcggtacctcgaaccggccgtcgcagacccgcagccgcggctcgtccaacccgacagtcgcggccagttgctggagggtgaggttgtcgtcgcattccaccaggacgaaccggccggggtgttccgactgggcagaccgcatcagacccgacaccccggcggtggccagcccggtgccggtccgcacgaccagcgtgctgtcggtgaaacgttccccggccagccatgcctgaactgcctggagaacctgggtggtcaacgtgcgggtctgtaccagcggatccggcggatccagcggggctagcgggttctcgctgctgtcggggagtgcggtgcagaccaccacctcgggcaccggcacatcaccgtccgccaggtcttcgaaccggccgacgctcaggccggtctgctggggggtggggatctccgtccagaccaccgtgagcaggtcgtcggcagcggaaccgagcgtgtcggccgttaccggccgcgttacgagcgagtcgacggtagccaccggtcggccgctgtcgtcggcgacccgcacactccagccgtccgtcactggtgttatcgcaacgcgtagcgtggccgtgccagtggcccggacctggacaccgttccaggagaacggcagccgcacaccctgctcggcggtgtcgagtgtgttcagaattccggcgtgcaaggcggcgtccaacagcgccggatgcaccgcgaaccgtgccgcctcctccgcctgctcctcggccagggccacctcggcgaagacggtgtccccgtcacgccacgcggcctgcagcccctggaacgcgggaccgtactcgtaaccggccccggtcagccggtcgtagaaatcgcccaggcccaccggctgtgcctgtgcgggaggccaagcagcaagctctggcagcgagagggcagcgccagagacgccgatcgtggccgaaacgtggcgagtccaggcgtcggcgccatccgcacgggaaaagaccgtcacaccccggcgcccggattcgtcagcctcgccgaccgacacggacaggtgcacgctagcggacgacggcagcagcagcggggtttcgatcaccagctcatcgacaacgtcacaccccacctcatcagcggcgcggacgaccagctccacaaacccggtaccgggcagcagcacactgccccgcaccgcgtgatcagccagccaggcatgcgtggccagtgatacccgcccggtcaacagcacaccatcggactcaggcaactccaccgccacacccagcaacggatgaccgcccgcaaccgaccggtccgcaccctcggcccagaagcgctggtgctggaacgcgtacgtcggaaggtccggtacccgtgtcgtggcggtgcccaggattgcgggccagtcgacggtcacgccctcgacaaacgcctgtgccagggcggtgagcatccgggtggcgtccccgtcgtcacgacgcagcgtggcaaccgtgacgacatcgtcgtccatcgcctgcaacaacaccggactcgcgctgacctccacgaacacggtgtcgccctcagcacgcaactggccgacggcggagtcgaagcccaccggttcacgcaggttccggtaccagtactccacatcgagcggaccctcgacccacgtcccatcaacggtcgacagccacggcaccaccggagcctgcgaaccgatacccgcggtgatgtcgagcagctcgtcacggatcagctcgacgtgcggggtgtgcgaggcgtagtcaacggcgatccgccgcacccgcaccccccgcgcctcatgaaccgccagcacacggtccaccgcctccggcgcacccgcaaccaccgtagacgcgggaccgttgtgggcggcgatccaggccccgtcgaccagctcgatctcgtgcgcgggcagggcgacggatgccatcgcgccccggcccgccaggccccgggcgatcgcctggctgcgcaaggtcacgatccgggcggcatcccgcaacgacaccgcaccggccacacacgccgcggcgatctcaccctgcgaatggccgatcaccgcatcaggccgcacaccggccgcctgccacaccgcggccagggaaaccatcaccgcccacgaagcaggctgaaccacatcaacccggtccaccaccgccggatcatccagcaccgcgaacagatcccagtccacaaactcactcaacgccgccgcacactcggccatccgctcggcgaacaccaccgacgaaccccgcagcgcactgcccatccccagccactgccacccctgccccggaaaaacgaacaccactcgggggttggacaccgccgtgccggtaccggtgacggtgacggtgtcgtggccgaggagtacggcacggtgctcgaacaccgaacgcgtcagcgccagcgtcgaccccacagcccgcatatccgccccgggcgacgccgccaggtaggcgcgcagccggtcctcgtgttcggtcagcgccgggagcgtcttggctgatatcaccagcggcaccagctccgaggccacaactggtgtcgctacgggctgcgcgttgttcacagactgggcgggtggtgcgctctcaaggacgacatgcgcgttcgtaccactgaccccgaaggccgacacacccgcccggcgcggccgaccggtgacgggccaggactggttttccgtcaccagctcaaccgcacccgccgaccagtccacatgccgcgacggctcgttcacatgcagggtgcgcggcaccgtgctgtgctgcagggccatcaccatcttgatgacacccgacacacctgcggcagcctgcgtatgtccgacgttcgacttcaatgtgcccagcagcagaggctgctcacggtcctgaccgtaggtggcgatcaccgcctgggcctcgatcgggtcacccagcgtcgtacccgtgccgtgtgcctcgaccacatccacctcggcggtggtcagacccgcgttggccagcgcctgccggatcacccgctgctgggcgggtccgttcggcgccgtgaagccgttggacgcgccgtcttggttgactgccgatccgcgaacgactgcgagcacctggtggccgttgcggcgggcgtcggagagccgctcgaccagcagcagacccgcgccttcggagaaaccggcaccatccgcactgtccgcgaacgccttgcaccggccgtcggaggccagaccacgctgccgggcgaactcggtgaagacctccggcgtggccatcaccgtcacaccaccggccaacgccatcgaacactcaccattgcgcaacgcctgcgccgccagatgcaaagcaaccagggacgaggagcacgccgtgtccatcgtgatcgccggaccctcaaggccgaagaagtacgacagccggccggacaacacgttgggagccgcggtcgcaccgtaaccctcgcgggcagcaccggcgccgtaaccaacggggaacgcgccaatgaagacgccggtgtcgctgccgcgtacggaaccgggctcgatcccggcccgctcgaaggcctcccaggacacctccaacagcagcctctgctgcggatccatcgccaacgcctcacgcggactgatcccgaagaatccggcgtcgaagccagccgccgtgtccaggaagccgccctcagcgcggtacgacttcccggccgcgtccggatccggatcgaacaggccctcgacgtcccagccacggtcggtgggaaagccggagaccgcgtccgtacccgactcgaccaagcgccacagatcctccggcgacgacacaccgcccggcagacggcacgccataccgacaatcgccaacggctcgtcctgcgccaccgccggcaccggctcgcgtaccggtgcagggttctcgccggtgaacaactcgtccaaccgagcagccaaggcggtcggggtgggatagtcgaacaccatcgtggcgggcagccgcagccccgtcgccttcaccaggctgttccgcagctccaccgcggtcagcgaatccacgcccaaatccttgaacgcaccggtcacgggaatggtgcgggcgtcggcgtgccccaggaccacggcggcactgtcacacaccaccttcagcagcgccttctcccgctcggccggtgccagggcggccagccactgcaccccaccgtcggcggaagcggcccgccgggcgaccgggcgacgcaacgaacgcagcaacgccggaacctccgcggcccgtgccggatccagcgacgcggccatcagaagcggctcgccacgatgcgatgcgctgtcgaacaggcgcatcccacgctcggcggagatggtccgcagaccgatcaggtcactggtctcttctgacgaaccccaggcgagtgcgagtgcgggcagcccctcggcacgacgttgctcggccagcgcgtccaagaacgcggtggccgccgcgaggttgccctgccccggattacccatgacaccggcggccgaggaatacatgacgaacgcggccaggccggtgttgcgagtgagttcgtgcaggtgccaggcgccgtcggctttgggccgtaggaccgtgtccagccgctgcgcggtcagggtctccaagacgccgtcgtcgagtgcgccggcggtttggatcaccgccgtcagcgggtgttccggtgatacgcccgccagcacccgcgccagcccggcacggtccgacacgtcgcagtccgccgtctcgacccgagcgcccagctcgccgagttcgttgagcaacccctcgtcggcagtggtgcgggacagcagcaggagatgacgtacgccacgttcggtgaccaggtggcgggcgagggcgcctgccaggacgccggagccgcctgtgatcaggaccgtgccatgcgggtcccacaccgtttcagattcgggctcagcggcgttcacccgggccagccgtggcacttcgaaccggccgtcgttgacccgcagccggggctcgtccagtccgacggtagcggccagttggtggagggtgaggctgtcgtcgtcgctttccaccaggacgaagcggccggggtgttccgactgggctgaacgcatcagccctgacaccgcggcggcggccagcccggtgccggtccgcacgaccagtgtgctgtcggtgaagcgctccccgccaagccacgcctgggccgtctggagcacctgggcggtcagcgtacgagtctgtaccagcggatcagggctgccgtcggcctcgggacgtgcggtgtagaccgccacgtcggggaccggcacgtcaccgtccgccaggtcttcgtacctgccgacgctcaggctgctctgctggggagtcgggatctccgtccataccagcgtgagcaggtcatcggcagcggagccgagtgtgtcggctgtcaccggccgggtgacgagggaaccgatggtggccacgggcctgccggtgtcgtcggcgacccgcacactccagccgtctgttgtcggtgttgccgcgacgcgcagcatggctgtgtcggtcgctcggatttggacgtggttccaggagaacggcatccgcacgctctgctggtcggtatcgggtgtgtgcaggacgctggcgtgcatggcggcgtccaacagcgccggatgcaccgcgaaccgtgccgcctcctgcgcctgctcctcagcgaggacgacctcggcgtagacggtgtccccgtcacgccacgcggcctgcagcccctggaacgcgggaccgtactcggtacccgcggcagccagccggtcgtagaggtcggccacgttcgtcggctgggcctgggctggcggccacgaggcaaactccggcagcgagaggggtgcatcagaggcgctgatcgtggcggaaacgtgccgggtccatgtgtccgcgttgtccgcccgggagaagaccgtcaccgcgcggtgtccggactcatcggctccgccgacggacacggacagttggacgcctccggtctgcggcagcaggagcggggtttcgatcaccagctcatcgataacgtcgcactcgacctcgtcggcggctcggacgaccagttccacaaatgcggtcccggggagcaggacactgccccggaccgcgtgatcggccagccatgtatgggtggccagcgacacccgcccggtcagcaccacaccattggagtccggcaactccacggccacacccagcaacggatgaccacccgcagccgagcgatccacaccctcgacccagtaccgctggtgttggaaggcgtaggtcggaaggtccagtacccgggttgtggtggtgccgaggatggcgggccagtcgacggtgacgccgtggacataggcctgtgccagggcggtgagcatccgggtggcgtcgccgtcgtcacgacgcagcgtggcaaccgtgacgacatcgtcgtccatcgcctgcaacagcaccgggctggcgctgacctcgacgaacacggtgtcgccctgggcctgcaactggccgacggcggggtggaaaccgaccggctcacggaggttccggtaccagtactccacatcgagcgggctgtcgacccaggagccgtccacggtcgacagccacggcacgaccggggcctgcgagctgctgtcgctagtgatgtcgagcagttcgtcgcggatcagctcgacgtgcggggtgtgcgaggcgtagtcgacggtgatccgccgcacccgcacccctcgcgcttcatgagcggtgaggacatggtcgaccgcttccggggtgcccgcgatcacggtggaggcgggaccgttgtgggcggcgatccaggccccgtcgaccagctcgacatcctgtgcgggcagggcgacggatgccatcgcgccccggcccgccaggccccgggcgatcgcctggctgcgcaaggtcacgatccgggcggcatcccgcatcgacaccgcacccgccacacacgccgcggcgatctcaccctgtgaatggccgatcaccgcatccggccgcacaccggccgcctgccacaccgcggccagggagaccatcatcgcccaggaagcgggctggaccacatcaacccggtccaccaccgccggatcatccagaaccgtgaacaggtcccagtccacgaactcgcgcaacgcggccgcgcactcggccatccgctcggcgaacacgatcgaggaatcgcgcagcgcactgcccatccccagccactgccacccctgccccgggaaaacaaacaccacccggggatcggacacagcggtgccggtgacggtgtcgtctccaaggagtacggcgcggtgctcgaacaccgaccgtgtcaccgcgagcgttgatgcaacagcccgtgtatccactccgggcgacgccgccaggtaggcgcgcagccggtcttcgtgttcggtcagggcgggctgggtcttggccgatatcaccagcggcagcacatccgaggccaccaccggcgtctcaacaggctgcgcctcctccgcgggctgagcggggggtgcgctctccaggatgacgtgggcgttggtgccgctgactccgaaggacgacacgcccgcccgacggggccggtcggtgccgggccagggctggttctccctcaccagctcaaccgcacccgccgcccagtccacgtgccgcgacggctcatccacgtgcagggtgcggggaaccgtgttgtgctgcagggccatgaccatcttgatgacacccgacacgcccgaggcggcctgagcatgaccgatgttcgacttcagcgagcccagcagcaggggccgttcccggtcctgaccgtaagtggcgatcaccgcctgggcctcgatcgggtcgcccagcgtcgtacccgtgccgtgggcctcgaccacatccacctcggccgtggtcagacccgcgttggccagcgcctgccgaatcaccccctgctgggaggggccgttcggcgcggacaggccgttcgacgcgccgtcctggttgaccgccgagctacggaccaccgccaacacctgatggcccttggcctgggcgtcggaaagccgctctaccagcagcacaccaacgccttcagcccatcccgtgccatccgcgctgtctgcgaacgccttgcaccggccatcggaggccagaccacgctgccgcgagaactccacgaaactttgcggcgtggccatcaccgtgacaccaccgaccagggccagggaacactccccctgccgcagggcataccccgcctggtgcaatgccaccagcgacgacgaacacgccgtgtccaccgtgatggcggggccttcgaggccgaagaagtacgacacccggccggacaggacactggtcgcgctggcggtggccgcgaagccgccgaggtcagcaccggcaccgtagccaccggcgtacgcgcccatgaagacgccggtgtcgctgccgcgcacggaaccgggctcgatcccggcccgctcgaaggcctcccaggacacctccatgatcagccgctgctgcgggtccatcgccagagcctcgcgtgggctgatgccgaagaatccggcgtcgaagctggctgcgctgtcgaggaagccgccctctacgcagtacgacttcccgaccgcgtccgggtccggatcgaacaggctctccgcgtcccacccacggtcggcggggaaaccggaaatcgcatccgtacccgactccacaaggcgccacagatcctcaggcgacgacaccccgccgggcagacggcaggccatacccacgatcgccagcggctcgtcctgccccaccaccgaggcggtgcgcaccaatacaggattctcgccggtgaacaactcccccagccgagcagccaagaccgccggggtcggatagtcgaacaccaacgtggccggcaaccgcaaccccgtcaccttcgccagaccgttccgtagttccaccgcggtcagcgaatcaatgcccagatccctgaacgctccggtcaccggaatggtgcgggcatcggcgtgccccagaaccgtcgcggcggtgtcacgcaccaccttcagcagtgccttcgcccgctcctccggcgccagggctgccagccactgcgctccaccggcgagagcggcacgccggacgttcggacgatgcaacgaccgcagcaatgccgggacttccgcgtcccgtaccggctccatcggcgcggccaccagaaccggttcactgtggcgtgacgcgttgtcgaacagccgcatcccgtgctcggcggagatggcccgcaggccaccgcgccggatccggtcacggtccgtgtcggtcagttgcgcggtcaggccgctggcgtcctcccagaggccccaggccaccgcgagtgcgggcaggccctcggctcggcgttcttcggcgagcgcgtccaggaacgcgtttgccgccgcgtagttaccctgacccgcaccgcccatgacaccggcagccgaggaatacatgacgaacgccgccaggtcggcgtcccgggtgagctcgtgcaaatgccaagcaccatcggccttgggccggaatacggtctccagccgctgcgcgctcagggactcaacgacaccgtcatccaccacgcccgcggtatgaatcaccgccgtcaacgggtgctccggcgacacacgcgccaacactcgcgccagctctgcacgatccgacacgtcacaggccgctgtgtccaccgcggcacccaactcggccaactcgctgagcagcgcccggtccggagtgccgcgagacagcagcaggagatgacgcacaccacgttcggccaccaggtgccgggcgacgatccccgccagaacgccggaaccgcccgtgatcaggaccgtgccatccggatcccacgccctttcaggctcaggctcatcggcatgtgcgcgggtcagccgtggtacttcgtaccggccgtcgctgatccgcagccgcggctcgtccaatccgacagtcgcggccagctggtccggagtgagggcgtcgtcatcactttccaccaggatgaaccggccggggtgttcggactgggccgagcgcatcaaccccgacaccgctgccgcggccaaaccagtaccggtccgcaccaccagcgtgctgtcgctgaagcgttccccgccaatccactcctggactgtccgcagaacctgcgcggtcagtatgcgagtctgtgccagcgggtcgtcgttgccggggagcgcggtgaacaccacgacatcgggcaccggcgcatcgccgtccgccgccaggtcttcaaacgcacccaccgtcagaccggtctgctgctggggggtggggattcctgcccaggtcagggtgagcaggtcggtggcactggtggccggttcgttgacgggaacggcgtccagcgcgagggagcgggcggtcaggacaggaagccctgtgctgtcgacggcctctaccgacagggtctcgtcatcctgtgaggtgaggcggacgcgcagctcggcagcgttgcgggcgtgcagggtcaagccctgccacgcggcgggagtctcctcgcctacggcggcgaaggcggcagtcatcaggccgggatgaagcaaaaacctggtggcgtcgtgggtgtcgtccagggcgacctcggcgaacacttcgctgccacggcgccacaccccgcgcagtgacggtactcgggcctcgaccgggaccgcaccggccggcggccacgcctcgccggcctgccagccggacaccctgccggtggtggtggccagggtcgcggtggccaggcgtgtccagagctcgtcgtcgctgtaacgggcgtggatactgagctggcggcggccggcatcgtcgggttcgctcacccaggtctgtacctggacggcgccatcgtcaggaagcagcagcaaggcttccacggtgagttcctcgaccaggccgcagccggtctcgtccgccgctgccagcgccatctcggcgaacgccgtcgcgggcaagaccgtctggtcctgcaaccacagatcaccgctccgggaaacttgcgaggtaaacagcactccactggcctcggccagccctaccgccggcccgagcagcggatgaccgcccgcggccgcccggtccgcaccctcaagccaataccgctggtgctggaaggcgtatgtcggaaggtccagcacccgtgttgccggagcatcgcccagaagcgcggtccagtcgacggtgacgccgttgacatacaggtgggccagggcactgatcgcggcctgtgcctcgtggtcggtgtgcagcatcgcgacaccgtcgaccaggcgggccagcgaccggtcggcacccagctcgatgaacaccgcgtcctcgtacgaggccacctgctcgccgaaccggaccgtgtcccggacctgccgcacccagtactcagccgtcgtcacccgatcaccagcggccatggcgaggcggggtgtttggtaggtgaggcgttcggcgaccgtccggaactcctccagcatgggttccatgcgggcggagtggaaggcatggctggttgccagacgtgttgacttgcccaaggctgctgcggcctgcagtactgcggtttcatcgccggagagaaccaccgacgacgggccgttgaccgcggcgatctccacaccctcacccagcacggcccgggcctcatcttcggagaccgggacggcgaccatcaccccacccgggggcagggcctgcatgaggcgggcccgcgccgacaccagcgtgcaggcatcctccagcgaccacaacccggagacgtatccggcggcgagctcaccgacagagtgaccgaccaccgcgtccggccgtacaccccacgattccagcaacccgaacagggccacctgcaaagcgaacagggccggctgggcatagccggtctcgttcacctccagatcgggcacgtccagcaggccccacacctgctgatggatccgcgcgaagacggggaacgcggcggccagttcctcacccataccagcacgctgcgacccctgacccgggaagacgaacaccacccgcggatcagacaccgtggtcccggtaacggtgtcatctccaaggagcaccgcccggtgctcgaacaccgaacgcgtcaccgccagcgttgacgccacaccccgcacgtccaccccaggcgacgccgccaaatacgcacgcagccggtcctcgtactcgaccagagctggcaacgtcctcgccgaaatcaccagcggcaccaactccgacgccaccaccggcgcctccgcaggcggcacatcgtccacagcctgcgtggggggtgcgctttccaggatgacgtgggcgttggtgccactgatcccgaaggacgacacacctgcccggcgaggccgacccatatccggccacggctggttctccgtcaccagcgcaaccgcgccatccgtccagtccacatgccgcgacggctcatccacatgcaacgtacgcggcaccagaccgtgctgcagcgccatcaccatcttgatgacaccagacacacccgcagcggcctgcgtatgacccagattcgacttcaccgaacccaacagcaacggctgctcacgatcctggccataggtcgccagcagcgcctgggcctcgatcgggtcacccagcgtcgtacccgtgccgtgcgcctcgaccacatccacctcagccgtactcagacccgcattgctgagagccgcccggatcacccgctgctgcgaaggaccgttcggcgcggacaggccgttcgacgcaccgtcctgattgaccgccgatccccgcaccaccgcaagcacctggtggcccttggcctgagcatccgacaaacgctctaccaacaggacgccggcgccttcagcccatcccgtgccgtccgcggcatcggcgaacgccttgcaccggccgtcggaggccaggccacgctgccgcgagaactccacgaaactttgcggcgtggccatcacagtgacaccaccgaccagggccagcgaacactcaccctgccgcagggcataccccgcctgatgcaacgccaccaacgacgacgaacacgccgtgtccaccgtgacagccggcccctcgagaccgaagaaatacgacacccgacccgacaggacactcaccgcaccggcagttgccccgaagccgccgaggtcggcaccaacgccgtagccaccggggtacgcacccatgaaaacaccggtgtcgctaccgcggagggaaccgggttcgatcccggcccgttcgaacgcttcccaggacgcctccaggaccagccgctgctgcggatccatcgccaacgcctcacgaggactgatcccgaagaacgaggcgtcgaaaccgcctgcggagtccaggaagccgccctgcacgcagtacgacttgcctgccgcctccggatcagggtcgtacaggttctcgacgtcccagccacggtcggcggggaatccggaaatcgcgtccgtacccgactcgacaaggcgccacagatcctccggggacgacaccccacccggcagacggcacgccatacccacaatcgccaacggctcgtccatggctgcccgcaacgacgtgttcgccttgcggagccgcgcgttctccttgaccgacttgcgcaatgcgtcgagaagctgatcttccctcatcgttcttcctccagcacgtgcttgatgagagcgtcagcgtccatctcatcgacgaaggcctcgtcatcggattccgaatcgggctttggaacggccaggccctccatggctgccagcctcagcagcgtgtccagcacaccggcctcccggaacttgtcaatcgagatccccgcgagcgctcggcgtagctcctgctcgtggaggcgaggttccggctccgccggagcgaacaactcgtccagccgggcggccagggccgtcggggtggggtagtcgaacaccaacgtggcgggcagccgcagccccgtcgccttcgccagactgttccgcagctccactgcggtcagcgaatccacacccagatccttgaacgccgcggtcacgggaatgctgtcgatgtcggcgtgtcccagaaccgtcgcggcgctgtcacacaccaccctcaacaacgccttcgcccgttcctccggtgccaaagctgccaaccactgcactgacgagtcaccggtagaggcggccctgcgagcaatcggccggtgcaacgaccggagcagcgccgggacttccgcgtcccgtaccggctccatcgccgcggccaccagaaccggttcactatggcgtgacgcgctgtcgaacagccccatcccgtactcggcggagatggcccgcaggccaccgcgccggatccggtcacggtccgtgtcggtcaggttcgcggtcaggccactggcgtcctcccacagaccccaggccaccgcgagtgcgggcagaccctcggcacgacgctgctcagccagcgcgtccacgaacgcattggccgccgcgtagttgccctgccccgcactaccgagcacaccggcggcggaggaatacatgacgaacgccgccaggtcggcatcccgggtgagctcgtgcaggttccacgcgccatcggccttgggccgcaacaccgcgtccagccgctgcgcggtcaaagattcgaccacaccgtcatcgagtgcgcccgcggtgtgaatcacggccgtcagggggtgctccggtgacacacccgccagcacctgggccagcgcggcacgatcggacacgtcacaggccgctgtctcgacccgggctcccagttcgccgagttggttgatcagtgcctcgtccggggcgctgcgggacagcagaaggagatgacgcacgccgcgttcggccaccaggtgccgggcggcgatccccgccagcacacccgaaccaccggtgatcaggaccgtgccatccggatcccacaccttctctgactcgggttcagcggcatgcgtacgggtcagtcgtggcgcttcgaagcggccgtcgctgatccgcagccgcggctcatccagtccgaccgccgccgccagctgatccggagcgagggcgtcgtcatcactctcgaccaggacgaaccggccggggtgttcggactgggccgaccgcatcaaccccgacaccccagcagcggccaaaccagtaccggtccgcaccaccagcgtgctgtcgctgaagcgctccccgccaagccactcctgaactgcctggaggacctgaccggtcagctttcgtgtttgctccagcgggtcgtcgtcgttgtcggggagtgcggtgaagaccgccacctcgggcaccggcacatcaccgtccgacaccagctcctcgaaccggccgaccgtcaggccggtctgctggggggtggggattcctgcccaggtcaaggcgagcaggtcatcggcagcggaaccgagcgcgtcggccgtcaccgggcgcgtcacgagcgaacccacgctagccaccggccgaccgatgtcatcagcgacccgcacactccagccatccgccgcctgcgtcaccgcgacacgcaacatggccgaaccaccagcatggacccgaacaccgttccaggagaacggcagccccacacccacctcggcatcggacgcattcaaggtgcaggcatgcagagccgcatccagtagagccggatgcacggcatatcgcgccgcctcccgcacctgctcctcagccagagcaacctcggcgaagacagtgtccccatcacgccacgcggcctgcaaaccctggaaggcaggtccgtactcgtaacccatcccggtcagctggtcgtagaagcccgccacgtccaccggcttggcctgcgcaggtggccacgcggtgaggtccgacggtgggacagtcgtgtcggaaaccctgacggtggcggaaacgtgccgggtccaggtatcggcgttgtccgcccgggagaagaccgtcaccacccggtgtccggactcgtcggcctcgccgacggacacggacaattgcacaccgccggtctgcggcagcaggagcggggtttcgatcaccagctcatcgacaacgtcacaaccgacctcgccagcggcgtggacaaccagctccacaaatgcggtaccgggcagcaggacactgccccagaccgcgtgatcagccagccacgcgtgggtggccagcgatacccgcccggtcaacagcacaccatcggattcaggcaactccaccaccgcacccagcaacggatgaccatccgcagccgcccggtccacgctcttgacccagtaccgctggtgttggaaggcgtaggtcggaaggtccagcacccgggccgtggtggtgccgaggatggcgggccagtcgacggtgacgccgtggacataggcctgtgccagggcggtgagcatccgggtggcgtccccgtcgtcacgacgcagcgtggcaaccgtgacgacatcgtcgtccatcgcctgcaacaacaccggactcgcgctgacctccacgaacacggcgtcgccctgagcctgcaactgactgacggcggggtggaaaccaaccggctcacgcaggttccggtaccagtactccccgtccagcgggctgtcgacccaggagccgtccaccgtcgacagccacggcaccaccggaggctgcgaaccgataccggcggtgatgccgagtagttcgtcgcggatcagctcgacgtggggggtgtgcgaggcatagtcgacggtgatccgccgcacccgcacccctcgtgcttcatgagcggtgaggacatggtcgactgcttccggggtgcccgcgatcacggtggaggcgggaccgttgtgggcggcgatccaggccccgtcgaccagctcgatctcgtgcgcgggcagggcgacggatgccatcgcgccccggcccgccaggccccgggcgatcgcctggctgcgcaaggtcacgatccgggcggcatcgcgtagtgacaccgcaccggccacacaggccgcggcgatctcaccctgcgaatgaccgatcaccgcatccggccgcacaccggccgcctgccacaccgcggccagcgaaaccatcaccgcccacgaagcaggctgaaccacatcaacccggtccaccaccgccggatcatccagcacggcgaacagatcccagtccacaaactcactcaacgccgccgcacactcggccatccgctcggcgaacaccaccgaggaatcccgcagtgcactgcccatccccagccactgccacccctgccccgggaaaacaaacaccacccgggggtcggtcaccgcggtgccggtgacggcgtcatctccaaggagtacggcgcggtgctcgaacaccgaccgcgtcaccgccagcgtcgatgccacagcccgcgtatccgccccgggcgacgccgccaggtaggcgcgcagccggtcttcgtgttcggtcagggcgggctgggtcttggccgatatcaccagcggcagcacatccgaggccaccaccggcgtctcaacaggctgctcctcttccgcgggctgtgcggggggtgcgctctccaggacgacgtgggcgttggtgccgctgactccgaaggatgacacgcctgcccggcggggccggtcggtgccgggccagggctggttctccctcaccagctcaaccgcacccgccgcccagtccacatgccgcgacggctcatccacatgcaacgtgcgcggcacgaaaccgtgccgcagcgccatcaccatcttgataacacccgacacacccgaggcagcctgcgtatgaccgatgttcgacttcaacgagcccagcagcaacggctgctcacggtcctggccataggtcgccagcagcgcctgggcctcgatcgggtcacccagcgtcgtacccgtgccgtgcgcctcgaccacatccacgtcagccgtactcagacccgcattgctgagagcggcccgaatcacccgctgctgcgaaggaccattcggcgcggtcagaccgttcgacgcaccgtcctggttcaccgccgatccacgcaccaccgccaacacctggtgaccgttacggcgggcgtcggacagccgctcgaccagcagcagacccgcgccttcggagaaaccggcaccgtccgcactgtccgcgaacgccttgcaccggccatcggaggccagaccacgctgccgggcgaactcggtgaagacctccggcgtggccatcaccgtcacaccaccggccaacgccatcgaacactcaccattgcgcaacgcctgcgccgccagatgcaaagcaaccagggacgaggagcacgccgtgtccatcgtgatcgccgggccctcaaggccgaagacgtacgacacccggccggacaacacgctggggccggacgtggcgccataaccttcgcgatcgaacccggcgccgtagccaacggggaatgcccctatgaaaacgccggtgtcgctgccgcgtacggaaccgggctcgatcccggcccgctcgaacgcctcccaggacacctccaacagcagccgctgctgcggatccatcgccaacgcctcacgcggactgatcccgaagaatccggcgtcgaagccagccgccgtgtccaggaagccaccctgcacgcaatacgacttccccgccgcctccggatcagagtcgtacaggttctccacgtcccagccacggtcagtgggaaatccggagaccgcatccgtacccgactcgaccaaacgccacagatcctccggcgacgacaccccacccggcaaacggcacgccatacccacaatcgccaacggctcgtcctgccccaccaccgacaccggcccgcgtaccggtgcagggttctcgccggtgaacaactcgtccaaccgagcagccaagaccgtcggggtcggatagtcgaacaccaacgtggccggcaaccgcaaccccgtcgccttcaccagaccgttccgtagttccaccgccatcagcgaatccacacccagatccttgaacgcgccagccgccggaatgctgcgtgcgtcggcgtgccccagaaccatggcggcactgtcacacaccagcttcagcagcgccttctcccgctcggccggtgccagggcggccagccactgcaccccaccgtcggcggaagcggcccgccgggcaatcgggcgacgcaacgaacgcagcaacgcagggacttcgccgtctcgggtcgggtccaacaccgcgggcaccaagagcggttcgccatggtgcgacgcggtgtcgaacagccgcattccgtcctcagcggtgatggttcgcatgccgctgcaccggatccggtcctggtcggtgccggtcagttgcgcggtcaggtccccggtgtactcccacggaccccacgccagcgcgagtgcgggcaggccctgggtacgccgttgttcggccagggcgtccaggaacgcgttcgccgccgcgtagttgccttgcccctcattacccagcacagcggcagccgacgagtacacgacgaatgccgccaggtcggcatcccgggtgagctcgtgtaggtgccaggcgccgtcggccttgggccgtagcaccgtgtcgagctgttgtgtggtgagggattccacgacgccgtcgtcgagtgcgccggcggtgtggatcaccgccgtcagcgggtgttccggtgatacgcccgccagcacccgtgccagcccggcacggtccgacacgtcgcaggccgctgtctcgacccgggcgcccagctcggcaagctccttgatcagtgcttcgtcgggagcggtgcgggacagcagcaggaggtgacgtgcaccgtgttcggctgccaggtggcgggcgaggatgcctgccagaacgccggaaccgccggtgatcaggatggtgccgtcggggtccggccggcgggggatggtgaggacgatcttgccgatgtgccgcgcgcggctcatccagctgaacgcatcgcgagcttggcgaatgtcccaagtttgtacgggcagcggctgtaggacgccttgtccgaacaggctgaggatttcggtaatgatctcccgcagccggtccgggccgacgtccatcaggtcgcacgcctggtacgtggtgccgggccggtcggcggtgatccggtgcgggtcgcggatgtcggtcttgccgatttccacgaaccggccgccgcggggcagcaggtcgagggaggcgtcgacgaagtcaccgctgagggagttcaggacgacatcgacaccccggccgtcggtagtgttcaggaacgcttcccggaagcccgtggtgcgggaatccgcgatccgagtgccgtccagacccgcctcgtacagaatgtgctgcttgcccgcgctggcggtcgcgtagatctgcgcgccgaggtggcgagcgatctgggtcgctgccatgcccacaccacccgcggcggcgtggatcagcacggactcacccgcactcagcccgcccagatccaccagcccgtaatacgcggtggcgaacacaaccggcacggacgccgccgtagtgaacgaccagccatcaggaatcacggcgagcattcgacggtcggcgatcgcgaccgccccgaatccgccgcccacaagaccgaacactcggtctcccggggccaggtcgtgggcctccgggccgacctcgagcaccacgcccgcagcttcagccccgatcacagcctcaccgggataagtgccgagcgcgataagaacgtcgcggaagttcaggcctgcggcgcgtacgtctatgcgtacttcgccgggtcgcaggggccgttcggcggtgtcggtgggtaccagagcgaggtcttgcagggttccgctgcggggttgttccagcagccaggcacgatcgtcggggatcgtcagcgggctgctctccggcgtattcgcccgcgccagccgcggtacctcgaaccggccgtcgcagacccgcagccgcggctcgtccagcccggcggtcgccgccagctggtccggggtgagggtgtcgtcgtcgccttccaccagaacgaaccggcccgggtgctccgactggaccgaacgcatcagccctgacaccgcggcggcggccaacccggtgccggtccgcacgaccagcgtgctgtcggtgaaacgttccccggccagccatgcctggactgcctggagaacctgggcggtcaacgtgcgagtctgtgccagcggatcagggctgccgtcggtgtcgggacgcgcggtgtagaccaccacctcgggcaccggcacatcggcgtccacgagatcctcgaaccggccagtggtcagaccggtctgctggggaatggggatctccgtccagaccaccctgagcaggtcatcggctgcggagccgagcgcgtcggctgtcaccgggcgcgtcacgaatgagccgatggtggccaccggccgaccgtcgtcatcggcgacgtgcacactccagccgtctgttgtcggtgtcatcgcaacacgcagcgtggccgagccagtggcatggacttggacgtggctccaggagaacggcatccgcaggctctgctgctcagcgtcgggtgtgttcaagacgctggcgtgcagggcggcgtccaacagcgccggatgcaccgcgaaccgtgccgcctcctgcgcctgctcctcggccagagcaacctcggcgtaggcggtgtccccgtcacgccacgcggcctgcagcccctggaacgcgggaccgtactcggtacctgccgcagccagccggtcgtagaagtcggccacgctcaccggccgggcctgggctggtggccacgaggcaaactccggcagcgagaccgtcgagtcagaggtgctgactgtcgccgtgacgtgccgggtccaggtatcggtgttgtccgcctgggagaagaccatcaccgcgcggtgcccggactcgtcggctccgccgacggacacggacagttggacgcctccggtctgcggcagcaggagcggggtttcgatcaccaactcgtcaacgacgtcgcactcgacctcgtcggcggcgcggacgaccagttccacaaatgcggtcccgggtagcaggacactgccccggaccgcgtgatcggccagccatgtatgggtggccagcgacacccgcccggtgagcaccacaccgtcggagccgggcagtgccactacggtgcccagcagtggatgaccgtcggcagccgcccggtccacgctcctgagccagtaccgctggtgctggaaggcgtaggtcggaaggtccagtacccgggttgtggcggtgccgaggatggcgggccagtcgacggtgacgccgtggacataggcctgtgccagggcggtgagcatccgggtggcgtcgccgtcgtcacgacgcagcgtggcaaccgtgacgacatcgtcgtccatcgcctgcaacaacaccgggctggcgctgacctccacgaacacggtgtcaccctcggcctgcaactggccgacggcggggtggaaaccgaccggctcacgcaggttccggtaccagtactccccgtccagcgggctgtcgacccaggagccgtccacggtcgacagccacggcacgagcggggcctgcgagctgctgtcgctagtgatgtcgagcagttcgtcgcggatcagctcgacatgaggggtgtgcgaggcatagtcgacggtgatccgccgcacccgcacccctcgcgcttcatgagcggtgaggacatggtcgaccgcttccggggtgcccgcgatcacggtggaggcgggaccgttgtgggcggcgatccaggccccgtcgaccagctccacatcctgcgcgggcagggcgacggatgccatcgcgccccggcccgccaggccccgggcgatcgcctggctgcgcaaggtcacgatccgggcggcatcccgcaacgacaccgcacccgccacacacgccgcggcgatctcaccctgcgaatggccgatcaccgcatccggccgcacaccggccgcctgccacaccgcggccagggaaaccatcaccgcccacgaagcgggctggaccacatcaacccggtccaccaccgccggatcatccagaaccgtgaacagatcccagtccacaaactcactcaacgccgccgcacactcggccatccgctcggcgaacaccaccgacgaatcccgcagcgcactgcccatccccagccactgccacccctgccccgggaaaacaaacaccacccggggatcggacacagcggtgccggtgacggtgtcgtctccaaggagtacggcgcggtgctcgaacaccgaccgtgtcaccgccagcgtcgatgccacagcccgcgtatccactccgggcgacgccgccaggtaggcgcgcagccggtcttcgtgttcggtcagggcgggctgggtcttggccgatatcaccagcggcagcacatccgaggccaccaccggcgtctcaacaggctgcgcctcctccgcgggctgagcggggggtgcgctctccaggatgacgtgggcgttggtgccgctgactccgaaggacgacacgcccgcccgacggggccggtcggtgccgggccagggctggttctccctcaccagctcaaccgcacccgccgcccagtccacgtgccgcgacggctcatccacgtgcagggtgcggggaaccgtgttgtgctgcagggccatgaccatcttgatgacacccgacacgcccgaggcggcctgagcatgaccgatgttcgacttcagcgagcccagcagcaggggccgttcccggtcctgaccgtaagtggcgatcaccgcctgggcctcgatcgggtcgcccagcgtcgtacccgtgccgtgggcctcgaccacatccacctcgtgcgcggcgaggccggcgttactgagagcggcttggatcacccgctgctgggaggggccgttcggcgcggtcagaccgttggacgcgccgtcctggttgaccgccgagctacggaccaccgccaacacctgatggcccttggcctgggcgtcggaaagtcgctctaccagcagcacaccaacgccttcagcccatcccgtgccatccgcgctgtctgcgaacgccttgcaccggccatcggaggccagaccacgctgccgcgagaactcggtgaagagctccggcgtggccatcacggtgacaccgccgaccagggccagcgagcactctccctgccggagggcataccccgcctggtgcagggcgaccagcgacgacgaacacgctgtgtcgactgtgaacgcggggccttcgaggccgaagaagtacgacacccggccggacaggacactggtcgcgctggcggtggccgcgaagccgccgaggtcagcaccggcaccgtaaccaccggggtacgcgcccatgaagacgccggtgtcgctgccgcgcacggaaccgggctcgatcccggcccgctcgaacgcctcccaggacacctccagcaccagccgctgctgcggatccatcgccaacgcctcacgcggactgatgccgaagaatccggcatcaaagccagccgcggcatccaggaagccaccctcagcacggtgcgattttccagccatgtcgtacaggttctcgacgtcccagccacggtcggtggggaaaccggagaccgcatctgtacccgactccaggagacgccacagatcctcaggcgacgagactccaccaggtaggcggcaggccattcccacgatcgccagcggctcgtcctgcgccaccgccgacaccggcccgcgtaccggtacggggttctcgccggtgaacaactcccccagccgaacagccaaagccgtcggagtcggatagtcgaacaccaacgtggcgggcagccgcaaccccgtcgccttcgccagactgttccgtagttccaccgcagtcagcgaatccacacccagatccctgaacacggcggctacggagacggtgctggtgtcggcgtgtcccagaaccgtcgcggcactgtcacacaccagcttcagcagtgccttctcccgctcggccggtgccagggcggccagccaccgcgctccaccggcggcagcggcccgccgagcaatcgggcggtgcaacgaccgcagcaatgccgggacttccgcgtcccgtaccggctccatcggcgcggccaccagaaccggttcactgtggcgtgacgcgctgtcgaacagccccatcccgtgctcggcggagatggcccgcaggccaccgcgccggatccggtcacggtccgtgtcggtcagttgcgcggtcaggccactggcgtcctcccacagaccccaggccaccgcgagtgcgggcagaccctcagcgtgacgctgctcagccagcgcgtccaggaacgcattggccgccgcgtagttggcctgccccgcactaccgagcacaccggcggcggaggaatacatgacgaacgcggccaggtcggcatcccgggtgagctcgtgcaggttccacgcaccgtcggccttgggccgcagtaccgtctccagccgctgcacggtcaaagactccacaacaccgtcatccaccacgcccgcggtgtgaatcacggccgtcaggggatgctccggcgagacacccgccagcacctgcgtcagcgcggcaggatcggacacgtcacaggccgctgtctcgacccgggcccccagttcaccaagctcgccgatcagcgcctcatccggggcgctgcgggacagcagcaggagatgacgcacgccgcgttcggtcaccaggtgccgggcggcgatccccgccagcacacctgaaccgcccgtgatcaggaccgtgccatccggatcccacgtcctttcaggctcaggctcttcggcatgtgtgcgggtcagccgtggtacttcgtaccggccgtcgctgacccgcagccgcggctcatccagtccgaccgccgccgccagctgatcctgggtgagggcgtcgtcgctttcgaccaggacgaaccggccggggtgttcggactgggccgagcgcatcagccccgacaccccagcagcggccaacccggtgccggtccgcaccaccagcgtgctgtcgctgaagcgctccccgccaagccactcctggagtgtgtggaggacctgagcggtcagctttcgtgtttgctccagcggatcgtcgctgtcggggagtgcggtgaagaccgccacctcgggtaccggcacatcgccgtccgccgccaggtcttcaaacgcacccaccgtcaggccggtctgctggggggtggggattcctgcccaggtcagggtgagcaggtcgtcggtgctggtggccggttcgtatacggggacggtgcgcagggtgagcgagcgggcggtcaggacggggaggcctgtgctgtcggtggcctctgccgacagggtgccgtcatcgtgtgaggtgaggcggacgcgcagctcgccgggattgccggcgtgcagggtcaggccctgccacacggcgggagtctcctcgcctacggtggtgagcgcggcggccatcagggcgggatgaagcacaaacctggtggcatcatgggcatcgtccagggccacctcggcgaagatgtcgctgcccaggcgccacaccgcccgcagtgagggtgccgatgtcccgaccgggaccgcaccggccggcggccacaccccaccggcctgccagccgaccgcggcagcggccacacccgtagtggtggccagggttgcggtggccaagcaggtccagggctcgccgtcaccgtaacgggcgtggacactgagccggcgccgaccgccctcatccggttcgcccacccaggtctgtacctcgacggcgccatcatcgggaagcagcagcaacgcttccacactcagatcctcaaccagaccgcagccgacctcgtccgccgctgccagcgccatctccacgaacaccgtcgcgggcagaaccgtctggtcccgcagccacagatcaccgctccgggaaacccgggcagtgaacaacaccccactggcctcggccagccgcaccaccgaacccagcaacggatgaccacccgcagtcgcccggtccgtgccctcaagccagtaccgctggtgctggaaggcgtacgtcggaagatccagcacccgggttaccgggacatcacccagcaccgcggaccactcgacactcacgccgttcacgtacaggtgagccagggcaccgacagcggcctgcgcctcatggtcaccgtgcagcatcgcgatgccatcgaccaggcgggccagtgaccggtcggcacccagctcgacgaacaccgcatcctcgaacgaggccacctgctcgccgaaccggaccgtgtcccggacctgccgcacccagtactcagcggtcatcacctgatcaccagcggccatggcgacctgcggcgtccggtaggtcaggccttcagcgaccgcccggaactcctccagcatcggttccatacgggcggaatggaacgcgtgactggtcgccagccgcgtccacttccccagcccctccgcggcctgcagcacggcggcctcatcaccggagagaaccaccgacgacggcccgttgaccgcggcgatctccacaccctcacccagcacggcccgagcctcatcctccgagaccgggacagcgaccatcaccccacccgcaggcagagcctgcatcagacgagcccgcgccgacaccaaagtgcaggcatcctccaacgaccacaacccggagacgtatccggcggcgagctcaccgacagagtgaccgaccaccgcatccggccgtacaccccacgattccagcaacccgaacagagccacctgcaaagcgaacagggccggctgggcatacccggtctcattcacatcgagatcgggcacatccagcagatcccacacctgctgatggatccgcgcgaagacggggaacgcggcggccagttcctcacccataccagcacgctgcgacccctgacccgggaagacgaacaccacccgcggatcggtcgccgcggtaccggtgacggtgtcatctccaatgagtacggcccggtactcgaacaccgaccgtgtcatcgccagcgtcgatgccacagcccgcgtatccgccccgggcgacgccgccagatacgcacgcaaccggccctcgtattcagccagtgccgactgggtcctcgccgaaatcaccaacggcacccactccggtgccgactcgaccactgtgttgcccgcaggctgtgtggggagtgcgctttccaggatgacgtgggcgttggtgccactgatcccgaaggacgacacacctgcccggcgaggccggtcggtgccgggccagggctggttctccgtcacgagctgcaccgcgcccgccgaccagtccacatgccgtgacggctcatccacatgcaacgtacgcggcacgaaaccgcgctgcagcgccatcaccatcttgatgacacccgacacacccgcagcagcctgcgtatgacccagattcgacttcaccgaacccagcagcaacggctgctcacgatcctggccataggtcgccatcaacgcctgggcctcgatcgggtcacccagcgtcgtacccgtgccgtgcgcctcgaccacatccacctcagccgtactcagacccgcgttgctgagagcggcccggatcacccgctgctgcgaaggaccattcggcgcggtcagaccgttcgacgcaccgtcctgattgaccgccgatccccgcaccaccgcaagcacctggtgaccgttacgacgggcatcggaaagccgctccaccaacagcacaccgacgccctcggagaagccggcgccgtccgcactgtccgcgaacgccttactgcgaccgtcaccggccaggccaccctgacgagcgaactccacgaaagtctgcggcgtggccatcacagtgacaccaccgaccagggccagcgaacactcaccctgccgcagggcataccccgcctgatgcaacgccaccaacgacgacgaacacgccgtgtcgaccgtgaacgccgggccctcaaatccgaagaaatacgacacccgacccgacaggacactcaccgcaccggcagttgtcccgaagccgccgaggtcggcaccgatgccgtagccaccggggtacgcacccatgaaaacaccggtatcagttccgcgtacggaaccgggcttgatcccggcccgctcaaacgcctcccaggacacctccagcaccaaccgctgctgcggatccatcgccaacgcctcacgcggactgatcccgaagaacgaggcatcgaaaccagccgcggcatccaggaagccaccctgcaagcgataggagttgccgactgcggggccgaacagattctccacatcccagccacgatcggtggggaatccggagaccgcgtccgtacccgactccagaagacgccacagatcctcaggcgacgacaccccacccggcaaacggcacgccatacccacaatcgccaacggctcgtcctgcgccaccaccgacaccgacgtacgtaccggtgcagggttctcgccggtgaacagctcctccaaccgcgcagccaaagcggccggggtcgggtagtcgaacaccaacgtggcgggcaaccgcaaccctgtcgccttcgccagaccgttccgcagctccaccgccgtcagcgaatccacgcccagatccctgaacgcaccagtcgccggaatcgtgctggcgtcggcatgtcccagaaccgtcgcggcgccatcacacaccagcttcagcagcgccttctcccgctccaccggcgccaaccgggccagccactgcaccgacgaatcaccggtagaagcagcccgccgggcaaccggacgatgcaacaaacgcagcagcgccggaacctcgacatcacgcacaggctccatcgacgcagcgagcagcaccggctcaccatgacgtgacgcggcatcgagcaagcccatcccccggcccgcggagatcgcccgcaggccaccacgccggatccggtcacggtccgtatcggtcatcttcgcggtcagaccactggtgtcctcccacagaccccacgccaccgccagcgcgggcagcccctcaccacgacgctgctcagccagcgcatccacgaacgcattggccgccgcataattaccctgccccgcactaccgagcacaccagcagccgaggaatacatgacgaacgccgccaggtcggtgttctgggtgagttcgtgtaggtgccaggcgccgtcggccttgggccgtagcaccgtgtcgagctgttgtgtggtgagggattccacgacgccgtcgtcgagtgcgccggcggtgtggatcacggcggtcagggggtgttcgggtgatacgcccgccagtacgcgcgccagcccggcacggtccgacacgtcgcaggtggctgtgtcgacgtgggcgcccagctcggcaagctccttgatcagtgcttggtcgggggtggtgcgggacagcagcaggaggtgacgtgcaccgtgttcggctgccaggtggcgggcgaggatgcctgccaggacgccggagccgccggtgatcaggatggtgccgtcggggtccggccggcgggggatggtgaggacgatcttgccgatgtggcgggcgcggctcatccagctgaacgcatcgcgggcctggcggatgtcccaggcctgcaccggcaggggcagtaggacgccttgcgtgaacagggtgaggagttcggtgatgatttcccgtagccggtccgggccggcgtccatcaggtcgaaggcctggtaggtggtgcccggccggtcggcggtgacctggtgcgggtcacggatgtcggtcttgcccatctcgatgaaccggccgccgcggggcagcaggtcgagggaggcgtcgacgaagtcaccgctgagggagttcaggacgacatcgacgccctggccgtgtgtgttgttgaggaaggtttcctggaaactcagtgtgcgggagtcggcgatgtgggtgtcttcgagaccggcttcgcgcaggatgtgttgtttcccggtgctggcggtcgcgtagatccgcgcgccgaggtggcgggcgatctgggtggctgccatgccgacaccacccgcggcggcgtggatcagcacggactcacccgcactcagcccggccagatccaccagcccgtagtacgcggtggcgaacacaaccgggacggacgccgccgtagtgaacgaccacccgtcaggaatcacacccagcattcggcgatcagcgatcgcgaccgccccgaacccaccgcccacaagaccgaacactcggtctcccggggccagatcctggacctccggaccgacctcgagcaccacgcccgcagcctcaccgcctgccaggcgtttgtcgtcaaccatgccgagggccacgacgacgtcgcggaagttgagtcctgcggcgcgtacgtctactcgtacttcaccggattgcaggggccgttcggcggtgtcggtgggtaccagagcgaggtcccgcaaggttccgctgcggggttgttccaacagccaggcacgactgtcggggatcgtcagcgggctgctttccggcgtattcgcccgcgccagccgcggtacctcgaaccggccgtcgcagacccgcagccgcggctcgtccaatccgacagtcgccgccaactggtccggggtgagggtgtcgtcgtcgcattccaccagaacgaaccggcccggatgctccgactggaccgaacgcatcaaccccgacaccccggcggcggccaaaccggtaccggtccgcacgaccagcgtgctgttcgtgaagccttccccggccagccatgcctgaactgcctggagaacctgggcggtcaacgtgcgagtctgtgccagcgggtccagcgggtccagcggagtctcgctgctgccgggaagtgcggtgaacaccaccacctcgggcaccggcacatcaccacccgacaccagatcctcgaaccggccgaccgtcagaccggtcccctgaggagccgggatctccatccacgtcagggcgagcagctcatcggcagcggaaccaagcgcgtcggccgtcaccacccgtgtcacgagcgaaccgacgctagccactggccggccactgtcgtcagcgaccagcacactccagccgtccgctgttcgtgttatcgcgacacgcagcatcaccggccctgtggcgtggacccaaacctggttccaggagaacggcagccgcacaccctgctcggcggtgtcgagtgtgctcaaattgctggcgtggagggcggcgtccaacagcgccggatgcaccgcgaaccgtgccgcctcctgcgcctgctcctcagccagagcaacctcggcgaagacggtgttgccgtcgcgccacgcagtccgcagcccctggaacgcgggaccgtactcgtaaccggccccagtcagctggtcgtagaaatcactaaggtcaagcggctctgcccctgcgggtgggggccaagcagcgaactccggctgcgagaccgtcgagtcagaggtgctgactgtcgccgtgacgtgccgggtccacgtatcggtgttgtccgcctgggagaagaccatcaccgcgcggtgcccggactcgtcggcctcggcgaccgatacggacatttgcacaccgccagtctgcggcagcaggagcggggtttcgatcaccagctcatcgacaacgtcacaccccacctcgccggcggcgcggacgaccagctcaacaaatgcggtcccgggcagcagcacactgccccgcaccgcgtgatcggccagccacgcatgcgtggccagcgacacccgcccggtcagcaccacaccatcggagccgggcagcgccactaccgtgcccagcagtggatgaccatcggcagccgcccggtccacgcccttgagccagtaccgctggtgctggaacgcgtacgtcggaaggtccagcacccgggctgcggtggtacccagcaccgcaggccagtcgacagtcacgccctcgacatatgcctgcgccagggcggtgagcatccgggtggcgtccccgtcgtcacgacgcagcgtggcaaccgtgacgacatcgtcgtccatcgcctgcaacaacaccgggctggcgctgacctccacgaacacggtgtcaccctgggcctgcaactggccggcggcgggctcgaaacccaccggctcacgcaagttccggtaccagtactccgcgtccaaggggccttcgacccacgtcccatccaccgtcgacagccacggcaccaccggagcccgcgaatcaacaccggcgatgacacccagcagctcgtcacggatcagctcgacgtgcggggtgtgcgaggcgtagtcaacggcgatccgccgcacccgcaccccccgcgcctcatgaaccgccagcacacggtccaccgcctccggcgcacccgcaaccaccgtagacgcgggaccattacgagccgcgacccacgccccgtcgaccagctcgacatcctgcgcgggcaacgcaaccgatgccatcgcaccccggcccgccagcccccgcgcgatcacctgactgcgcaacgtcacaacccgcgccgcatcccgcaacgacaccgcacccgccacacacgccgcggcgatctcaccctgcgaatgaccgaccaccgcatccggccgcacaccggccgcctgccacaccgcggccagcgaaaccatcaccgcccacgaagcaggctgaaccacatcaacccgggccaccaccgccggatcatccagcacggcgaacagatcccagtccacgaactcactcaacgccgccgcacactcggccatccgctcggcgaacaccatcgacgaagtgcgcagcgcactgcccatccccagccactgccacccctgacccgggaaaacaaacaccacccgaggatcagacacagcggtgccggtgccggtgacgctgtcatcccccagcagcacggcacggtgctcgaacagcgaccgcgtcaccgccagcgtcgaccccacagcccgcacatccgccccgggcgacgccgccagatacgcacgcaaccggccctcgacctcggccagagcaggcaacgacttcgccgacaccaccaacggcaccagctccgaatccactacgggtgttgagccggccgcgtcgtccacggactgtgccggtgcaccctccaggatgacgtgggcgttcgtaccgctgaccccgaaggccgacacacccgcccggcgcggccgaccggtggcgggccaggaccggttctccgccaccagctcaaccgcgcccgccgaccagtccacatgccgcgacggctcatccacatgcagtgtgcgcggcacgaaaccgtgccgcagcgccatcaccatcttgatgacacccgacacacccgccgcggcctgcgcatgaccaatgttcgacttcaacgaccccagcaacacaggctgctcacggtcctggccgtaagtggcgatcaccgcctgggcctcgatcgggtcacccagcgtcgtacccgtgccgtgcgcctcgaccacatccacctcgtgcggcgccagaccggcgttgctgagagcggcccggatcacccgctgctgcgaaggaccgttcggcgcggacagaccgttcgacgcaccgtcctggttcacagccgagccccgcaccaccgccaacacctggtggcccttggcctgagcatccgacaagcgctccaccagaagcacgccgacgccctcggcccaccccgtgccatccgcggcgtcggcgaaggctttacaccggccatcggctgacaggccgcgctggcgggagaactccacgaaactttgcggcgtagccatcaccgtgacaccgccgaccagggccagcgagcactcaccctgccgcagggcataccccgcctggtgcaacgccaccagcgatgacgagcaggccgtgtccaccgtgatggcggggccttcgaggccgaagaaatacgacacccggccggacaagacactcgccgcaccagcggtcgcgccgaaaccctcaaggtcggcgccggcgccgtagccgccggggaaaccgcccatgaagacaccggtgtcgctgcctcgcacggagccgggctcgataccggcccgctcgaacgcctcccaggacacctccagcaccagccgctgctgcggatccatcgccaacgcctcacgcgggctgatcccgaagaacgaggcatcgaagccggccgcggtgtccaggaagccaccctgcacgcagtacgacttcccggacgcgtccgggtccggatcgaacaggccgtcgacgtcccagccacggtcggtgggaaaaccggaaatcgcgtccgtacccgactcgaccaagcgccacagatcctccggcgacgacacaccgcccggcagacggcacgccatacccacgatcgccaacggctcgccttgccccaccgccgacaccgcgcgctccggggcggggctctcgcccgtgaacaactcccccaaccggacgacgagcacggccggggtcggatagtcgaacaccaacgtggctggcagccgcagacccgttgccttggccaggctgttgcgcagttccaccgaggtcagcgagtcgatgcccatctcgcggaacgtcttgtcaggggcgtaccgatcggccgcggagaggcccaggatgtccgtgacgctgctgcggacgagatcgcgcaacgcctgttctctggcagcctccgcggctaggaactgccgcgaccatgcctgctgttcggtggtgtcggtgtgcgcaccgaccaagttctggaggagcgggggaagcgtgtcggccgcggccagtgctcgcagaccgacggtgttcagacgggtgacgatgtgcgccgagtcggcggaagccatcgcgcggcccagcaggggtcccctgaccacgaccgcttcggcttcgggtatgccccaggcgatcgccgtgatcggcaggccttcggcgcggcggcgccgggcgagggcgtccacggaggcgggcgcgtcttggctgaagacgacgaaggcggcgaggtcgaggcctttcgtgaggcggtgcagctggtggctctcgtgggcaacggcgggaccgccggtgtggatcactgcggtgagagggcgttccggtgctatcccggccagtgtccgcaccagcccggcacgatccgacacgtcacagaccgccgtgtcgacccgggcgcccgactcgatcagctcgttgatcagtgcttcgtccggggtgctgcgggacagcagcagcagatgacgcacgccctgttcggccaccagatgccgggcgacggttcccgtcagggcgcctgctccgtcggcgatcaggaccgtaccgtcggggtcgagtcgccggggaatggtgaaggtgatcttgccggtgtggcgggcgcggctcatccagctgaacgcgtcgcgggccttgcggatatcccaggtcagtacgggcagcggccgcagcacgccctgtgcgaacagttcgagcagctcggcgatgatttcccgcagccggtccgggccggcgtccaggaggtcgaacgcttgataggtggtaccaggccggtccgcggtgatccggtccgcgtcgcggatgtcggtcttgcccatttccaggaaccggccaccgcggggcagcaggtcgagcgaagcatcgacgaagtcaccgctgagagagttgaggacgacgtcgacaccctggccgtcggtggtgttcaggaacgcttcccggaatgcgagtgtgcgggagtccgcgatccgggtgtcctccaggcccgcctcacgcaggatgtgctgcttgcccgcgctggcggtcgcgtggatccgcgcgccgaggtggcggcaagcgatctgggtcgctgccatgcccacgccacccgccgcggcgtggatcagcacggactcacccgcactcagcccggccaggtccacgagcccgtagtaggcggtggcgaacacgatggggatggacgccgccgtcgcgaaggaccacgtatcgggtatcgcaccgagcagacggcgatcggtgatcgcgaccgccccgaacgcacttcccacgagcccgaagacccggtctcccggggtcaggtcgtgtacttccgggccgacctcgagcaccacgcccgcggcctcacctcccatcagtccctcacctgggtacgtgcccagagcgatgagaacatcccggaagttcagacccgcggcgcgcacggctatccgcacctcgccggggcgcaagggccgctcagcggtatcggcgggtaccagggcgaggtctcgcagggttccgctgcgggagtgctccagcagccacgcacggtcctcggggatcgtgagcgagtcgctgtccgcggtgttcgcgtgggccagtcgcgggacgagcggattaccgtcgcgcagcgcgagctgtggttctccgagcccggcgagggcccgcaggagggtgtcgtcggggtcggggtcggcatcgtcgaagtcgacgtcgatgacgacgaagacgccggggttctccgtctgttcccggcggatcgcgtcccacgccgctgcgtgctcggggaccgggaggtcggagggaccagtgtgcacggcacggcgggtgacgaagacgagtgtcgaatcggcacgccgcttgtcggcgagccaggactgcgcgagatcgcggaccgcgccggccaggtcggcggcgtcggacggcagggagtcgacgcgtacgaccacatggcggccgtccggggaggcctgtttcggcgggtcgaggggaatccactccagccggtgcagggtttcgccgccgctcgctccggcgagcaattcggccggtttctccgtgagggaggagcgcttcaccttgccggacgcggtccgcgggatggcgtcgacccggtagatctcctctggcaccttgaagtacgacaaccgttcgcggcacacggcgagcaccatgtcagtgtcgacaccgcccgtttccggcacgacgtacagcacgggaatctcgccgagtacggggtgctgcttgcctgccgctgccgcatccttgaccccgggcacgtcgagcgccactgcctcgatctcgtgcgggtggatgttctcaccaccgcggatgatgagttccttgatccggccggtgatggtgacgtgtcccgtctcgctgcggcgggccaggtcaccggtgcggtaccacccgtccgaaagcacctctgccgtggcttcgggctgctcgtggtagccgatcatgatgctcggtccgctggcccacagttcgccctcttcgccgtccgcgacgtcggcgccgctgatgggatcgacgaagcgcagggacagccccgggacggggaggccgcacgaaccggggatccgttgcccgctgagccagttgacggtgagggatccgctggtctcggtgcagccgtaggtgtcgagcagcggtacgccgaagacgtcctcgaagccctcgatgagggaagccggcgcgacggagccggcgaccaggcccattttcagcgacgacgtccgtggttcgaacgtgtcggcggctcccaggagatagcggaacatcgtcggcacgccgacgagcacggtgcaccgatgctccgtgagctcttcgagcacctcgctcgccacaaagccgttcatgatccgcgcagtggcgccgacggcggtggtggccaagagacacaggtggagggcgaggctgtggaacagcggcgcgggccagagcagaacgtcgttctcggtcagctcccaggcggggatatcgcaatacgccgcggaccagaggccgctgccctgggtcgacaggacgcccttcggggtgcccgtggtgccggaggtgtagagcatccaggctggttcatcgagccccaggtcgtcgcgtggcggccggaggggctcggtggtggccagctcggcgaatgaccgtactcctgggggagttcgttcctcgcccaccagaacgatggtcgctgcgggggccgtgcgacgcacatcgtcgagatgagcggagtcggtgatgacggctacggcgccgctgtccgacaggaagtgggcgacctcggcctcggtcgcgtccgggttgagcggtacggcaaccgcgctggcccgggcgatcgccagatagctttcgatgttctcgacgcggttccccagcaggatcgcgacccggtcgccccggtccacacccatgtccaccaggtgcccggcgatccgtccggtccgcgtggccagttcggcatgcgtcagtgcgagccgcgagtccacgtacgctattttgtcgccgagccgttcagcattcgcgcgaagcagatcataaataggccgtaccagttccgcgcgtgacattaatacaccttccggcaaagtacggtgtttctgataactctaaaccgactgcaatcagccaccagcgacgcaccaccgacaatcccatccgaacgatagcagtctcgcggagaagttctccatacccctccaccccctatgttgatcacccttctgcgcacgattcagcaggcaggacgtctagggggttgcgtggactgcggcctgaggtgtcttcttggatcaaggcatgctcggcccttcgggccatccattacaccgcagtccagccgtgccacaagaaggtgcacataggtcaccgggaggacccggaaaccctccattggatttgtttgggaaggccgtctccgaccgcagataacgaacgcctttccaggagaggtgaatggcttccgacagtccccgcccaaggccgctcaaaccggctttcgctgtttcgcggacgcagcgtcagcgtcgcagacttgacgagcgcggcatcagtcgcgccgatcggaacgggccacttccggcgtctttctcgcagcaacgtctatggctcatgcagcagctcgcacccgacagcaattcctataatctgccgctcgtgcagcggctgcggggcgccctggacaccgtgatcctcaggcgcgctctcggcctcgtcgcaagccgccacgaggcgcttcggaccgtattcgacgcggaggacggcgagccgctccagcggatccagcccgcggatgtcgtgcgcctgcgcgaactggacgcccgggacgaggaccacgccagagcgttggtccggcgggagacggccgagccgttcgacctgcacgccggtcctgtgatgagagcgttgttggtacgactcgccgacgatgaccacgtgctggcgctcacggtgcatcacatcgcgggcgacggctggtcgctggccatcctgcggatggagctctcggcgcagtacgcggcgctgcttcgcggtgcggaggccgatctgcccgcactcccgtcgcagtacgcggacttcgcccagtgggaacgcaaggcgttgtcgggcaccaagctgcaaaagcggctggactactggcaggagaacctgcgggacgccccgatggtgctgggattgccggccgaccggccacgaccgccggtcggcagttcggacgcgggcacggtggcctggtgcccgccgacggaattggtgaccgcggccaaagaactgagccggtcgacgggcaccacgctgttcatgtcgctgctggccgccttccaggtggtgctgtcccactacgcgcgcaccgatgacgtcctggtcggcaccccgatcgccaaccgcaatcacgcggaggtggaggggctgatcggcatgttcgtgaacacggtggtgctgcgcggcgatctgtcaggtgatccgaccttccgcgagctcctcctgcggacccgtacgaacgccatgggcgcgttcgcgaacgccgagctgccgttcgacctcctcgtcgagaaggtcgcaccgcagcgcgacctgtcggtgaacccggtcgtgcaggtgctcttccagctcatgccgctggcgtcgaacacgtttacgctgccgggaatcgcggtcgagccgttcgatatgaaccagttcttcacccgcatggatctggagttccatgtctacgaggatcccgccggggacaggctgacgggcgaggtctggtacagccgagcgctcttcgacgggcctcgtatagaacgaatgctggatcagttcactctcgtcctgcgcagcgtactcgacaacccggacgttccgatctcgcagatttcactcgcgccggagatcggtaccgacctgccgacggtcgaagcgaacgaaacagaccgagacttgccgttcgattcgctgccggaattgctggcggacgcggcagcaagaaaccccgacgcgatcgccgtcgtcgacgaacgggtcacactcacctatgccgaactcggtgggcgggccaataagatggcgcatctgctgcgacacaaaggcgttcgccccggcgaactagtcggtctttgcgtcgaccgaggcgccgatatgatccgtgggcatgctcggcattctcaaagccggcgcggcctatgtgcccatcgaccccgagcatcccatcgagcgcacccgtttcgtgttggacgattccagcatatccacggtcatcgctcaggagacgtaccggactcggttccccgatgtccgcgacatcattctcccggacgaccccggcctcgagaatcagcccgcaagcccgcccgatgtgacgaccgaccgcaacagcctggcctatgcgatttacacctccggttccacgggccgtcccaaggccgtgctcatgcccggcatctgcgtcgtcaacctgctgctgtggcaggaacgcacgatgggtcgcgaaccggccagtcgcaccgcccagttcatcaccgcgaccttcgactactcggttcaggaaatcttctccgcgctgctcgggggcacgcttgtcattccccccgacgacatacggctggaccccgcgcgactcgcacaatggatcgacgacagccgtatcacgcgcatctacgcacccaccacggtgctgcgcgcgctggtcgagcacgtcgacccgcacgggcacgggcttgtcgacattgcgtcacctctgccagggcggcgaggcgctcgtgctggacggcaagttgcgagaggtgtgccctgcaccgcccgcatctgcgggtgcacaaccactatggcccggcggagagccaactcgtcacgggctacacactgcccgaggacgtctccgcctggccggccacgacccctatcgggaagccgatcgacaacacgcgcatccaccttctcgacgacgcgcttcgtcccgtacccgacggcgtatgcggcgcaggtctgcatatcggggatcggtctggcccgcggctacctggcccggcccgaactgacccggcagcgattcatcaccgaaggtaccggctcggagccgaggatgtacctctccggtgacctggcccggcggctgccggacggaaatctcgagtttctcgggagaatcgacgaccaggtcaagatccggggcatccggatcgaactcggcgagatcgagacggccctgtccgaacacgcggccataactcaggccgcggtgaccgtccgagaagacgaccgcggcgacaaacggctggtcgcctacgtggtgccccactcggatgccggggacctcgccgtcgaattgcgcggtcatgtcgaggcgcggctcccctcttacatggtcccgtccgcgttcatcgtcctggacaagctgccgctgacgacgagcggaaagaccgaccggcgtgcgctccccgccccggaaagctggtcaccggcccccacctcgccagtctccccgcgcaacgcgaccgaatccacggtttgcgggatctacgctgacgtgctcgaggtgcaaaccgtcggcgtgcacgacgatttcttcgctctcggcggccactcgctgctcgcctcccgcgccgtctcgcgtatccgtgccgagctgggctgcgatctaccgcttcgtacgctgttcgacgtccggactcccgccttgctcgcccaggccatcgggtcgatgtcgtcgcgggacatgcccgtgctcgcgccatcgccgcggaccacgcccgcaccgatctcgctggcccagcagcagctgctcgggatcagtgattcgctgctcgacctgggcgcatttcccgtctgcccgtacggattccggctgcgcggggagatcgaccgcaccgtactcgacaccgctctgacccggatcgtcgcgcgtcacgagccactgcgaacaggcttccgcgacgacggcgacggcttcgtgcagatcgtccgcgagcccgcgccggtgagggccgagttcatcgaggtgaccgggaaagacacgaccgaccgtgaagcggcggcggcagagatcgccaagaacgaacttgaacgtcccttcgacctggtcgacggtattctgctccgcgccgttctgctgcggctggagcaagacgaccacatcctcatcctgatgttgcaccacatcgcgggcgacggctggtccttcgacgttctggtacgcgagctgtccgcgctgtacgccgaattggccggcggtcccgtcgcgggactgacggaagtcggcacgacatattcggacttcgcccgctgggagcacgaggccctgtccgggtccatccgcgaggaacacgacgcctactggcgagagcacctcaagggcgctgtcgcactggaactgcccaccgacaggccgcgaaagcttgcggatccgatcgggaaatccctcgagtggacggtttccccggatgcgacagccgcagcgcggaggctcgcgcaggcagagggcgtgaccctttacgaaaccatgctcggagccttcacgctggccgcagccgggttgtcaggcctggacgacattctggtggcaacccctttcgccaatcgtggccgacccgagatcgaccacctgatcggcttcttcgccaaggttgccgccctccgtgtcgatctctccgacgatccgacgttccgcgaggtcctgcgacgcgtccagaccgcggtcctgggtgctcacgcgcaccaggacctcccctatgcggcgatacaggccacggccacggccacggccacggccacggccacggccacggcatccgaactccccccgccgctcgtgcagttccagctgatcagctcgctgacctcggatctcgtcctgcctggcaccaccatcgagcgcttcggcatggtcgaggccgatctgggggtcgggaacggcgaactcgccatctggctcttcgacgatgacagcgccctgcacggaactgtcgtattcgacggatccctattcgaccccgcgagaattcagagtctgctttcctcggtcgagtcgacgttgcacgttgtctctgccaccccctccgtacgcgtatccaaggcatacgcagcaaacggccacgcaaggagatagcgccatgcctgagcaagacaaagtggtcgaatatcttcgctgggcgactgcggagttgcacaccacccgcgccaaactggaagcacttgccgccgccaacaccgagccgttggcgattgtgggtatggcgtgccgtctgccgggtggggtgtcgtcgccggaggatctgtggcgcttggtcgagtcgggtacggacgcgatttccgacttccccgccgaccgtggctgggacgtcgagaacctgtacgaccctgatccggacgcgtccgggaagtcgtactgcgtgcagggtggcttcctggactccgcaggcggtttcgacgcctcgttcttcgggatcagcccgcgtgaggcgttggcgatggatccacagcagcgactggtcctggaggtgtcctgggaggcgttcgaacgggccgggatcgagcccggttcccttcgcggcagcgacaccggcgtcttcataggcgcttacccaggcggctacggcgccggtgccggtgctgaccttgagggttatggcaccacatccggtcccagcgtgctctccggccgggtgtcgtacttcttcggcctcgaaggccccgccatcacggtggacaccgcatgttcttcgtcgctggtggcgttgcatcaggcggggtacgccctgcggcagggggaatgttccctggccctggtcggcggtgtcaccgtgatggccacgccggacgtcttcaccgaattcgcccggcagcgtggcctggccaccgacggccgcagcaaagccttcgcggacagtgcggacggtgccggattctccgagggcatcggtgtgctgctggtggagcgtttgtcggacgctgaggccaagggtcaccaggtgctggcggtggtccgcagttcggcggtcaaccaggacggcgcgtccaacggcctgaccgcgccgaacggcccctcccagcagcgagtgatccagaccgcgctcagcaacgccggtttgaccacagccgaggtggacgtggtcgagggccacggcacgggtacgacgctgggcgacccgatcgaagcccaagccgtgatcgccacctacggccaggaccgtgaacagcccctgctgctcggatcgctgaaatcaaacatcggtcatacccaggccgccgctggcgtatcgggtgtcatcaagatggtgatggccctgcggcatgctttggtgccgcgtacgttgcatgtggatgagccgtcgcggcatgtggactggacggcgggtgcggttgagctggtgacggaaaaccagccgtggccggagattggccggccgcgccgggcaggcgtgtcctcctttggagtcagtggcactaatgcccacgtcatcctggagagcgcaccccccacgcaggctgaagacgccgcccagcccgtagaagcgccggtgatgggctcggagccggtgcctctggtgatttccgccaagacgctaccggccctgaacgcacacgaggaccggctgcgcgcgtatctggcggcgtcgcccggggtggatatgcgggctgtcgcgtcaacactggcgatgacacggtcgatgttcgaacaccgtggggtgctgctgggtgacggcactgtctccggtaccgcggtgtctgatccgcgggtggtgttcgtcttcccgggtcaggggtcgcagcgtgctggtatgggtgaggaactggccgccgcgtttcccgtcttcgcgcggatccatcagcaggtgtgggatctgctggatgtgcccgatctcgatgtgaatgagaccggctatgcccagccggccctgttcgctttgcaggtggctctgttcgggttgctggaatcgtggggtgtacggccggacgcggtgatcggccattcggtgggtgagcttgcggctgcgtatgtgtccggggtgtggtcgttggaggatgcctgcactttggtgtcggcgcgggctcgtctgatgcaggctctgcctgcgggtggggtgatggtcgctgtcccggtctcggaggatgaggctcgggccgtgctgggtgagggtgtggagatcgccgcggtcaacggcccgtcgtcggtggttctctccggtgatgaggccgccgtgctgcaggccgcggaggggctggggaagtggacgcggctggcgaccagtcacgcgttccattccgcccgtatggaaccgatgctggaggagttccgggcggtcgctgaaggcctgacctaccggacgccgcaggtcgccatggccgctggtgatcaggtgatgaccgctgagtactgggtgcggcaggtccgggacacggtccggttcggcgagcaggtggcctcgttcgaggatgcggtgttcgtcgagctgggtgccgaccggtcactggcccgcctggtcgacggtgtcgcgatgctgcacggtgaccatgaggcgcaggccgctgtcggtgccctggctcacctgtacgtgaacggcgtgagtgtcgagtggtccgcggtgctgggtgatgtcccggtaacccgggtgctggatcttccgacgtacgccttccagcaccagcggtactggcttgagggcacggaccgggcgactgcgggtggtcatccgttgctgggttcggtggtgcggctggccgaggccagtggggtgttgttcactgcccgggtttcccggagcggtgatctgtggctgcgggaccagacggttctgcccgcgacggtgttcgtggagatggcgctggcagcggcggacgaggtcggctgcggtctggttgaggatctgagtgtggaagcgttgctgctgcttcccgatgatggcgccgtcgaggtacagacctgggtgggcgaaccggatgagggcggtcggcgccggctcagtgtccacgcccgttacggtgacggcgagccctggacctgcttggccaccgcaaccctggccaccactacgggtgtggccgctgccgcggtcggctggcaggccggtggggtgtggccaccggccggtccggtcccggtcgggacatcggcaccctcactgcgggcggtgtggcgcctgggcagcgacatcttcaccgaggtggccctggacgatgcccatgatgccaccaggtttgtgcgtcatcccggcctgatgaccgccgcgctcaccaccgtaggcgaggagactcccgccgtgtggcagggcctgaccctgcacgccggcaatcccggcgagctgcgcgtccgcctcacctcacacgatgacggcaccctgtcggcagaggccaccgacagcacaggcctccccgtcctgaccgcccgctcgctcaccctgcgcaccgtccccgtatacgaaccggccaccagcaccgacgacctgctcaccctgacctgggcaggaatccccaccccccagcagaccggcctgacggtgggtgcgtttgaagacctggcggcggacggcgatgtgccggtacccgaggtggcggtcttcaccgcactccccgacagcgacgatccgctggagcaaacacgaaagctgaccgctcaggtcctccacacactccaggagtggcttggcggggagcgcttcagcgacagcacgctggtggtgcggaccggcaccgggttggccgctgctggggtgtcggggttgatgcgctcggcccagtccgaacaccccggccggttcgtcctggtcgaaagcgacgacgccctcacccaggatcagctggcggcggcggtcggactggatgagccgcggctgcgggtcagcgacggccggtacgaagtaccacggctgacccgcacacatgccgaagagcctgagcctgaaaggacgtgggatccggatggcacggtcctgatcacgggcggttcaggtgtgctggcggggatcgccgcccggcacctggtgaccgaacgcggcgtgcgtcatctcctgctgctgtcccgcagcgccccggatgaggcgctgatcggcgagcttggtgaactgggggcccgggtcgagacagcggcctgtgacgtgtccgatcctgccgcgctgacgcaggtgctggcgggtgtctcgccggagcatcccctgacggccgtgattcacaccgcgggcgtggtggatgacggtgttgtggagtctttgaccgtgcagcggctggagacggtactgcggcccaaggccgacggtgcgtggaacctgcacgagctcacccgggatgccgacctggccgcgttcgtcatgtattcctccgccgccggtgtgctcggtagtgcggggcagggcaactacgcggcggccaatgcgttcctggacgcgctggctgagcagcgtcacgctgagggtctgcccgcactcgcggtggcctggggtctgtgggaggacgccagtggcctgaccgcgcaactgaccgacacggaccgtgaccggatccggcgcggtggcctgcgggccatctccgccgagcacgggatggggctgttcgacagcgcgtcacgccacagtgaaccggttctggtggccgcgccgatggagccggtacgggacgcggaagtcccggcattgctgcggtcgttgcaccgcccgattgctcggcgggccgctgccgccggtggagcgcggtggctggccgccctggcaccggccgagcgggagaaggcactgctgaagctggtgtctgacggcgccgcgacggttctgggacacgccgacaccagcacgattccggcaaccacggcgttcaaggatctgggcatcaattcgctgaccgcggtggaactgcgcaacagcctggcgaaggccacggagctgcggctgcccgccacgctggtgttcgactaccccaccccggccgccttggctgcccggttggacgagttgttcaccggcgagaaccccgtaccggtacgcgggccggtgtcggcggtggcgcaggacgagccgctggcgatcgtgggaatggcctgccgcctacccggtggagtctcgtcgcctgaggatctgtggcgtctcctggagtcgggtacagatgcggtctccggtttccccaccgaccgtggctgggacgtcgagaacctgtacgacatggctggaaaatcgcaccgtgctgagggtggcttcctggatgccgcggctggctttgatgccggattcttcgggatcagtccgcgtgaggcgttggcgatggatccgcagcagcggctggtgctggaggtgtcctgggaggcgttcgagcgggccgggatcgagcccggttccgtacgcggcagcgataccggcgttttcatgggtgcgtaccccggtggctacggcatcggtgccgacctcggcggcttcggggccaccgccagttcggtcagtgtcctgtccggccgggtgtcgtacttcttcggcctcgagggtcccgcgttcacagtcgacacggcctgctcgtcatcgttggtggcgttgcatcaggcggggtatgccctccggcagggagagtgttcgctggccctggtcggcggtgtcactgtgatggccacgccacagactttcgtggagttctcccgccagggcggcctggcctccgacggccgctgcaaagcgttcgccgacgccgcggacggcacgggatgggctgaaggtgtcggtgtcctgctcgtagagcgactctccgatgcccgccgtaacggtcaccaggtgttggcggtggtgcgtggatcagcggtgaaccaggacggtgcgtcgaacggtctgaccgcgccgaatggtccttcgcagcagcgggtgatccgggccgctctcagcaacgcgggtctgagcacggctgaggtggatgtggtcgaggcgcacggcacgggcacaacgctgggtgacccgatcgaggcccaggcgctgatcgctacctatggccaggaccgtgaccagcctgtgctgctgggttcggtgaagtcgaacctgggtcatacgcaggccgctgcgggtgtgtccggtgtcatcaagatggtgatggccctgcaacacggtctggtgccgcgcacgttgcatgtcgatgagccgtcacggcatgtggactggtcggcgggcgcggtgcagctcgtgacggagaaccagccgtggccggatatgggccgagcgcgccgggcaggcgtgtcgtccttcgggatcagtggcaccaacgcccacgtcatcctggaaagcgcaccccccactcagcctgcggacaacgcggtgatcgagcgggcaccggagtgggtgccgttggtgatttcggccaggacccagtcggctttgactgagcacgagggccggttgcgtgcgtatctggcggcgtcgcccggggtggatatgcgggctgtggcatcgacgctggcgatgacacggtcggtgttcgagcaccgtgccgtgctgctgggagatgacaccgtcaccggcaccgctgtgtctgaccctcgggcggtgttcgtcttcccgggacaggggtcgcagcgtgctggcatgggtgaggaactggccgccgcgttccccgtcttcgcgcggatccatcagcaggtgtgggacctgctcgatgtgcccgatctggaggtgaacgagaccggttacgcccagccggccctgttcgcaatgcaggtggctctgttcgggctgctggaatcgtggggtgtacgaccggacgcggtgatcggccattcggtgggtgagcttgcggctgcgtatgtgtccggggtgtggtcgttggaggatgcctgcactttggtgtcggcgcgggctcgtctgatgcaggctctgcccgcgggtggggtgatggtcgctgtcccggtctcggaggatgaggcccgggccgtgctgggtgagggtgtggagatcgccgcggtcaacggcccgtcgtcggtggttctctccggtgatgaggccgccgtgctgcaggccgcggaggggctggggaagtggacgcggctggcgaccagccacgcgttccattccgcccgtatggaacccatgctggaggagttccgggcggtcgccgaaggcctgacctaccggacgccgcaggtctccatggccgttggtgatcaggtgaccaccgctgagtactgggtgcggcaggtccgggacacggtccggttcggcgagcaggtggcctcgtacgaggacgccgtgttcgtcgagctgggtgccgaccggtcactggcccgcctggtcgacggtgtcgcgatgctgcacggcgaccacgaaatccaggccgcgatcggcgccctggcccacctgtatgtcaacggcgtcacggtcgactggcccgcgctcctgggcgatgctccggcaacacgggtgctggaccttccgacatacgccttccagcaccagcgctactggctcgagggcacggaccgggcgactgcgggtggccatccgttgctgggttcggcggtgcggctggccgaggccagcggggtgttgttcactgcccgggtttcccggagcggcgatctgtggctgcgggaccagacggttctgcccgcgacggtgttcgtggagatggcgctggcagcggcggacgaggccggctgcggtctggttgaggacctgaacgtggaagcgttgctgctgcttcctgacgatggcgccgtccaggtacagacctgggtgagcgaaccggacgaggccggtcgccaccggctcagtatccacgcccgttacagcgacagcgagccctggacacgcttggccaccgcaaccctcgccaccaggggaacggtatccggctggcaggccggggaggcgtggccgccgaccggtgcggtcccggtcgagaccggagtaccgtcactgcgcggggtgtggcgccgaggcaacgaagtgttcgccgaggtcgccctggacagcacccacgacgccaccacatatgccctgcaccctgccctcctgaccgccgccctcaccaccgccggtgaggaaacccccgccgcgtggcaggcgctgaccctgcacgcccgcaaccctgccgagctgcgcgtccgcctcatctcacacgatgacggcaccctgtccgtggacgccaccgacagcacaggcctccccgtcctgaccgtccgctccctcaccctgcgcaccgtccccgtctacgaacctgccaccagcaccgacgacctgctcaccctgacctgggcggagatcccggcccctcaggaaaccggcctgacggtcggccggttcgaggacctggtgtcggacgctgatgtgccggtacccgaggtggcggtcttcaccgcactccccgacagcagcgagaacccgctggaacagacccgcgtactgaccgctcaggtcctccaggcagtccagacctggcttggcggggaacgtttcaccgacagcacgctggtcgtgcggaccggcacccggttggccgccgctggggtgtcggggttgatgcgatcggctcaatcggaacaccccggccggttcgtcctggtcgagagcgacgacgacacgctcgccccggaccagttggccgccaccgtcgggctcgacgagccgcggctgcgggtcagcggcgaccggtacgaggcaccgcgactggctcgtgtgaacgccagtgggtctgagcctgaagcggtttgggatccggatggcacggttctgatcaccggtggttcgggtgtgctggcggggatcgccgcccggcacctggtggccgaacgcggcgtgcgtcatctgctgctgctgtcccgcagcgccccggacgaggcactgatcaaccaactcggcgaactgggcgcccgggtcgagacagcggcctgtgacgtgtccgatcgtgccgcgctggcccaggtgctggcgggtgtgtcaccggagcaccccctgacggcagtgattcacaccgcgggcgtactcgatgacggtgttgtcgagtccctgaccgcgcagcggctcgacacggtactgcggcccaaggccgacggcgcctggcatctgcacgaactcacccgcaacaccgacctggccgccttcgtcatgtactcctccgccgccggtgtcatgggcggtggggggcaaggtaactacgcggcggcaaacgcgttcctggacgcgctcgccgaagaacgccgcgccgagggcctgcccgcaactcgcggtggcctggggtctgtgggaggacgccagtggcctgaccacgcaactgaccgacacggaccgtgaccggatccggcgcggtggcctgcggactatcaccgccgagtacgggatgcggctgttcgacaccgcatcacgccatggcaacccgattctggtcgccgcaccgatggacccggtttgggacgcggaagtccccgcgctcctccgctcgttgcatcgtcccgtcgcccggcgggccgcctctaccagcgactcgtcagcgcggtggctggcggccctggcaccggccgagcgggaagacgcactgctgaagctggtgcgtgacagcgccgctctggtcctgggacacgctgacgccagcaccatccccgcagccgccgcattcaaggatctgggtatcgattcgctgaccgcggtggaactgcgcaacagcctggcgaaagccacagggctgcggctgcccaacacgacggtgttcgactaccccaccccggccatcctggccacccggctgggtgagctgttcaccggcgagaaccctgcaccggtacgcccgtcggtgtcggtggtggggcaggacgagccgctggcggtcgtgggtatggcctgccgtctgcccggcggggtgtcgtcgcctgaggatctgtggcgccttgtggagtcgggtacggatgcgatttccggtttccccgccgaccgtgggtgggacgcggagagcctgttcgatccggacccggacgcggtcgggaagtcgtactgcgtagagggcggcttcctcgacagcgcagccagcttcgacgccggattcttcggcatcagcccacgcgaggctctggcgatggacccgcagcagcggctgatcatggaggtgtcctgggaggccttcgagcgggccgggatcgagcccggttccgtgcgcggcagcgacaccggcgtcttcatgggcgcgtacgccggtggctacggtgccggtgctgacctcggcggcttcgcggccaccgccagcgcgaccagtgtcctgtccggccgggtgtcgtacttcttcggcctcgaaggccccgccatcacagtcgacacagcctgctcgtcatcactggtggcactgcaccaggccgggtatgccctccggcagggagagtgttccctggccctggtcggcggcgtcaccgtgatggccacaccacaaagcttcgtggaattctcccgccagcgtggtctggcctccgatggccggtgcaaggcgttcgcagacagcgcggacggcacgggatgggctgaaggcgttggtgtgctgctggtagagcggctttccgacgcccaggccaagggccatcaggtgttggcggtggtccgtagctcggcggtcaaccaggacggcgcgtccaacggtctgaccgcgccgaacggtccttcgcagcagcgggtgatccaagccgctctcagtaacgccggcctcgccgcgcacgaggtggatgtggtcgaggcccacggcacgggcacgacgctgggcgacccgatcgaggcccaggcgctgatcgccacttacggtcaggaccgggaacggcccctgctgctgggttcgctgaagtcgaacatcggtcatgctcaggccgcctcgggcgtgtcgggtgtcatcaagatggtcatggccctgcagcacaacacggttccccgcaccctgcacgtggatgagccgtcgcggcacgtggactgggcggcgggtgcggttgagctggtgagggagaaccagccctggcccggcaccgaccggccccgtcgggcgggcgtgtcgtccttcggagtcagcggcaccaacgcccacgtcatcctggagagcgcaccccccgctcagcccgcggaggaggcgcagcctgttgagacgccggtggtggcctcggatgtgctgccgctggtgatatcggccaagacccagcccgccctgaccgaacacgaagaccggctgcgcgcctacctggcggcgtcgcccggggcggatatacgggctgtggcatcgacgctggcggtgacacggtcggtgttcgagcaccgcgccgtactccttggagatgacaccgtcaccggcaccgcggtgaccgaccccaggatcgtgtttgtctttcccgggcaggggtggcagtggctggggatgggcagtgcactgcgcgattcgtcggtggtgttcgccgagcggatggccgagtgtgcggcggcgttgcgcgagttcgtggactgggatctgttcacggttctggatgatccggcggtggtggaccgggttgatgtggtccagcccgcttcctgggcgatgatggtttccctggccgcggtgtggcaggcggccggtgtgcggccggatgcggtgatcggccattcgcagggtgagatcgccgcagcttgtgtggcgggtgcggtgtcactacgcgatgccgcccggatcgtgaccttgcgcagccaggcgatcgcccggggcctggcgggccggggcgcgatggcatccgtcgccctgcccgcgcaggatgtcgagctggtcgacggggcctggatcgccgcccacaacgggcccgcctccaccgtgatcgcgggcaccccggaagcggtcgaccatgtcctcaccgctcatgaggcacaaggggtgcgggtgcggcggatcaccgtcgactatgcctcgcacaccccgcacgtcgagctgatccgcgacgaactactcgacatcactagcgacagcagctcgcagaccccgctcgtgccgtggctgtcgaccgtggacggcacctgggtcgacagcccgctggacggggagtactggtaccggaacctgcgtgaaccggtcggtttccaccccgccgtcagccagttgcaggcccagggcgacaccgtgttcgtcgaggtcagcgccagcccggtgttgttgcaggcgatggacgacgatgtcgtcacggttgccacgctgcgtcgtgacgacggcgacgccacccggatgctcaccgccctggcacaggcctatgtccacggcgtcaccgtcgactggcccgccatcctcggcaccaccacaacccgggtactggaccttccgacctacgccttccaacaccagcggtactggctcaagagcgtggaccgggcggctgccgacggtcatccactgctgggcaccgtagtggcactgcccggctccgacggtgtggtgctcaccgggcgggtgtcgctggccacccatacatggctggccgatcacgcggtccggggcagtgtcctgctccccgggaccgcatttgtggaactggtcgtccgcgccgccgacgaggtcgagtgcgacgtcgttgacgagttggtgatcgaaaccccgctcctgctgccgcagaccggaggcgtccaactgtccgtgtccgtcgccgaggccgacgagtccggacaccgcacggtgacggtcttctcccaggcggacaacacggacgcatggatccggcacgtttccgccactatcagcacctctgacacacccctctcgctgccggagtctgacacctggccgccagcccaggcccagccgatgaacgtggccgacttctacgaccggctgatggcagaaggctacgagtacggtcccgccttccaaggtctgcaggctgcgtggcgtgacggggacaccgtctacgccgaggttgctctcgccgaggagcaggcgcaggaggcggcacgatacgacgtgcacccggccctgctcgatgcggccctgcacgcctgcaccctgaacacacccgacgccgaggagggtgtggggctgccgttctcctggaaccacgtccaggtccatgccactggcccggccacgctgcgggtcgcggtgacacaggaagccgacggctggagtctgcgggtcgccgacgacaccggcaggcccgtagccaccatcggctccctcatcacccggccggtaaccgccgacacactcggctccaccacggacgacctgctcaccctgacatggacggaaatcccggccccccaggagaccagcctgaccatgggcaggtacgaagacctggcggacggcaacatgccggtacccgacgtggtggtcttcaccgcactccccaacagcagcgagaacccactgggtccgccggccccgctggacccgccggacccactggcacagacccgcacactgaccgcccaggtcctccaaaccgtccagacctggctggccggggaacgcttcaccgacagcacgctggtcgtgcggaccggcaccgggttggccgccgccgccgtgtcggggttgatgcggtcggcccagtccgaacaccccggccggttcatcctggtcgaaagcgacgacgacaccctcaccctccgccaactggccgccaccgtcggactggacgagccacggctgcggatcaacgacggccggtacgaggcagcacgactggcccggaccaacaccacccccgaagacaccccgctgacgatccccgacagtcgtgcctggctgctggaacaaccccgcagcggaaccctgcaagacctcgccctggtacccaccgacaccgccgaacagcccctgcgacccggcgaagtacgcatagacgtacgcgccgcaggcctgaacttccgcgacgtcgtcgtggccctcggcatggtcaatgacaaccggccgacgggcggcgaagcggccggcgtcgtgctcgaggtcggccccgatgtacaggatctggccccgggagaccgcgtcttcggcgtggtggcaggctcattcgggccggccgcgatcgccgaccgtcgaatgctcgccgtgattcctgatggctggtcgttcactacggcggcgtccgtcccggttgtgttcgccaccgcgtactacgggctggtggatctggccgggctgagtgagaggctcattcgggccggccgcgatcgccgaccgtcgaatgctcgccgtgattcctgatggctggtcgttcactacggcggcgtccgtcccggttgtgttcgccaccgcgtactacgggctggtggatctggccgggctgagtgagggtgagtccgtgctgatccacgccgccgcgggtggtgtcggcatggcagccacccagatcgcccgccacctcggcgcgcggatctacgcgaccgccagcaccgggaaacaacacatcctgcgcgaagccggtctcgaagacacccacatcgccgactcccgcacactgagtttccaggaaaccttcctcaacaacacacacggccagggcgtcgatgtcgtcctgaactccctcagcggtgacttcgtcgacgcctccctcgacctgctgccccgcggcggccggttcgtcgagatgggcaagaccgacatccgtgacccgcaccaggtcaccgccgaccggccgggcaccacctaccaggccttcgacctgatggacgccggcccggaccggctacgggaaatcatcaccgaactcctcaccctgttcacgcaaggcgtcctactgcccctgccggtgcaggcctgggacatccgccaggcccgcgatgcgttcagctggatgagccgcgcccgccacatcggcaagatcgtcctcaccatcccccgccggccggaccccgacggcaccatcctgatcaccggcggctccggcgtcctggcaggcatcctcgcccgccacctggcagccgaacacggtgcacgtcacctcctgctgctgtcccgcaccacccccgaccaagcactgatcaaggagcttgccgagctgggcgcccacgtcgacacagccacctgcgacgtgtcggaccgtgccgggctggcgcgcgtactggcgggcgtatcacccgaacaccccctgaccgccgtgatccacaccgccggcgcactcgacgacggcgtcgtggaatccctcaccacacaacagctcgacacggtgctacggcccaaggccgacggcgcctggcacctacacgaactcacccagaacaccgacctggcggcattcgtcatgtactcctccgccgccggtgtcataggtggcgcggggcagggcaactacgcggcggccaacgccttcctcgatgccctggccgaacagcgccgagccgagggcctgcccgcactcgcgctcgcctggggcctatgggacgacaccagcggcctcacctccgggctgaccgacacggaccacgatcggatccggcgcagcggcatgcgcaccatcaccgccgagcacggaatgcggctgttcgacggcgcgtcacgccacggtgagccggtcctgttcgccgcggcgatgtctccgctacggggggacgtcgaggtccccgcgctgctgcgcggacttcaaacggtgaagcgtccgcagtccaggaccgcggcacgcaacgaggtcggctcgcagcccctgagcgcccgtctcaccggccgtacgagcgtcgagcagcaccgcatcatgctcgaactcgtgctgggagaggccgcctccgtactcggccacagctcggcggatgcgatcgcgacggacacgtcgttcaaggatctgggcatggattccctgacggcgatcgaactccgtaaccgcctcgtggcggagaccgggctgcagcttcccgccacgatggtgttcgactacccgacggcgaacgctctggccgcgcatctgctgggcaagctcgatatcccgcccgtacagcaacgcctcgaagctccggcgccgagcacggtcaccggcccggccgatccggtcgcggatgagccgtcggcgaatgagccgatcgccatcgtggcgatggcgtgccggctgccaggcggggtcagttccccggagggtttgtggcaccttgtggagtccggcactgacgcgatctccggtttccccaccgaccgtggctgggacgtcgagggcttgttcgatccggatccggacgccgccggtaagtcgtactgcgtgcagggcggcttcctggacaccgcggccgacttcgacgctcccttcttcggcataagcccgcgtgaggcgctgggcatggatccgcagcagcggctgttgctggaaaccacctgggaggcgatcgagcgcgcgcagatcgatccgaagtccctccggggtcgcgatgtgggcgtgtacgtgggtggcgcggcacagggttacggagtgggtgtcgaccagcagcacgacaacgggatcaccggtagctcggtcagcctgctgtcggggcgggtgtcatacgcgctcgggttggaggggccgggggtcaccgtggacaccgcttgctcctcctccctggtggcgctgcacctcgcgagccaggcgttgaggcagcgcgagtgctcgctggcgctggtgagcggggtctcggtgatgtcctcgcccgcgatgttcgtggaattctcccgccagcgcggcctgtcgtccgatggccggtgcaagtcgttcgccgcgtcggcggacggcacgatctggtccgagggcgtgggtgtcctggtggtggaacggctttccgacgcccgtcggctggggcaccgggtcctcgccaccgtccggggcagcgcggtgaactccgacggcgcgtcgaacgggctgaccgcaccgaacggcacgtcccagcagcgggtgatccggcaggcgctggccaacgccggtctcaccgcgtcggacgtggatgtggtcgaggcgcacggcaccggcacgaagctcggcgacccgatcgaggcagaggccattctggcgacgtacgggcaagagcggtcggcgcccgcctggctcggctcgctgaagtcgaacatcggccacgccatggcggcgtcgggcgtgctcagcgtgatcaagatggtggaggcgatggggcacggttcgctcccccgtacgctccatgtggacgcgccatcgcctcacgtggactggacatcgggaagcgtcgcgctgctgacggagcaccagccgtggccggatgacaccaagctgcgcagggccggcgtgtcgtcgttcgggctcagcggtaccaacgcgcacgtggtgctggagcagtaccaggcgcccgccccgccggtcacgccggtcacgcccgccccgccggtcacgccggtcacgccggtcacgccgaatgagcccggaccgctgccgtgggtcttgtccgcgcagagcccgaaagcgctacgggagcaagccgggcggctgtacgcctccctcgccggggactcggagtggaactcgctggacatcggatactccctggcgaccacacgatcggacttcgcccaccgggcagtcgcggtgggatccggccgggaggacttcctgcgcgcgctgtccaagctggcggacggcgccccttggcccgggctcacgaccgcgaccgcgaccgccaaggcccggcgcgttgccttcctgttcgacgggcaaggcacccaacggctgggaatgggcaaggagctgtacgacagctaccccgccttcgcacgtgcctgggacacggtgagcgccgggttcgacaagcacctcgaccactcgctgacggacgtctgcttcggtgagggcggttcgacgacggccgggctcgtggatgacaccctctacgcgcaagcgggcattttcgcgatggaggccgcgcttttcgggctgctggaggactggggcgtgcgccccgacttcgtcgccgggcactccatcggtgaggcaaccgccgcgtacgccagcgggatgctctcgctcgaaaacgtcaccactctgatcgtcgcccggggtcgagcgctgcggacgacaccgccgggagccatggtcgcgctgcgggcaggtgaggaagaggtgagggagttcctcagccggaccggtgcggcgctggatctggcggcggtcaacagccccgaggccgtcgtcgtgtcgggtgagccggagccggtggcggacttcgaggccgcgtggacggcgtccgggcgggaggccaggaaactgaaggtccgccatgccttccactcccgccatgtggaggccgtgctggatgagttccgtacggcactggagtccctgaagttccgtgcccctgcgctgcccgtggtatcgaccgtgaccgggcgtctgatcgaccaggatgagatgggcacgccggagtattggttgcgccaggtccggcgacccgtccgcttccaggacgccgtccgcgaactggccgagcagggtgtgggcacattcgtcgaggtagggccttcgggggcgctcgcgtcggccggagtggagtgtctgggcggcgacgcctcgttccacgccgtgctacgcccgcggtcgccggaggacgtctgtctgatgacggccatcgcggagttgcacgcgggcgggacggcaatcgactgggcgaaggtcttgtccggcggccgggcggtggatctgccggtctatccgttccagcaccagtcctactggctggccccggccgccccggatgccacagctgtggcgcccgtggtcgaggaggagggcggcgagtatgacgagccctcctatgcggatgagccccgcaccatgctggaactcgttcacatggaggtggcgagcctcctgggcatggccgaccccggcgtcatactcgacgactcgtccttcctggagttgggattcgactcgctcagcgccgttcggctgcgcaaccggctgtcgaaggccaccggactcgatctaccgtccacgcttctcttcgagcaccccacgtcggcggaattggccgcgcacttggatgcgttgcttgattccgacatcgacgcggccggcgtttatgcgctgctggaagagatcaacgaactggacgcagaagccgtcgacatgaccgcggcggaacataaggccatttccgaactgctggaacagctctcggcaaaatggaggaccaggaacgaataagagggtgtcacacgtggtgtgtgtatacccgtacttggcacatcggctgggcaagaataatttaaggtgatgttggaattgggtaaccgcctgaagttcagatttaccggaccgctcctcgaagctgtcaatccgcgcctgcagggtcacccctacgacgtactgatgcgcctcctcgagggcggtcgcatcgaaaacgtgctggaattgtgcggcgggaccggattcgcgtcgcgaatgctggccgaaaggcattcgaaggtacaggccacctccatcgatctgtcacccgaactgaccgcggtaggccgccgcaagttggcctcgcgggggatcgataacgtcaccctggtcgagggtgacgtttccaccttgccttaccccgacgactcgttcgacaccgtgatgtcggccttcggtctgcacgaggtcccgaccgcgggccgcctcagcgcgatccgcgagtccgtgcgggtgctcaagcccggcggccgcttcgtcatcgtcgatctcgaccgccggacaaagtacgggtggacgatggacctcttcatgaaggtcatggagccgaagttcgcccccgaggtgttcggaaccggcctggtggaccggctgaaagagaacggtttcaccatcgaccatcacgagtcggccgggccgaacggatggacacagtcgatcgtcgccaccctggaggcgtgagccgtcgtcatacgtccgaggggcggcctcttgtgaggccgcccctcggacgtatgacgacaagccgcacggacggcttcctcagtcctggggttcgattcctcagtcctggggttcgagaagctcgccggtctccttgacccggacgtggatcgcccccgacgggcaggcctgcgccaccgcgatcacgtgctcgtccgcgtcgatctcgctcgcgcgggcgcgcgactggtgcccgtgcatctcgaagtacgcgcccgcgctggccacgcagacggccgagccgatgcactcggtccggtcgatccacagctcccatcgcgccatgccactcaccatgtcaggggcaacgcttcgaggctgggcaccagccgcccgcgcaggaacttgatccgcggctccggcaccgcgaaccgcagcgccgggatacgggagatgagcgagccgagcgcggtctgcatctccacccggacgagttgagcgcccatgcagtggtgcgcgccgtgtccgagggacatgtgcgggttgctcgtccggtggaagtcgatctcctccggatggtcgtagaccttggggtcggtgttggccgagtgcgtctggatcgccaccgcctcaccggccttgacgatcgtgccgctgagctcgacgtcctccaccgccatccggacccagctcgcctgcttggcgatcgggatgatccggagcaactcgtcgaccgccttcgggatcagcgagaggtcctttcgcagttcggcgagtttctccgggtgggtcagcagggtgtagacgctgttgccgatcgcgttggcggtggtctcgtatcccgctatcagcagtgtgacggcgaagccgaccagctccgtctcgctgagccggtcgtccttgtcgcgggcccgcaccagcgacccgagcagatcgttggtgtcctgcttgcggcgttccgcgatcagctcggagaggtagaggtagagcgactggccggccgccatcacgacttcctgtgagtaggcgccgttcgagagcatctggtccgaccaggtccggaacttggtctggtcctcgatcggcacgcccagcagctcacagatcatgatgatcggcagaggcagggcgaagtcctccatcagatcggcgggggcgcccttggccagcattttgtcgatcagatcgtcggcgacctcctgggtgcgcggacgcagggcctccatccggcggctggtcagcgccttggtcgccaaccggcgcagccgggtgtgttccggagggtccatcagcatgatgacgggctggtcctggatcgccgggaggacccggggcacgtccttgccgagcgtcgcgctgcggctgaaccgcgggtccacgaacaccttggcgacgtcctcccagctggtggccagccaggtctccccgccgtacggcatcaggacccggccgagctcaccggcgtcccgcagccggttgtactcggggtggatctcgagtcgctccatttcggcgaaaggataagggcaggcctttccggtctcaccctgatcggtcgtcgacattctttgtctccgttcacacgcggacggcgatctggtgccgataggtttccatcggcaggctgtccatcggcggcacgaggccgagccggctgagcattttgaccgaagcgggcggctggtagtccgcgaacgtgccgccgaacaccgtgatctggctgtgcagggcgagcgtgcgcgcaagccggttcatccgttgtttgttcagggtgctgcggaggtgactgtccggggccatccgccggcgcaccgcggcacggacttgatcggagatgtcgtgatcgctgacccacttcagttcgggtatttccgttgtgatccgacgcatcgcctcaaggcgctgccgcgtgaacacgtcgatgtgcgaaagggcgccacccgggcgcagcacgcgcgcggcctcccgcaggaaacgtcccagattggggtaggtgtgcgagctctcgatgttgacgagcacatccaccgaggagtcctcgaagggcagttcctcggcgtcgccctggacgaaccgcagggtatcgccgcgggacagcgtggcggtggcgctggcgatcgccttcggcgccaggtccagcccggtcatccgggcggtggggacgaggcgggacaggaagttgagcccctcccccattccgcagccgacctccaggaccgtccggccgtcgcagctctccaagcccttcggaaggtcgcgcagggccaggtagtagagctgctcgctgaatccgtcggtgccgtactcggtgaatccgggcagcctggcctcgatctcggcgacgaactcggaatcgtgcacaccccagttccacagctggccctttgccgacatgctggcggcgaggtcgtagatggaggagctggcggacttgaaggtggcggccttcgtctccgcctgcggggtgccggattcgtcgagattgatgtcggcgacaccgctggtgaaggcggtcacgacgtcgggttggatcatgcgggtctccgaattcgccggatcggttcgatcattgctctgaagatgactcggtgtggatgaacgcgcgcgagatggtcggtcaccactacgtcggtcatcactacagcgagtacggatcgaggacgtcctcgggcgtcgactcgatcgacatcttgtggccgaggccgagttcgtcggcgaagccgagcagtacgtcgagggcgatgtggtcggcgaacgcgctgccggtcgagtcgaagacgctcaggcgctccgggtgcggagcggcgatctcgggtgccgcgcacaggtcggccagcgacgggccgagttcccgatcgggcagctgctggaactcgccctcggcgcgggcctgcccggggtggtcgacgcagatgaacgcgtcgtcgagcagcgccttgggcagctcggtcttgccctgctcgtccgcgccgatgccattgacgtgcagatgtgcctggcgcggttcggccggcaccaccggcccgccgccgaccggtaccgatgtgacggtgcacaggacgtccgcggtcgccatcgcggtcgccgcgtcggtgacctcgacgggcagctcgagaaacgcgacccggccggcgaacgactccgcgtgctcagctttgatgtcactgatcaggatgcgttccagtgggaggacgcggctgagggcgtgtgcctgggtgaccgcctgggcgcccgcgccgatcagggcgagggtggtgctccccggccgggcgagcagccgggtggcgacggccgcgaccgcgccggtccgcatggcggtgatggtggccgcgtcggcgagcgcgaccatgcttccgctgtcgtcgtcgagccgcgacacggtcccgacgatggtgggcaggttgaagcgctcgaagttctgcggactgtagctgaccgtcttcatcgtcacaccgatgcccgacgcgcggtgcggcatgaactcgatgacgcccggaacgtcgccgccgcgggcaaagccggtacgcggtggcggctcgtcggtctcgccgcggcccaatcgcgcgaaaccggcgtgcacctcgctgatcagccgatccatcatgacgtcgcggccgaccaccgagaggatccgcttgatgtcacgctggcacagaaccttggtctgcatgtcacctctctttcgtgagcgtggcccggcggggtctcagggatctcgtcctcaccggtgccggcccacgtcgccgcgaccggcctcggccgaccgcccggtcgcgccgctgccggtgtgggcgatacggtcgccgcagcttcggcgcacctcccctgccgacctccgccccgccggcacgcaaggcaggtcacgagcggacatcggcgacgctagcagtcacctgacgaccctcacccccccaacgcccccctggatgattccgcagctccgggcgcccgtttgtagggggttgcgggatctggcgcgcccgtattttcttcctggtacgcgccagggaacggcggggaagggtgaggcaattgactccgccggtcacggcaccgtactgccgcttcgagaagctcggcgcctccgatctcgacggcgacgagacacttctcggcgtcatcgaacatcgcaccggacacaccggggtttcactggccgagggctgtccccggacggccgtacacacgacgacccgcgaggacgagtcgttcgccgaggcgtggcacgcggaggggccgaaggagtcgggcagccatgatggcgtcgcctgggctcggacacctgactacctcttcggtgtcgcgcgggtgcccgagggcggccggtacgcggccggcaccgcggccgtctacaccggaatcttcgacctgatcgggacgctggggtaccccagtctggcccgcacctggaactacgtcagcggaatcaacacgccgaacgccgatggcctcgaggtctaccgggacttctgtgtgggccgcgccgaggcgctggacgcccgtgggatcgacccggcgaccatgccggcggcgaccggcatcggcgcccacggcggcggcatcacgtgctacttcatcgccgcacgcgccggtgaccgggtcaacatggagaacccggccgtgctcacggctcaccgctacccgcagcggtacggcccccgcccgccggtcttctcccgggccacctggctctcgccgccgggggcggacgacggccggctcttcgtctccgcgaccgccggcatcgtcggtcacgagacggtgcaccacggcgacgtcgccgcgcaatgcgaggtgtcgctcgagaacatcgcccgggtgatcggtgccgagaacctgggacggcacgggctccgccggggctacgccctcgccgacgtcgaccatttgaaggtctatgtacgccaccgcgaggatatttcgaccgtacgtcggatctgcgccgagcgcctctcccgcgaggccacggttgccgtactgcacaccgacatcgcgcgcaccgatctgctggtcgagatcgagggtgtggtggcgtgaccccgccgagctgacctggaaggaccaccgatgagcaccgaagctcagcaagagagcacgcccaccgcacgctgccccttctcgatccaggacgggcaccgcacgatcctggagaccggcacggtcggcgcgcacgaactcttcggcgtcaagcaatggctggtggccgcggccgaggacgtcaagctggtcaccaacgacccccggttcagctcggcggcgccgtccgggatactcggcgaccggcggccgggctggttctccgggatggactcgccggagcacaaccgctatcggcagaagatcgcccgggacttcaccctgcgggccgcgcgcaagcaggaggagttcatcgtccgggcggcggactcctgcctcgacgacatcgaggcgtccggtcccggcaccgatctggttcccgggtacgccaagcgcctggcgtccctggccatccacgacctctacggcctgaatgaggaggaggggcccgtactcgagggccagatgcgggccatggagggcggcaccgacatggagagcatcaagaggctgaccgacgaattcttcggtcacgtcctggcgctggtgcgtgccaagcgggagcaggcgggcgacaggcttctgcaccggctggccgagtccggcgaggacgagatcctgctcagcgacgaggaggcgaccggggtgttcgccactctgctgttcgccgggcacgactcgatgcagcagatggtcggctactgtctgtacgcgctgctctcccatcccgagcagcgggcggcgctgcgggagaacccggacctgatcgacggcgcggtcgaggagctgctgcgcttcctgccgctcaaccagctcggcgtgccgcgggtctgtgtcgaggacgtcgagctgcacggccagaccatcagcgccggcgacaacgtgatcccgctctactcgacggccaaccgcgaccccggcgtcttcgccgaccccgacacgttcgacatcacgcgtaagcccgaacacaacttcgctttcgggtacggcatccacaagtgcccggggcagcacctcgcccgcgtgttgatcaaggtcgccacgctgcgcctgttcgagcgcttcccggatgtgcgactggcgggcgacgtgccgatgaacgagggtctgggcctgttcagcccggccgagctccgggtcacctggggagcggagtgagcgcgtccgtgcagaccatcaagctgccgaacggcaagaccgtcggcccacgtcaacccgggcgaggcgcagttcctctaccaggagatcttcgccgagcggtgctacttgcggcgcggccttgagctgcgagcgggtgacgtggtcttcgacgtcggcgcgaacatcggcatgttctcgctcttcgcccacctggagtgccccgatgtcacggtgcacgccttcgagccggcgccggtgccgtacgccgcgctcagggccaatgccgagcggtacggcatcgcgggccggttcgagcagtgcgcggtctcggacgtggccggccgcggcaagatgacgttctacacggataccacgatgatgtcgggcttccacccggatccggcgacccgcgcggagctgctgcgcaggctcgccatcaacggcgggtacagtgccgaggccgccgaccggatgctggccgagctgccggacaccagccaggtgatcgagacgtccgtcgtacgcctctccgacgtcatcgcggagcggggcatcacctcgatcggactgctcaagatcgatgtggagaagaacgagcggcatgtgatggccgggatcgacgcggccgactggccgcgcatccgccaggtcgtcaccgaggtgcacgacatcgacggcaggctcgacgaggtcctcacactgctgcgcgggcagggcttcacggtcctcagcgagcaggagccgctcttcgccggcaccgacatctaccaggtcgtcgcccgccgcggggacgcctgaccacacgccgcccgttccccctcccaagcggcgcggtcccgtggcttctgcggctattccgccttgacgagttcggccggcaggtgctggcggcccttgacccccagcttgcggaagacgttcgtgaggtgctgctccaccgtgctggccgtgacgaagagctggtcggcgatcacgcggttggtctgtccgccggcggccagcgacgccacccgccgctccgcgtcggtcagcgacttgatccgctgcggcaggccggattcctccagccagccggggcctccgggcttggccccgagtcggcgcagcagcggtatcgcgccgcgctgggcggccaggtgccgggcctgccgaagcagtccccgcgcccggctgtggtgccggagcgcgctgaacgtctcgctcaggtcggcgagcacccgtgcccgctcgtactggtcgttgcaggagagcagcaggtcggccgcttcatcgagcaggtcgacccgcttcgccggcgggctgtacgccgcctgcacccgcagcgtcaaggcgcgggaacggggtcccatcgcacgggtgagttgctcgtcgacgagccgtcggccctcgtcccggtcgcggccgtgcagcagcgcctcggcggcgtcgacgcgccacagggacagacccggcacgtcaacgccccagttccgcatacgttccccgcaggtgcgaaacgccgtgtacgccgactcgtgctggcccgtggcgaggctgtactggccgcgcgccgagagcaggtgcaggccgaacaggccgtcctggatggcctgcggcagcggctgggcgagccacttctccgcccgctcggtctcgcccagccggatggcggcggccaccgcgctgctcagcggtagcgcggcggtgatgccgagcgtcgagagccgccggtggtccagaacggtgctgcctgcctcgaccgccgcggccaggtcgccgcgacgcaacgcggcttcggcgcgcggccccgcgtagcccgcctcctcctcgttcgacgccccggtctcggagaccagcttgtcggcccagagctgagcggtgtcggtgttctccgcgtacagcaaggcggtcagcgcggcgagggcggtggtgccggccgccgccgagtcggcgtgctgcaggacgttctccgcctcggccgcggcgagctcgggacgaccgcgcagggccgacatgacggcattgtcggcgatcgcgcagagccgggccgccttcggcaggttcgcgagctcatccggcgtgagcggacgcggccgccggtccgggccggacgtgccggggctgaccagcgcgagcgatacctcggcggcccgcatatgggccctgtcctggtcggtgaccgcacgggggcgctgcgaggccagcagcccggctgcctcgccggaccgcccgccgacgcccagccagcggatcatcagcgccgtgtgctcggggaagagcagcccggcacgggcggcgacggtcagctcgtcgaggtgccggctcgaggccagcgggttgcggcggcgctcgatcgccacggcctcgacccgcagcccggcccgggtgacggcgtcgtcggaggcctggtgggccagttgcagatagtcggacgcggtgtccaactcgccgcgcacatgcagctcctggccggccttggcgaatatctcggcggccttcgggccgtgcaggagaccggcgccgacctggtggtgcgcgaccacttccacgggcacgtcgtccagcacttccagagcggaccggtgcaggcggcgtcgttcctgggcggacatgtcgttgagtaccaccgaacgggccgccgggtgcgggaaccgcaggtcgtgcagcaggccgccttcggtgaggatctcggttgcctcgcccaccgcgtccgcgttgagcccgctgatccagcgcaccagggtggtgttggcgctctggcccagcacggcggccacccgtgcgacccgcaacgggaccgggccgcagcggtagagggaaccgaggtatgccagccggtacgcgcggcccgcgctgatgcccgtccctcccgtctcccaggcctcccgcacatcgttgatcagtccgcgggtgaggttcaggttgccgccggtcgcccggtacaaccccgtgagcacgtcctcgtcgacgtcgggaccgagaccggcgcggaccagctcggcggtgtgcttcgggctgagcgggcgcagcgcgatctcgcggtggtgccccaggctgagcagctcggcctggaactgtgagtgggccggggtgagccggcgcaactcggtgaggaccatggcgatccgggccgagccgacccttcgggccaggtggagcaggcagttcaaagaggcggtgtcggcgtggtgcacgtcgtcgatgccgatcagcaccggccgttcggcggcgagcgccagcagggtacgggtgaggtcgctgcgcagccggttctcggcctccggcgacaggtgctcgcccgggagggtcgggcaggccggctccagcgcgtgggaggcgagcgccgggttgtcgatgagctgcccgatcagggcgtacgggagtgcctgttcctccgcggagcagaccgctcgtaacgtgatggcgtcggccttcgcggcggctgcgtcgagcagttcggtcttgccgcaggcgatcggtccggtgatggtgaccaccacaccccggccgttggcccgcccggtccacaacctcctccagttttctgagctcgtcatcgcgggcgtccagttcatagcactccacggcaggcatcacctcacgtcggacaacccaattgctttccaccgcaaagtatgtgaccgcggactcttctgcggctgatcgacgagtacattgcggagctctagtggggttcaggccaagcggtctgttcgggccttcatccgcccggaacagcccggggttgccctgtttcgcagcccaggaaggtgcacacaacacgaacttgaaccacaccgccgtctggtggaatatgaccgccacaagacaaagccggagcaacacaggcgaagggaccggcggatgaccaacggcgctggagcggaggccgacggatcgcgcggggcgttctccgtggactccaccgtcccgggcgcggcaccacaaggattcttccaggccttccagcgcgggtgggacgcggagctcggcgacggcttgccgctgcggagcttcaaccgggatgtgaccggtgacttccgggtcaggagccgcgccgccaaggtccgcgacctggcgttcaccgatctccacagcatgtcggccatccggaccgcacacgcgctgggcggtgtggaggaccgggtacggatgtacgtcgtgaaacgcggcgcatggacgttggcaggttcgcgcgatcgggaccggcacaccgtggcggccgggcagttcctcctccggcacgtcgggcagccgtggcgcttcgaggcggcgccgcacacgacggtgaagatccttctgctgcctcccgggccgctcaccccactgctgggaaaccgggccgtcaccgggcccgcggattcggccgaggtacgcctgctggtggcccacgcgaatatggtgtacgagaccatgaccggcctgggcccggccggtgtgcaagccgcccacagcaccctgctcgaactggcccagtcggtggcgaggcgtcggttcgacgatgtggagccccggctggcgcccgcgctcgccggggccgcgaaggacctcgcggacagccacctcaccgaccccgagctctcccccacgatgctggcgcgtgaactcaacgtctccttacgcaccttgcagcgggcgttcaccgtggcgggagagtcgttgatcgcctacatccgtcaccggcggctggaagaggcccgccgcgctctcatcgcctcggccggcaggctgagcgtctcggaactcgccgcccactggcagttcgccgacagcagccacttcatccgggtcttcaagaagacctacggccagacgcccaccgagtacgcccgctcaaccgggctgaccgacagctgaacccgggcaggctcagccgacggggccgccgtcgagttgctccacggaggcggcgtccccggccggtgcggcctcgctcgtggccgcgggccgccggtcgagcccgagcacgagcggtacggccagcacgctgatcgcggtgaggaacacccagggcagcgccgcaccgccctccagcagcgctgtgagcagcgccggggcgacggcaccgccgaccgaccaggacagctggtagacgcccagatagcggccccgcagatgctcgggcgccatcaccacgctcagttcgctggtcggcggcgagccgaggatctcgcccacggtgtagagcaccatcgcgatcagcagcccggcgatcaccgcccagccgggcagcacgagcagcgtgccgaagacggcgaacgccgccgcgttgaacagcagggccagcaccagcaccgccgccggtctgcgggcctcgatccaccggctggcgagcgtctggagcagcgcgaccatgaccgtgttcagcacgacgagcacgccgacgatccaggagccgacagcgaggttgtccgccacgtagacggcgagcagcaccgcgaccaccgtggaggcgaaggcgaaggcgatgttcgtcgcgaccagccgcagataccgggtgtccttcagtacggtgcgaaagccgcccgagggttgtccggtggcttcgcccgccgcccgctccggctcggcgggcttcgcctccgcggggccggcatcggccgccccgctcccgtccgcgccccggaaggtgatcacgagccagcccgcgacggcgaaggtgaccacgttgatcaccatcaccgcccgcagccaggtgacggttccgatgctcagggccaccgcggacgcggcgccgccgaaaccgacgccgacattgcggagggcacgcacgaagccgaaccagcgggcccgctcgacgccccgggcgaccagcacgatgagcggactgttgcccatccagaaggcggtggaacccagttgcaccacgaactgccacagcacgatctgccaggccgcgtgcgagaacaggaaaccgatgaatccggccaccgaaatccagttgcccgcagcgaccacgatacgcggaccgtatttgtccaacagggacccgataaagggcggaacgggcagggcgagcagctgtccggcggtcagcgcggcgccgacggcgaccagtgacagcgacgtcgtcttcacgaagaagacgacggtgaacgccatcaccaacccgttgcccaggctgtcgatcagattggccgtgacgaagcggccctttcccgcgaggtccggcagaccccaggccgtctgaatccgccgccacgccgaaggcgcgggggaatgccgcgacatcatgacttctctttacttcggcgcatcggcgggcaattcgcccacggcaggcacttcgccccagacgggcagctggctctgatggaagacggccgcgctgcgcgccttggcgtgcgccgcgatggaaatgatgccgcccgaaccggtcggcaccaccttggcgaacgctccggcacagccgtgcggggtgtagaacatgccataggccgcatgccattcctcggtttccccggataccgggtccaccacgagctcgggccgggggatcacccgctgttgtacgacacatccttcggcggcggagtcctccagagcccgccgccaagtggcgtcgtccgtttcccagcccaccacggcgcccactcccccgccgcgcccgttgaccttcaggatcaactgctcgcgccgcgccacgcattcgtcgacgaggtcggtcttgagcgcgtccgtgttgatcagccgggtccagggcagcacccggtcgatgagccggcactcgtcctcgccgaggtcgcgccgccacgccggatccgaaagcagcgccatacaggccttgttggcgaacatgttgctggaaagcggggtccatgtcagcgtctcgcccgcccggtgcgcacgcagcaggggcgcgaccaggtcccagccgtcgggctcctcggcgatctggtccgggacgaaggagcggtagaccacgtcgatcttcgtgtccccgaggtagagcgccccgccgcgttcgtgcacgtcggagacctcggccaccaggatgtccaggcccgccgagcgcagtgcggactggaagttgagccagagttcgccgtagtccttcagacccccacgccattcaagcagggccaccaccggttcgcgcccgggtgccacccgggcggcgaagtggcgcagcgcctccaccacggcgcgaatgggatgcacgtaaccaagccgattccggcgcgcgaagtcggcgaaggcgtcaatctccatcgtcgccctggagatttcactcgcgaggccccagccgccggcctcgctccccagattgaactcgaggagtttgaacgaggtgccgtcgtggtacatgtcggcccggccgaactggggtggcggtacgtcaccgaaccgccgtatcgcttcccagcggccgtcgctgatgccgagcaccgaccggaatgcgtccagatcgccgccgaacatccggtccggcaaggaaaaaaccgtgtcgatgagatgctgcaggtcatcggcgaactcgcgcatctcacgctcggcgacgaagaggggcctggccaaatactcgccatgccagggggcgagcgtcgggaagtccttgacgtgctgggcgaccgtgggcaagtcactgcccggtatgcggaactcctcgttgtactgggctgctacccggtccgccgacatgaagtctccaactcccgctcgatggtgactgagcccctcaagcccgcgggaatcaacgtttcaccccgatcttcatgcgcgactcaactgcgactcaactgccgctcaatggccccatgtggacgtggagcatctgtgttaacactagatacttgaacagccgaaagaaatggctgtgcggcaatgggggaacgttactcggcgtccgatcgcgcagcgagctcgcgggcacggtcaggcagagactatgggaggcgccatgaccgccacgacggacactttcgccgacgccatcggcgggctcggggacaccgagtgggaccggctcgccgacggcaggttctactcgtcggcgctgtggctgcggcagtgcgccttcctcgagcccgggtcggtctccggcggactgcacgtggagaccccgggcggcggcagggccgcgatccccgtggcggcggtgaccgacgaagccaacccgcagctgcggtggcacgacctgctgaccgcgcgcggcctgccggcgcccgagccgggcggcctgctcatcggccagcgccgcggctacctggcgcatctgctcaccggcgagggaaccgatcggaccgaggccgccgccgcggtgctcgacgcggtccgcgcgctgcgctcccccctgcacgaggactcgaagatcgcccgcgtggcgatgtacctgaccaccgaggatgtgcgggcgctgcgggcggcgggggtgcggaccgccccggtcgcgctgagtaccgacgcctggatcgggatcccgcccggcggctaccacgcctggctggaatcgctgtcggcgcaccgggcccgccgtatccgcagcgaggtacggaagttcgagagggccggatacgaggtggagcaccggacgctcgccgactgctggcaggacgtcgcccggctgctggcccgttcgctactgcgctacggcaggactgtcgacgtggccccgatcgccgagtccttccgcgagcagggtgaactcgccggttcgcgcgccgaggtggtgctgtgctcgcgcgccggcgaaccgcccgtcggcttctgcctgtactaccgatgtggcgacaccgtgtacctgcgggcgctcggcttcgaccacgaccaactcgtctcggccgccgagtacttcaacctcacctattacacgcaggtacggctcccgggagtgcgctggctgcacgcgggcaccgagaccgcggaaggaaaggcactgcgcggcgcgacgctccggccgctgtggctgctggacctgtccgaggacagcccgctggccgggtacgacgaggagatccgcgagcacaatcgcgccatgctcgaccgcctgcgcgactcctcgcccgcgatttccgacgccctggagtacgagctctggaagccgtactgctgactcctttttctgtcttcgggtgtttgtcgtagtgcgcttgagtcgcggttgagccgcttcgaccggttccgtgccagactcggcccatgctccacgggcgtgcggatcaatctctgatcgaacgggcaaggcaaattcgcaggggccttatgggaaccccgctgattcctctggcggacgagcagtgtgacatcgtcgcgaagctggaattctgcaatcccacgggaagcagcaaagaccgttcggcgctgtggattctggaacaggcgatcggccgcggggagatagcgcggggcacgacggtggtcgagtcctcgtccggcaatttcgcgctggccctggccttctactgccggatgctcggcatctcgttcgtcccggtcatcgatccgaactgcaacgaggccaccgaggcccagctccggctgctgtgcgagcgggtggagaaagtcgccatccgtgacgggtcgggcagctacctgaagaaccggctgtcccgggtccgggaactgctggtcgaactcgactcggcctattggcccaaccagtacgccaatcccgatgcccgcgacgcgcattaccacttcaccgcgggcgagctgatcgcacaggccggcccgctggattatctcttcgtcggcgtgggcaccggcggcacgatcgccgggctctcccgccggatcaaggaaacctaccccgactgcgtcgtcgtcgccgtcgacgccgaaggctcggtgatcttcggcggcccgccgaagaagcggcggatccccggcatcggctccagcatcgtgccaccgctgtgcggccaggcgctgatcgaccacgtcgagatcgtctccgaggcacgagccgtcgaggcctgctccgccctggccgccgagtacggcctgtacgcgggcggctcgaccggcagcacctacgccgccgtccaggactacttcgcccggcaccgccccggcacccaccgtccccgggtcgccttcatcgccgccgaccgcggccacgcctacgcccagaccgtctacgaccccctatgggtccagcaactgcacgcggaaacggcacaaacccccggcgtcgaaaccctggccgtcaactgaaggagaaccaccgccgtccgccgcgcggctacggcagtggtgtacgcgtgaaagagagagaacagatgcagcagagcaagatacggctgagcggacagcaggccgatccggagttcttccgcttcgaggagcgcggcgaactggactacgagctcgttctggacgtcctgcacggccgcaggctgggcgtgatcttccgggatgtcataccgacggcggcgcgcaaggaactgaccgaacgattctgggccagccccgcccggcagcatcgggagggcgagccctcgcactacgtcggcgcctaccactggaacaaggacgccgacacctatctcgacgagtccgccgaggtcgccgagcacgtccagcaggtcatcgacgccccgaactcgccctggcacgagttccgccagggcgtggacgaggccctgcggcaggagggcgcgtccctgcgggtggcgcggatgaacggccgctcggcgtgcgccgcgctgatccgcgcgtgggacaaggagggtgacttctccctcgacccgcacgaggacgaggcgcagtgcaaggacccgcgacaggccggtttcgagatccagcgggtcctggaccaccagatctgcgcggtgaacatgtgcaccgaacacagcgccggagggcggctggtgatctggaacatccgccccgacgacgcgacccggcgctcgctcgacatcgagctgacggggctgccctaccaggccgaggccctggcggacttcgaggaactgcgcctggacatccgggagggcgacgtctacgccttcaacggtgcgttcgtgcacgcggtggacgccaacaccggcaaccgcacgacggtgtccttcatcatggggttcaccgatccccgaacggtcgtgagctggacctgagccctaccgggccggcggcctagtcgacggcgtcgactaggcaccgaccgggacgacgctcgcgccgtcggtgccgctcatcgccgcgtttcctccccgagggcctccccgagggcctcggcgaggccctcgacggtggggttcctgaagaccaggaccggcgagacccggcgcccgagccgctcgctgagccggcccgccaccgtggtgatggtcaacgaggtgccgccgacgtcgaagaagtcgtcctgtggcccgaagtcctcctcgacgcccaggcactcccgccacacgtcggcgacgaggagctgctgtttggtgacggcccggcgggtggcgggggccggggcgggcaccgggcgcgggtcgggcagtgcgtcccggtcgaccttgccgttcggggtgagggggatctcgtcgatcacggtgtagctctgcggcagcgtgtacggcggcagtcgctccgccaggtaggcgcgcagttccgccgggtccggggtgggccggtcggggtgttcgaggatcaggtaggccgccagctggtccacggcggtgctggtgcggcgcacggccaccacgcagtcggcgcaggccgggtgggtctggagggcggactcgatctcgccgagctcgatgcggaaaccgcgcaccttgacctggtggtcgttgcggccgaggaagaccagatttccgtccggcaacagccggaccacgtcacccgtggcgtacatgcgggagccgggctccgcggcgtacgggtcgggcagatacgcccctgccgtacgggccggcctgccgagatagccgcggccgaccgcgctgccgccgacgtacagttcgccgcggcagcccggggcgaccgggcgcagccgcgggtccagcacgtagaggtgcgcgttgtcggtcgcccggccgaacgggacgctcgggcgatggctgtgccggtcgagcagctcctcggtgatcgggcagaacgccgaggtgacggtcgcttcggtgggcccgtactcgttgaagacctggcatcccggcgcggtggcggacaggatgcggcgggccacgtcgtgcggcacggcctcggcgccggtgaacaccaggcgtacggaggcgagccggggagcggggtccgtgccggactccagcaggatccgcatctgtgagggcgtcgcgttgagcgtggtgatgccgcgctcggcgacctctcgcaggaactcctcggggtcgtaggcggcggtggggctcgccaggtgcagttcggcgccggacaccaggcactggaagatctcccagaccgagttgtcgaacgccaccgagtggttgagcagggcccggtcccgcccggtgagccggcacagccgctggttccactccaggaacccggtgatgttggcgtgggtgatcgcgacgcccttgggattgccggtcgagccggacgtgtagatcacgtaggcgaggtggtgtggttgcgcctcctcggccggcccggggtccgccccgggctcctggtccaccggcacgatccgcgcgccgtcgccgaacagggccgaggtggggtgtcccgggtccgcgacgaccaggcgtacgcccgagtcggccagcaggtaccgcagccgctccggcgggtagccggggtcgagcgggacgtaggcgccgcccgccttcagcacacccagcagggcgaccaccagttccaccgagcgttccaggcagacgccgaccaggctttcggggcgcacgcccccggcccgcagctcccgggccagggccgaggcccgcgcgtcggtctcggcgaacgtcagcgaccggccgtcgcccacgacgcagagcgcgtccggggtggtccgcacctggcggtcccagaggcggaccagggtggtcggctcgggcagcggctcggcccggtcgccgatggagagggcgcgcagctcatcggccgagacgatctccagctcggacagccgggccgtggcaccggacaccatctgctcgatcacccgcaggaagtgccggccgaggccccggacgaagtccgcgtcgaacaggtcggtggcgtactcgatggagatgctgaggtcaccggctccggcggcgtggtccgtcacgtcgagggcgaggtcgaagcgggcggtgccgagccgtccctccagctccttgcggtcgtacagctcggtgaggtgaccggccctcggctcctcgtgcagggcgagcaggacctggaagagcgggttgcggctcaggtcgcgctccgggctcagctgcgccaccagcttctcgaacggcagctcctggtgcgcgtacgcctgccgcgcgacctcgcggacgcggcccagcagctcgcgccaggtcgggtcgccggacaggtcggtgcgcagcacgacggtgttcacgaagtagccgaccacgtcgtccagttcggggcgcggacggccgcctgtcagggtgccgacggcgacatcgctctcgtcggcgtaccggccgagcagcacctgcaacggggccagcaggcccatgaaaagggtgacgccctcggcccggcacagtgtgcgcagccggtgcgccaggtccgcggggacgcgcagcagttcgcggcggccgcggtggctcgcgacggccggccgggggcggctggtcggcagttcgaggacgggcacaccggacagcgccgcgcgccagtggtcgagcagccgctccagctcggcgtcggtgagtcggtcgcgctgccaggccgcgtagtcggcgtacgtcacgggcagcggtgcgggggccgggcggcggccggcggcgagctcggtgtagagctcgcccagttcccggaccatcaggtccatcgacgcgccgtcgcaggctatgtgatggatcgtcaacagagtgatgtcacggctcggggtgcgcagcacgtccgcgcgcagcagcggtgggcggtcgagggcgaagggggcgcggatccgctcggtcaccagcgcgtccagatcctcctcggcgacctcgatgcggcgcagcggcaccgaggtcgcctcgcgcggttcggggcggacccggccgtcgacggagacgagggccgtgcgcagcgccgggtgccggtgcaccagcgcggacaggcagtcctccagcaccgccaggtcgacccgcccgccgggcaggcccagggcgaccggcaggtggtaggcgggcaccgcgtcgttcagctgctcgaagaaccagatccgctcctggccgaaggagagctcggcggtcccggccgcgggaatcggctcgtcgtccgagggcgccacgccgtccagggcggcggcgagcgtggcgggcgtcggatgcgcgaagaccgactgggcgtcgacccgcacgccgagctccgcacgcagccgggccacggcccgtaccgcggtgagcgactggccgccgaggccgaagaagtcgtcgtacggggacacgtcgtccagcccgagcacctcggcccatacgcggcgcaccgccgcggtgtgcggcggcagcggggcgtcgtcgggcatcggcccgctcgcccggaccgggctgtgcgggatccgccgcacagcgccgcggtcggccttgcccgcgggggtgcggggcagggcgtcgagcaccgcgtagccctgggccaccatatgggcgggcagccgccgcgcgaggtggtcacggacctggcggggcagctcctcggacacgttccgcgcgccgggcgcgggcacgaggtacgccaccagctggtcgccgccggccgcctcgcggtcgacggccaccaccgcgtccgtgacgagggggtggctgcgcacacaggcctcgatctcgccgagttcgatgcggaagccgcgcaccttgacctggtcgtcgtcgcgccccagcacctccagggtgccgtcggcggaccaccgggcgaggtcgccggtccggtacaggcgcccggaggcgtccccgaacggatcgggcacgaaccggtcggcggtcagcgcgggccggttgaggtagccgcgcgccacgcccgcgccgccgatccacagctcgccggtgactcccgggccggcctcccgcccggtctcgtcgacgacgtgcagccgggtgttggacagggccgtgccgaccgtgacccggtcggccgcggagtcgacgcgggcggcggtggcccacaccgtcgcctcggtcggcccgtacaggttccacagggtggagccggtggcgagcaggtcgcgggccagctgcggatcgagcggctcaccgcccgacagcagtacccggccgtgcccgtccgtccagccggacgccagcagaatccgccaggtggtgggggtcgcctggacgacgctcacagccgcctcgtcgatggtggcggccagcagctcggggtcggtgttggcggcggccggggccatcaccaccgtcgctccccgggtcagcggcaggaacagctccagcgccgagatgtcgaacgacagcggggtcagcgcgaggacccggtcgtccgcccggaggccggggcgttcggccacacttcgcaggaacgaggcgatcgccgcgtggtcgacggccacgcccttgggcaggccggtggaaccggaggtgtagatgacgtaggccaggtggtgcgggtgcaccaccggggccaccggggcggcaggttccgcggggatgtcctcgaccagcaggacgtcggccgcgccggacgccagggcccgcgcgcggtggtccgcgtcgcacagccacagcccggcgcccgagtcggtggtcatgtaccgcagccggtcgggcgggtagctcgggtcgaggggcaggtacgcggcgcccgacttcaggatgccgagcagggccacgaccatctcgaccgagcggccgagacagacgccgaccaggtcctccggtccgatgccgcgcgcccgcagctcggcggcgagtgccgtggcccggcggtccacgtcggcgtagccggcgcggtgtgctcccgcgacgacggcgaccgcgtcgggtgtcgcagctgcctgccgctcccacagtgcgaccagggtggtcggcgccatgaacggatcccttctcagccccgtacgggcccttctcagccccgtgcggggaaggtacgcgtgatcgtctctgccagctgtcgttccgcgccgcgcacatacaggtggggggcgcccgggaacaggtgcagttcgaagtcggccgaggtgtgctcgcgccagccgctgagctccgtccggtcgacgctttcgtcgtccgagccggccagaacggtcaggccacagggcaggggtgcctcggcccggtgcgtgtacgtctcgcacagcgtcaggtccgcgcgcagcagcggcagcagcagctcctgcaactcggggtccacgccgtcgaattcggccgtggcgagcagcttgtcgtcgggcagcgtgtgccgcgggtcgcggtccggcaggccgggggcgcgcatgctcgacagaaacaggcggtcgggcaggcgtgtgccgagcgcacgcagccgccgggccgtctcgaaggcgatcagggcgcccatgctgtgcccgaagaaggcgtacggcaggtccgtgtggtccgcgatgagcggcgcgagcgcggccacggcgtcgtccagcgacgtcagggccggttcggcgagccggttctcccggccgggcaactggaccgtgcagacttcgacggtttccggcaggtgcgccgccagccggtaggcggaggcgcccgccccggcgtgcgggaagacgaacaggcgccaccgcgcggtgtgttcacggccgccgagcccgcgcagccaggccgtcgcgagggcacccgccgtcatcgtgcggctcgtacggagcgcggggtcaggtcggtccacacctgctcgatgtggtccaggcactcctggcgggtgccctgcgcgccggtggcggtccagccgtcgggcagctcacggtcggcgaaccagaccgagtactgctcctcgtggttcaccacgacgacgtaccggtccccgtcggggcgcgcggcgggtgaggcgctcatgcgtcagacccccaccccggtgaagaagtccctctcgcggtggccccggcccgcggcgacgacccggtcgtgcacccacttgccgacggcgaggtcgagtacgccgagtccgaacggcgagaagatcgccgggcgtccgtgctgccggtgcagccgcccgcgcagcaggtcggcgagggtgccgtcgacgaagtcccggttgccggtgcgctgttcggtcaggtgcagcgaggtgcgctcgcgtacggcgtggtcgacgtcgtcggtgaagttctgggcggcgaggatcagctcgggggcgaggtcgcgcagggacagatgcaggaccaccgggttgtgggccagcagcccggggtcgtgcagatgcggttcgccggccaccgtggtcagcaccaccaggtcgcagtcggtgaacgcgtcctccaccgcctcggcgaccacgatctcggcctcggtgtgctcggccacgacggcggcgaagcggtccgccgccgccgggtcgaggtcgaagagccggaagccgtcgatctcccagtcgagatcacgcaggaaccgccatacgtgctgggcgatcagacccgtgcccacgatgccgacccggcgggcacggcgcgcgccgagcagcgtctgcgcgcccagtaccgcggaggccgcggtccgcgtcgccgagacgatggaggactccatgcaggcgaacggatagccggtgacgcagtcgttgaggatcagcgtggccgaggcgcgggggatcccgtgctgggtgttgtccgggaagctggcgatccacttgttgccggcgacctcgaactcgccgcccagataggcgggcagggagatgatgcgcgaccggctctggtgggggaagcgcaggaacgagctgtgcgggttcgagctctgtccggcatcgtgcgcgaggtaggcggcacggacgacctcgatgagctctggacggtgacgatccgtctcggcggcgatgtcgcccgcgccgatcacggtcatgggcggcgggaccatggtggacacggggtggttctccttcagttgacggccagggtttcgacgccgggggtttgtgccgtttccgcgtgcagttgctggacccatagggggtcgtagatggtctgggcgtaggcgtggccgcggtcggcggcgatgaaggcgacccggggacggtgggtgccggggcggtgccgggcgaagtagtcctggacggcggcgtaggtgctgccggtcgagccgcccgcgtacaggccgtactcggcggccagggcggagcaggcctcgacggctcgtgcctcggagacgatctcgacgtggtcgatcagcgcctggccgcacagcggtggcacgatgctggagccgatgccggggatccgccgcttcttcggcgggccgccgaagatcaccgagccttcggcgtcgacggcgacgacgacgcagtcggggtaggtttccttgatccggcgggagagcccggcgatcgtgccgccggtgcccacgccgacgaagagatagtccagcgggccggcctgtgcgatcagctcgcccgcggtgaagtggtaatgcgcgtcgcgggcatcgggattggcgtactggttgggccaataggccgagtcgagttcgaccagcagttcccggacccgggacagccggttcttcaggtagctgcccgacccgtcacggatggcgactttctccacccgctcgcacagcagccggagctgggcctcggtggcctcgttgcagttcggatcgatgaccgggacgaacgagatgccgagcatccggcagtagaaggccagggccagcgcgaaattgccggacgaggactcgaccaccgtcgtgccccgcgctatctccccgcggccgatcgcctgttccagaatccacagcgccgaacggtctttgctgcttcccgtgggattgcagaattccagcttcgcgacgatgtcacactgctcgtccgccagaggaatcagcggggttcccataaggcccctgcgaatttgccttgcccgttcgatcagagattgatccgcacgcccgtggagcatgggccgagtctggcacggaaccggtcgaagcggctcaaccgcgactcaagcgcactacgacaaacacccgaagacagaaaagggagagaggctgttcagcccgtccacaacggacgactgaacagcccctcgggtcactcggtggcgtgtgctcaaccctgctcttcgtactcgacgtgcagcgtccgctcgaggtgggcgcggtactcctcggcctcggcatccgactcatgacgggagatcaccatgaccgggtatccggagtggtccgggtagggggccagcagtgtgccggggctcacggcctccaccacgtagtccacccgcgggtcgtcgcgcaccgcgtccaagccgcgtatccgcttcatccgtccaccgctgccgaccgccacctggtaggccgccacgtggccgaccggttccggctcctcactccagcagagcggttcctggccgaggtggatgcgcatcgcctcggcggccaggtcgatcccgctgctgaggtggacgaggtggtgcatgaagccgccgccgccgatccgcgcgcccatctccagcagcaccggccgcaccccgccgaccagccgcacttcggtgtgcgtggtgccctccgtgacgccgaaggcgtggtgggcaccgatcacctccgcctcgatcgcggcgagtatctcggcgggcagctgggcgggggcccgcagcgtggtctcctcgaagtagggacccgccagttcccccttccagctgatgccacagacccgtacccgcccgtcgcgcacgagggattcggcggagacctccggcccatccaggtactcctcgaccagcacaccggattccggggccgatccgtcggcggcccccaggtggtgggtttcgatgagcttccaggtgcgggccacggcctgctcaaggctgtccttgtcatccacacggatcacaccgaggctggagtggccgttcgccggcttgaccacgacggggaaccgcaaggtcagcgcctcggtccagccgtccggatcctcgacggccacgaagccgggcgcgcccagacccgcctcccgcaggtgctggcgcatgctgcgcttgtcgcgcaccacggccgccgcctcctcggtcaggccggtgaggcccagctctcgggcggcgcgcgccacgaacggcacgaccctctcgttgcccgccatgattccgccgaagggctcccgccggtagcgctcggcgagcgcgatcagcgcaccttccgggttttcctcgatgtccagtggcagcacttcgaccaccgccttggcccgcccggcggactccgccgagtcgtcccccctggggatgaggatcaggtcgataccggcacgctcggctgcttcgaacacccagggatacacggacaactggccgacgaggacgagtttcgggcgctgtggcatgacggttctccaagggttcggttgcttgggcggttcgggcgaggcgggccggccggtgcggtcccgcatgagatcatcccggcgagcacggagccgcgccacgcgctggagacgcgcggcgcgacgggggaagtcgtcctggagccgtgagcctcaggccgagcacatgaccggcgcgttgctccgccgcgccgcgaacgggcgtaccccaggctcccactcgtaggagaaccggccgttcacgcacaggtgccgcgcgcgcagcaggtagctctccgacagttcctccagcagatcctcggcccgctggtcgctgatccccagggccacggcggcaagcgccggcgtgagccggtcggggacgccgggggacaacagcgcgtacgccagggaccgcgactcggcgggcagcgtcgcgagcgtccggtcgaatccggcccgcacgcccagcggatcgctgtcgccgcccccggacgcgaggacccaggcagtggcctgccgggccgtccagtgcgggcggcggcgcagctgcgaagccaccgcgaagagcgcgcccggcagcccccggcacagcccggccagccgccgcagcccctccgggtccatggtggtccggtggttcggcacggaggatgccagcaggtgcagacagtcgtcgaaggacagtccggccagttcgacgtggaccgccgacgaggacacgaacagccgccgccggctgccgatcaccacgccgcagccaccgccggacggcagcagcggcgtcaggtcccgtacgtcgcacacgtcgtcgagcaccaccaggacgcgcaggtcggcggtccagctgcggaacagcaggctcaggtcggtggtgcccagccgcaactgctcctccgtcgcgccgagtccacgcaggaacgagcggaggatctcggcgggccgcacccgccgcccggcgccgtcacggagacgtgcgtagagccgtccgtccgggtagtggccgctcacctgatgcgcggcgtgcaggcacagctcgctcttccccgtgccgggagcgccccccacgatcacgagcggccggtcctcccgcgcccggcccgccagcgccgcggtgatgcgcgccagttggccggcgcgtccggtcaggcgttcgcgcggggcgggcagatggcagggcgcccgggtggcggcggtcacggccgtcagccgcccggcgggacgcagcagctcctcgtccgacgtgagcacggccgtgtgcagggcctggagttcgggggaagggtcgaggcccagctcggtggcgatccgctcgcgtgcccgctggtagacctggagcgcctccacccgccggtccatgcggtagagagccgtcatcagattgcgctgggcccactcgttcgtgggttcccgcccggccatcgagctgagttcgccgagcacctcgtggtgccggccgaggtgcaggtcggcttcgatccgcaggtggtgtgcgctggtacgcagctccttcaggcggaccgcctcggcctgcagcgcgggccccttgcgtacgtccaccagggcatcgtcccgccacagctgcagcgccgcggcggccgtgtcacgggcccgggcgaagtcctcgtcacggaactccgcacgggcgcgggcgagcagcggctcgcagcggaagacatccacgtggtcggcgggcagcgaaagctggtagccgccgtcgaccgtgcccagcagcgggccggccgcccgtaccgcgccgccgctcctcgggttgccgagggagaaccgcttcctgagctggtacacatacgtctgcagggcggtgtgcgcgctggccggtgggtcgtcgtcccacagttcgtccatgagttgctcgaagcgcaccacccggcccgcgttcagcacgagcagtgccagtgtctgacgtactttctgggcggccggcgccaggtcgacgcggtgatcgtccccgtaggcctggagcggcccgagaaggttgattctcatgaaattcgccctgcctcatgttcgttcggtccccgtcagaggagttcggtgccggatcgcagccgccctgcccggcgtcaccccggccggcgacgcggtccgcccggacgttcgtccccggcgagtgccggcccgcgtcgctcgtggaacgctgcctgtccgtgccgtccgtggccgcgtggatggcgcgtgcctcgggtccgaccagggtcggggcgcttcgtgggcgcctcctccgggcgagtggcccggcgaacgaacaatctcttcggcgtgacctcgccggcggcgatctgctccagaacgaggtcacgggggcttccgtgctcggcgatgagccggccggcctcggcccgcaccgcgctcatcgcctccgccggcacgagccgcgccctatcgtcgaacttcagcactccgacgcggctgaatcggccggccggcccggcggcctgctcgggtgtgcggctccaggtcaggcactcgtggacttggccgccgcggccaggttcgggtggtgccgggccacgatgtcggggaaggagcggaccagccccttcatgacgttgtcgccgtacagcgcgaacagcgggttgtcggcgacgtcgatgactccgccggcgccctcgacatatgggctgggcagcggctgcatcgtggcgtccagccgagggttgtagaagaagggaatggagtagcggttgacgcccggggcgggccggaccacccggtgcagggtggcacgcacatagcccctcgtcgccacctccagcagctcccccaggttgaccacgaacgcccccgggatcaccggcacatcgatgaactggtcttcctcgaccgcgacctggaggccaccgttgtcgtcctggacgagcagggtgagcagcccgaggtcgttgtggatgccggtcccctggccggagccgtcgtcctcgctcgtccgcgtgggaccgggatagtgcagaagcttcaggtggatctgcggatccggatcgaccacgtcgtccaggaacccggcgggggcgtcgagcgactcgagcatcagccgtagcagtcgatgtgacagatcggtcagccgggtgatccaagactcgatggccgggcgcagctcgggcatcgccttgggccactcgttcgggccgatcaggcaccggtaggcgggatcgccgggcgcggacgtcctcgtgggctgctccggcccgatgtccagctgctggcgctggtcggggattcccttggtgtgttcgcggccgagttcggagtagccgcggaacagcggcgagtcgaggatgttcagcgcgagccgatcggcctcgggcagggcgaagaactggcgggccagctcgagcatgtcgtcggagtcctcgacaccgtgaccgacgagctggaagaagccgacctcgtggatggccttgcgcaaccggttcaggaactctgcccgggtttcttccgaaccggcggcgtcacggaggtcgagcaccggaaaggggtcggtcatggcagcaccttcgcattgctggtctggtaggtcgtcgggtcggtcacgccggcgtcttggaacgcttcctggcggtcccagcaggaagcgcaccgtccgcagtgcagcgggccgctacgcatgcaggtccaggtatcggtccaggggacgccgagctcctcgccgagcgacaccacgccgctcttgcgcagttcggccagcggcgccgtcaaggtcagctcggggtgcgcgtatccgtgggtcgcgatcttctccatggcgaggaaggagtcgagaaacgcgctattggcgtcgggcgcggcctgtgggtcgtcgatcaccccgatggccaccgcttccgccttctcggcgacggcgaccgcgaaggcgacggacagcagcagggcgttgcggttgggcacgacgttcgggctctcgccgggcgagtccctcttctgatccggcacgtcgacgtccgggtcggtgagcgaggagccgtgcatgagcttgccgaccgggctgaggtcgacgacgtcgtgcggcaccccgagggttcgagccgccttcgcggcgaactcgtgctcgatgaggtggcgctgcccgtagttcacggacagcagtcggagcgtgtgtccttgtgcctggaggtgataggccatggtgaccgagtccaggccgccggacacgattgcgacagttttgggcatgcgtcgccttccggcggatggaaatggttgtggatcagtgcctggggatcagagcggcggtgtgccgtgcgcggacacggcggcggacgcgtggctcggcgccgccgtgcacagcggttgccgcacggtgccggtcatgccgccggcgtggccgggacgaccagccgggtcgcccgccggcccctgcggtaggtcagctcgacctcgggccgcggagcgtccagccgcatggtcacggttcgctcctcgctccacccgaccaggcggtactcgtctccgccgagggcaaccagcgggaacaggtggtcccggggggatcggatggcgagctccgtgccggtgtcgacgacgtggaaggtggcctgcggttgcgggcaggtgagcgacccgaactcctccacctccagcaggtgctccggcgcgtcgaacgaacacggggccgcttcccgctcgccgtcggtcaaggccaccgccggccggttgtagagcacgtccgggccgttgaagtagtcggcccagtcagccgggaacagctgcgcgagctccggtgtgacaagtacgtccacgaccaggtgcacccgatgctcgggaccgaggttctcgatctggtgcatgcggctgaagtcaccgaaccagaactcgccgggctgccagcgataggtcaccccgtcaagctccagcgtggccttctcgtgggtcatgaccgggatgtgcagccgggccattccccagtccggggcgaacttgaagtcgcaatggcggtagccgacggtgtcggggccgagatccatgaaccgggccgcgaagaggggtccggggatcgaacgcaggatctcccggacgtacggcatatgggccagccactcggtgtcggcgaactccgcaccaccggggccgcccgggtcggtacgtgtcttgtcaccaccggggctgcgcaccgcgaggcagcgccagtcctcctcggcctgcctgaccacagccccgtctcggtacacgcgctgttggtcccacaggtggtcgcgcgccgtcatcaggtcggccaccagccggtcggcgtccagcgcggggagcaggcgaaccgcttccgcgggcgtcaaagagtcattgtcgttcatcgttgtcctttcatgacgagaaaacccaccgctccggcgatgcgctggtgaagtcggtcagcgccgattcgaatcggatggctcgtcacgcccggagcaggtcgaacagctcgccccggagtccggcgtccgtgctgaactccccgcgatacgcggtggatgtcatctgcgcggcggccttgatgccgcgcatcgacatgcagaggtgctcgcccttcgctatgaccgcgacgtccggggtgccgctcagggtgctgatctcgagcgcgatctggtccacgaggcgctcctggacctgcagcttgtgcgcgtactgatgggcgaccctggcgaacttcgacaggccgagcagttgccccgccgggcggtacgcgatcgtcaccgagcagttgaacggcaggaggtggtgctcgcacagggaccagacctgaatgtccgagacgactacgagctggctcgtgccgatggactcgaagagagtgcccagcgaacccgggtcgtagttgatgaactctcgccaccagcgggcgaatcgcgcgggcgtgtcgcgcagtccgtcgcgattcgggtcctcgccgatctcctccagcagttggcgggcgatcccgaccagagggtcctcggtcgtcctcgacgccgatgagtcaccatcgaggtccaattctgtcaccgacatgcggtcacctttcgtccgggcttcggagtcactgacctcacggccggtactcggcccaagtcttgggagtttccgagacacgcacgcaggtcaactcgggaaactccatcgaccatgtcttgtagacccaggctgccatattctcagccgtcggattttcgtccatcacatcgttcagatgccgatgatcaagatggttgtccagccagaccttgaacgcggacaagtctccgtagtctcgaacgaagcccacgggcaacagatccgcgtcatcggccgcgagctccaactcgatcacatagttgtgcccgtgcattcgggcacactggtgctcaggagcaaggccggtaagccgatgactggccgagaattcaaatttcttcgtgattcgaaatgtcatcgatccgagttcccttctcctttgcgtgtgccggtgctcacacgccgcgtttttcgtcccacaacaaggtgtgaagtctggtcgtcaggttccagccgcgctccaccacggcgtcggccagctcgctcatgtgttttgtgacatcgtcggcggtacgcccgatcggcatgatccataccgaggacaggtcgaaggtggcgaccagctgggccacttcgtcgagatcgcgggtgttctggcagacgaacttgaacgtgcttcccggcagtgccgagaggctggtgagtgcctcagggacgatcctgcgctgcagggtgtcgcccgagtgcgacagcttcggcgagacgttgaacctgacgccggcggcgatgagttcggggtgcgcggcgtgcgtcccgttggtctcgatctcgatctctttaccgtgtccggtcagtgccgccaccagaggaatcagccgctcctgctggcccaggggctcaccgccggagatcacgatcagctcggtgtcgaatgcgagcaattccgtgaggacatctccgaccgggcggcggtgcagttccttgcgcggatcgtgggcgaccccgctctcactggctccggtccagtcccacgtgtacggcgtatcgcaccacgagcacgacaggttgcacccgccgagtcgcaggaacgcgcatctgcgtcccagggaccggccctcgccctggacggtcggaccgaagatttcgttgactacgagatcctgctccatggcgcaccgtttcctatgagtgggacgactcgacagccgtcggcttctgcgcctgcacgatgaaccaatgccggctcagccggacgccctgatcggtggtcgccgcggtgacatatcgatcgaatgcctcctcgtccgccgccgaatcgtagttgtcgatgattggcacctggtgcagaaaagtgtccagatcgcccggcgagtggtagaactcctcgaaccggaacgcctggtttcttgtcacgacgaagcccgcgtcggacagttcttgagcgatctcctccgacaatggcctgccgttccagctgtcgaagtcctggcctcggccgaacgtttctttcagctcgcgcgcgtcctgctccccgattcccatgtacaccacggcaccgccggggcggagcacccgcttgaactcttgggcgatcagcggaccgcggcgggaagtgaccacatcgacgtccccatcgggtagtccggtgtgggcggcatcgcacagctcgaacgtgacgtgcgccaggcccgcctccgctcggtaccgcgcggcgcattccagcatcgagggcgacagatccatcccggtcgcggcgccgtacgcggaagagacgcgcagcagactacgcccgtcggcacagccgacatccagcagtcgggcctgcggggcgcccagcgcggtcaccagctcatcgaagacggcatccgggcggccatcggggaatacgtcggtgctccgcgcatcgctgaagtagccgccgaaccgctcggccacccgggagtagaactccgcgtccatcgtcatcggccgtgcaccttcccctgagtcacggtggtcactacggtccttcggaccaccccttgagagccgtcccccgaaggggctgaaggggctccgaggtcggcaggacccaatctccggtgtcgccctatcggcccggcatcacggcgcatccacggcgggacggggccgggataggacggtgatagcgcgagcatgcggccgcgatgggcacctggttcacgctggttccagcaacgtccgaacggatcgcgaggcgaggagaactgaagatggccatcgagaagtcaccggctgccgcggtagccgtgacaccggccgacgaaaagcccctcgccaacacgccgtggacatgatggaccagcgtgccccctggtatcagccgaccgacgtcatcggcctgcccaggatggcaacgatgtcagcgcgtacggcggcggccccgccgtggtccaggatccgctcgcggaccctggccgactgcgagtagtactggacggcctgttccacgcggcgcaggtgggcgttgacccggccgaggtcgtgcagcacgtacccctctccgaccaggtcgccgctggctctgaccatcgcgaggacctcgagcagggtccgttccgccgcctcgtaccggtgctggtgcatcagcatttggcccaggcgccgcagtgtgagggcctggccccggtcgaagccggctgaccggcagatcccgagagcctcgtcgagatacgaacgggtccgcgcgaagtctgctcgccgcatcgtgatgtgtgccatctcgccgagcacatacgcccggccgatgacgtcctgcgcccgtgagaagttgcgctcggcgctctcgtagagggccagcgcccggtcgtcgtcaccatggtgccgctcgatgcgcgccaggtcacgcagacacagcgcttggccactcagctccccgatccgctcgaagatctccaaagcggcgctgaggtaagggcctgccgcctcatgctcgccccggtgcaggtgcagggtccccaacgagccgagaaccgccgcctggccccgctcgttaccggcctcctgtaccgcggtcagcgctatccgatgggtgcgtgcccagagttcgggatagccacgggcctcgaagagcgtcaccagggtcgtggccagctcccagcacaactcgtccagcccggaccgggccgccaactcgaccgcgttcaacaggttggtctgctcgctctccagccatccgagcgggtcgcgcaggcaccgccggacatgctcctccggcggatgccaccgttcgccccggcccatcacgatggtgtacgcaccgccgtagatctctttgtgcgccctctccgcgagcgccatccagccaccgaccatccgccgcaccgctccggcgcgttccgcctcggggatctccaggggcagccgttcgtgcgcgaacgtccgcaccatctgcgacagtccgcatcggaactccccgccctggtccatggcggtgatgtcgaggagccgcctgtcgatgagcggttccagcaggtcggcagggtgcggcgtgcggtcgtcgatgagggcggcacccagccagcccggaatctccgtcccctcggccaggctgatcagacataacaggcgacggtcggccgccgccagcccgtcgtagctgagggaaagactcgcccggatcgacagctcgccgtatgccagctcgtccaaccggcggtgctcgttctcaagccggtgccacatggaggtcagtgaccagtgcgggcgcgcggccagccgcgcgccgatgatgcgcagcgcgaggggcagcccgcccaccaggcgtatgagggcccgtgccgcctcccgctcgccctccacacgatgctctccgatgatccgccccaggagctggacggcctgctcctggcccagcggctccaagtcgaaccggcgggtgcccggcagcgccgtgagctggccgcggctggtgaccagcacaccgctgctgccggttccgggcagcagcggcatcacctgcgactcgctcaccgcgtcgtccagcaacaccaggatccggcgatcggccagcagactgcggtacatcttcgcgcgctcatccagcgcgtcgggtatggcctggcccggtacgcccagcgcccggaggaaccgccccagcgcctcggcggagcccaccggttgtccgcccggccctcgcaggtcgcagtagagctgtccgtccgggaagtacccggccgcgagccggtgcgccacatgcctggccaacgtgctcttgccgacaccaggtccgccgagcaccaggaccacgttcagttgccccttctcctgtcccccggtgacggcgtcgcagaccgccgcaacgatcacgtcatcggctaccaggtcgccgatgtcggcgggaagttgcctcggggtcacgaccggattgggatcggcaccacgctcttgcaccgcctcggggagcggcgtcgccacgagagaacgtggctcggccggttcggccagtgccaggtcggcgtcgtcggtcaggatcgcttgcgccaggtcgcgcagttcccttcccgggtccaggcccagttcttcggtgagcaacgcgcggccctggtgataggcgtccagcgcctccgcctgtcggcccgagcggaataacgcgaccatgaggtgcccccgcagccgctcgcgcagcggatgcccggcgacaagctgctgcagttcgccgaccaggtgctcgtgacggcccagctccagttccagccgcatgcaggtctccaccgcctccaggcggctctcgtccaactgcgccgccgtaccggccaggacctcgctgagtacaccggccaggcaccggccccgccacaggcctgtcgccgcgcggagcgcgtccaccgcggccaccttcaggccctcacgggccagggcatgcgcctcagtcagcaggcggtggaagttgccgacgtcggtcgcgtcctccggtgccctcaggatgtacccgtgggcgcgggtctcgatggtggcgtcgactcccccgtccgccagcgccttgcggagccgggagacgcagatctgcacctgggtgcgtgcggtcttcggcggatcgtgcgcccagatgacatccaccagataggtggtctccaccacccggttcagttcgagcaccagagccccgaggacgatctcctgccgccccggtgccacgtacacggcccccagctcaccgctcacctcaagcggcccgagcacccggaagggagccgccgtcacgccactgccgtcgtagtctgccgaagctgccatgcacaaccctgtttcctgtggtacccgccagacctggtccggctcgcgcaattgggcggcggaactcatgccgacctggctactcattcgcaaccgttggccaacgttagtgctgccacagtcagatacatgtgtatctgacacgaaaggttccataattggccacggatatcactagacttctcggcgggcgacgcgccgcgctttccggcccacttccatggcacatccggccaggccgttgatcagtacggtggacgggccccggttggcgatgcgcttcgacggccgccggtgcgaacgtgtgcgcgacagcggccccacgaggccggaaccggcgcccgtgatgacggccacgcggccggaatttcctggttcacgggtctctcgtctttcacgtacgcggggcttacatccgctcgggtctgcgctgcttccgaagcctaggaagatcggcacggcgcggacttgaaccacagcgacgttccgatagaacatcagcgccacaaacgggtgtcacagcagcggagcacttcgagcgaccgccggccgcagacgcgcctccagcgggttcagcccgatcgccctgacggggaatgcgcccagtcggagagcagggtgcggatcgcggcgacaagcgcgaggggctggtcgagcatgatgtgatgtctcgcgccggggacctcgatgaccgaggcgacccggccgagctggtcatacatggcttcgcggcgttccgccgagacgatgccacgctccgcgtgcaccagcgccacccggcagtccagtgggatcaacgggttttcgacacgctcccgcgcgaaaatggcggggtcccatttccaggtccaaccaccggccacctcgcggatggacgtggcggcgacatggtcggcgacgtaaccgagcaccggttggctgggcatgggactgaactgcccgatgatcgccgcacgcgaggaatagaggcgtgtcgccgccccgaaggtgcgctgttcccgcgtcgcgcgtttcgccgccaccgggccgatcatgggggaatcgatcaggacggccccctcgatgcggtgcccggacagcgcggccagccggagcgtcaccgcgcctcccaggctgtgcccgatcaccaccggcggcacgtcgagccccgcgtccgcgatgacggcgagcacttcacgcatccagatctcgaggccgtacgcgtcgcgcctgccgctgtcaccgtgtccggacaggtccagcgcgatgacccgccggccgctgaccaactgcgggccgacatggtcccaccagcgagaatgtgcggcggccccatgtacgagtacgatgccgcgcccggtcggctcgccccaggcccggtagacgatcggggtgcccgccaccgtgatctcatgttcggtgaccggcgtagccagcgcggcggtgtaccacgcgggcggcgcggtggaatccggatcagccgatgtggtcatgcgggaaagtcaaccagagggcggcggccgctcgagcgggatcctcacctcaagtccatttcgtcgcctatgatttcgaagatccctgcccatcccaccatcgggccactgagccagttggcaccaccgctagggtccttctgacggatctccgtggaggaccggagcggcgtcagggggcacctcccagcggtagctgggggagcgtgctctcggcgtgccgcgcggagaccctcgtagcggagctacttgggttttcgtgcggtgcggcgagaggacgtgccggacgccgccacgccgcggagatccgtcagaaggaccctaaggccagggacgccaggagagaggcggagcaccacatgtccgatgagttcgggatcgccgggaagacgctgctcgtcaccggatccggccgtaatctcggcaaggcgatcgtcctcgagttcgctgcccgcggcgcgaacgtcgtcatcaattcaaggtcgaacgcggacgaggccaacgaggtcaaggacgaggcggaggccctcggcgcgaaggccatcgtcgtgctcggcgacgcggccgagaagtcgacggtgctggagttgaaggcccgggcggagcaggcgttcgggcgtgtcgacatctatgtgagcaacgccgcccggcggctgcacaaggacttctgggacacgacggacgaggactggcaccatcacctcaaccagcagctgaccgcgtcgtggtacctcgccaaagcgttcgcgccggggatgcgcgagcacggctggggccggatcgtgcacatgaacgggccggacggctggatcggcggcccgacccggataccccactccaccgccaagggcggattgcggacccttacccgctccctgtcccgcagcctcgggccgttcgggatcacggtgaacgacatcaacccgggattcgccgacaccgtccgcgacctggagacccatccggggatgactcccgggcggtcggagcggttcgcccgggaaaccatcgcgatcaagcggcagccgcagcccgcggagatcgcctgggcgtgcgcgttcctcgcggccgagcggtccgcggccatcactggcgccgccattcacgtcgacggcggccagttcatgatcggctgattacccgctcatcggaagcgtttgcggaagtcggacagcgccacgtccgggtcctgtgccactgccgaaactattgatgcgtacgtgtccgccaaatcggccacgaattccggctcgtagacgccctcggcccagcgcagcgcgccgcccgggccgtcagggccgggcgtgatgaacagttccaggtcgaagtggctgctcacaccggcggggaagggcagcgccaccgtctgcagaccgtggaagtccgccccgtggagcccgtccgctcccggcggcgtggccagcatgttgaacgccacctcgccggcccgggacacggtccgcggcgtcgtgccggggtgggtcacgacattgaggagcgggtactcctgatgggccagcgcgtccagtgacgtggtccgggtcgccctcagcacgtcccggaacgcgggctcggggggcaggtccacccgcagataggtcagattggcgaactgtcccacggtgttccaggtctccgggcggatccggttggcgtggatcgagcagaccaccagagcgcgctccccggtcgcggcgtgcagcacgccgaacagcagggccagcaacaccgcgtacatcgtggtgcgctcggcggcggcaagctcccgcagcgcggccgcgtcggcgggggagaacgggagatccaccgtccggaacggtggccgccgagcgccgaacttctcggggtggcggcgcagcccggcccatttcagcaggggcgcctgccggtcccagtactcggcgtggcggcgacccggctcgccggccatccgggtgtcccgccaggcgtagtagtcgccgggcagccattcgatgtcggggagcggctcggtctccacaccggtttcggcgttgtagaaccgggccaggtcacggcggatcagggcgttcgaccacgcgtcggtgacccggtggttcacgttgatcagcagcagatgctcggtgtcggccagccggtacaggtcggccgtgagcggcaggccgtccgggtcgaacatctcggccatgcgcgcgacgatcagccgctcggcctcgtccgggtcggatacgacggtctcgcccagcggcaggtgaacgccgggtggcaggacgtgctcgcgcagcctccgcagaccggcccgctcgtaacggatccgcagcgatacgtgccggtccaccacccggtcgagcgcacggctgagcgcggcccggtccagtggtccgcggaaccggtagagaatgggcacgctcatcaggccaccggcgaggactttgtcgtccggcaccatcaaccggtgcttgtccgactcggtcatcggccggctccttccgcggtgccggaggtgcgttcgctgatcgccgcggccatcgcctcgatcgtcgtgatgtcggtgaagagctcagccggggtgatggccacgccgaggctgtcctcgaccaggctcagcagcgccatcccggccatcgagtccccgcccagggccatgaagtcgttgtccacgccgatgtcctcccgcttgagcacgagcgaccacagccccttgaccatggcctcgaccggcgtccgtggtgcggtccgcccgtccgattccgccatcggcccggccgcgtccaactcgtcggccagcaccttggccagcgagccgcgctggatcttgccggattcggcgaccgggatgtccgcgcggcgcaggaagaaccggggaaccttgaccggagcgagccgctgcccgaggtacgtccggaactcctcgtcacccagcgtcgctcccggcctgagcacgagcacggtaccgagttgttcgtcgcggaccgggtcggggatgccgaacgcgaccgcctcggccaccgcgggatggctcagcatcgcggcctccacctcgaacggcgacaccttctcgccacccacgttgatcatctccttggcccgcccggtgatcaggaacgagccgtccggcagcaaccggcccaggtcaccggtgcgcagccagccggccccttccgtgtcaccggcggaaccgccgcgcacgaacggggactccaggccgggcggccacacatacccgtgcatgacgttggcgccgcggatgacgatctcgccggacgcgtccggcgcggtgctgacccggccgtgctggtcgaggaccgcgacctcggagccggtggggacgagcccggcggcgtccggatcgcccggcagcggggtcgaggcgatctgatgcgcctcggacatcccgtacgactcgaccaccggcaggccgtaaccggcctcgagttcggcccgcaggcccgccgtcagggctccggagccgcagcggacgaagcgcagcgggccgggccactcgcggtcccgggggacgtaccgcaccatcagccggtgcatggtcggggtggccgagagccaggtcgcctcgctgtccagcgcgtgccgcagtgcggtggccgggtccggggccggcgcgcagacgatgctgccgcccgggagcagcgaggcgatcagcgaaccgaccaggccctggacgtggaagaacggcatgatgttgaaccggcggtcggagtcggtcaggtggtaggcgtccaccgtggcctgggcggccgcgaccacgccggcggcgctgagggacacgaccttgccgatggacgtcgtacccgaggtccgcagcagcaccgcctcgtccggatacggcccggtcgccgccgtctcggccgggcggccgccgccccgcggctccaccaccacgtcctcgccgctgaaccggagcgtcaccgcggctgccttggccgccgccacctgctgcgcggcggcggttccggctccgtcggcgagcacgatcagcgcgacgtcgagcgtgtccaggtagtcctgggtctcggcggccgaccagagcggattgaccgggcagcaggccgcggcggcgaccaccccgagcagcagcgcggcgctgtcggcgtcctctccggggaccaccgcgacccggtgcccggggcgcaccccgagcccggtcagcgccgtgcggacggcgtgcatgcggccggacagatgccggtagctcagcccggccggttcgcccggcacgatgatcgcgggcgcgtcaccccgggtctgcgcatgacgctcaatgaccgcgagcaccgggctgacggcatcgaggttcgacatggaacaacctttccttggacttggatcgtgggtgagggcctgtgtcatatcgccgcacgcccggccggggatatgacacaggctctgagtgcctcggtcatgccgtcgccttcgggtggcaccgcaggccggttatccgcgcgaccggggcgccgtcggccgacaccaggtccaggtcgctcaggtccccgcgaccgcgcacccggcaccagacctcccccggcacgtcgtcgaagagttcgagccgctcggcagaggccacgctccccccggcggcggccagccggaggcacccgtcgagcacctcggcggcggagtcgcccgtgacctgggtcaggccgccgtcggcgcggtccgggaggtcatcggccggttccggctcggcgatctccgcacccaggtacgccgtccacccggcaccggacgcgccggacgcacggacgtacccgtcgatccgcatcggcgccgcgccggtacgcaccgcggtctgcacggcggccggtcccaccgactcggtgtccggctccgggaagaccaggtcgcgcagggcggccggcgcaccgacagcgcgagcggcggccaccgcccagtcggcgcgcgcaccggcggacagcaccggtgtgatgtccgtccggccgaaccaccgctgggcggggtcggccagttcaacggccgtcccgagcaacgggtgcgcaccggccgcggccggtgcggacggccagaagcgacggcgctggaacgggtaggtgggcagtccggcccggctccggccggagccggccagcagcgcggtccagtccaccgacgcgccgcccacgtaggcctcggccaggaagctcagcacgcgcgaggcgtcgccctggtcgcgcagcagcacgctggtcgcccagccccggtcgtcgacatcggccagggcgccgctgaccgccgggctgagcacaccgtccgggccgacctcgaggaagccggtgacgccccggtcgcgcagcagccgtacgccgtcctccagtccgtccggcgcctcggccgggcggctccagtagccggggctcgcgatctccgagggatcgagaagcttgccggtcaccgcggaaaccatcgggacggaaggtgcggagaaggtgatctggtccaccgtttcccgcagcccggtcccgtcggcccgcgcgaccaccagccggccggcgtcggccagggagaacacaccggccaggtacgccgctgccacctcgcccaccgagtcgcccagcaggaagtcggggcgcggtccgtaggaccgcagcagcgccgccagtgccacctcgaccgcgaagaccccggcgtgctccgcccgtcgccgcgtgtccccctcggaaccgatcagcggctccagggcggcctgtgcctccgtcagcgcgtccgcgaagaccgctgactgctcggcgagccggagagccatgccgggccaggaggactcggcgccggggaagaggaacgcgacgcgcccgggccgggtcaccgccgtacgggcgtactcgaccggcagccgctcggcgacgccgcgcagcccggcgacgaggtcttcccggtcggccgcccacaccaccgcgcggcagcggaagccggcccggttcgcggccagcgatgcggccacgtccgccatcggcacgtcctggttccgcccggcccagtcggcgacgtcggccgccagggcccgcagcgcaccgtcggtacgcgcggacaccgcccatggcaccaggtcgcccagggccgagcgggccggggtgtcgccgcggggcggcaccggcacggcgggggcctgttccaggatcacgtgcgcgttggtgccgctgatcccgaacgtcgatacgcccgcgcgccgcggacgtccgtcggactcccacggccgggccccggtcagcagccgtacgtcaccggccgaccagtcgacctgccgtgacggctcgtccacgtgcagcgtcgccggcagcgtcccgtgccgcatcgcctcgaccatcttgatcaccccggccacgcccgacgccgcctgcgtgtggccgatgttggacttcaccgaccccagccacagcggccggtcctcggggcggtcctggccataggtggcgagcatcgcctgggcctcgatcgtgtcgcccagatgggtccccgtaccgtgcccctccgccgcgtcgacatcggagcccgacaggcgggcgtcggccagggcccggcggatcacccgctcctgcgcggggccgttcggcgccgaaagaccgttgctcgccccgtcctggttgaccgcgctgccgcgcagcaccgccagcaccggatgcccgagccgttgcgcctccgacagccgctccagcagcaccacgcccacgccctcggacattcccatgccgtcagcggcctcggagaaggacttggaccgtcctaccgccgagatggcgcgttgcatcgcgagttccgcgagcggtcccggcgtggacatcaccgtcactcccccggccacggccagcgagcactccccgctgcgcagcgcctgggccgcgaggtgcatcgccaccaacgacgacgaacaggcggtgtcgatcgtcaccgcgggaccttcgaagccgaacgtgtacgccacccgaccggacgcgacgctcgacgcgttgcccgtcatcagatagccctcggcccccggctgcgcggcgagcgcggccgcgtagtcctggccggccaggcccgcgtagacggcggtgtcggtgcccttcatcgcggtcgggtccagaccggcccgttccagcgcctcccagacgacctcgagcagcagccgctgctgcggatccattgccagcgcctcacgcggggacaccccgaacagcaccgggtcgaagtccgccaccccgtccaggaagccgccctcccgcgtgtatacgcggcccggtgtgcccggcgtcggatcgtagatggccggcaggtcccagccgcggtcctccgggaaggctccgaccgcgtcctcgccgtccatgacgagccgccacagatcctcgggcgaggctactccgccggggtagcggcaggccatcgacacgatcgcgatcggttcgcccaccgccgccggggccggggcggccttctcggcggccgcggccgcgccggtcacctgggccaccagatactcggccaggtcctgcggcgtcgggtagtcgaagaccagcgtggccggcagcgtcagcccggacaccgcggccagccggttgcggaactccaccgccgtcagcgagtcgaagccgaggttgcggaacgcctgatcggcttcgatgccgctgtcatcactgtggccgaggaccaccgcggcgtgctcgcgcaccaggtccagcaccacccgccaatgctcgccgggcggcaactgagccagccggcgggccagcgcaccgccgccggcaccggcgacagccgcccgccggggcgcacggcccagtccctgcaacgccgtcggcaccggcgtccccgccgcgatcgcctgacggatcgcaccgacgtcgagacccgccgccagcaccggccggcccaggcccagcgcccggtccagcagggccagcgcctgctccgtactcagcaccacaccaccgctgcgggcctggcgggcccgatccgcctcggtgagcgcggcggtcatgccgctcgtctgctcccacatgccccaggcgatggagacggcgggcagcccttcggccatccggtgcgcggcgagggcgtccaggtaggcgttggccgccgcgtagttgccctgcccggggttgcccagcagccccgacagcgaggagaacaacacgaacgtccgcagatcccggtcccgcgtcgcctcgtgcaggtaccgagcggcatccgccttggcggccagcacccggcccagcgactcaccggtcaacgaggtcaccggggtgtcctccagcacaccggtcgcgtgcaccactccagccagttccgggatgtccgccaccagcgcgtccaccgcgacccggtcggacacatccgccgcgacgatccgcacccgagccccgagcgccgccaactctgtggtcaactcggcggcgccgggcgcatccggtccggagcggctggccaggaccaggtcccggacgccgtgccggaccaccagatgccgagcgaccaggccacccaacgttccggtcccgcccgtgatcagcaccgtgccgtcgcccaccccgggcatccgcagcacgatcttgcccacgtgccgggcctggctcatgtaccgcagcgccgcaccggcccgagcggccgggaacaccgtgaccggcagcggaacggcctgctccaggacccgccgccagatcaccgcgagccggtccgggtcgaggtccgccagatcgaacggcacctgccgcggatcacggatatcgatcttgccgagctccacgaaccggccgccctcggccaacatccccagcgacgcgtccagcaactcgccggtcagcgagttcaacaccacgtcgacctggccgaactcagccgcgaagtccgccgtacgcgaagaagcgatgtgcgtcacgcccagctcggccaccgccgcctgcttgggcgggctcgccgtcgcgaacacccgcgccccggccgccaacgccagccggaccgccgccatgcccacgccgccggaggccgcgtggatcagcacccgatcgcccgaacgcacccggcccagatcgaacagcgcgtggtaggcggtcaggtacgcggtcgccacccccgccgcctcgacatcgctccacccgtccgggaccggcgcgaccagccgcgcgtcgatcaccgccagcgggccgaacgccgcgaacatccccatcacccgcgtgccgaccggcaggccccgcacttggagccccgtctcgaccaccacaccggtgccctcgccgcccggaggacgggggtcgccgggaagcatgcccagcgatgagaccacgtcgcggaagttcactccggccacgcggaccgcgatgcggatctcatgcggctccagtggccgttcggcgtccggggccggcaccgcacgcacctcgtccaggtcgccgtccggaccccattggagccgccatgccccctcgcccatcggcagctccggcccgttcgggaccggacgcagccggggtaccagcaccgccccgtcgcgcagggccatttctcgttcgccggtggccagggacgcggcgacggccgcgtcggacccggggcggccatcgtcgtcgagcagcaccaggcccgcgtcggggtgctcggactgtgccgaccggaccaggccccagacgccggcctcggtcagaccggccacggtgtcgcccggcaccgccgccaccgcgtcacgggtacggaccgccagccgcgaccccgccggatagtccgcccccagccacgcctgtaccagagccagcgccgcgggcacgtcggccgggtcggcccacgcgagcaacggctcgcccaccgcgtcgggcagttccggtcccacccggacccaggcctgctccggtgcgggcccgggagagatcggcgcccattccagtgacagcagcccggcgggggcggcgcccagacggctggtgtcggcggggcgggagatgatcgcgcccgccgatatcaccggctgcccctcgccgtcggcggccagcagggagaacgcgtccggcccggccgggctcagccgcacccgcaacgccgacgcaccggtcgcgtgcaaccgcacgtcccgccacccgaacggcagcagaacctggtcgctgtccccgcccgcgtcgcggatcgcggcgacgaggagggcgtgcagcgcggcgtccagcagcgccggatg

SEQ ID NO: 23 - DNA, 47 bp, Artificial Sequence
  Synthetic Sequence - Rap_M7ER_GG-SP_Left_Fw
cgatgagctggtgatcgaaaccccgctgctgctgccgtcgtccgcta

SEQ ID NO: 24 - DNA, 41 bp, Artificial Sequence
  Synthetic Sequence - Rap_M7ER_GG-SP_Left_Rv
ccatgccgacaggactagcggcggcgtggatcagcacggac

SEQ ID NO: 25 - DNA, 39 bp, Artificial Sequence
  Synthetic Sequence - Rap_M7ER_GG-SP_Right_Fw
gccgccgctagtcctgtcggcatggcagccacccagatc

SEQ ID NO: 26 - DNA, 45 bp, Artificial Sequence
  Synthetic Sequence - Rap_M7ER_GG-SP_Right_Rv
aaccaccggtgaccagaaccgtgccgttggcgtcgagttgctgag

SEQ ID NO: 27 - DNA, 2153 bp, Artificial Sequence
  Synthetic Sequence - polynucleotide for modifying rapamycin (adding double bond)
cgatgagctggtgatcgaaaccccgctgctgctgccgtcgtccgctagcgtgcacctgtccgtgtcggtcggcgaggctgacgaatccgggcgccggggtgtgacggtcttttcccgtgcggatggcgccgacgcctggactcgccacgtttcggccacgatcggcgtctctggcgctgccctctcgctgccagagcttgctgcttggcctcccgcacaggcacagccggtgggcctgggcgatttctacgaccggctgaccggggccggttacgagtacggtcccgcgttccaggggctgcaggccgcgtggcgtgacggggacaccgtcttcgccgaggtggccctggccgaggagcaggcggaggaggcggcacggttcgcggtgcatccggcgctgttggacgccgccttgcacgccggaattctgaacacactcgacaccgccgagcagggtgtgcggctgccgttctcctggaacggtgtccaggtccgggccactggcacggccacgctacgcgttgcgataacaccagtgacggacggctggagtgtgcgggtcgccgacgacagcggccgaccggtggctaccgtcgactcgctcgtaacgcggccggtaacggccgacacgctcggttccgctgccgacgacctgctcacggtggtctggacggagatccccaccccccagcagaccggcctgagcgtcggccggttcgaagacctggcggacggtgatgtgccggtgcccgaggtggtggtctgcaccgcactccccgacagcagcgagaacccgctagccccgctggatccgccggatccgctggtacagacccgcacgttgaccacccaggttctccaggcagttcaggcatggctggccggggaacgtttcaccgacagcacgctggtcgtgcggaccggcaccgggctggccaccgccggggtgtcgggtctgatgcggtctgcccagtcggaacaccccggccggttcgtcctggtggaatgcgacgacaacctcaccctccagcaactggccgcgactgtcgggttggacgagccgcggctgcgggtctgcgacggccggttcgaggtaccgcggctggcgcgggcgaatacgccggaaagcagcccgctcacgattcccggggatcgtgcgtggctgctggagcagtcccgcagcggaaccttgcgggacctcgcgctggtacccgccgaaaccgccgaacggcccctgcaatccggtgaagtacgagtagacgtacgcgccgcaggcctgaacttccgcgatgttctcatcgcgctcggcacttaccccggtgaggctgtgatcggggctgaggctgcgggcgtggtgctcgaggtcggtccggaggtccaggatctggccccgggagaccgagtgttcggtcttgtgggcggtgggttcggggcggtcgcgatcgctgatcgccgaatgctgggtgtgattcctgacgggtggtcgttcactacggcggcgtccgtgccggttgtgttcgccaccgcgtattacgggctggtggatctggccgggctgagtgcgggtgagtccgtgctgatccacgccgccgctagtcctgtcggcatggcagccacccagatcgcccgccacctcggcgcgcggatctacgcgacggccagcaccggtaagcagcacgtcctgcgcgaggcgggtctggaggatgcccggatcggggactcgcgtaccactggcttccgggaaatggttctggacaccactgacagccggggtgtcgatgtcgtcctgaactccctcagcggtgactttgtcgatgcttcgcttgatctgctgcctcgtggtggccggttcgtcgagatgggcaagaccgacatccgtgacccgcaccaggtcaccgccgaccggccgggtaccagctaccaggcgttcgatctgatggacgccggtccggaccggctgcgggagatcatcgccgatttgctcgccctgttcgcgcagggtgtgctattgcccctgccggtgcgggcctgggacatccgtcaggcccgtgaggcgttcagctggatgagccgtgcccgccacatcggcaagatcgtcctcaccgtccctcagcaactcgacgccaacggcacggttctggtcaccggtggtt

SEQ ID NO: 28 - DNA, 24 bp, Artificial Sequence
  Synthetic Sequence - rapF1
aacagccgaaagaaatggctgtgc

SEQ ID NO: 29 - DNA, 24 bp, Artificial Sequence
  Synthetic Sequence - rapR1
ggccctctcgaacttccgtacctc

SEQ ID NO: 30 - DNA, 24 bp, Artificial Sequence
  Synthetic Sequence - rapF2
ggtggtttcgtcatgcctgttctg

SEQ ID NO: 31 - DNA, 24 bp, Artificial Sequence
  Synthetic Sequence - rapR2
gctctccttgagcatcagccactg

SEQ ID NO: 32 - DNA, 55 bp, Artificial Sequence
  Synthetic Sequence - rap_M9_KS_3′_sgRNA
ttctaatacgactcactatagaaccagtcctggcccgaagcgttttagagctaga

SEQ ID NO: 33 - DNA, 55 bp, Artificial Sequence
  Synthetic Sequence - rap_M9_DH_5′_sgRNA_2
ttctaatacgactcactataggaccggcggtgtgcaggtgtgttttagagctaga

SEQ ID NO: 34 - DNA, 131 bp, Artificial Sequence
  Synthetic Sequence - Rap_M9mAT->M6mmAT_Left_Fw
gctggtgacggagaaccagtcctggcccgaagccggtcggccgcgccgggcaggcgtgtcgtccttcggagtcagtggcactaatgcccacgtcatcctggagagcgcaccccccgctcagcccgcggagg

SEQ ID NO: 35 - DNA, 57 bp, Artificial Sequence
  Synthetic Sequence - Rap_M9mAT->M6mmAT_Left_Rv
caccaccgcacccagcaacggatgcccacccgcagccgagcgatccacaccctcgac

SEQ ID NO: 36 - DNA, 36 bp, Artificial Sequence
  Synthetic Sequence - Rap_M9mAT->M6mmAT_Right_Fw
gggcatccgttgctgggtgcggtggtggcgttgccg

SEQ ID NO: 37 - DNA, 25 bp, Artificial Sequence
  Synthetic Sequence - Rap_M9mAT->M6mmAT_Right_Rv
gtgtccggactcgtcagcctcacca

SEQ ID NO: 38 - DNA, 1684 bp, Artificial Sequence
  Synthetic Sequence - polynucleotide for modifying rapamycin (Adding methyl group as a side chain)
gctggtgacggagaaccagtcctggcccgaagccggtcggccgcgccgggcaggcgtgtcgtccttcggagtcagtggcactaatgcccacgtcatcctggagagcgcaccccccgctcagcccgcggaggaggcgcagcctgttgagacgccggtggtggcctcggatgtgctgccgctggtgatatcggccaagacccagcccgccctgaccgaacacgaagaccggctgcgcgcctacctggcggcgtcgcccggagtggatacacgggctgttgcatcaacgctcgcggtgacacggtcggtgttcgagcaccgcgccgtactccttggagacgacaccgtcaccggcaccgctgtgtccgatccccgggtggtgtttgttttcccggggcaggggtggcagtggctggggatgggcagtgcgctgcgcgattcctcgatcgtgttcgccgagcggatggccgagtgcgcggccgcgttgcgcgagttcgtggactgggacctgttcacggttctggatgatccggcggtggtggaccgggttgatgtggtccagcccgcttcctgggcgatgatggtctccctggccgcggtgtggcaggcggccggtgtgcggccggatgcggtgatcggccattcacagggtgagatcgccgcggcgtgtgtggcgggtgcggtgtcgatgcgggatgccgcccggatcgtgaccttgcgcagccaggcgatcgcccggggcctggcgggccggggcgcgatggcatccgtcgccctgcccgcacaggatgtcgagctggtcgacggggcctggatcgccgcccacaacggtcccgcctccaccgtgatcgcgggcaccccggaagcggtcgaccatgtcctcaccgctcatgaagcgcgaggggtgcgggtgcggcggatcaccgtcgactacgcctcgcacaccccgcacgtcgagctgatccgcgacgaactgctcgacatcactagcgacagcagctcgcaggccccggtcgtgccgtggctgtcgaccgtggacggctcctgggtcgacagcccgctcgatgtggagtactggtaccggaacctccgtgagccggtcggtttccaccccgccgtcggccagttgcaggcccagggcgacaccgtgttcgtcgaggtcagcgccagcccggtgctgttgcaggcgatggacgacgatgtcgtcacggttgccacgctgcgtcgtgacgacggcgacgccacccggatgctcaccgccctggcacaggcctatgtccacggcgtcaccgtcgactggcccgccatcctcggcaccaccacaacccgggtactggaccttccgacctacgccttccaacaccagcggtactgggtcgagggtgtggatcgctcggctgcgggtgggcatccgttgctgggtgcggtggtggcgttgccgggttcggatggtgtgctgttgaccgggcgggtgtcgttggccacgcatgcgtggctggctgatcacgcggtgcggggcagtgtgctgctgcccggtaccgggtttgtggagctggttgtccgcgcggctgatgaggtgggctgcgacgtcgttgacgagctgatcgtcgaagccccgcttctgctgccgcagaccggcggtgtgcaggtgtcggtatcggttggtgaggctgacgagtccggacac

SEQ ID NO: 39 - DNA, 55 bp, Artificial Sequence
  Synthetic Sequence - rap_M5_KR_3′_sgRNA
ttctaatacgactcactatagagcggctggagaccgtattcgttttagagctaga

SEQ ID NO: 40 - DNA, 55 bp, Artificial Sequence
  Synthetic Sequence - rap_M6_KR-ACP_sgRNA
ttctaatacgactcactatagcagcaacgccggaacctccggttttagagctaga

SEQ ID NO: 41 - DNA, 44 bp, Artificial Sequence
  Synthetic Sequence - Rap_M5KR_YF_Left_Fw
tgtcgttgagtccctgagcgcgcagcggctggagaccgtattcc

SEQ ID NO: 42 - DNA, 100 bp, Artificial Sequence
  Synthetic Sequence - Rap_M5KR-ACP-M6KR-ACP_Rv
accgggcgacgcaacgaacgcagcaacgccggaacctccgcgtcccgtaccggctccatcggcgcggccaccagaaccggttcactgtggcgtgacgcgt

SEQ ID NO: 43 - DNA, 459 bp, Artificial Sequence
  Synthetic Sequence - polynucleotide for modifying rapamycin (shrinking a macrolide ring)
tgtcgttgagtccctgagcgcgcagcggctggagaccgtattccggcccaaggccgatggtgcttggcatttgcacgagctcacccgggacgccgacctggcggcgttcgtcatgtattcctcggctgccggtgtcatgggcggtgcgggtcagggtaactacgcggcggcaaacgcgttcctggacgcgctcgccgaagaacgccgagccgagggcctgcccgcactcgcggtggcctggggcctctgggaggacgccagcggcctgaccgcgcaactgaccgacacggaccgtgaccggatccggcgcggtggcctgcgggccatctccgccgagcacgggatgcggctgttcgacaacgcgtcacgccacagtgaaccggttctggtggccgcgccgatggagccggtacgggacgcggaggttccggcgttgctgcgttcgttgcgtcgcccggt

SEQ ID NO: 44 - DNA, 55 bp, Artificial Sequence
  Synthetic Sequence - rap_M2_KS_3′_sgRNA
ttctaatacgactcactataggcactccccacacagcctgcgttttagagctaga

SEQ ID NO: 45 - DNA, 55 bp, Artificial Sequence
  Synthetic Sequence - rap_M3_DH_3′_sgRNA
ttctaatacgactcactatagcgtggccaccagcccaggccgttttagagctaga

SEQ ID NO: 46 - DNA, 57 bp, Artificial Sequence
  Synthetic Sequence - Rap4309_fra1-2_pRed_Fw
ggagtgcgctttccaggatgacgtgggcgttctagatgccaggaagatacttaacag

SEQ ID NO: 47 - DNA, 57 bp, Artificial Sequence
  Synthetic Sequence - Rap4309_fra1-2_pRed_Rv
ctgttcgcaatgcaggtggctctgttcgggctctagaccattcatccgcttattatc

SEQ ID NO: 48 - DNA, 65 bp, Artificial Sequence
  Synthetic Sequence - Rap4309_fra3-5_pRed_Fw
cccacgattccagcagcccgaacagagccacctgcatttctagatgccaggaagatacttaacag

SEQ ID NO: 49 - DNA, 57 bp, Artificial Sequence
  Synthetic Sequence - Rap4309_fra3-5_pRed_Rv
gtgagcgtggccgacttctacgaccggctggtctagaccattcatccgcttattatc

SEQ ID NO: 50 - DNA, 42 bp, Artificial Sequence
  Synthetic Sequence - Rap4309_fra1_pRed_Fw
ggatgacgtgggcgttctagatgccaggaagatacttaacag

SEQ ID NO: 51 - DNA, 41 bp, Artificial Sequence
  Synthetic Sequence - Rap4309_fra5_pRed_Rv
tctacgaccggctggtctagaccattcatccgcttattatc

SEQ ID NO: 52 - DNA, 53 bp, Artificial Sequence
  Synthetic Sequence - 4309_fra1_M2_Fw
acgcccacgtcatcctggaaagcgcactccccacacagcctgcgggcaacaca

SEQ ID NO: 53 - DNA, 30 bp, Artificial Sequence
  Synthetic Sequence - 4309_fra1_M2-M11_Rv
ccaccggcggcagcggcccgccgagcaatc

SEQ ID NO: 54 - DNA, 30 bp, Artificial Sequence
  Synthetic Sequence - 4309_fra2_M11_Fw
attgctcggcgggccgctgccgccggtgga

SEQ ID NO: 55 - DNA, 23 bp, Artificial Sequence
  Synthetic Sequence - 4309_fra2_M11_Rv
gcccgaacagagccacctgcatt

SEQ ID NO: 56 - DNA, 46 bp, Artificial Sequence
  Synthetic Sequence - 4309_fra3_M12_Fw
aatgcaggtggctctgttcgggctgctggaatcgtggggtgtacga

SEQ ID NO: 57 - DNA, 22 bp, Artificial Sequence
  Synthetic Sequence - 4309_fra3_M12_Rv
tgcggcgaccagaatcgggttg

SEQ ID NO: 58 - DNA, 22 bp, Artificial Sequence
  Synthetic Sequence - 4309_fra4_M13_Fw
caacccgattctggtcgccgca

SEQ ID NO: 59 - DNA, 41 bp, Artificial Sequence
  Synthetic Sequence - 4309_fra4_M12-M13_Rv
tggaaggcgtaggtcggaaggtccagtacccgggttgtggt

SEQ ID NO: 60 - DNA, 61 bp, Artificial Sequence
  Synthetic Sequence - 4309_fra5_M3_Fw
ccacaacccgggtactggaccttccgacctacgccttccagcaccagcggtactggctcag

SEQ ID NO: 61 - DNA, 28 bp, Artificial Sequence
  Synthetic Sequence - 4309_fra5_M3_Rv
ccagccggtcgtagaagtcggccacgct

SEQ ID NO: 62 - DNA, 11301 bp, Artificial Sequence
  Synthetic Sequence - polynucleotide for modifying rapamycin (expanding a macrolide ring)_full length
acgcccacgtcatcctggaaagcgcactccccacacagcctgcgggcaacacagtggtcgagtcggcaccggagtgggtgccgttggtgatttcggcgaggacccagtcggcactggctgaatacgagggccggttgcgtgcgtatctggcggcgtcgcccggggcggatacgcgggctgtggcatcgacgctggcgatgacacggtcggtgttcgagtaccgggccgtactcattggagatgacaccgtcaccggtaccgcggcgaccgatccgcgggtggtgttcgtcttcccgggtcaggggtcgcagcgtgctggtatgggtgaggaactggccgccgcgttccccgtcttcgcgcggatccatcagcaggtgtgggatctgctggatgtgcccgatctcgatgtgaatgagaccgggtatgcccagccggccctgttcgctttgcaggtggctctgttcgggttgctggaatcgtggggtgtacggccggatgcggtggtcggtcactctgtcggtgagctcgccgccggatacgtctccgggttgtggtcgttggaggatgcctgcactttggtgtcggcgcgggctcgtctgatgcaggctctgcctgcgggtggggtgatggtcgctgtcccggtctcggaggatgaggctcgggccgtgctgggtgagggtgtggagatcgccgcggtcaacgggccgtcgtcggtggttctctccggtgatgaggccgccgtgctgcaggccgcggaggggctggggaagtggacgcggctggcgaccagtcacgcgttccattccgcccgtatggaaccgatgctggaggagttccgggcggtcgctgaaggcctgacctaccggacgccgcaggtcgccatggccgctggtgatcaggtgatgaccgctgagtactgggtgcggcaggtccgggacacggtccggttcggcgagcaggtggcctcgttcgaggatgcggtgttcgtcgagctgggtgccgaccggtcactggcccgcctggtcgatggcatcgcgatgctgcacggtgaccatgaggcgcaggccgctgtcggtgccctggctcacctgtacgtgaacggcgtgagtgtcgagtggtccgcggtgctgggtgatgtcccggtaacccgggtgctggatcttccgacgtacgccttccagcaccagcggtactggcttgagggcacggaccgggcgactgcgggtggtcatccgttgctgggttcggtggtgcggctggccgaggccagtggggtgttgttcactgcccgggtttcccggagcggtgatctgtggctgcgggaccagacggttctgcccgcgacggtgttcgtggagatggcgctggcagcggcggacgaggtcggctgcggtctggttgaggatctgagtgtggaagcgttgctgctgcttcccgatgatggcgccgtcgaggtacagacctgggtgggcgaaccggatgagggcggtcggcgccggctcagtgtccacgcccgttacggtgacggcgagccctggacctgcttggccaccgcaaccctggccaccactacgggtgtggccgctgccgcggtcggctggcaggccggtggggtgtggccgccggccggtgcggtcccggtcgggacatcggcaccctcactgcgggcggtgtggcgcctgggcagcgacatcttcgccgaggtggccctggacgatgcccatgatgccaccaggtttgtgcttcatcccggcctgatggccgccgcgctcaccaccgtaggcgaggagactcccgccgtgtggcagggcctgaccctgcacgccggcaatcccggcgagctgcgcgtccgcctcacctcacacgatgacggcaccctgtcggcagaggccaccgacagcacaggcctccccgtcctgaccgcccgctcgctcaccctgcgcaccgtccccgtatacgaaccggccaccagcaccgacgacctgctcaccctgacctgggcaggaatccccaccccccagcagaccggcctgacggtgggtgcgtttgaagacctggcggcggacggcgatgtgccggtacccgaggtggcggtcttcaccgcactccccgacagcgacgatccgctggagcaaacacgaaagctgaccgctcaggtcctccacacactccaggagtggcttggcggggagcgcttcagcgacagcacgctggtggtgcggaccggcaccgggttggccgctgctggggtgtcggggttgatgcgctcggcccagtccgaacaccccggccggttcgtcctggtcgaaagcgacgacgccctcacccaggatcagctggcggcggcggtcggactggatgagccgcggctgcgggtcagcgacggccggtacgaagtaccacggctgacccgcacacatgccgaagagcctgagcctgaaaggacgtgggatccggatggcacggtcctgatcacgggcggttcaggtgtgctggcggggatcgccgcccggcacctggtgaccgaacgcggcgtgcgtcatctcctgctgctgtcccgcagcgccccggatgaggcgctgatcggcgagcttggtgaactgggggcccgggtcgagacagcggcctgtgacgtgtccgatcctgccgcgctgacgcaggtgctggcgggtgtctcgccggagcatcccctgacggccgtgattcacaccgcgggcgtggtggatgacggtgttgtggagtctttgaccgtgcagcggctggagacggtactgcggcccaaggccgacggtgcgtggaacctgcacgagctcacccgggatgccgacctggccgcgttcgtcatgtattcctccgccgccggtgtgctcggtagtgcggggcagggcaactacgcggcggccaatgcgttcctggacgcgctggctgagcagcgtcacgctgagggtctgcccgcactcgcggtggcctggggtctgtgggaggacgccagtggcctgaccgcgcaactgaccgacacggaccgtgaccggatccggcgcggtggcctgcgggccatctccgccgagcacgggatggggctgttcgacagcgcgtcacgccacagtgaaccggttctggtggccgcgccgatggagccggtacgggacgcggaagtcccggcattgctgcggtcgttgcaccgcccgattgctcggcgggccgctgccgccggtggagcgcggtggctggccgccctggcaccggccgagcgggagaaggcactgctgaagctggtgtctgacggcgccgcgacggttctgggacacgccgacaccagcacgattccggcaaccacggcgttcaaggatctgggcatcaattcgctgaccgcggtggaactgcgcaacagcctggcgaaggccacggagctgcggctgcccgccacgctggtgttcgactaccccaccccggccgccttggctgcccggttggacgagttgttcaccggcgagaaccccgtaccggtacgcgggccggtgtcggcggtggcgcaggacgagccgctggcgatcgtgggaatggcctgccgcctacccggtggagtctcgtcgcctgaggatctgtggcgtctcctggagtcgggtacagatgcggtctccggtttccccaccgaccgtggctgggacgtcgagaacctgtacgacatggctggaaaatcgcaccgtgctgagggtggcttcctggatgccgcggctggctttgatgccggattcttcgggatcagtccgcgtgaggcgttggcgatggatccgcagcagcggctggtgctggaggtgtcctgggaggcgttcgagcgggccgggatcgagcccggttccgtacgcggcagcgataccggcgttttcatgggtgcgtaccccggtggctacggcatcggtgccgacctcggcggcttcggggccaccgccagttcggtcagtgtcctgtccggccgggtgtcgtacttcttcggcctcgagggtcccgcgttcacagtcgacacggcctgctcgtcatcgttggtggcgttgcatcaggcggggtatgccctccggcagggagagtgttcgctggccctggtcggcggtgtcactgtgatggccacgccacagactttcgtggagttctcccgccagggcggcctggcctccgacggccgctgcaaagcgttcgccgacgccgcggacggcacgggatgggctgaaggtgtcggtgtcctgctcgtagagcgactctccgatgcccgccgtaacggtcaccaggtgttggcggtggtgcgtggatcagcggtgaaccaggacggtgcgtcgaacggtctgaccgcgccgaatggtccttcgcagcagcgggtgatccgggccgctctcagcaacgcgggtctgagcacggctgaggtggatgtggtcgaggcgcacggcacgggcacaacgctgggtgacccgatcgaggcccaggcgctgatcgctacctatggccaggaccgtgaccagcctgtgctgctgggttcggtgaagtcgaacctgggtcatacgcaggccgctgcgggtgtgtccggtgtcatcaagatggtgatggccctgcaacacggtctggtgccgcgcacgttgcatgtcgatgagccgtcacggcatgtggactggtcggcgggcgcggtgcagctcgtgacggagaaccagccgtggccggatatgggccgagcgcgccgggcaggcgtgtcgtccttcgggatcagtggcaccaacgcccacgtcatcctggaaagcgcaccccccactcagcctgcggacaacgcggtgatcgagcgggcaccggagtgggtgccgttggtgatttcggccaggacccagtcggctttgactgagcacgagggccggttgcgtgcgtatctggcggcgtcgcccggggtggatatgcgggctgtggcatcgacgctggcgatgacacggtcggtgttcgagcaccgtgccgtgctgctgggagatgacaccgtcaccggcaccgctgtgtctgaccctcgggcggtgttcgtcttcccgggacaggggtcgcagcgtgctggcatgggtgaggaactggccgccgcgttccccgtcttcgcgcggatccatcagcaggtgtgggacctgctcgatgtgcccgatctggaggtgaacgagaccggttacgcccagccggccctgttcgcaatgcaggtggctctgttcgggctgctggaatcgtggggtgtacgaccggacgcggtgatcggccattcggtgggtgagcttgcggctgcgtatgtgtccggggtgtggtcgttggaggatgcctgcactttggtgtcggcgcgggctcgtctgatgcaggctctgcccgcgggtggggtgatggtcgctgtcccggtctcggaggatgaggcccgggccgtgctgggtgagggtgtggagatcgccgcggtcaacggcccgtcgtcggtggttctctccggtgatgaggccgccgtgctgcaggccgcggaggggctggggaagtggacgcggctggcgaccagccacgcgttccattccgcccgtatggaacccatgctggaggagttccgggcggtcgccgaaggcctgacctaccggacgccgcaggtctccatggccgttggtgatcaggtgaccaccgctgagtactgggtgcggcaggtccgggacacggtccggttcggcgagcaggtggcctcgtacgaggacgccgtgttcgtcgagctgggtgccgaccggtcactggcccgcctggtcgacggtgtcgcgatgctgcacggcgaccacgaaatccaggccgcgatcggcgccctggcccacctgtatgtcaacggcgtcacggtcgactggcccgcgctcctgggcgatgctccggcaacacgggtgctggaccttccgacatacgccttccagcaccagcgctactggctcgagggcacggaccgggcgactgcgggtggccatccgttgctgggttcggcggtgcggctggccgaggccagcggggtgttgttcactgcccgggtttcccggagcggcgatctgtggctgcgggaccagacggttctgcccgcgacggtgttcgtggagatggcgctggcagcggcggacgaggccggctgcggtctggttgaggacctgaacgtggaagcgttgctgctgcttcctgacgatggcgccgtccaggtacagacctgggtgagcgaaccggacgaggccggtcgccaccggctcagtatccacgcccgttacagcgacagcgagccctggacacgcttggccaccgcaaccctcgccaccaggggaacggtatccggctggcaggccggggaggcgtggccgccgaccggtgcggtcccggtcgagaccggagtaccgtcactgcgcggggtgtggcgccgaggcaacgaagtgttcgccgaggtcgccctggacagcacccacgacgccaccacatatgccctgcaccctgccctcctgaccgccgccctcaccaccgccggtgaggaaacccccgccgcgtggcaggcgctgaccctgcacgcccgcaaccctgccgagctgcgcgtccgcctcatctcacacgatgacggcaccctgtccgtggacgccaccgacagcacaggcctccccgtcctgaccgtccgctccctcaccctgcgcaccgtccccgtctacgaacctgccaccagcaccgacgacctgctcaccctgacctgggcggagatcccggcccctcaggaaaccggcctgacggtcggccggttcgaggacctggtgtcggacgctgatgtgccggtacccgaggtggcggtcttcaccgcactccccgacagcagcgagaacccgctggaacagacccgcgtactgaccgctcaggtcctccaggcagtccagacctggcttggcggggaacgtttcaccgacagcacgctggtcgtgcggaccggcacccggttggccgccgctggggtgtcggggttgatgcgatcggctcaatcggaacaccccggccggttcgtcctggtcgagagcgacgacgacacgctcgccccggaccagttggccgccaccgtcgggctcgacgagccgcggctgcgggtcagcggcgaccggtacgaggcaccgcgactggctcgtgtgaacgccagtgggtctgagcctgaagcggtttgggatccggatggcacggttctgatcaccggtggttcgggtgtgctggcggggatcgccgcccggcacctggtggccgaacgcggcgtgcgtcatctgctgctgctgtcccgcagcgccccggacgaggcactgatcaaccaactcggcgaactgggcgcccgggtcgagacagcggcctgtgacgtgtccgatcgtgccgcgctggcccaggtgctggcgggtgtgtcaccggagcaccccctgacggcagtgattcacaccgcgggcgtactcgatgacggtgttgtcgagtccctgaccgcgcagcggctcgacacggtactgcggcccaaggccgacggcgcctggcatctgcacgaactcacccgcaacaccgacctggccgccttcgtcatgtactcctccgccgccggtgtcatgggcggtggggggcaaggtaactacgcggcggcaaacgcgttcctggacgcgctcgccgaagaacgccgcgccgagggcctgcccgcactcgcggtggcctggggtctgtgggaggacgccagtggcctgaccacgcaactgaccgacacggaccgtgaccggatccggcgcggtggcctgcggactatcaccgccgagtacgggatgcggctgttcgacaccgcatcacgccatggcaacccgattctggtcgccgcaccgatggacccggtttgggacgcggaagtccccgcgctcctccgctcgttgcatcgtcccgtcgcccggcgggccgcctctaccagcgactcgtcagcgcggtggctggcggccctggcaccggccgagcgggaagacgcactgctgaagctggtgcgtgacagcgccgctctggtcctgggacacgctgacgccagcaccatccccgcagccgccgcattcaaggatctgggtatcgattcgctgaccgcggtggaactgcgcaacagcctggcgaaagccacagggctgcggctgcccaacacgacggtgttcgactaccccaccccggccatcctggccacccggctgggtgagctgttcaccggcgagaaccctgcaccggtacgcccgtcggtgtcggtggtggggcaggacgagccgctggcggtcgtgggtatggcctgccgtctgcccggcggggtgtcgtcgcctgaggatctgtggcgccttgtggagtcgggtacggatgcgatttccggtttccccgccgaccgtgggtgggacgcggagagcctgttcgatccggacccggacgcggtcgggaagtcgtactgcgtagagggcggcttcctcgacagcgcagccagcttcgacgccggattcttcggcatcagcccacgcgaggctctggcgatggacccgcagcagcggctgatcatggaggtgtcctgggaggccttcgagcgggccgggatcgagcccggttccgtgcgcggcagcgacaccggcgtcttcatgggcgcgtacgccggtggctacggtgccggtgctgacctcggcggcttcgcggccaccgccagcgcgaccagtgtcctgtccggccgggtgtcgtacttcttcggcctcgaaggccccgccatcacagtcgacacagcctgctcgtcatcactggtggcactgcaccaggccgggtatgccctccggcagggagagtgttccctggccctggtcggcggcgtcaccgtgatggccacaccacaaagcttcgtggaattctcccgccagcgtggtctggcctccgatggccggtgcaaggcgttcgcagacagcgcggacggcacgggatgggctgaaggcgttggtgtgctgctggtagagcggctttccgacgcccaggccaagggccatcaggtgttggcggtggtccgtagctcggcggtcaaccaggacggcgcgtccaacggtctgaccgcgccgaacggtccttcgcagcagcgggtgatccaagccgctctcagtaacgccggcctcgccgcgcacgaggtggatgtggtcgaggcccacggcacgggcacgacgctgggcgacccgatcgaggcccaggcgctgatcgccacttacggtcaggaccgggaacggcccctgctgctgggttcgctgaagtcgaacatcggtcatgctcaggccgcctcgggcgtgtcgggtgtcatcaagatggtcatggccctgcagcacaacacggttccccgcaccctgcacgtggatgagccgtcgcggcacgtggactgggcggcgggtgcggttgagctggtgagggagaaccagccctggcccggcaccgaccggccccgtcgggcgggcgtgtcgtccttcggagtcagcggcaccaacgcccacgtcatcctggagagcgcaccccccgctcagcccgcggaggaggcgcagcctgttgagacgccggtggtggcctcggatgtgctgccgctggtgatatcggccaagacccagcccgccctgaccgaacacgaagaccggctgcgcgcctacctggcggcgtcgcccggggcggatatacgggctgtggcatcgacgctggcggtgacacggtcggtgttcgagcaccgcgccgtactccttggagatgacaccgtcaccggcaccgcggtgaccgaccccaggatcgtgtttgtctttcccgggcaggggtggcagtggctggggatgggcagtgcactgcgcgattcgtcggtggtgttcgccgagcggatggccgagtgtgcggcggcgttgcgcgagttcgtggactgggatctgttcacggttctggatgatccggcggtggtggaccgggttgatgtggtccagcccgcttcctgggcgatgatggtttccctggccgcggtgtggcaggcggccggtgtgcggccggatgcggtgatcggccattcgcagggtgagatcgccgcagcttgtgtggcgggtgcggtgtcactacgcgatgccgcccggatcgtgaccttgcgcagccaggcgatcgcccggggcctggcgggccggggcgcgatggcatccgtcgccctgcccgcgcaggatgtcgagctggtcgacggggcctggatcgccgcccacaacgggcccgcctccaccgtgatcgcgggcaccccggaagcggtcgaccatgtcctcaccgctcatgaggcacaaggggtgcgggtgcggcggatcaccgtcgactatgcctcgcacaccccgcacgtcgagctgatccgcgacgaactactcgacatcactagcgacagcagctcgcagaccccgctcgtgccgtggctgtcgaccgtggacggcacctgggtcgacagcccgctggacggggagtactggtaccggaacctgcgtgaaccggtcggtttccaccccgccgtcagccagttgcaggcccagggcgacaccgtgttcgtcgaggtcagcgccagcccggtgttgttgcaggcgatggacgacgatgtcgtcacggttgccacgctgcgtcgtgacgacggcgacgccacccggatgctcaccgccctggcacaggcctatgtccacggcgtcaccgtcgactggcccgccatcctcggcaccaccacaacccgggtactggaccttccgacctacgccttccagcaccagcggtactggctcaggagcgtggaccgggcggctgccgacggtcatccactgctgggcaccgtagtggcactgcccggctccgacggtgtggtgctcaccgggcgggtgtcgctggccacccatacatggctggccgatcacgcggtccggggcagtgtcctgctacccgggaccgcatttgtggaactggtcgtccgcgccgccgacgaggtcgagtgcgacgtcgttgacgagttggtgatcgaaaccccgctcctgctgccgcagaccggaggcgtccaactgtccgtgtccgtcggcggagccgacgagtccgggcaccgcgcggtgatggtcttctcccaggcggacaacaccgatacctggacccggcacgtcacggcgacagtcagcacctctgactcgacggtctcgctgccggagtttgcctcgtggccaccagcccaggcccggccggtgagcgtggccgacttctacgaccggctgg

SEQ ID NO: 63 - DNA, 3217 bp, Artificial Sequence
  Synthetic Sequence - polynucleotide for modifying rapamycin (expanding a macrolide ring)_fraction1
acgcccacgtcatcctggaaagcgcactccccacacagcctgcgggcaacacagtggtcgagtcggcaccggagtgggtgccgttggtgatttcggcgaggacccagtcggcactggctgaatacgagggccggttgcgtgcgtatctggcggcgtcgcccggggcggatacgcgggctgtggcatcgacgctggcgatgacacggtcggtgttcgagtaccgggccgtactcattggagatgacaccgtcaccggtaccgcggcgaccgatccgcgggtggtgttcgtcttcccgggtcaggggtcgcagcgtgctggtatgggtgaggaactggccgccgcgttccccgtcttcgcgcggatccatcagcaggtgtgggatctgctggatgtgcccgatctcgatgtgaatgagaccgggtatgcccagccggccctgttcgctttgcaggtggctctgttcgggttgctggaatcgtggggtgtacggccggatgcggtggtcggtcactctgtcggtgagctcgccgccggatacgtctccgggttgtggtcgttggaggatgcctgcactttggtgtcggcgcgggctcgtctgatgcaggctctgcctgcgggtggggtgatggtcgctgtcccggtctcggaggatgaggctcgggccgtgctgggtgagggtgtggagatcgccgcggtcaacgggccgtcgtcggtggttctctccggtgatgaggccgccgtgctgcaggccgcggaggggctggggaagtggacgcggctggcgaccagtcacgcgttccattccgcccgtatggaaccgatgctggaggagttccgggcggtcgctgaaggcctgacctaccggacgccgcaggtcgccatggccgctggtgatcaggtgatgaccgctgagtactgggtgcggcaggtccgggacacggtccggttcggcgagcaggtggcctcgttcgaggatgcggtgttcgtcgagctgggtgccgaccggtcactggcccgcctggtcgatggcatcgcgatgctgcacggtgaccatgaggcgcaggccgctgtcggtgccctggctcacctgtacgtgaacggcgtgagtgtcgagtggtccgcggtgctgggtgatgtcccggtaacccgggtgctggatcttccgacgtacgccttccagcaccagcggtactggcttgagggcacggaccgggcgactgcgggtggtcatccgttgctgggttcggtggtgcggctggccgaggccagtggggtgttgttcactgcccgggtttcccggagcggtgatctgtggctgcgggaccagacggttctgcccgcgacggtgttcgtggagatggcgctggcagcggcggacgaggtcggctgcggtctggttgaggatctgagtgtggaagcgttgctgctgcttcccgatgatggcgccgtcgaggtacagacctgggtgggcgaaccggatgagggcggtcggcgccggctcagtgtccacgcccgttacggtgacggcgagccctggacctgcttggccaccgcaaccctggccaccactacgggtgtggccgctgccgcggtcggctggcaggccggtggggtgtggccgccggccggtgcggtcccggtcgggacatcggcaccctcactgcgggcggtgtggcgcctgggcagcgacatcttcgccgaggtggccctggacgatgcccatgatgccaccaggtttgtgcttcatcccggcctgatggccgccgcgctcaccaccgtaggcgaggagactcccgccgtgtggcagggcctgaccctgcacgccggcaatcccggcgagctgcgcgtccgcctcacctcacacgatgacggcaccctgtcggcagaggccaccgacagcacaggcctccccgtcctgaccgcccgctcgctcaccctgcgcaccgtccccgtatacgaaccggccaccagcaccgacgacctgctcaccctgacctgggcaggaatccccaccccccagcagaccggcctgacggtgggtgcgtttgaagacctggcggcggacggcgatgtgccggtacccgaggtggcggtcttcaccgcactccccgacagcgacgatccgctggagcaaacacgaaagctgaccgctcaggtcctccacacactccaggagtggcttggcggggagcgcttcagcgacagcacgctggtggtgcggaccggcaccgggttggccgctgctggggtgtcggggttgatgcgctcggcccagtccgaacaccccggccggttcgtcctggtcgaaagcgacgacgccctcacccaggatcagctggcggcggcggtcggactggatgagccgcggctgcgggtcagcgacggccggtacgaagtaccacggctgacccgcacacatgccgaagagcctgagcctgaaaggacgtgggatccggatggcacggtcctgatcacgggcggttcaggtgtgctggcggggatcgccgcccggcacctggtgaccgaacgcggcgtgcgtcatctcctgctgctgtcccgcagcgccccggatgaggcgctgatcggcgagcttggtgaactgggggcccgggtcgagacagcggcctgtgacgtgtccgatcctgccgcgctgacgcaggtgctggcgggtgtctcgccggagcatcccctgacggccgtgattcacaccgcgggcgtggtggatgacggtgttgtggagtctttgaccgtgcagcggctggagacggtactgcggcccaaggccgacggtgcgtggaacctgcacgagctcacccgggatgccgacctggccgcgttcgtcatgtattcctccgccgccggtgtgctcggtagtgcggggcagggcaactacgcggcggccaatgcgttcctggacgcgctggctgagcagcgtcacgctgagggtctgcccgcactcgcggtggcctggggtctgtgggaggacgccagtggcctgaccgcgcaactgaccgacacggaccgtgaccggatccggcgcggtggcctgcgggccatctccgccgagcacgggatggggctgttcgacagcgcgtcacgccacagtgaaccggttctggtggccgcgccgatggagccggtacgggacgcggaagtcccggcattgctgcggtcgttgcaccgcccgattgctcggcgggccgctgccgccggtgg

SEQ ID NO: 64 - DNA, 2041 bp, Artificial Sequence
  Synthetic Sequence - polynucleotide for modifying rapamycin (expanding a macrolide ring)_fraction2
attgctcggcgggccgctgccgccggtggagcgcggtggctggccgccctggcaccggccgagcgggagaaggcactgctgaagctggtgtctgacggcgccgcgacggttctgggacacgccgacaccagcacgattccggcaaccacggcgttcaaggatctgggcatcaattcgctgaccgcggtggaactgcgcaacagcctggcgaaggccacggagctgcggctgcccgccacgctggtgttcgactaccccaccccggccgccttggctgcccggttggacgagttgttcaccggcgagaaccccgtaccggtacgcgggccggtgtcggcggtggcgcaggacgagccgctggcgatcgtgggaatggcctgccgcctacccggtggagtctcgtcgcctgaggatctgtggcgtctcctggagtcgggtacagatgcggtctccggtttccccaccgaccgtggctgggacgtcgagaacctgtacgacatggctggaaaatcgcaccgtgctgagggtggcttcctggatgccgcggctggctttgatgccggattcttcgggatcagtccgcgtgaggcgttggcgatggatccgcagcagcggctggtgctggaggtgtcctgggaggcgttcgagcgggccgggatcgagcccggttccgtacgcggcagcgataccggcgttttcatgggtgcgtaccccggtggctacggcatcggtgccgacctcggcggcttcggggccaccgccagttcggtcagtgtcctgtccggccgggtgtcgtacttcttcggcctcgagggtcccgcgttcacagtcgacacggcctgctcgtcatcgttggtggcgttgcatcaggcggggtatgccctccggcagggagagtgttcgctggccctggtcggcggtgtcactgtgatggccacgccacagactttcgtggagttctcccgccagggcggcctggcctccgacggccgctgcaaagcgttcgccgacgccgcggacggcacgggatgggctgaaggtgtcggtgtcctgctcgtagagcgactctccgatgcccgccgtaacggtcaccaggtgttggcggtggtgcgtggatcagcggtgaaccaggacggtgcgtcgaacggtctgaccgcgccgaatggtccttcgcagcagcgggtgatccgggccgctctcagcaacgcgggtctgagcacggctgaggtggatgtggtcgaggcgcacggcacgggcacaacgctgggtgacccgatcgaggcccaggcgctgatcgctacctatggccaggaccgtgaccagcctgtgctgctgggttcggtgaagtcgaacctgggtcatacgcaggccgctgcgggtgtgtccggtgtcatcaagatggtgatggccctgcaacacggtctggtgccgcgcacgttgcatgtcgatgagccgtcacggcatgtggactggtcggcgggcgcggtgcagctcgtgacggagaaccagccgtggccggatatgggccgagcgcgccgggcaggcgtgtcgtccttcgggatcagtggcaccaacgcccacgtcatcctggaaagcgcaccccccactcagcctgcggacaacgcggtgatcgagcgggcaccggagtgggtgccgttggtgatttcggccaggacccagtcggctttgactgagcacgagggccggttgcgtgcgtatctggcggcgtcgcccggggtggatatgcgggctgtggcatcgacgctggcgatgacacggtcggtgttcgagcaccgtgccgtgctgctgggagatgacaccgtcaccggcaccgctgtgtctgaccctcgggcggtgttcgtcttcccgggacaggggtcgcagcgtgctggcatgggtgaggaactggccgccgcgttccccgtcttcgcgcggatccatcagcaggtgtgggacctgctcgatgtgcccgatctggaggtgaacgagaccggttacgcccagccggccctgttcgcaatgcaggtggctctgttcgggc

SEQ ID NO: 65 - DNA, 2677 bp, Artificial Sequence
  Synthetic Sequence - polynucleotide for modifying rapamycin (expanding a macrolide ring)_fraction3
aatgcaggtggctctgttcgggctgctggaatcgtggggtgtacgaccggacgcggtgatcggccattcggtgggtgagcttgcggctgcgtatgtgtccggggtgtggtcgttggaggatgcctgcactttggtgtcggcgcgggctcgtctgatgcaggctctgcccgcgggtggggtgatggtcgctgtcccggtctcggaggatgaggcccgggccgtgctgggtgagggtgtggagatcgccgcggtcaacggcccgtcgtcggtggttctctccggtgatgaggccgccgtgctgcaggccgcggaggggctggggaagtggacgcggctggcgaccagccacgcgttccattccgcccgtatggaacccatgctggaggagttccgggcggtcgccgaaggcctgacctaccggacgccgcaggtctccatggccgttggtgatcaggtgaccaccgctgagtactgggtgcggcaggtccgggacacggtccggttcggcgagcaggtggcctcgtacgaggacgccgtgttcgtcgagctgggtgccgaccggtcactggcccgcctggtcgacggtgtcgcgatgctgcacggcgaccacgaaatccaggccgcgatcggcgccctggcccacctgtatgtcaacggcgtcacggtcgactggcccgcgctcctgggcgatgctccggcaacacgggtgctggaccttccgacatacgccttccagcaccagcgctactggctcgagggcacggaccgggcgactgcgggtggccatccgttgctgggttcggcggtgcggctggccgaggccagcggggtgttgttcactgcccgggtttcccggagcggcgatctgtggctgcgggaccagacggttctgcccgcgacggtgttcgtggagatggcgctggcagcggcggacgaggccggctgcggtctggttgaggacctgaacgtggaagcgttgctgctgcttcctgacgatggcgccgtccaggtacagacctgggtgagcgaaccggacgaggccggtcgccaccggctcagtatccacgcccgttacagcgacagcgagccctggacacgcttggccaccgcaaccctcgccaccaggggaacggtatccggctggcaggccggggaggcgtggccgccgaccggtgcggtcccggtcgagaccggagtaccgtcactgcgcggggtgtggcgccgaggcaacgaagtgttcgccgaggtcgccctggacagcacccacgacgccaccacatatgccctgcaccctgccctcctgaccgccgccctcaccaccgccggtgaggaaacccccgccgcgtggcaggcgctgaccctgcacgcccgcaaccctgccgagctgcgcgtccgcctcatctcacacgatgacggcaccctgtccgtggacgccaccgacagcacaggcctccccgtcctgaccgtccgctccctcaccctgcgcaccgtccccgtctacgaacctgccaccagcaccgacgacctgctcaccctgacctgggcggagatcccggcccctcaggaaaccggcctgacggtcggccggttcgaggacctggtgtcggacgctgatgtgccggtacccgaggtggcggtcttcaccgcactccccgacagcagcgagaacccgctggaacagacccgcgtactgaccgctcaggtcctccaggcagtccagacctggcttggcggggaacgtttcaccgacagcacgctggtcgtgcggaccggcacccggttggccgccgctggggtgtcggggttgatgcgatcggctcaatcggaacaccccggccggttcgtcctggtcgagagcgacgacgacacgctcgccccggaccagttggccgccaccgtcgggctcgacgagccgcggctgcgggtcagcggcgaccggtacgaggcaccgcgactggctcgtgtgaacgccagtgggtctgagcctgaagcggtttgggatccggatggcacggttctgatcaccggtggttcgggtgtgctggcggggatcgccgcccggcacctggtggccgaacgcggcgtgcgtcatctgctgctgctgtcccgcagcgccccggacgaggcactgatcaaccaactcggcgaactgggcgcccgggtcgagacagcggcctgtgacgtgtccgatcgtgccgcgctggcccaggtgctggcgggtgtgtcaccggagcaccccctgacggcagtgattcacaccgcgggcgtactcgatgacggtgttgtcgagtccctgaccgcgcagcggctcgacacggtactgcggcccaaggccgacggcgcctggcatctgcacgaactcacccgcaacaccgacctggccgccttcgtcatgtactcctccgccgccggtgtcatgggcggtggggggcaaggtaactacgcggcggcaaacgcgttcctggacgcgctcgccgaagaacgccgcgccgagggcctgcccgcactcgcggtggcctggggtctgtgggaggacgccagtggcctgaccacgcaactgaccgacacggaccgtgaccggatccggcgcggtggcctgcggactatcaccgccgagtacgggatgcggctgttcgacaccgcatcacgccatggcaacccgattctggtcgccgca

SEQ ID NO: 66 - DNA, 2946 bp, Artificial Sequence
  Synthetic Sequence - polynucleotide for modifying rapamycin (expanding a macrolide ring)_fraction4
caacccgattctggtcgccgcaccgatggacccggtttgggacgcggaagtccccgcgctcctccgctcgttgcatcgtcccgtcgcccggcgggccgcctctaccagcgactcgtcagcgcggtggctggcggccctggcaccggccgagcgggaagacgcactgctgaagctggtgcgtgacagcgccgctctggtcctgggacacgctgacgccagcaccatccccgcagccgccgcattcaaggatctgggtatcgattcgctgaccgcggtggaactgcgcaacagcctggcgaaagccacagggctgcggctgcccaacacgacggtgttcgactaccccaccccggccatcctggccacccggctgggtgagctgttcaccggcgagaaccctgcaccggtacgcccgtcggtgtcggtggtggggcaggacgagccgctggcggtcgtgggtatggcctgccgtctgcccggcggggtgtcgtcgcctgaggatctgtggcgccttgtggagtcgggtacggatgcgatttccggtttccccgccgaccgtgggtgggacgcggagagcctgttcgatccggacccggacgcggtcgggaagtcgtactgcgtagagggcggcttcctcgacagcgcagccagcttcgacgccggattcttcggcatcagcccacgcgaggctctggcgatggacccgcagcagcggctgatcatggaggtgtcctgggaggccttcgagcgggccgggatcgagcccggttccgtgcgcggcagcgacaccggcgtcttcatgggcgcgtacgccggtggctacggtgccggtgctgacctcggcggcttcgcggccaccgccagcgcgaccagtgtcctgtccggccgggtgtcgtacttcttcggcctcgaaggccccgccatcacagtcgacacagcctgctcgtcatcactggtggcactgcaccaggccgggtatgccctccggcagggagagtgttccctggccctggtcggcggcgtcaccgtgatggccacaccacaaagcttcgtggaattctcccgccagcgtggtctggcctccgatggccggtgcaaggcgttcgcagacagcgcggacggcacgggatgggctgaaggcgttggtgtgctgctggtagagcggctttccgacgcccaggccaagggccatcaggtgttggcggtggtccgtagctcggcggtcaaccaggacggcgcgtccaacggtctgaccgcgccgaacggtccttcgcagcagcgggtgatccaagccgctctcagtaacgccggcctcgccgcgcacgaggtggatgtggtcgaggcccacggcacgggcacgacgctgggcgacccgatcgaggcccaggcgctgatcgccacttacggtcaggaccgggaacggcccctgctgctgggttcgctgaagtcgaacatcggtcatgctcaggccgcctcgggcgtgtcgggtgtcatcaagatggtcatggccctgcagcacaacacggttccccgcaccctgcacgtggatgagccgtcgcggcacgtggactgggcggcgggtgcggttgagctggtgagggagaaccagccctggcccggcaccgaccggccccgtcgggcgggcgtgtcgtccttcggagtcagcggcaccaacgcccacgtcatcctggagagcgcaccccccgctcagcccgcggaggaggcgcagcctgttgagacgccggtggtggcctcggatgtgctgccgctggtgatatcggccaagacccagcccgccctgaccgaacacgaagaccggctgcgcgcctacctggcggcgtcgcccggggcggatatacgggctgtggcatcgacgctggcggtgacacggtcggtgttcgagcaccgcgccgtactccttggagatgacaccgtcaccggcaccgcggtgaccgaccccaggatcgtgtttgtctttcccgggcaggggtggcagtggctggggatgggcagtgcactgcgcgattcgtcggtggtgttcgccgagcggatggccgagtgtgcggcggcgttgcgcgagttcgtggactgggatctgttcacggttctggatgatccggcggtggtggaccgggttgatgtggtccagcccgcttcctgggcgatgatggtttccctggccgcggtgtggcaggcggccggtgtgcggccggatgcggtgatcggccattcgcagggtgagatcgccgcagcttgtgtggcgggtgcggtgtcactacgcgatgccgcccggatcgtgaccttgcgcagccaggcgatcgcccggggcctggcgggccggggcgcgatggcatccgtcgccctgcccgcgcaggatgtcgagctggtcgacggggcctggatcgccgcccacaacgggcccgcctccaccgtgatcgcgggcaccccggaagcggtcgaccatgtcctcaccgctcatgaggcacaaggggtgcgggtgcggcggatcaccgtcgactatgcctcgcacaccccgcacgtcgagctgatccgcgacgaactactcgacatcactagcgacagcagctcgcagaccccgctcgtgccgtggctgtcgaccgtggacggcacctgggtcgacagcccgctggacggggagtactggtaccggaacctgcgtgaaccggtcggtttccaccccgccgtcagccagttgcaggcccagggcgacaccgtgttcgtcgaggtcagcgccagcccggtgttgttgcaggcgatggacgacgatgtcgtcacggttgccacgctgcgtcgtgacgacggcgacgccacccggatgctcaccgccctggcacaggcctatgtccacggcgtcaccgtcgactggcccgccatcctcggcaccaccacaacccgggtactggaccttccgacctacgccttcca

SEQ ID NO: 67 - DNA, 534 bp, Artificial Sequence
  Synthetic Sequence - polynucleotide for modifying rapamycin (expanding a macrolide ring)_fraction5
ccacaacccgggtactggaccttccgacctacgccttccagcaccagcggtactggctcaggagcgtggaccgggcggctgccgacggtcatccactgctgggcaccgtagtggcactgcccggctccgacggtgtggtgctcaccgggcgggtgtcgctggccacccatacatggctggccgatcacgcggtccggggcagtgtcctgctacccgggaccgcatttgtggaactggtcgtccgcgccgccgacgaggtcgagtgcgacgtcgttgacgagttggtgatcgaaaccccgctcctgctgccgcagaccggaggcgtccaactgtccgtgtccgtcggcggagccgacgagtccgggcaccgcgcggtgatggtcttctcccaggcggacaacaccgatacctggacccggcacgtcacggcgacagtcagcacctctgactcgacggtctcgctgccggagtttgcctcgtggccaccagcccaggcccggccggtgagcgtggccgacttctacgaccggctgg

SEQ ID NO: 68 - DNA, 5229 bp, Artificial Sequence
  Synthetic Sequence - polynucleotide for modifying rapamycin (expanding a macrolide ring)_fraction1-2
acgcccacgtcatcctggaaagcgcactccccacacagcctgcgggcaacacagtggtcgagtcggcaccggagtgggtgccgttggtgatttcggcgaggacccagtcggcactggctgaatacgagggccggttgcgtgcgtatctggcggcgtcgcccggggcggatacgcgggctgtggcatcgacgctggcgatgacacggtcggtgttcgagtaccgggccgtactcattggagatgacaccgtcaccggtaccgcggcgaccgatccgcgggtggtgttcgtcttcccgggtcaggggtcgcagcgtgctggtatgggtgaggaactggccgccgcgttccccgtcttcgcgcggatccatcagcaggtgtgggatctgctggatgtgcccgatctcgatgtgaatgagaccgggtatgcccagccggccctgttcgctttgcaggtggctctgttcgggttgctggaatcgtggggtgtacggccggatgcggtggtcggtcactctgtcggtgagctcgccgccggatacgtctccgggttgtggtcgttggaggatgcctgcactttggtgtcggcgcgggctcgtctgatgcaggctctgcctgcgggtggggtgatggtcgctgtcccggtctcggaggatgaggctcgggccgtgctgggtgagggtgtggagatcgccgcggtcaacgggccgtcgtcggtggttctctccggtgatgaggccgccgtgctgcaggccgcggaggggctggggaagtggacgcggctggcgaccagtcacgcgttccattccgcccgtatggaaccgatgctggaggagttccgggcggtcgctgaaggcctgacctaccggacgccgcaggtcgccatggccgctggtgatcaggtgatgaccgctgagtactgggtgcggcaggtccgggacacggtccggttcggcgagcaggtggcctcgttcgaggatgcggtgttcgtcgagctgggtgccgaccggtcactggcccgcctggtcgatggcatcgcgatgctgcacggtgaccatgaggcgcaggccgctgtcggtgccctggctcacctgtacgtgaacggcgtgagtgtcgagtggtccgcggtgctgggtgatgtcccggtaacccgggtgctggatcttccgacgtacgccttccagcaccagcggtactggcttgagggcacggaccgggcgactgcgggtggtcatccgttgctgggttcggtggtgcggctggccgaggccagtggggtgttgttcactgcccgggtttcccggagcggtgatctgtggctgcgggaccagacggttctgcccgcgacggtgttcgtggagatggcgctggcagcggcggacgaggtcggctgcggtctggttgaggatctgagtgtggaagcgttgctgctgcttcccgatgatggcgccgtcgaggtacagacctgggtgggcgaaccggatgagggcggtcggcgccggctcagtgtccacgcccgttacggtgacggcgagccctggacctgcttggccaccgcaaccctggccaccactacgggtgtggccgctgccgcggtcggctggcaggccggtggggtgtggccgccggccggtgcggtcccggtcgggacatcggcaccctcactgcgggcggtgtggcgcctgggcagcgacatcttcgccgaggtggccctggacgatgcccatgatgccaccaggtttgtgcttcatcccggcctgatggccgccgcgctcaccaccgtaggcgaggagactcccgccgtgtggcagggcctgaccctgcacgccggcaatcccggcgagctgcgcgtccgcctcacctcacacgatgacggcaccctgtcggcagaggccaccgacagcacaggcctccccgtcctgaccgcccgctcgctcaccctgcgcaccgtccccgtatacgaaccggccaccagcaccgacgacctgctcaccctgacctgggcaggaatccccaccccccagcagaccggcctgacggtgggtgcgtttgaagacctggcggcggacggcgatgtgccggtacccgaggtggcggtcttcaccgcactccccgacagcgacgatccgctggagcaaacacgaaagctgaccgctcaggtcctccacacactccaggagtggcttggcggggagcgcttcagcgacagcacgctggtggtgcggaccggcaccgggttggccgctgctggggtgtcggggttgatgcgctcggcccagtccgaacaccccggccggttcgtcctggtcgaaagcgacgacgccctcacccaggatcagctggcggcggcggtcggactggatgagccgcggctgcgggtcagcgacggccggtacgaagtaccacggctgacccgcacacatgccgaagagcctgagcctgaaaggacgtgggatccggatggcacggtcctgatcacgggcggttcaggtgtgctggcggggatcgccgcccggcacctggtgaccgaacgcggcgtgcgtcatctcctgctgctgtcccgcagcgccccggatgaggcgctgatcggcgagcttggtgaactgggggcccgggtcgagacagcggcctgtgacgtgtccgatcctgccgcgctgacgcaggtgctggcgggtgtctcgccggagcatcccctgacggccgtgattcacaccgcgggcgtggtggatgacggtgttgtggagtctttgaccgtgcagcggctggagacggtactgcggcccaaggccgacggtgcgtggaacctgcacgagctcacccgggatgccgacctggccgcgttcgtcatgtattcctccgccgccggtgtgctcggtagtgcggggcagggcaactacgcggcggccaatgcgttcctggacgcgctggctgagcagcgtcacgctgagggtctgcccgcactcgcggtggcctggggtctgtgggaggacgccagtggcctgaccgcgcaactgaccgacacggaccgtgaccggatccggcgcggtggcctgcgggccatctccgccgagcacgggatggggctgttcgacagcgcgtcacgccacagtgaaccggttctggtggccgcgccgatggagccggtacgggacgcggaagtcccggcattgctgcggtcgttgcaccgcccgattgctcggcgggccgctgccgccggtggagcgcggtggctggccgccctggcaccggccgagcgggagaaggcactgctgaagctggtgtctgacggcgccgcgacggttctgggacacgccgacaccagcacgattccggcaaccacggcgttcaaggatctgggcatcaattcgctgaccgcggtggaactgcgcaacagcctggcgaaggccacggagctgcggctgcccgccacgctggtgttcgactaccccaccccggccgccttggctgcccggttggacgagttgttcaccggcgagaaccccgtaccggtacgcgggccggtgtcggcggtggcgcaggacgagccgctggcgatcgtgggaatggcctgccgcctacccggtggagtctcgtcgcctgaggatctgtggcgtctcctggagtcgggtacagatgcggtctccggtttccccaccgaccgtggctgggacgtcgagaacctgtacgacatggctggaaaatcgcaccgtgctgagggtggcttcctggatgccgcggctggctttgatgccggattcttcgggatcagtccgcgtgaggcgttggcgatggatccgcagcagcggctggtgctggaggtgtcctgggaggcgttcgagcgggccgggatcgagcccggttccgtacgcggcagcgataccggcgttttcatgggtgcgtaccccggtggctacggcatcggtgccgacctcggcggcttcggggccaccgccagttcggtcagtgtcctgtccggccgggtgtcgtacttcttcggcctcgagggtcccgcgttcacagtcgacacggcctgctcgtcatcgttggtggcgttgcatcaggcggggtatgccctccggcagggagagtgttcgctggccctggtcggcggtgtcactgtgatggccacgccacagactttcgtggagttctcccgccagggcggcctggcctccgacggccgctgcaaagcgttcgccgacgccgcggacggcacgggatgggctgaaggtgtcggtgtcctgctcgtagagcgactctccgatgcccgccgtaacggtcaccaggtgttggcggtggtgcgtggatcagcggtgaaccaggacggtgcgtcgaacggtctgaccgcgccgaatggtccttcgcagcagcgggtgatccgggccgctctcagcaacgcgggtctgagcacggctgaggtggatgtggtcgaggcgcacggcacgggcacaacgctgggtgacccgatcgaggcccaggcgctgatcgctacctatggccaggaccgtgaccagcctgtgctgctgggttcggtgaagtcgaacctgggtcatacgcaggccgctgcgggtgtgtccggtgtcatcaagatggtgatggccctgcaacacggtctggtgccgcgcacgttgcatgtcgatgagccgtcacggcatgtggactggtcggcgggcgcggtgcagctcgtgacggagaaccagccgtggccggatatgggccgagcgcgccgggcaggcgtgtcgtccttcgggatcagtggcaccaacgcccacgtcatcctggaaagcgcaccccccactcagcctgcggacaacgcggtgatcgagcgggcaccggagtgggtgccgttggtgatttcggccaggacccagtcggctttgactgagcacgagggccggttgcgtgcgtatctggcggcgtcgcccggggtggatatgcgggctgtggcatcgacgctggcgatgacacggtcggtgttcgagcaccgtgccgtgctgctgggagatgacaccgtcaccggcaccgctgtgtctgaccctcgggcggtgttcgtcttcccgggacaggggtcgcagcgtgctggcatgggtgaggaactggccgccgcgttccccgtcttcgcgcggatccatcagcaggtgtgggacctgctcgatgtgcccgatctggaggtgaacgagaccggttacgcccagccggccctgttcgcaatgcaggtggctctgttcgggc

SEQ ID NO: 69 - DNA, 6095 bp, Artificial Sequence
  Synthetic Sequence - polynucleotide for modifying rapamycin (expanding a macrolide ring)_fraction3-5
aatgcaggtggctctgttcgggctgctggaatcgtggggtgtacgaccggacgcggtgatcggccattcggtgggtgagcttgcggctgcgtatgtgtccggggtgtggtcgttggaggatgcctgcactttggtgtcggcgcgggctcgtctgatgcaggctctgcccgcgggtggggtgatggtcgctgtcccggtctcggaggatgaggcccgggccgtgctgggtgagggtgtggagatcgccgcggtcaacggcccgtcgtcggtggttctctccggtgatgaggccgccgtgctgcaggccgcggaggggctggggaagtggacgcggctggcgaccagccacgcgttccattccgcccgtatggaacccatgctggaggagttccgggcggtcgccgaaggcctgacctaccggacgccgcaggtctccatggccgttggtgatcaggtgaccaccgctgagtactgggtgcggcaggtccgggacacggtccggttcggcgagcaggtggcctcgtacgaggacgccgtgttcgtcgagctgggtgccgaccggtcactggcccgcctggtcgacggtgtcgcgatgctgcacggcgaccacgaaatccaggccgcgatcggcgccctggcccacctgtatgtcaacggcgtcacggtcgactggcccgcgctcctgggcgatgctccggcaacacgggtgctggaccttccgacatacgccttccagcaccagcgctactggctcgagggcacggaccgggcgactgcgggtggccatccgttgctgggttcggcggtgcggctggccgaggccagcggggtgttgttcactgcccgggtttcccggagcggcgatctgtggctgcgggaccagacggttctgcccgcgacggtgttcgtggagatggcgctggcagcggcggacgaggccggctgcggtctggttgaggacctgaacgtggaagcgttgctgctgcttcctgacgatggcgccgtccaggtacagacctgggtgagcgaaccggacgaggccggtcgccaccggctcagtatccacgcccgttacagcgacagcgagccctggacacgcttggccaccgcaaccctcgccaccaggggaacggtatccggctggcaggccggggaggcgtggccgccgaccggtgcggtcccggtcgagaccggagtaccgtcactgcgcggggtgtggcgccgaggcaacgaagtgttcgccgaggtcgccctggacagcacccacgacgccaccacatatgccctgcaccctgccctcctgaccgccgccctcaccaccgccggtgaggaaacccccgccgcgtggcaggcgctgaccctgcacgcccgcaaccctgccgagctgcgcgtccgcctcatctcacacgatgacggcaccctgtccgtggacgccaccgacagcacaggcctccccgtcctgaccgtccgctccctcaccctgcgcaccgtccccgtctacgaacctgccaccagcaccgacgacctgctcaccctgacctgggcggagatcccggcccctcaggaaaccggcctgacggtcggccggttcgaggacctggtgtcggacgctgatgtgccggtacccgaggtggcggtcttcaccgcactccccgacagcagcgagaacccgctggaacagacccgcgtactgaccgctcaggtcctccaggcagtccagacctggcttggcggggaacgtttcaccgacagcacgctggtcgtgcggaccggcacccggttggccgccgctggggtgtcggggttgatgcgatcggctcaatcggaacaccccggccggttcgtcctggtcgagagcgacgacgacacgctcgccccggaccagttggccgccaccgtcgggctcgacgagccgcggctgcgggtcagcggcgaccggtacgaggcaccgcgactggctcgtgtgaacgccagtgggtctgagcctgaagcggtttgggatccggatggcacggttctgatcaccggtggttcgggtgtgctggcggggatcgccgcccggcacctggtggccgaacgcggcgtgcgtcatctgctgctgctgtcccgcagcgccccggacgaggcactgatcaaccaactcggcgaactgggcgcccgggtcgagacagcggcctgtgacgtgtccgatcgtgccgcgctggcccaggtgctggcgggtgtgtcaccggagcaccccctgacggcagtgattcacaccgcgggcgtactcgatgacggtgttgtcgagtccctgaccgcgcagcggctcgacacggtactgcggcccaaggccgacggcgcctggcatctgcacgaactcacccgcaacaccgacctggccgccttcgtcatgtactcctccgccgccggtgtcatgggcggtggggggcaaggtaactacgcggcggcaaacgcgttcctggacgcgctcgccgaagaacgccgcgccgagggcctgcccgcactcgcggtggcctggggtctgtgggaggacgccagtggcctgaccacgcaactgaccgacacggaccgtgaccggatccggcgcggtggcctgcggactatcaccgccgagtacgggatgcggctgttcgacaccgcatcacgccatggcaacccgattctggtcgccgcaccgatggacccggtttgggacgcggaagtccccgcgctcctccgctcgttgcatcgtcccgtcgcccggcgggccgcctctaccagcgactcgtcagcgcggtggctggcggccctggcaccggccgagcgggaagacgcactgctgaagctggtgcgtgacagcgccgctctggtcctgggacacgctgacgccagcaccatccccgcagccgccgcattcaaggatctgggtatcgattcgctgaccgcggtggaactgcgcaacagcctggcgaaagccacagggctgcggctgcccaacacgacggtgttcgactaccccaccccggccatcctggccacccggctgggtgagctgttcaccggcgagaaccctgcaccggtacgcccgtcggtgtcggtggtggggcaggacgagccgctggcggtcgtgggtatggcctgccgtctgcccggcggggtgtcgtcgcctgaggatctgtggcgccttgtggagtcgggtacggatgcgatttccggtttccccgccgaccgtgggtgggacgcggagagcctgttcgatccggacccggacgcggtcgggaagtcgtactgcgtagagggcggcttcctcgacagcgcagccagcttcgacgccggattcttcggcatcagcccacgcgaggctctggcgatggacccgcagcagcggctgatcatggaggtgtcctgggaggccttcgagcgggccgggatcgagcccggttccgtgcgcggcagcgacaccggcgtcttcatgggcgcgtacgccggtggctacggtgccggtgctgacctcggcggcttcgcggccaccgccagcgcgaccagtgtcctgtccggccgggtgtcgtacttcttcggcctcgaaggccccgccatcacagtcgacacagcctgctcgtcatcactggtggcactgcaccaggccgggtatgccctccggcagggagagtgttccctggccctggtcggcggcgtcaccgtgatggccacaccacaaagcttcgtggaattctcccgccagcgtggtctggcctccgatggccggtgcaaggcgttcgcagacagcgcggacggcacgggatgggctgaaggcgttggtgtgctgctggtagagcggctttccgacgcccaggccaagggccatcaggtgttggcggtggtccgtagctcggcggtcaaccaggacggcgcgtccaacggtctgaccgcgccgaacggtccttcgcagcagcgggtgatccaagccgctctcagtaacgccggcctcgccgcgcacgaggtggatgtggtcgaggcccacggcacgggcacgacgctgggcgacccgatcgaggcccaggcgctgatcgccacttacggtcaggaccgggaacggcccctgctgctgggttcgctgaagtcgaacatcggtcatgctcaggccgcctcgggcgtgtcgggtgtcatcaagatggtcatggccctgcagcacaacacggttccccgcaccctgcacgtggatgagccgtcgcggcacgtggactgggcggcgggtgcggttgagctggtgagggagaaccagccctggcccggcaccgaccggccccgtcgggcgggcgtgtcgtccttcggagtcagcggcaccaacgcccacgtcatcctggagagcgcaccccccgctcagcccgcggaggaggcgcagcctgttgagacgccggtggtggcctcggatgtgctgccgctggtgatatcggccaagacccagcccgccctgaccgaacacgaagaccggctgcgcgcctacctggcggcgtcgcccggggcggatatacgggctgtggcatcgacgctggcggtgacacggtcggtgttcgagcaccgcgccgtactccttggagatgacaccgtcaccggcaccgcggtgaccgaccccaggatcgtgtttgtctttcccgggcaggggtggcagtggctggggatgggcagtgcactgcgcgattcgtcggtggtgttcgccgagcggatggccgagtgtgcggcggcgttgcgcgagttcgtggactgggatctgttcacggttctggatgatccggcggtggtggaccgggttgatgtggtccagcccgcttcctgggcgatgatggtttccctggccgcggtgtggcaggcggccggtgtgcggccggatgcggtgatcggccattcgcagggtgagatcgccgcagcttgtgtggcgggtgcggtgtcactacgcgatgccgcccggatcgtgaccttgcgcagccaggcgatcgcccggggcctggcgggccggggcgcgatggcatccgtcgccctgcccgcgcaggatgtcgagctggtcgacggggcctggatcgccgcccacaacgggcccgcctccaccgtgatcgcgggcaccccggaagcggtcgaccatgtcctcaccgctcatgaggcacaaggggtgcgggtgcggcggatcaccgtcgactatgcctcgcacaccccgcacgtcgagctgatccgcgacgaactactcgacatcactagcgacagcagctcgcagaccccgctcgtgccgtggctgtcgaccgtggacggcacctgggtcgacagcccgctggacggggagtactggtaccggaacctgcgtgaaccggtcggtttccaccccgccgtcagccagttgcaggcccagggcgacaccgtgttcgtcgaggtcagcgccagcccggtgttgttgcaggcgatggacgacgatgtcgtcacggttgccacgctgcgtcgtgacgacggcgacgccacccggatgctcaccgccctggcacaggcctatgtccacggcgtcaccgtcgactggcccgccatcctcggcaccaccacaacccgggtactggaccttccgacctacgccttccagcaccagcggtactggctcaggagcgtggaccgggcggctgccgacggtcatccactgctgggcaccgtagtggcactgcccggctccgacggtgtggtgctcaccgggcgggtgtcgctggccacccatacatggctggccgatcacgcggtccggggcagtgtcctgctacccgggaccgcatttgtggaactggtcgtccgcgccgccgacgaggtcgagtgcgacgtcgttgacgagttggtgatcgaaaccccgctcctgctgccgcagaccggaggcgtccaactgtccgtgtccgtcggcggagccgacgagtccgggcaccgcgcggtgatggtcttctcccaggcggacaacaccgatacctggacccggcacgtcacggcgacagtcagcacctctgactcgacggtctcgctgccggagtttgcctcgtggccaccagcccaggcccggccggtgagcgtggccgacttctacgaccggctgg

(19) United States
(12) Patent Application Publication
(10) Pub. No.: US 2022/0090100 A1
(43) Pub. Date: Mar. 24, 2022

(54) METHOD FOR PRODUCING COMPOUND WITH MODIFIED MOTHER NUCLEUS

(71) Applicant: JAPAN BIOLOGICAL INFORMATICS CONSORTIUM, Tokyo (JP)

(72) Inventors: Kazuo SHINYA, Tokyo (JP); Haruo IKEDA, Tokyo (JP); Mamoru KOMATSU, Tokyo (JP); Junko HASHIMOTO, Tokyo (JP); Ikuko KOZONE, Tokyo (JP); Takuya HASHIMOTO, Tokyo (JP); Kei KUDO, Tokyo (JP)

(73) Assignee: JAPAN BIOLOGICAL INFORMATICS CONSORTIUM, Tokyo (JP)

(21) Appl. No.: 17/426,621

(22) PCT Filed: Jan. 30, 2020
(86) PCT No.: PCT/JP2020/003309

§ 371 Date: Jul. 28, 2021

(30) Foreign Application Priority Data
Jan. 31, 2019 (JP) 2019-016531

(51) Int. Cl. C12N 15/76; C07K 7/54; C12P 17/18; C12N 15/11; C12N 9/22

(52) U.S. Cl. CPC C12N 15/76; C07K 7/54; C12N 2310/20; C12N 15/11; C12N 9/22; C12P 17/188

(57) ABSTRACT

The present invention provides a method for producing a modified compound, including the following steps:
(1) a step of cleaving in vitro using a CRISPR/Cas9 system, a target site in a gene cluster involved in the biosynthesis of a compound,
(2) a step of connecting using Gibson assembly in vitro the gene cluster cleaved in step (1) and a polynucleotide for modification, and
(3) a step of expressing the modified gene cluster obtained in step (2) in a microorganism expression system.

### TECHNICAL FIELD

The present invention relates to methods for producing compounds having desired mother nucleus modifications.

### BACKGROUND ART

Schreiber at Harvard University proposed the term chemical genetics by establishing a method for identifying a target molecule such as FK506 (Tacrolimus, CAS No.: 104987-11-3) and the like. At the same time, based on the idea of reverse chemical genetics, he proceeded with the construction of a diverse-oriented synthesis compound library, aiming at knockout of all gene products by compounds instead of gene knockout. However, a library for the compounds with strong activity like natural compounds could not be constructed, and this idea was not realized. This indicates that a compound library covering various target molecules could not be created by compound library construction using the conventional organic synthesis method. FK506 and the like are compounds representing natural compounds, and are compounds called “middle molecules” having a large molecular weight. The total synthesis per se of such middle-molecular-weight natural compounds is possible by the current organic synthesis chemical techniques. However, it is not possible to supply a totally synthesized product as a pharmaceutical product, and compounds are still supplied by fermentation methods using microorganisms. One of the disadvantages of natural compounds is difficulty in developing derivatives for the purpose of enhancing specificity, avoiding side effects, improving metabolism, and the like. In some cases, clinical development is abandoned due to such disadvantage, thereby forming the largest bottleneck in the discovery of a natural product drug. Given this background, modification of the mother nucleus by modification of a biosynthetic gene has been studied as a technique for modifying the mother nucleus of a middle-molecular-weight natural compound.

As regards macrolide compounds and cyclic peptide compounds that are biosynthesized by type I polyketide synthase (type I PKS) and non-ribosomal peptide synthetase (NRPS), what unit of partial structure is bound to each module in the process of constructing the structure of the mother nucleus is strictly controlled by the gene sequence. Therefore, it is possible to modify the mother nucleus of a compound by modifying, deleting, or adding a gene of the functional domain region within this module. However, a biosynthetic gene cluster of such compounds generally consists of a large gene group over 100 kb and is constituted of highly homologous repeat sequences. Therefore, a plurality of sequences having high similarity exist in the sequence of the gene cluster, and many restriction enzyme sites important for gene manipulation also exist. Thus, when a genetic modification technique using homologous recombination in a producing bacterium or a gene modification technique using a restriction enzyme, which has conventionally been used in the art, is applied, it is almost impossible to modify a gene “as intended”. Although such concept has been proposed for a long time (non-patent document 1), no one could succeed not only in Japan but also in the world. This is clearly shown in a paper (non-patent document 2) published in Nature Communications most recently.

In non-patent document 2, Gregory and Wilkinson et al. of the United Kingdom tried to modify mother nucleus by replacing the DH-ER-KR sequence of module 3 of the biosynthesis gene cluster of rapamycin with the KR sequence of module 11 or the DH-ER-KR sequence of module 13.

They constructed a new construct by using a restriction enzyme site to a fragment amplified by PCR from a fragment of a rapamycin biosynthesis gene collected using a cosmid vector, introduced the same into a rapamycin-producing microorganism, and tried domain swapping by applying a homologous recombination mechanism.

As a result, the compounds of interest were not obtained but a large number of PKSs were obtained in which recombination occurred at unexpected sites. They obtained 667 colonies and subjected them to compound production. Among them, 421 clones (63.1%) produced the original rapamycin, 150 clones (22.5%) produced novel analogs (only 8 compounds were identified), and 96 clones (14.4%) produced nothing. It is assumed that the results obtained by them in this study are regarded as a successful case of domain swapping study of type I PKS in this technical field, considering the fact that it was published in Nature Communications. However, thioesterase is not present in the rapamycin biosynthetic gene. It is therefore considered that cyclization occurs by chance in the method they used, and it was fortunate to some extent that the allowable range was large. It is expected to be difficult to create an analog compound with such high probability when other type I PKSs are used (Furthermore, the biosynthesis genes of rapamycin have many extremely high homologous regions and thus homologous recombination is considered to occur easily. As described above, when domain swapping of type I PKS is performed using conventional technology, the result obtained is only a “product of chance” even though huge cost and effort are required. If the structure of a compound-target factor is obtained in the future by analysis such as cryo-electron microscope and the like, the accuracy of the docking simulation is improved. Therefore, it is inevitable that the modification of the mother nucleus of middle molecular compounds is demanded more purposively.

### DOCUMENT LIST

### Non-Patent Documents

non-patent document 1: Kim E et al., Nat Chem Biol. 2015 September; 11(9):649-59.
non-patent document 2: Wlodek A. et al., Nat Commun. 2017 Oct. 31; 8(1):1206.

### SUMMARY OF INVENTION

### Technical Problem

In genome editing of prokaryotic organisms, genome editing using homologous recombination is often performed because the efficiency of homologous recombination is high. However, particularly in a gene containing many highly homologous sequences, a desired sequence is often not obtained because recombination occurs in many unintended regions. When the CRISPR/Cas9 system developed in recent years is used, the desired sequence can be cleaved. However, the problem of recombination in unintended regions cannot be solved since subsequent recombination requires homologous recombination in prokaryotic organisms, which do not have a non-homologous end-joining mechanism.

In particular, derivatives of useful natural compounds produced by microorganisms (e.g., middle molecular compounds, etc.) are extremely difficult to artificially synthesize because of the complexity of the structures thereof. Therefore, there is an extremely high need for the development of a means of producing derivatives by modifying a gene or gene cluster involved in the biosynthesis of such compounds. As shown in non-patent document 2, previous studies have reported that the mother nucleus structure of middle molecular compounds can be modified, even though extremely inefficiently, by editing the genes involved in the biosynthesis of middle molecular compounds module by module. Therefore, an object of the present invention is to provide a method capable of producing with higher efficiency a middle molecular compound or the like having a desired mother nucleus modification.

### Solution to Problem

As a means to solve this problem, the present inventors have invented a new technical development using the CRISPR/Cas9 system, which is one of the genome editing techniques. The CRISPR/Cas9 system is capable of cleaving genes at the intended zo site without being limited by restriction enzyme sites, and was considered to be suitable for application to gigantic biosynthesis gene clusters. Research is also being actively conducted to increase the success rate of the CRISPR/Cas9 system. However, it has been reported that the success rate in the case of application to an actual disease model is about 40%, of which the complete mutant is about 30% (chimeric mutation is 70%). As described above, even if CRISPR/Cas9 technique that enables gene cleavage at an accurate sequence position is used, genome editing in vivo is not highly efficient as the situation stands. Besides the targeted biosynthesis genes, an extremely large number of other biosynthesis genes are present in the genome of actinomycetes to be the main target of type I PKS domain swapping. Furthermore, also due to the background of biased GC content and the like, it is almost impossible to overcome the problems of the design of gene cleavage site and uncutting in consideration of the whole genome sequence. Therefore, it can be said that in vivo genome modification is extremely difficult in actinomycetes.

Under these circumstances, the present inventors have constructed a novel method including a combination of CRISPR/Cas9 system, Gibson assembly, a gigantic biosynthesis gene cluster obtaining technique using BAC library, and a heterologous expression technique for a medium-molecular-weight natural compound, and overcome these problems. To be specific, instead of conducting the genetic modification of the target compound in the producing microorganism (that is, genetic modification in vivo), a BAC vector into which a gene cluster involved in the biosynthesis of a middle molecular compound had been inserted was modified in vitro using the CRISPR/Cas9 system and Gibson assembly, and then, instead of the strain that originally produces the middle molecular compound, a special expression strain was transformed using the BAC vector into which the modified gene cluster had been inserted, whereby a middle molecular compound having the intended mother nucleus modification could be produced with extremely high efficiency as compared with the method taught in non-patent document 2.

Accordingly, the present invention provides the following.

[1] A method for producing a modified compound, comprising the following steps:
(1) a step of cleaving in vitro using CRISPR/Cas9 system, a target site in a gene cluster involved in the biosynthesis of a compound,
(2) a step of linking in vitro using Gibson assembly, the gene cluster cleaved in step (1) and a polynucleotide for modification, and
(3) a step of expressing the modified gene cluster obtained in step (2) in a microorganism expression system.
[2] The method of [1], further comprising the following step (A) before step (1):
(A) a step of inserting a gene cluster involved in the biosynthesis of a compound into an expression vector.
[3] The method of [2], wherein the expression vector is a chromosome-integrated expression vector.
[4] The method of [3], wherein the expression vector is selected from the group consisting of a Cosmid vector, a BAC vector, and a YAC vector.
[5] The method of any of [1] to [4], wherein the microorganism expression system is a heterologous expression system.
[6] The method of any of [1] to [5], wherein a Streptomyces lividans or SUKA strain is used in the microorganism expression system.

### Advantageous Effects of Invention

According to the present invention, a gene (or gene cluster) having a long chain (e.g., 40 kbp or more) and many similar sequences, which has been difficult to modify so far, can be modified as intended. Therefore, according to the present invention, for example, a biosynthesis gene cluster in a natural middle molecular compound produced by a microorganism can be modified as intended module by module. According to the present invention, moreover, a middle molecular compound having a desired modification can be created highly efficiently by expressing a modified gene cluster by using a specific microorganism expression system.

### BRIEF DESCRIPTION OF DRAWINGS

FIG. 1 shows the biosynthesis pathway of actinomycin X2 in Streptomyces xanthochromogenes.

FIG. 2 outlines the flow of modifying an actinomycin X2 biosynthesis gene cluster into an actinomycin D biosynthesis gene cluster by using gene editing including CRISPR/Cas9 system and Gibson assembly in combination.

FIG. 3 shows an analysis of metabolic product by actinomycin D selective accumulation strain by gene editing.

FIG. 4 shows the biosynthesis pathway of Rapamycin.

FIG. 5 is a schematic diagram of construction of the modified compound of Rapamycin with increased double bonds.

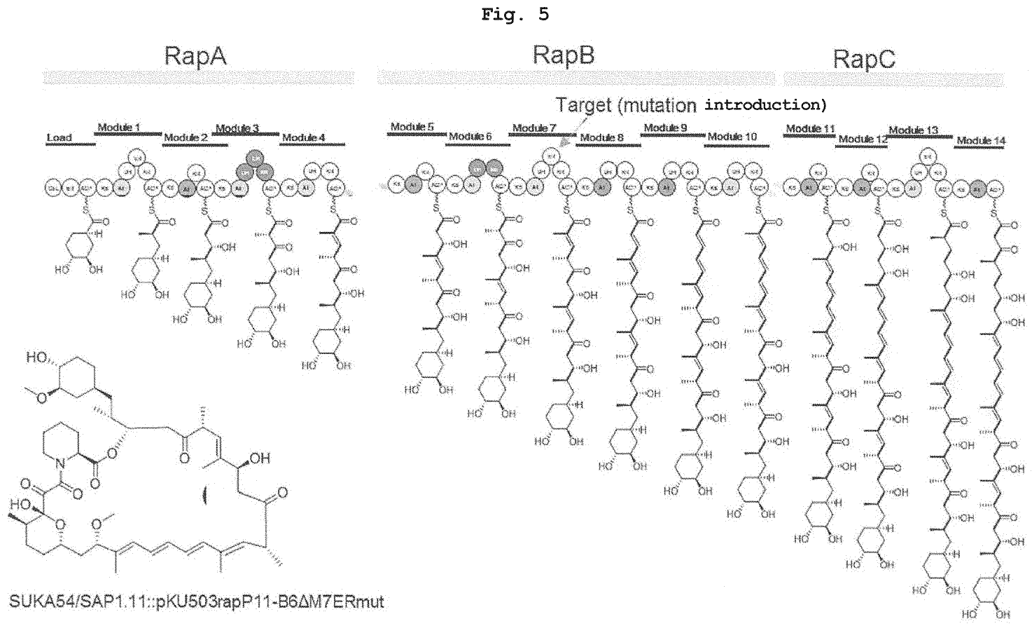

FIG. 6 shows an electrophoretogram after ER domain cleavage in module 7 of Rapamycin by CRISPR/Cas9. In each lane, the concentration of CRISPR/Cas9 used was constant, and the amount of BAC vector used was examined.

FIG. 7 shows the result of confirmation of the production of modified rapamycin (tetraene derivative) by using a mass spectrometer.

FIG. 8 shows the result of confirmation by using UV spectrum that the obtained modified rapamycin has a tetraene structure.

FIG. 9 is a schematic diagram of construction of a mother nucleus modified compound of Rapamycin with a modified methyl group side chain.

FIG. 10 shows an electrophoretogram after cleavage of AT domain at module 9 of Rapamycin by CRISPR/Cas9.

FIG. 11 shows the result of confirmation of the production of modified rapamycin (modification of methyl group side chain) by using a mass spectrometer.

FIG. 12 is a schematic diagram of construction of a module-lacking compound of Rapamycin.

FIG. 13 shows an electrophoretogram after cleavage of module 6 (M5ACP-M6KR) of Rapamycin by CRISPR/Cas9.

FIG. 14 shows the result of confirmation of the production of modified rapamycin (module-lacking) by using a mass spectrometer.

FIG. 15 is a schematic diagram of construction of a module-adding compound of Rapamycin.

FIG. 16 shows an electrophoretogram after cleavage between modules 2-3 of Rapamycin by CRISPR/Cas9.

FIG. 17 shows the result of confirmation of the production of modified rapamycin (module-adding) by using a mass spectrometer.

FIG. 18 shows one embodiment of the mother-nucleus modified compounds of rapamycin produced by the present invention.

### DESCRIPTION OF EMBODIMENTS

The present invention is described in detail in the following.

The present invention provides a method for producing a modified compound, comprising the following steps (hereinafter sometimes to be referred to as “the method of the present invention”):

(1) a step of cleaving in vitro using CRISPR/Cas9 system, a target site in a gene cluster involved in the biosynthesis of a compound,
(2) a step of linking in vitro using Gibson assembly, the gene cluster cleaved in step (1) and a polynucleotide for modification, and
(3) a step of expressing the modified gene cluster obtained in step (2) in a microorganism expression system.

According to the present invention, a compound having a modified mother nucleus can be produced extremely efficiently. A modified compound that can be produced by the present invention includes compounds having a molecular weight of not more than about 4000. Such compounds can be divided into low-molecular-weight compounds and middle molecular compounds. In the present specification, the “low-molecular-weight compound” means a compound having a molecular weight of less than 400 (e.g., not more than 350, not more than 300, not more than 200, or not more than 100). In the present specification, the “middle molecular compound” means a compound having a molecular weight of about 400-4000 (e.g., molecular weight of 400-3500, 450-2500, 500-2000, or 500-1500). In one preferred embodiment, the method of the present invention is used for producing a middle molecular compound with a modified mother nucleus. Examples of the middle molecular compound include, but are not limited to, natural compounds represented by antibiotics (also referred to as “natural middle molecular compounds” in the present specification), peptides, nucleic acids, and the like. Examples of the natural middle molecular compound include compounds biosynthesized by type I PKS and NRPS. Specific examples of such compound include, but are not limited to, rapamycin (molecular weight 914.172 g/mol), actinomycin D (molecular weight 1255.438 g/mol), tacrolimus (molecular weight 804.018 g/mol), erythromycin (molecular weight 733.937 g/mol), pikromycin (molecular weight 525.683 g/mol), leucomycin A1 (molecular weight 785.969 g/mol), spiramycin (molecular weight 843.065 g/mol), tylosin (molecular weight 916.112 g/mol), and the like which are pharmaceutically useful as antibiotics.

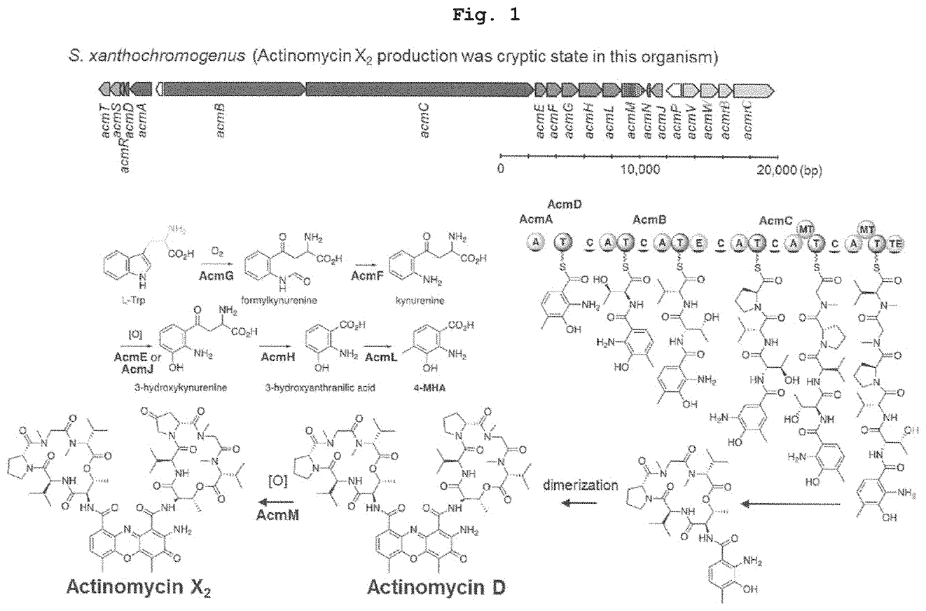

For many of the aforementioned natural middle molecular compounds, their gene cluster information involved in the biosynthesis has been known. For example, it is known that 17 genes (acmT, acms, acmR, acmD, acmA, acmB, acmC, acmE, acmF, acmG, acmH, acmL, acmJ, acmP, acmW, acmrB, acmrC) of Streptomyces parvulus are involved in the biosynthesis of actinomycin D. It is known that 20 genes (acmT, acmS, acmR, acmD, acmA, acmB, acmC, acmE, acmF, acmG, acmH, acmL, acmM, acmN, acmJ, acmP, acmV, acmW, acmrB, acmrC) of Streptomyces xanthochromo genus are involved in the biosynthesis of actinomycin X₂ (FIG. 1). Furthermore, it is known that 3 genes (rapA, rapB, rapC) of Streptomyces hygroscopicus are involved in the biosynthesis of rapamycin (FIG. 1 in non-patent document 2, etc.). When the information of a gene cluster involved in the biosynthesis of a target middle molecular compound is not known, a biosynthesis gene cluster may be identified using a method known per se. In one embodiment, a draft genomic data of a microorganism producing the target middle molecular compound is obtained, and the gene cluster region considered to be involved in the biosynthesis is assumed based on the structure of the target middle molecular compound, and the like. Then the candidate region of the assumed gene cluster is inserted into an expression vector such as BAC vector and the like. The obtained vector is introduced into a suitable microorganism expression system, a synthase group encoded by the candidate region is expressed in a microorganism, and a compound biosynthesized by the synthase group is produced by the microorganism. The structure of the produced compound is confirmed by a method known per se such as UV spectrum, NMR, and/or mass spectrometry or the like, whereby the gene cluster involved in the biosynthesis of the target middle molecular compound can be identified.

In step (1) of the method of the present invention, a target site in a gene cluster involved in the biosynthesis of a middle molecular compound is cleaved in vitro using CRISPR/Cas9 system. The CRISPR/Cas9 system used in the method of the present invention is not particularly limited as long as a desired target site of a gene cluster involved in the biosynthesis of a middle molecular compound can be accurately cleaved, and any type of CRISPR/Cas9 system may be used. The CRISPR protein (also called CRISPR effector protein, etc.) used in the method of the present invention is not particularly limited as long as it belongs to the CRISPR system and, for example, Cas9 can be recited as an example. Examples of the Cas9 include, but are not limited to, Cas9 derived from Streptococcus pyogenes (SpCas9), Cas9 derived from Streptococcus thermophilus (StCas9), and the like. In the present specification, the CRISPR protein also includes Cpf1 (CRISPR from Prevotella and Francisella 1) and the like. These CRISPR proteins may have a modified amino acid sequence or any modification as long as they can accurately cleave the target site of interest. The target site of the gene cluster cleaved by the CRISPR protein may be one or more (1, 2, 3, 4, or more). As shown in the Examples described later, the number of the target sites is generally two when the sequence of the nucleotide for modification is appropriately designed.

In the CRISPR/Cas9 system, a guide RNA (gRNA) or a single-stranded guide RNA (sgRNA) for recruiting a CRISPR protein into the target site may be designed to introduce a mutation that affords an intended modification into a gene cluster. A plurality of examples of methods for designing sgRNA and the like are specifically shown in the Examples described below, and those skilled in the art can design an appropriate sgRNA by referring to them.

The conditions for cleaving a gene cluster involved in the biosynthesis of a middle molecular compound in vitro using the aforementioned CRISPR/Cas9 system are not particularly limited as long as the aforementioned two DNA fragments can be linked and any conditions may be adopted. In the method of the present invention, when a commercially available CRISPR/Cas9 system is used, the manufacturer's recommended cleavage conditions can be adopted. A fragment of a gene cluster involved in the biosynthesis of a middle molecular compound which is cleaved at desired target site by the CRISPR/Cas9 system can be recovered and purified by a method known per se.

In one embodiment, a gene cluster involved in the biosynthesis of a middle molecular compound may be inserted into an expression vector in advance in consideration of step (3) of the method of the present invention. Such expression vector may be any as long as the full-length of the gene cluster involved in the biosynthesis of a middle molecular compound can be inserted. Examples of such expression vector include Cosmid vector, BAC vector, YAC vector and the like. Considering a general nucleotide length of a gene cluster involved in the biosynthesis of a middle molecular compound (50 kbp or more), and some exceed the upper limit of insert length (about 40 kbp) that the Cosmid vector can carry, a BAC vector or a YAC vector, which are expression vectors that can carry longer inserts, may be preferred, and a BAC vector is particularly preferred. In consideration of step (3) of the method of the present invention, the expression vector is sometimes more preferably of a chromosome-integrated type. In one preferred embodiment of the method of the present invention, the expression vector is a chromosome-integrated BAC vector.

A gene cluster involved in the biosynthesis of a middle molecular compound can be inserted into an expression vector by a method known per se. A case using a BAC vector is explained briefly in the following. A microorganism having a desired gene cluster in the genome (e.g., actinomycetes) is proliferated by a culture method known per se. The proliferated microorganisms are embedded in a gel containing a substance that digests the cell wall of the microorganisms (e.g., actinomycete) such as Lysozyme, SDS, Proteinase K and the like, and a restriction enzyme that can produce a desired DNA fragment. The cell wall of the microorganism is lysed in the gel, and the genome contained therein is cut by the restriction enzyme into DNA fragments of an appropriate size. The genomic fragments are then recovered by a method known per se and separated by size using pulsed field electrophoresis. DNA fragments of the desired size is extracted and purified from the gel. A BAC vector into which a gene cluster involved in the biosynthesis of a middle molecular compound has been inserted can be prepared by ligating the obtained DNA fragments to the BAC vector by a method known per se.

In step (2) of the method of the present invention, the gene cluster cleaved in step (1) and a polynucleotide for modification are linked using Gibson assembly in vitro.

In the present specification, the “polynucleotide for modification” means a polynucleotide capable of introducing the desired modification into a gene cluster involved in the biosynthesis of a middle molecular compound. The nucleotide sequence of the gene cluster is modified by the polynucleotide for modification. As a result, the functional “domain” and/or “module” composed of multiple domains of a biosynthetic protein of medium molecules encoded by the gene cluster is modified. This causes modification of the biosynthetic pathway of the middle molecular compound and results in the creation of a modified middle molecular compound. The nucleic acid sequence of the polynucleotide for modification may be appropriately determined according to the type of intended modification of the medium molecules, as exemplified in a plurality of examples described later. Examples of the type of modification include, but are not limited to, addition, deletion, or substitution of one or more amino acid residues in the amino acid sequence in one or more domains, addition, deletion, substitution of one or more domains or modules, and the like.

The method for preparing the desired polynucleotide for modification is not particularly limited, and polynucleotide can be prepared by using a method known per se. In one embodiment, PCR primers having nucleotide sequences that can introduce a desired mutation into the aforementioned gene cluster and, if necessary, enable ligation by Gibson assembly with the fragment of the gene cluster after cleavage obtained in step (1) are designed, and PCR is performed using an appropriate template (e.g., a gene cluster involved in the biosynthesis of a wild-type middle molecular compound or a fragment thereof, etc.), whereby the desired polynucleotide for modification can be prepared.

In the method of the present invention, the fragment of the gene cluster obtained in step (1) and a polynucleotide for modification are linked in vitro using Gibson assembly. The conditions used for Gibson assembly are not particularly limited as long as the aforementioned two DNA fragments can be linked and may be any. The Gibson assembly can be performed under the manufacturer's recommended conditions using a kit and the like commercially available from reagent manufacturers such as New England BioRabs Japan, and the like.

By this step (2), a polynucleotide encoding a biosynthesis protein capable of producing a middle molecular compound having a desired modification, or an expression vector having the polynucleotide inserted thereinto are prepared.

In step (3) of the method of the present invention, the modified gene cluster obtained in step (2) is expressed in a microorganism expression system. When the obtained modified gene cluster is not inserted into an expression vector, the modified gene cluster is first inserted into an expression vector by using the method described above or the like. The expression vector into which the modified gene cluster is inserted is introduced into a microorganism of an appropriate microorganism expression system. The microorganism expression system that can be used in the method of the present invention may be any system as long as it can efficiently produce a middle molecular compound having a desired modification. In one embodiment, such microorganism expression system may be a heterologous expression system (i.e., expression system using microorganism strain other than microorganism from which gene cluster is derived). As a host strain for heterogeneous expression of microorganisms preferably used in the method of the present invention, Streptomyces lividans or SUKA strain which is a chromosome-large deletion strain of Streptomyces avermitilis developed by the present inventors can be used. Streptomyces lividans has been reported to secrete heterologous proteins into culture supernatants. The SUKA strain is a variant in which the chromosome of S. avermitilis is reduced to about 80% of that of a wild-type strain by large reconstruction in genome in order to maximize the substance production capacity of S. avermitilis. The SUKA strain lacks all biosynthesis gene groups of the major products of S. avermitilis including avermectin, and scarcely produces secondary metabolic products in a common culture. It has been reported that the SUKA strain carries out production of a biosynthesis gene group of various secondary metabolic products by introducing the gene into the SUKA strain of S. avermitilis (Proc Natl Acad Sci USA. 2010 Feb. 9; 107(6):2646-51, ACS Synth Biol. 2013 Jul. 19; 2(7):384-96, J Ind Microbiol Biotechnol. 2014 February; 41(2):233-50). In addition, an extremely simplified secondary metabolism profile of the SUKA strain is preferable in that it enables easy analysis and purification of the target compound, in addition to high substance productivity. While the SUKA strain includes SUKA17, SUKA22, SUKA34, SUKA54 and the like, any of these may also be used. The SUKA17 strain is registered under Deposit No. “JCM18251” at RIKEN BioResource Center.

The expression vector prepared in step (2) may be introduced into Streptomyces lividans or SUKA strain by a method known per se. It is known that the introduction efficiency of a huge DNA molecule into S. avermitilis is low. As a method for compensating for this shortcoming, a method utilizing, as a vector, the linear plasmid SAP1 (94287 bp) possessed by S. avermitilis is preferably used. It is known that SAP1 is easily transferred between the genus Streptomyces bacteria by conjugational transfer and is stably retained in cells. Therefore, first, a BAC vector is introduced into Streptomyces lividans, which has a relatively high introduction efficiency of a huge DNA molecule, such that the vector is incorporated into SAP1. The obtained S. lividans is used as a donor strain and conjugated with the SUKA strain which is a recipient strain. By conjugation, the BAC vector incorporated into SAP1 is transferred to the SUKA strain by conjugational transfer and is stably maintained. By using such method, a BAC vector into which a gene cluster involved in the biosynthesis of a medium molecule having a desired modification has been inserted can be introduced into the SUKA strain highly efficiently.

In one preferred embodiment of the present invention, a middle molecular compound having a desired modification can be efficiently produced and recovered by culturing a SUKA strain with a BAC vector introduced therein by a method known per se.

The present invention is explained more specifically in the following Examples; however, the present invention is not limited at all by these examples.

### EXAMPLE

### [Example 1] Mother Nucleus Modification of Actinomycin X2

In NRPS and type I PKS compounds, homologous recombination easily occurs because of the gigantic size of the biosynthesis gene groups thereof, the repeat reactions in the generating process of the mother nucleus, and the sequence repeats in the mother nucleus biosynthase genes thereof. In fact, modification of the region encoding the production of the polyketide part of the biosynthesis gene group of type I PKS compounds is extremely difficult, and recombination occurs in unintended homologous regions. Therefore, it is judged that a method using homologous recombination is extremely inefficient for gene editing of these compound groups.

On the other hand, the present inventors have conducted intensive studies and developed a heterologous expression system of the biosynthesis gene group of the secondary metabolic products in many actinomycetes (Actinomycetales actinomycetes). In the method developed by the present inventors, even a huge biosynthesis gene cluster with a full-length of 60 kbp or more that encodes NRPS and biosynthases of polyketide compound can be cloned by using a Streptomyces chromosome-integrated BAC vector. The obtained BAC clone is introduced most efficiently into S. lividans and can transform it. However, the introduced huge biosynthesis gene cluster often may not be expressed; in particular, the expression of type I PKS biosynthesis gene cluster is inefficient, and accumulation of metabolic products produced by the biosynthase is often not confirmed. On the other hand, the genome-reduced strain of S. avermitilis (i.e., SUKA strain) that do not produce major metabolic products showed no problem in the introduction of DNAs of up to about cosmid clone (50 kbp), but it showed a problem in introducing a DNA larger than this. However, the expression of biosynthesis gene clusters contained in the introduced DNA fragments was often very efficient, and good production of metabolites could be confirmed. Thus, a series of methods for introducing BAC clones containing the above-mentioned huge DNA fragment via S. lividans and confirming the product have been established. This made it possible to use BAC clone into which an intact gene cluster of NRPS and polyketide compound is inserted, in an efficient microorganism expression system. By modifying these NRPS and type I PKS compound biosynthesis gene clusters, the biosynthase encoded by the modified gene cluster can be efficiently expressed in the microorganism expression system by the above-mentioned method. Therefore, theoretical prospects for the creation of non-natural middle molecular compounds were established. Thus, a novel technique that can afford a non-natural metabolic product was constructed by modifying in vitro a full-length biosynthesis gene cluster contained in a huge DNA fragment and expressing a gene cluster modified using a microorganism heterologous expression system developed by the present inventors. In the following, the method of the present invention is specifically explained by using an example in which an actinomycin X2 gene cluster is modified in vitro to obtain actinomycin D as a modified middle molecular compound.

Repeat reactions occur in the production process of a compound biosynthesized via NRPS and type I PKS. Therefore, sequence repeats exist in the process of catalyst reaction in the crude reaction thereof, which induces unintended recombination in the general modification by homologous recombination, and finally, the production of the desired compound cannot be achieved. In addition, it is necessary to cleave at a specific position on a huge DNA fragment, and to accurately and efficiently ligate a DNA fragment obtained by editing the cleaved fragment. For these purposes, a BAC clone containing a full-length biosynthesis gene cluster was used, and a method including a combination of cleavage by CRISPR/Cas9 in a test tube, and Gibson assembly to link and repair based on the cleaved fragment was established.

In the biosynthesis of Actinomycin X2, 4-methyl-3-hydroxyanthranilic acid (4-MHA) is produced from tryptophan via several reaction steps. This is activated by specific peptidyl carrier proteins and adenylating enzymes, 4-MHA-Thr-Val-Pro-Gly-Val (SEQ ID NO: 1) is produced by two huge non-ribosomal multifunctioning enzymes (actinomycin synthesizing NRPS, AcmC and AcmD), the TE domain on the C-terminal side of AcmD hydrolyzes its thioester from the PCP domain and it forms a lactone with the hydroxyl group of Thr to produce precursor A. This precursor forms a dimer and produces actinomycin D. In the final step, AcmM, which is cytochrome P450, oxidizes Pro residue to produce actinomycin X2. Therefore, actinomycin D is expected to be accumulated in a culture medium by inactivating the reaction of AcmM in the final step (FIG. 1).

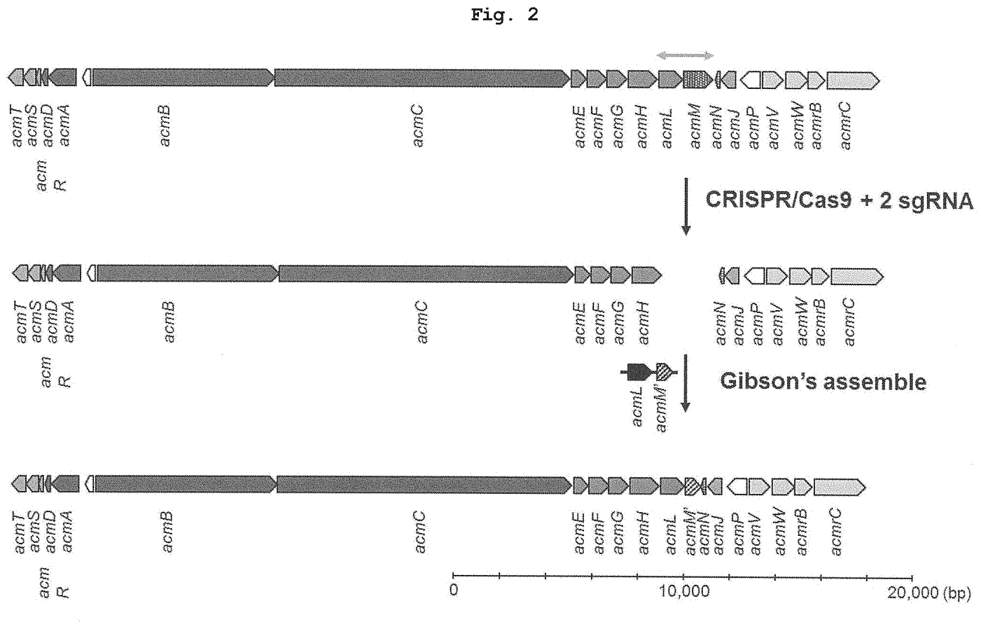

While the soil-isolated actinomycete Streptomyces xanthochromogenes is a strain isolated as a reductinomycin-producing bacterium, it was found to possess a biosynthesis gene group of actinomycin by genome analysis. Therefore, as a result of culturing under various culture conditions, an extremely small amount of actinomycin X2 could be detected. Furthermore, when a BAC clone containing the same full-length gene group was subjected to a heterologous expression system with S. avermitilis SUKA54 strain, a production amount of 1.1-1.6 g/L could be confirmed. Therefore, actinomycin D alone could be accumulated by gene editing to inactivate acmM gene from a BAC clone containing the above-mentioned biosynthesis gene group. Considering the arrangement and transcription direction of the genes in the biosynthesis gene group, in FIG. 2, the production of the actinomycin skeleton includes transcription in the right direction from acmB to acmM and transcription in the opposite direction from acmP to acmN. It is therefore expected that these transcriptions in both directions will be terminated by mutual transcriptions between acmM and acmN from the both directions, and editing of acmM without failing the balance of respective transcriptions was considered to be essential. The acmM is a gene encoding cytochrome P450. Thus, gene editing was performed such that an inactive enzyme in which several amino acids on the N- and C-terminal sides are deleted from the cysteine residue, which is the active center of cytochrome P450, is transcribed and translated (FIG. 2).

### Preparation of Recombinant BAC Clone

A strain of E. coli DH10B into which pKU508acmCW was introduced was transplanted into 500 mL of L broth (containing 1% tryptone, 0.5% yeast extract, 0.5% NaCl, pH 7.5; 25 μg/mL apramycin) and cultured overnight at 37° C. The bacterial cells were collected by centrifugation (5,000 rpm, 10 min), suspended in 100 mL of TE (10 mM Tris-HCl, 1 mM EDTA, pH 8.0), and then collected again by centrifugation. The bacterial cells were suspended in 45 mL of TE, 35 mL of alkali solution I (1% sodium dodecyl sulfate; SDS, 0.2 N NaOH) was added, and the mixture was mildly mixed at room temperature for 15 min. To a viscous, slightly cloudy solution was added 21 mL of neutralizing solution (prepared by sequentially adding 480 mL of 5M potassium acetate solution, 320 mL of acetic acid, 99 mL of phenol, 0.1 g of 8-hydroxyquinoline, 99 mL of chloroform, 2 mL of isoamyl alcohol, pH approx. 5.0), and the mixture was gently suspended to allow for precipitation of denatured high molecular weight DNA. The precipitate and supernatant were separated by centrifugation (5,000 rpm, 10 min), the obtained supernatant was placed in a new tube, 10 mL of TE and 56 mL of 2-propanol were added, and the mixture was allowed to stand at room temperature for 5 min. The obtained precipitate was collected by centrifugation (5,000 rpm, 10 min), washed with 70% ethanol, and collected again by centrifugation (5,000 rpm, 10 min). The obtained precipitated DNA was dissolved in 25 mL of STE (25 mM Tris-HCl, 25 mM EDTA, 0.3 M sucrose, pH 8.0), and RNase A was added to 20 μg/mL. The mixture was incubated for 60 min at 37° C. to degrade RNA. After completion of the reaction, 12.5 mL of alkali solution II (1% SDS, 0.3 N NaOH) was added, and the mixture was mildly mixed for 10 min. To this mixture was added 15 mL of a phenol:chloroform solution (8-hyxroxyquinoline was dissolved in phenol:chloroform=1:1 to 0.1%), and the mixture was mildly mixed for neutralization. After separation by centrifugation (5,000 rpm, 10 min), the supernatant was transferred to a new tube. To this supernatant were added 3.75 mL of 3M sodium acetate and 37.5 mL of 2-propanol, they were mixed well and left at room temperature for 5 min. Precipitated DNA was collected by centrifugation (5,000 rpm, 10 min), washed with 25 mL of 70% ethanol, and then collected by centrifugation (5,000 rpm, 10 min). The precipitated DNA was dissolved in 25 mL of TE, 12.5 mL of PEG solution (30% polyethylene glycol #6,000, 1.5M NaCl) was added, they were mixed well and left at room temperature for 15 min. The precipitated DNA was collected by centrifugation (5,000 rpm, 10 min), washed with 50 mL of 70% ethanol, and collected again by centrifugation. After evaporation of ethanol, the residue was dissolved in 3 mL of TE, and 3 g of CsCl was further added and dissolved therein. To this solution were added 0.15 mL of 10 mg/mL ethidium bromide solution and 0.06 mL of 25% lauroyl sarcosinate, and the mixture was dispensed into a Beckman ultracentrifugation tube (OptiSeal No. 361621) and further filled with a CsCl solution (5 g CsCl, 5 mL TE). The tube was placed in a TLA 100.4 rotor and ultracentrifuged at 75,000 rpm for 4 hr and at 55,000 rpm for 12 hr to isolate pKU508acmCW from the chromosome fragment. After completion of the ultracentrifugation, the tube was irradiated with UV light at 365 nm. Of the two DNA bands emitting fluorescence, the lower DNA band was collected with a syringe equipped with a 19-gauge needle. TE saturated n-butanol was added to the dispensed solution and ethidium bromide was extracted. This operation was repeated 3-4 times to completely remove ethidium bromide in the solution. A 3-fold amount of TE and further 6-fold amount of ethanol were added to the solution after the removal of ethidium bromide, and the mixture was left standing at room temperature for 15 min to allow for precipitation of plasmid DNA. The precipitates were collected by centrifugation (5,000 rpm, 10 min) and washed with 70% ethanol. Ethanol was removed, and the residue was dissolved in an appropriate amount of TE. Approximately 50-100 μg of pKU508acmCW (SEQ ID NO: 2) could be obtained by culturing on the above-mentioned scale.

Preparation of sgRNA

The region from acmL to acmM of the Actinomycin biosynthesis gene group was cleaved with CRISPR/Cas9, and an artificially produced “acmL-inactive acmM gene” was ligated to this part by Gibson assembly. Cas9 nuclease recognizes a DNA sequence complementary to the region encoded by sgRNA in coexistence with sgRNA, and performs double strand cleavage. The sequence 5′-ACCTCACCACCCACCCGATA-3′(SEQ ID NO: 4) (hereinafter PAM sequence; cGG) which is from 29921 bases to 29940 bases upstream of acmL in the full-length sequence (SEQ ID NO: 3) of the Actinomycin biosynthesis gene cluster, and the sequence 5′-GCGGCCCCTGTCCGCGACCG-3′ (SEQ ID NO: 5) (5′-side PAM sequence of reverse strand; tCC) which is from 32314 bases to 32333 bases downstream of acmM were used as the target sequences. As the template nucleotide required for the preparation of sgRNA, a nucleotide containing, from the 5′ side, T7 promoter sequence (5′-TTCTAATACGACTCACTATA-3′ (SEQ ID NO: 6)), target sequence (5′-ACCTCACCACCCACCCGATA-3′ (SEQ ID NO: 7) or 5′-GCGGCCCCTGTCCGCGACCG-3′ (SEQ ID NO: 8)), and a sequence complementary to the loop structure part on the 3′-side of sgRNA (5′-GTTTTAGAGCTAGA-3′ (SEQ ID NO: 9)) was used. sgRNA can be efficiently synthesized by inserting a single base G between the T7 promoter sequence and the target sequence. From the above, acmL upstream primer (5′-TTCTAATACGACTCACTATAgACCTCACCACCCACCCGATAGTTTTAGAGCTAGA-3′(SEQ ID NO: 10) and acmM downstream primer (5′-TTCTAATACGACTCACTATAgGCGGCCCCTGTCCGCGACCGGTTTTAGAGCTAGA-3′(SEQ ID NO: 11)) were prepared.

A kit of New England Biolabs, EnGen sgRNA synthesis kit, was used for preparation of sgRNA synthesis. Milli-Q water (RNase-free) (3 μL), 2-fold concentration of sgRNA reaction mixture (10 μL), acmL upstream or acmM downstream primer (1 μM) (5 μL), sgRNA enzyme mixture (2 μL) were mixed and reacted at 37° C. for 30 min. After completion of the reaction, Milli-Q water (RNase-free) (30 μL) was added, 2 μL DNase I (10 mg/mL) was added, and the mixture was incubated at 37° C. for 15 min to degrade DNA. A 25 μL solution of acidic phenol-chloroform (phenol:chloroform=1:1 was saturated with distilled water) was added and mixed well to denature the enzyme. The mixture was separated into two layers by centrifugation (14,600 rpm, 5 min). The upper aqueous phase was transferred to a new tube, 5 μL of 3M sodium acetate and 125 μL of ethanol were added. They were mixed well and left at −20° C. for 30 min. RNA was precipitated by cooling (4° C.) centrifugation (14,600 rpm, 5 min), and the precipitated RNA was washed with 70% ethanol and recovered by centrifugation (14,600 rpm, 5 min). The obtained sgRNA was dissolved in 25 μL of DNase-free water.

Cleavage of pKU508acmCW by Cas9 Nuclease

For cleavage at specific locations upstream of acmL of pKU508acmCW and downstream of acmM, RNase-free distilled water (20 μL), 10-fold concentration of Cas9 buffer (3 μL), two types of sgRNA (300 nM) (3 μL) prepared above, Cas9 nuclease (M0386S manufactured by NEB; 1 μM) (1 μL) were added, and the mixture was reacted at 25° C. for 10 min. Thereafter, the pKU508acmCW solution (5 nM) (3 μL) purified above was added and the mixture was incubated overnight at 37° C. The next day, RNase-free distilled water (23 μL), 10-fold concentration of Cas9 buffer (3 μL), the above-mentioned sgRNA (300 nM) (3 μL), Cas9 nuclease (1 μM) (1 μL) were added, and the mixture was incubated at 37° C. for 2 hr to completely cleave them. To the reaction mixture was added 30 μL of phenol.chloroform to discontinue the reaction, and the mixture was separated into the aqueous phase and the organic phase by centrifugation (14,600 rpm, 5 min). The upper aqueous phase was transferred to a new tube, 6 μL of 3M sodium acetate and 60 μL of 2-propanol were added. They were mixed well, left standing at room temperature for 5 min, and DNA was precipitated by centrifugation. The precipitate was washed with 70% ethanol, the ethanol was removed, and the precipitate was dissolved in 10 μL of 0.1×TE. To confirm whether the above-mentioned cleavage by Cas9 was sufficient, a part (0.25 μL) of the sample dissolved in 0.1×TE was electroporated into E. coli DH10B. If cleavage is sufficient, pKU508acmCW changes from a cyclic structure to a linear structure and cannot transform E. coli. As a result, it was confirmed that the number of transformants was not more than 10.

Production of Polynucleotide for Modification (acmL-acmM (Active Center Deletion Type))

Using pKU508acmCW as a template, acmL-acmM (active center deletion type) was prepared by two-step PCR. In the acmM region, a fragment was constructed in which the 216th amino acid to the 416th amino acid were deleted. 4 μL of 5-fold concentration of Q5 Reaction Buffer (manufactured by NEB), 0.4 μL of 10 mM dNTPs (dATP, dGTP, dTTP, dCTP), 1 μL of 10 μM primer 1 (5′-CTCGGGGCCACCGCCTTGCCCGCACCTCACCACCCACCCGATACGGAGTGC-3′ (SEQ ID NO: 12)), 1 μL of 10 μM primer 2 (5′-TCAGGGCCGGAGCCGAAGGCGAAGCGAGTTCAGCCGCCAACTGCCCGGATCGATCATTACGGG GAAGGAGTG-3′ (SEQ ID NO: 13)), 1 μL of pKU508acmCW (5 ng/μL), 4 μL of 5-fold concentration of Q5 High GC Enhancer (manufactured by NEB), 0.2 μL of Q5 High-Fidelity DNA polymerase (manufactured by NEB), 8.4 μL of sterilized water were added, denaturation was performed at 98° C. for 30 sec, and 25 repeats of the following cycles (98° C. for 10 sec, 60° C. for 30 sec, 72° C. for 20 sec) were performed, incubated at 72° C. for 2 min, and cooled to 4° C. After completion, a treatment with 0.15 μL of restriction enzyme DpnI (10 U/μL) was performed, and the template was removed. This amplified fragment was diluted 50-fold with sterilized water and used as the template for the second step in the PCR. In the second step of the PCR, 4 μL of 5-fold concentration of Q5 Reaction Buffer (manufactured by NEB), 0.4 μL of 10 mM dNTPS (dATP, dGTP, dTTP, dCTP), 1 μL of 10 μM primer 3 (5′-CTCGGGGCCACCGCCTTGCCCGCACCTCACCACCCACCCGATACGGAGTGCCCATGACCGACA CATCGCCGCTC-3′ (SEQ ID NO: 14)), 1 μL of 10 μM primer 4 (5′-ACAGGGGCCGCCCGATGCCGGGCGGCCCCTGTCCGCGATCAGGGCCGGAGCCGAAGGCG-3′ (SEQ ID NO: 15)), 1 μL of the above-mentioned diluted amplified fragment, 4 μL of 5-fold concentration of Q5 High GC Enhancer (manufactured by NEB), 0.2 μL of Q5 High-Fidelity DNA polymerase (manufactured by NEB), 8.4 μL of sterilized water were added, denaturation was performed at 98° C. for 30 sec, and repeats of the following cycles (98° C. for 10 sec, 60° C. for sec, 72° C. for 20 sec) were performed, incubated at 72° C. for 2 min, and cooled to 4° C. The base sequence of the obtained amplified fragment was confirmed and it was confirmed that the sequence shown below was obtained.

[Polynucleotide for Modification (acmL-acmM (Active Center Deletion Type))]

(SEQ ID NO: 16)
ctcGGGGCCACCGCCTTGCCCGCACCTCACCACCC
ACCCGATACGGAGTGCCCATGACCGACACATCGCC
GCTCACCACCGACGGCCTGGTACGGATCCTGTTCG
GCTCCTCGGCCTTCCAGATGCTCAACGCGGGCCGC
AACCTGGGTCTGTTCGCGCTGCTCAGTCGGCAGTC
CGGGCTGACCGCTCAGGAGATCGGACGTGAACTCG
GCCTGGCGGAACGCCCGGTGCAGATCCTGCTCCTG
GGTACTACAGCTTTGGGGCTGACGGTCCGTCAGGG
CGAGGGCTACCTCAATGCCGCTGTCCTGAACAACA
CGTTTGAGGACGGCACTTGGGAGATCATCGAGGAT
CTGATCGAGTACGAGGAGCGGATCGTCCGCCCCGC
CGAGGTGGACTTCACGGAGTCGCTGCGCCAAAACA
CCAACGTCGGGCTGCGCCGGATCGACGGGACCGGC
ACCGACCTCTACCACCGGCTGTCCGCGAACCCCGA
GCTTGAGCAGTTGTTCTACCGCTGCATGCGGTCCT
GGTCACGGCTGTCGAATCCCGTCCTGATCGAGCAG
GCCGACCTGACCGGGGTGCGCCGGGTCCTCGACGT
CGGGGGCGGCGACGGCGTGAACGCCATCGCCCTCG
CCCAGGCCAACCCCGGCGTCGAGTTCACCGTCCTC
GACCTCCCCGGCACCGTGGAGATCGCGCGACGCAA
GATCGCCGAGCACGGCTTGGCCGAACGGATCTCCG
TCCGGGCGGCGGACATCTTCGCCGACGACTACCCG
GCGGGGCACGACTGTGTGCTGTTCGCCAACCAGTT
GGTGATCTGGTCACCGGAGGAGAACGTGCGCCTGC
TGCGCAAGGCCCACGCGGCGCTGCCCGACGGCGGG
CGCGTGCTGGTGTTCAACGCCATGTCCGACGACAG
CGGCGACGGCCCCCTGTACGCGGCCCTGGACAACG
TGTATTTCGCGACGCTGCCGGCCGCGAGCAGCACC
ATCTACCGATGGGGCCAGTACGAGGAGTGGTTCGC
CGCGGCCGGGTTCGTGAAGCCCGAGCGGCTGCCGG
GCGGCCGGTGGACGCCGCACGGCGTGATCAGCGCG
GTCAAGTGACGCCCCAGCGAGAACCGGAGTCGGCC
ATGTCCCTCAAGTCCCACGACGCCCCGCCGACCGG
TGGGGCCGCGGCGTGCCCCGCCGGTCCGCACATGA
TGGATCCGGATCTGCTCCGGGACCCTTTCGGCGGC
TACGGCCGGCTGCGCGAACAGGACCCGGTGGTGCA
CGGCAGGTTCGTCGACGGCACCCCGGTGTGGTTCG
TGACCCGCTACGACGACGTCCGCGCGGCGCTGCGC
GACCCGCGGTTCGTCAACACCCCCTCCCACGTGCC
GGGCGAGAAGGGCGCGGACCCGCGCGAGGGCATGA
TGGAACTCCTCAAGGTCCCCGAGCATCTGCGCGGC
TATCTGCTCGGCTCCATCCTGGACAGCGACCCGCC
GGACCACCCGAGGCTGCGCCGCCTGGTGACCCGGG
CGTTCGCGGCCCGCCGGGTCCTGGATCTGCGCCAG
GACATCGAGCGGATCGCCGACCGGCTGCTGGCCGA
GCTGCCGCACCGGGAGGAGGACGGGACGGTCGATC
TCCTGGAGCACTTCGCGTATCCGCTGTCGATCACG
GTGATCTGCGAGCTCGTCGGCATCCCGGCGGCCGA
CCTCGGCCGGTGGCGGGAGTGGGGCGGCGACCTGG
TGTCGATGCGGCCCGAACGACTCCAGCACTCCTTC
CCCGTAATGATCGATCCGGGCAGTTGGCGGCTGAA
CTCGCTTCGCCTTCGGCTCCGGCCCTGATCGCGGA
CAGGGGCCGCCCGGCATCGGGCGGCCCCTGTCG

Linking of Cas9 Fragment of pKU508acmCW and Polynucleotide for Modification (acmL-acmM (Active Center Deletion Type)) by Gibson Assembly

pKU508acmCW fragment linearized using Cas9 and two kinds of sgRNAs was linked to a modified polynucleotide (acmL-acmM (active center deletion type)) having the aforementioned sequence by Gibson assembly. pKU508acmCW (about 1 μg) cleaved using Cas9 and sgRNAs and polynucleotide for modification (about 0.1 μg) were dissolved in sterile distilled water (10 μL), and mixed with 10 μL of 2-fold concentration of Gibson's mixture (10% polyethylene glycol #8000, 200 mM Tris-HCl (pH 7.5), 20 mM MgCl₂, 20 mM Dithiothreitol, 0.4 mM dNTPs (dATP, dGTP, dTTP, dCTP), 2 mM NAD⁺ 8 U/mL T5 exo nuclease, 8000 U/mL Taq DNA ligase, 50 U/mL Phusion DNA polymerase), and the mixture was incubated at 50° C. for 45 min. To digest a fragment not participated in the DNA fragment, 0.125 μL of T5 exo nuclease (10 U/μL) was added and the mixture was incubated at 37° C. for 1 hr. After completion of the reaction, the mixture was treated at 65° C. for 5 min to discontinue the reaction, and mixed with 2 μL of 3 M sodium acetate and 20 μL of 2-propanol. The mixture was left standing at room temperature for 5 min, and DNA was precipitated by centrifugation (14,600 rpm, 5 min). The precipitate was washed with 70% ethanol and dissolved in 10 μL of 0.1×TE.

E. coli DH10B was cultured in L broth (1% tryptone, 0.5% yeast extract, 0.5% NaCl, pH 7.5) at 37° C. and proliferated to OD₆₀₀=0.5-0.7. The bacterial cells were collected by centrifugation (5,000 rpm, 10 min), washed twice with cooled sterile distilled water, and collected by centrifugation. Finally, the bacterial cells were washed with cooled sterile 10% glycerol solution and suspended in 10% glycerol at a ratio of 1/200 of the culture medium. To this suspension (50 μL) was added the DNA fragment (5 μL) linked above, and introduced using Bio-Rad Gene Pulser with a pulse of 1.8 kV (25 μF, 2000) in a 1 mm gap cuvette. 1 mL of SOC was added, and the mixture was incubated at 30° C. for 90 min and cultured at 30° C. overnight in LA (L broth added with 1.5% agar) medium containing 25 μg/mL apramycin. The transformant produced the next day was transplanted into a 96 well plate containing 150 μL of L broth (containing 25 μg/mL apramycin), and cultured overnight at 30° C. After the completion of culture, PCR was performed using 12 types of mixed vertical series of culture medium contained in each well and 8 types of mixed horizontal series as templates and the following primers (forward: 5′-GATCGGTCTGTCGCCCCTCTACAC-3′ (SEQ ID NO: 17), reverse: 5′-GATACTCGGAGTTGGTGCCCGAAG-3′ (SEQ ID NO: 18)). In the case of a wild-type gene segment of pKU508acmCW, a fragment of about 2.7 kb is amplified. In the case of a fragment with desired modified nucleotide sequence linked thereto, an amplification fragment of about 2.1 kb is detected. Finally, 18 clones (pKU508acmCWΔacmM (SEQ ID NO: 19)) containing the desired DNA sequence could be obtained.

Production of Substance by Heterologous Expression of Actinomycin Biosynthesis Gene Cluster with Introduced Modification

Heterologous expression of Actinomycin biosynthesis gene cluster is scarcely observed in S. lividans. Therefore, heterologous expression by genetically-modified S. avermitilis (SUKA strain) was performed. However, since introduction of a DNA fragment exceeding 50 kb is not performed efficiently in S. avermitilis, S. lividans showing effective DNA introduction was used to introduce desired pKU508acmCWΔacmM into a transferable linear plasmid vector SAP1.13. S. lividans was transformed by a known method (Practical Streptomyces Genetics. Norwich, U.K.: The John Innes Foundation).

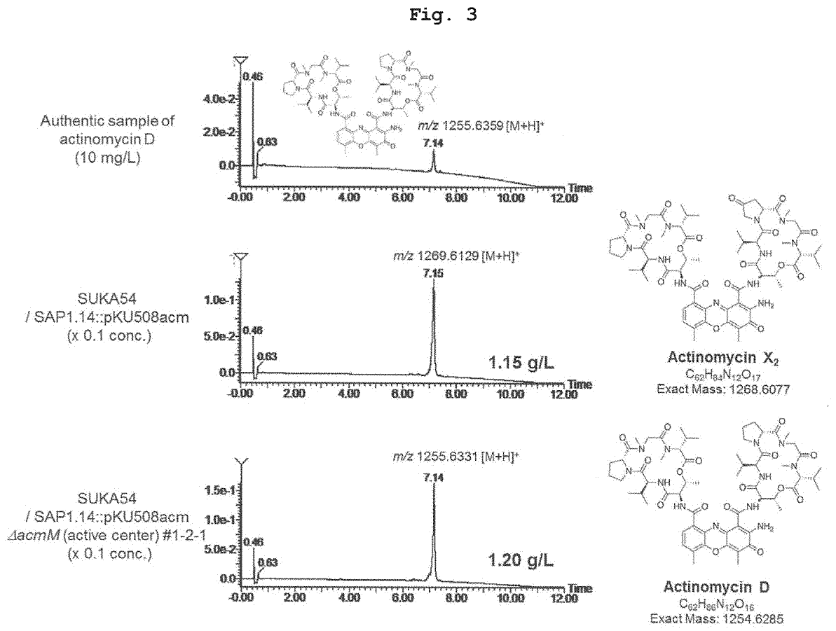

For heterologous expression of pKU508acmCWΔacmM obtained above in Streptomyces actinomycetes, the obtained gene edited clone was prepared from 50 mL of L broth. Using 0.5 μg of the obtained pKU508acmCWΔacmM, 0.5 mL of 25% polyethylene glycol #1,000 was added to 50 μL of protoplast of S. lividans TK24 ΔattB_{φC31} ΔattB_{TG1} ΔattB_{φBT1} ΔattB_{φK38-1}::aadA/SAP1.13, and the mixture was treated at room temperature for 1 min, and then 0.5 mL of P medium was added. This mixture (0.1 mL) was spread on 20 mL of R2YE agar medium, cultured at 30° C. for 18 hr, and 2.5 mL of soft agar medium (0.4 g Difco Nutrient broth, 0.5 g agar) containing 500 μg/mL apramycin and incubated at 45° C. was layered thereon. After the soft agar was solidified, it was cultured at 30° C. for another 4-6 days. The obtained transformants were cultured in SFM agar medium (20 g defatted soy flour, 20 g mannitol, 20 g agar were suspended in 1 L of ion exchange water, pH not adjusted) containing 25 μg/mL apramycin, at 30° C. for 4 days. The linear plasmid contained in each transformant was confirmed by CHEF electrophoresis, each spore suspension and spore suspension of S. avermitilis SUKA54 strain were applied onto SFM agar medium or M4 agar medium (10 g soluble starch, 1 g K₂HPO₄, 1 g MgSO₄.7H₂O, 1 g NaCl, 2 g (NH₄)₂SO₄, 2 g CaCO₃, 1 mL trace element solution (1 g FeSO₄.7H₂O, 1 g MnSO₄.4H₂O, 1 g ZnSO₄.7H₂O) were dissolved in 1 L of ion exchange water), 15 g agar was suspended in 1 L of ion exchange water, adjusted to pH 7.0), and mixed culture was performed. Spores were engrafted by incubating at 30° C. for 4-7 days, spores on the surface of the agar medium were scraped together with sterile distilled water, passed through sterile defatted cotton, and hyphae and agar medium were removed. The spores were spread on a YMS agar medium (4 g Yeast extract, 10 g malt extract, 4 g soluble starch, 20 g agar, adjusted to pH 7.4, sterilized in autoclave, MgCl₂ and Ca(NO₃)₂ were added to 10 mM, 8 mM, respectively) containing hygromycin B (100 μg/mL) which is a selection marker of S. avermitilis SUK54 strain, SAP1.13, and viomycin (30 μg/mL) and apramycin (25 μg/mL) which are selection markers of pKU508acmCWΔacmM, whereby a clone in which SAP1.13::pKU508acmCWΔacmM was conjugationally transferred from S. lividans TK24 ΔattB_{φC31} ΔattB_{TG1} ΔattB_{φBT1} ΔattB_{φK38-1}::aadA to S. avermitilis SUKA54 was obtained. The obtained conjugate was spread on a YMS agar medium containing 30 μg/mL viomycin and 25 μg/mL apramycin and cultured at 30° C. for 4 days for spores to engraft. The linear plasmid contained in each conjugate was confirmed by CHEF electrophoresis, and the conjugate having SAP1.13::pKU508acmΔacmM was confirmed. These spore suspensions were transplanted to 10 mL of a seed medium (5 g glucose, 15 g defatted soybean, 5 g yeast extract, pH 7.0) in a 50 mL large test tube, and shake cultured at 30° C. for 2 days to give a seed culture medium. 0.15 mL of the seed culture medium was transplanted into 15 mL of a production medium (60 g glucose, 2 g (NH₄)₂SO₄, 0.1 g MgSO₄.7H₂O, 0.5 g K₂HPO₄, 2 g NaCl, 0.05 g FeSO₄.7H₂O, 0.05 g ZnSO₄.7H₂O, 0.05 g MnSO₄.4H₂O, 2 g yeast extract, 5 g CaCO₃ were suspended in 1 L of ion exchange water and pH was adjusted to 7.0) in a 125 mL Erlenmeyer flask, and cultured at 28° C., 200 rpm for 5 days. After completion of the culture, an equal amount of methanol was added, and the mixture was shaken for 15 min for extraction. The bacterial cells were precipitated by centrifugation (3,000 rpm, 10 min), the supernatant was diluted 10-fold with methanol, and 5 μL thereof was used for analysis. The metabolic products contained in the culture medium were analyzed by Acquity ultraperformance LC system, Waters Xevo G2-S Tof. As analysis conditions, UPLC BEH C18 2.1φ×50 mm; 1.7 μm column was used, and elution was performed with a linear gradient of a 0.05% formic acid solution containing 5-95% acetonitrile as the mobile phase. In addition, actinomycin was quantified by calculating from the value of maximum absorption in the visible part obtained by analysis of a standard solution (10 mg/L) in which the standard product actinomycin D (manufactured by Sigma-Aldrich) was dissolved in methanol under the above-mentioned conditions. As shown in the Figure, S. avermitilis SUKA54 containing pKU508acmCW accumulated 1.15 g/L of actinomycin X2. On the other hand, S. avermitilis SUKA54 containing pKU508acmΔacmM obtained by gene editing produced actinomycin D at 1.20 g/L. Any components other than actinomycin D (including actinomycin X2) did not accumulate in this culture medium, and a gene-edited strain that selectively produces only actinomycin D could be obtained (FIG. 3).

### [Example 2] Mother Nucleus Modification of Rapamycin

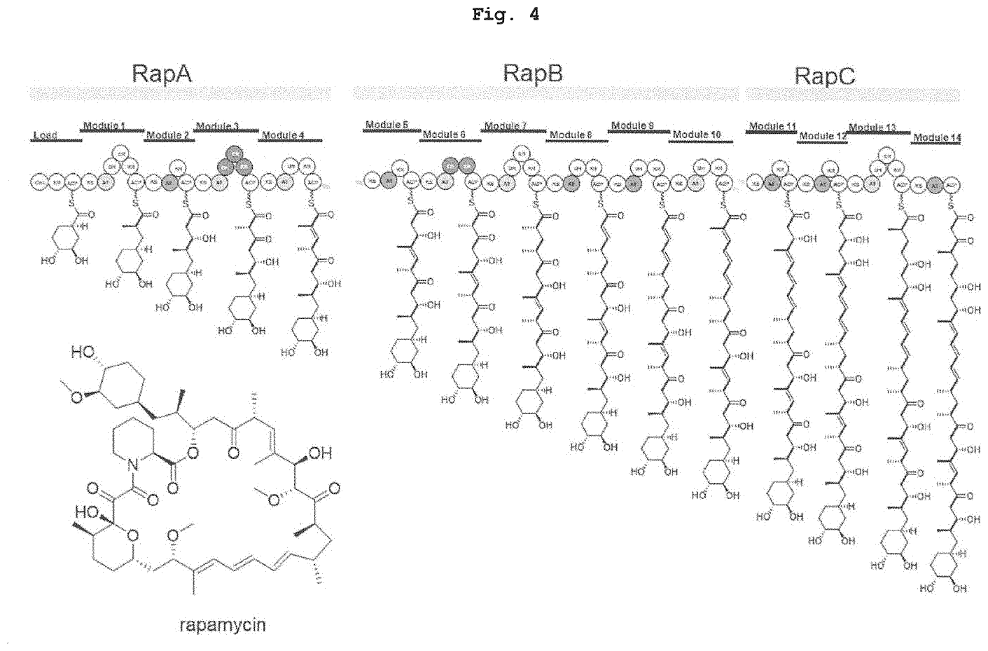

With regard to rapamycin, which is clinically applied as an immunosuppressant and an antitumor agent, it requires several years to produce a compound thereof by organic synthesis. Therefore, development of a derivative is difficult, although it is an important compound for clinical application. The biosynthesis gene cluster of rapamycin is 107.4 kb, and the BAC insert length used in this example is 156.6 kb, which is a huge gene. In addition to loading precursors, this biosynthesis gene cluster consists of 14 module groups each having extremely high homology (FIG. 4). Thus, it is not possible to accurately perform genetic modification by conventional methods, and it is not possible either to develop derivatives by biosynthesis.

By applying the cloning techniques for huge biosynthesis genes and the heterologous expression production techniques applying them developed by the present inventors, and adopting the latest gene manipulation techniques, a technique that enables modification of the mother nucleus of a complicated medium-molecular-weight natural compound such as rapamycin has been successfully developed. Rapamycin is a group of compounds called macrolides that are biosynthesized by a biosynthesis pathway called type I polyketide. In type I polyketide, the carbon chain is extended by each module, and the structure to be constructed is determined by the modified domain or gene sequence that constitutes the module. Therefore, the superiority of this technology development can be proved by four representative examples of mother nucleus modification technique shown below.

### [Example 2-1] Production of Modified Rapamycin (Double Bond-Adding Compound)

Macrolide compounds produced by actinomycetes have, in addition to the carbon chain extension domain, modifications by modified domains that significantly change the structure of each module, and the combination thereof makes it possible to construct modules having hydroxyl groups, double bonds, alkyl chains, or ketone group. Such module modification is a reaction that is impossible in organic synthesis, and enables development of derivatives that significantly changes the compound structure such as improvement of solubility. Therefore, as Example 2-1, a compound having a tetraene structure having one more double bond than the triene structure was created by mutating the modified domain of module 7 of rapamycin.

The following method (protocol) was used for cleavage by CRISPR/Cas9 and preparation of modified biosynthesis gene cluster by Gibson assembly.

1. sgRNA is prepared according to the protocol of EnGen sgRNA Synthesis Kit (NEB: E3322S).
2. BAC is cleaved with 20 μM Cas9 Nuclease according to the protocol of Cas9 Nuclease, S. pyogenes (NEB: M0386M) (BAC concentration is final 0.5 nM).
3. After phenol-chloroform treatment of 2, isopropanol precipitation is performed, washed with 70% ethanol, air dried, and dissolved in 10 μM 0.1×TE.
4. BAC 1 μl of 3, 100 ng/μl polynucleotide for modification 3 μl, 2-fold concentration of Gibson's mixture (see Example 1) 10 μl, water 6 μl are mixed and incubated at 50° C. for 50 min.
5. After phenol-chloroform treatment, isopropanol precipitation is performed, washed with 70% ethanol, air dried, and dissolved in 5 μM 0.1×TE.
6. Using total amount of 5, Escherichia coli NEB 10-beta is transformed by electroporation.
7. Hit clones are screened for by colony PCR.
8. Hit clones are cultured, BAC is extracted, target region is sequenced, and clone is confirmed.

For cleavage by CRISPR/Cas9 in Example 2-1, sgRNA produced by transcription from the following oligonucleotide with T7 RNA polymerase was used.

<sgRNA oligo>
rap_M7_DH_3′_sgRNA:
(SEQ ID NO: 20)
TTCTAATACGACTCACTATAGAGGTGCACGCTAGCGGACGAGTT
TTAGAGCTAGA
rap_M7_ER-KR_sgRNA:
(SEQ ID NO: 21)
TTCTAATACGACTCACTATAGCCGTTGGCGTCGAGTTGCTGGTTTTAGAG
CTAGA

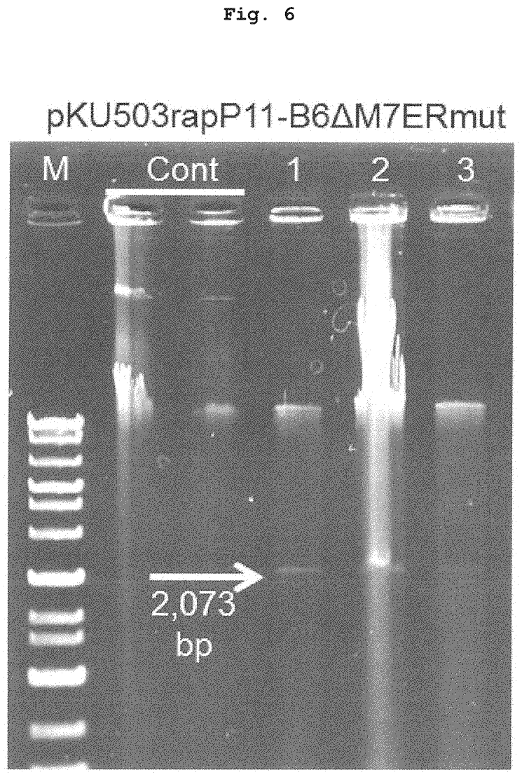

Using this sgRNA, the 2073 bp cleaved fragment shown in FIG. 6 was prepared by BAC cleavage by CRISPR/Cas9 reaction.

Then, preparation of altered biosynthesis gene by Gibson assembly was performed according to the following method.

### <Primer List>

Template: pKU503rapP11-B6 (BAC vector inserted with polynucleotide encoding rapamycin biosynthesis gene cluster: SEQ ID NO: 22)

### (1) Rap_M7ER_GG-SP_Left_Fw:

(SEQ ID NO: 23)
CGATGAGCTGGTGATCGAAACCCCGCTGCTGCTGCCGTCGTCCGCTA

### Rap_M7ER_GG-SP_Left_Rv:

(SEQ ID NO: 24)
CCATGCCGACAGGACTAGCGGCGGCGTGGATCAGCACGGAC

### (2) Rap_M7ER_GG-SP_Right_Fw:

(SEQ ID NO: 25)
GCCGCCGCTAGTCCTGTCGGCATGGCAGCCACCCAGATC

### Rap_M7ER_GG-SP_Right_Rv:

(SEQ ID NO: 26)
AACCACCGGTGACCAGAACCGTGCCGTTGGCGTCGAGTTGCTGAG

<Protocol>

1. Using primers (1) and (2), polynucleotide for modification is amplified in two divided fragments.
2. The two fragments of 1 which have been cut out from the gel and purified are mixed to give a template, and PCR is performed using the forward primer of (1) and the reverse primer of (2).
3. PCR product cut out from the gel is purified, and used as polynucleotide for modification.

Specific polynucleotide sequence for modification is as follows:

(SEQ ID NO: 27)
CGATGAGCTGGTGATCGAAACCCCGCTGCTGCTGC
CGTCGTCCGCTAGCGTGCACCTGTCCGTGTCGGTC
GGCGAGGCTGACGAATCCGGGCGCCGGGGTGTGAC
GGTCTTTTCCCGTGCGGATGGCGCCGACGCCTGGA
CTCGCCACGTTTCGGCCACGATCGGCGTCTCTGGC
GCTGCCCTCTCGCTGCCAGAGCTTGCTGCTTGGCC
TCCCGCACAGGCACAGCCGGTGGGCCTGGGCGATT
TCTACGACCGGCTGACCGGGGCCGGTTACGAGTAC
GGTCCCGCGTTCCAGGGGCTGCAGGCCGCGTGGCG
TGACGGGGACACCGTCTTCGCCGAGGTGGCCCTGG
CCGAGGAGCAGGCGGAGGAGGCGGCACGGTTCGCG
GTGCATCCGGCGCTGTTGGACGCCGCCTTGCACGC
CGGAATTCTGAACACACTCGACACCGCCGAGCAGG
GTGTGCGGCTGCCGTTCTCCTGGAACGGTGTCCAG
GTCCGGGCCACTGGCACGGCCACGCTACGCGTTGC
GATAACACCAGTGACGGACGGCTGGAGTGTGCGGG
TCGCCGACGACAGCGGCCGACCGGTGGCTACCGTC
GACTCGCTCGTAACGCGGCCGGTAACGGCCGACAC
GCTCGGTTCCGCTGCCGACGACCTGCTCACGGTGG
TCTGGACGGAGATCCCCACCCCCCAGCAGACCGGC
CTGAGCGTCGGCCGGTTCGAAGACCTGGCGGACGG
TGATGTGCCGGTGCCCGAGGTGGTGGTCTGCACCG
CACTCCCCGACAGCAGCGAGAACCCGCTAGCCCCG
CTGGATCCGCCGGATCCGCTGGTACAGACCCGCAC
GTTGACCACCCAGGTTCTCCAGGCAGTTCAGGCAT
GGCTGGCCGGGGAACGTTTCACCGACAGCACGCTG
GTCGTGCGGACCGGCACCGGGCTGGCCACCGCCGG
GGTGTCGGGTCTGATGCGGTCTGCCCAGTCGGAAC
ACCCCGGCCGGTTCGTCCTGGTGGAATGCGACGAC
AACCTCACCCTCCAGCAACTGGCCGCGACTGTCGG
GTTGGACGAGCCGCGGCTGCGGGTCTGCGACGGCC
GGTTCGAGGTACCGCGGCTGGCGCGGGCGAATACG
CCGGAAAGCAGCCCGCTCACGATTCCCGGGGATCG
TGCGTGGCTGCTGGAGCAGTCCCGCAGCGGAACCT
TGCGGGACCTCGCGCTGGTACCCGCCGAAACCGCC
GAACGGCCCCTGCAATCCGGTGAAGTACGAGTAGA
CGTACGCGCCGCAGGCCTGAACTTCCGCGATGTTC
TCATCGCGCTCGGCACTTACCCCGGTGAGGCTGTG
ATCGGGGCTGAGGCTGCGGGCGTGGTGCTCGAGGT
CGGTCCGGAGGTCCAGGATCTGGCCCCGGGAGACC
GAGTGTTCGGTCTTGTGGGCGGTGGGTTCGGGGCG
GTCGCGATCGCTGATCGCCGAATGCTGGGTGTGAT
TCCTGACGGGTGGTCGTTCACTACGGCGGCGTCCG
TGCCGGTTGTGTTCGCCACCGCGTATTACGGGCTG
GTGGATCTGGCCGGGCTGAGTGCGGGTGAGTCCGT
GCTGATCCACGCCGCCGCTAGTCCTGTCGGCATGG
CAGCCACCCAGATCGCCCGCCACCTCGGCGCGCGG
ATCTACGCGACGGCCAGCACCGGTAAGCAGCACGT
CCTGCGCGAGGCGGGTCTGGAGGATGCCCGGATCG
GGGACTCGCGTACCACTGGCTTCCGGGAAATGGTT
CTGGACACCACTGACAGCCGGGGTGTCGATGTCGT
CCTGAACTCCCTCAGCGGTGACTTTGTCGATGCTT
CGCTTGATCTGCTGCCTCGTGGTGGCCGGTTCGTC
GAGATGGGCAAGACCGACATCCGTGACCCGCACCA
GGTCACCGCCGACCGGCCGGGTACCAGCTACCAGG
CGTTCGATCTGATGGACGCCGGTCCGGACCGGCTG
CGGGAGATCATCGCCGATTTGCTCGCCCTGTTCGC
GCAGGGTGTGCTATTGCCCCTGCCGGTGCGGGCCT
GGGACATCCGTCAGGCCCGTGAGGCGTTCAGCTGG
ATGAGCCGTGCCCGCCACATCGGCAAGATCGTCCT
CACCGTCCCTCAGCAACTCGACGCCAACGGCACGG
TTCTGGTCACCGGTGGTT

Cleaved BAC (1 μl), 100 ng/μl polynucleotide for alteration (3 μl), 2-fold concentration of Gibson's mixture (see Example 1) (10 μl), water (6 μl) were mixed and incubated at 50° C. for 50 min to prepare a desired construct.

Introduction of the constructed mother nucleus modification construct into a host, conjugation with heterologous expression strain and the like were performed according to the method of Example 1. Introduction of the mother nucleus modified construct into the donor bacteria was confirmed by PCR using the primer sequences shown in the following paragraph.

### PCR Primer

| | | |
|---|---|---|
| rapF1 | AACAGCCGAAAGAAATGGCTGTGC | (SEQ ID NO: 28) |
| rapR1 | GGCCCTCTCGAACTTCCGTACCTC | (SEQ ID NO: 29) |
| rapF2 | GGTGGTTTCGTCATGCCTGTTCTG | (SEQ ID NO: 30) |
| rapR2 | GCTCTCCTTGAGCATCAGCCACTG | (SEQ ID NO: 31) |

In Example 2, the following 4 types of donor bacteria were produced:
S. lividans TK24 ΔattB_{φC31} ΔattB_{TG1} ΔattB_{φBT1} ΔattB_{φK38-1}::aadA/SAP1.11/SAP1.11:: pKU503rap4309
S. lividans TK24 ΔattB_{φC31} ΔattB_{TG1} ΔattB_{φBT1} ΔattB_{φK38-1}::aadA/SAP1.11/SAP1.11:: pKU503rapP11-B6ΔM7ERmut
S. lividans TK24 ΔattB_{φC31} ΔattB_{TG1} ΔattB_{φBT1} ΔattB_{φK38-1}::aadA/SAP1.11/SAP1.11:: pKU503rapΔM9AT::M6AT(m)
S. lividans TK24 ΔattB₉C31 ΔattB_{TG1} ΔattB_{φBT1} ΔattB_{φK38-1}::aadA/SAP1.11/SAP1.11:: pKU503rapΔM5ACP-M6KR

These were conjugated with the following recipient bacteria to obtain transformed strains that produce rapamycin with modified mother nucleus.
Streptomyces avermitilis SUKA54
Streptomyces avermitilis SUKA34

The transformed strains were cultured by a method similar to that in Example 1. After completion of the culture, the confirmation of the compound production by mass spectrometry was performed as follows.

### Preparation of Sample for Mass Spectrometry

n-BuOH (5 ml) was added to a culture medium (5 ml), extracted, and an extract (1.5 ml) was recovered, and dried to solidness. The dried solid sample was dissolved in 400 μl of DMSO solution, and 2 μl from the sample was analyzed under the following conditions.

### Mass Spectrometer, Column and Analysis Conditions Used

mass spectrometer
   LC/MS ACQUITY UPLC system (Waters, Taunton, Mass.), XevoG2 Tof system.
column
   ACQUITY UPLC BEH C18 column 1.7 μm, 2.1φ×100 mm (Waters, Taunton, Mass.),

Compound Detection Conditions

column temperature 55° C.

eluent

eluent A 0.1% formic acid aqueous solution

eluent B 0.1% formic acid acetonitrile

gradient conditions

Eluting time, 0-5 min

gradient concentration 5-100% eluate B, flow rate 0.8 ml/min

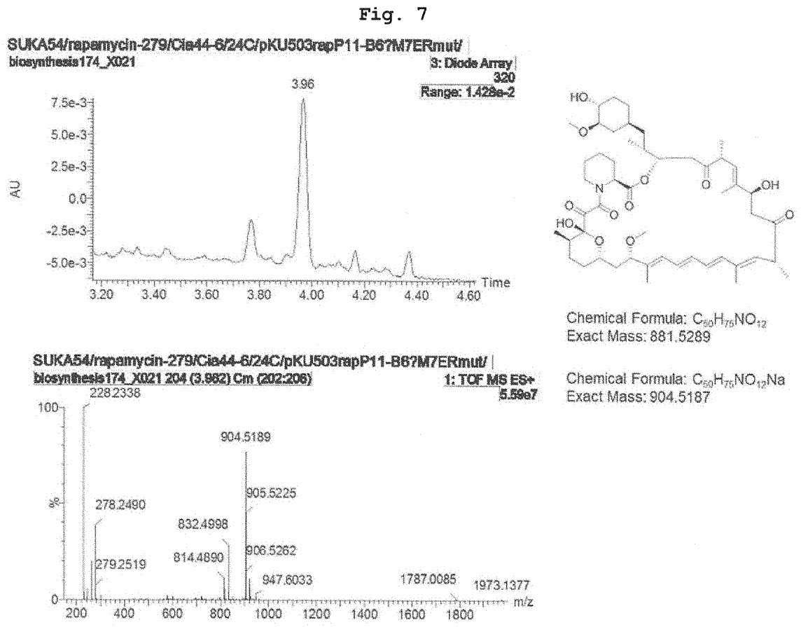

From the above results, novel mother nucleus modified rapamycin was detected as a peak of sodium-added type salt (FIG. 7, C₅₀H₇₅NO₁₂Na, measurement value: 904.5189, calculated value: 904.5187).

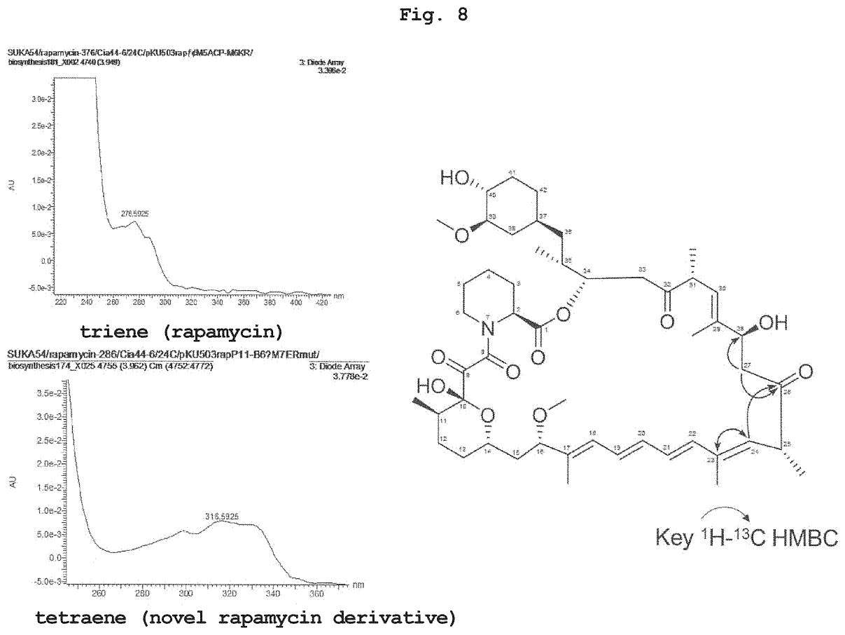

This structure was confirmed to have a tetraene structure as a result of the analysis by ultraviolet visible absorption spectrum (FIG. 8) and NMR (Table 1).

TABLE 1

| No. | δH | δC |
|---|---|---|
| 1 | — | 170.2 |
| 2 | 5.299 (m) | 52.7 |
| 3 | 1.863 (m), 2.323 (m) | 27.4 |
| 4 | 1.597 (m), 1.817 (m) | 21.2 |
| 5 | 1.613 (m), 1.814 (m) | 25.7 |
| 6 | 3.457 (m), 3.710 (m) | 44.7 |
| 7 | — | — |
| 8 | — | 167.2 |
| 9 | — | 195.3 |
| 10 | — | 99.8 |
| 11 | 2.103 (m) | 34.7 |
| 12 | 1.617 (m) | 27.8 |
| 13 | 1.41 (m), 1.686 (m) | 31.9 |
| 14 | 3.874 (m) | 67.7 |
| 15 | 1.709 (m), 1.826 (m) | 39.7 |
| 16 | 3.78 (m) | 85.0 |
| 17 | — | 137.1 |
| 18 | 6.25 (m) | 130.8 |
| 19 | 6.609 (m) | 128.6 |
| 20 | 6.53 (m) | 135.5 |
| 21 | 6.49 (m) | 129.3 |
| 22 | 6.34 (m) | 138.3 |
| 23 | — | 137.6 |
| 24 | 5.26 (m) | 132.6 |
| 25 | 3.72 (m) | 47.7 |
| 26 | — | 208.6 |
| 27 | 2.66 (m), 2.96 (m) | 47.4 |
| 28 | 4.54 (m) | 73.2 |
| 29 | — | 141.0 |
| 30 | 5.15 (m) | 126.0 |
| 31 | 3.47 (m) | 47.1 |
| 32 | — | 208.1 |
| 33 | 2.62 (m), 2.75 (m) | 43.1 |
| 34 | 5.22 (m) | 76.4 |
| 35 | 1.91 (m) | 34.8 |
| 36 | 1.22 (m), 1.37 (m) | 39.1 |
| 37 | 1.48 (m) | 34.1 |
| 38 | 0.74 (m), 2.15 (m) | 35.5 |
| 39 | 2.94 (m) | 85.5 |
| 40 | 3.31 (m) | 74.7 |
| 41 | 1.87 (m), 1.30 (m) | 33.2 |
| 42 | 1.02 (m) | 32.7 |
| 43 | 0.88 (d, 6.5) | 16.4 |
| 44 | 1.72 (s) | 10.4 |
| 45 | 2.02 (s) | 12.9 |
| 46 | 1.12 (d, 6.5) | 15.6 |
| 47 | 1.70 (s) | 11.6 |
| 48 | 1.04 (d, 6.5) | 15.9 |
| 49 | 0.94 (d, 6.5) | 16.1 |
| 50 | 3.37 (s) | 57.2 |
| 51 | 3.15 (s) | 56.0 |

### [Example 2-2] Production of Modified Rapamycin (Modification of Methyl Group Side Chain)

As the feature of the macrolide compounds produced by actinomycetes, whether or not a carbon chain has a side chain structure on the extended chain is determined by the gene when the carbon chain is extended. Since the presence or absence of this side chain significantly changes the structure of the whole compound, for example, based on the docking analysis with a target factor, a mother nucleus modification technique that fills an open space in order to achieve stronger binding is considered to be effective. Therefore, a mother nucleus modified compound was constructed as Example 2-2 to determine whether a side chain can be added or removed during carbon chain extension of rapamycin.

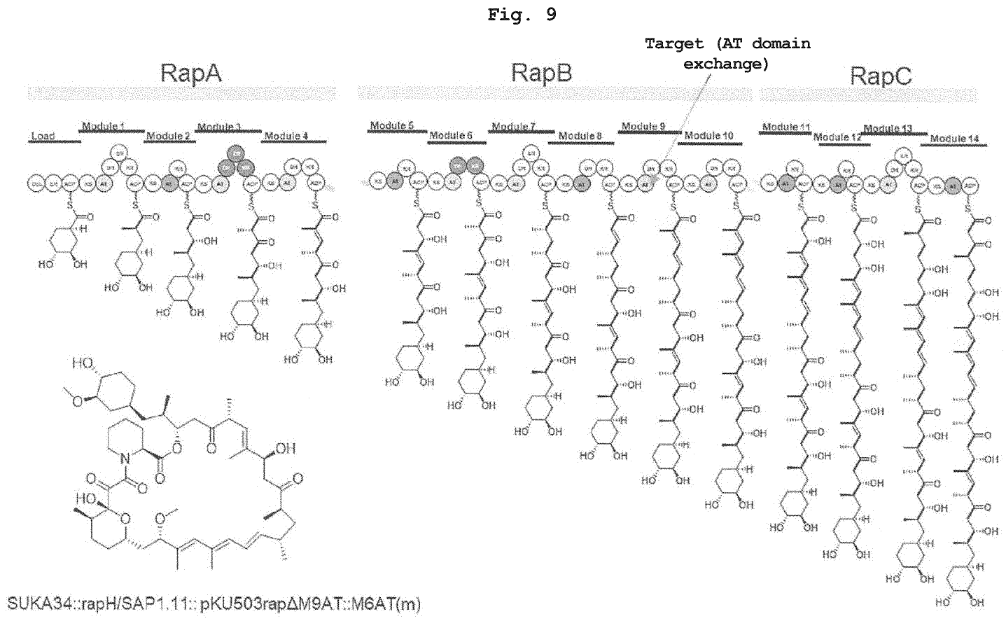

While the AT (acyltransferase) domain of module 9 of Rapamycin biosynthesis gene cluster naturally constructs a structure without a side chain, this AT domain was exchanged for an AT domain that constructs a methyl group side chain (FIG. 9).

For the cleavage by CRISPR/Cas9 and the module editing by Gibson assembly, methods similar to those of Example 2-1 were used.

In Example 2-2, sgRNAs produced by transcription from the following oligonucleotide by T7RNA polymerase were used for the cleavage by CRISPR/Cas9.

<sgRNA Oligo>
rap_M9_KS_3′_sgRNA:

(SEQ ID NO: 32)
TTCTAATACGACTCACTATAGAACCAGTCCTGGCCCGAAGCGTTTTAGAG
CTAGA

rap_M9_DH_5′_sgRNA_2:

(SEQ ID NO: 33)
TTCTAATACGACTCACTATAGGACCGGCGGTGTGCAGGTGTGTTTTAGAG
CTAGA

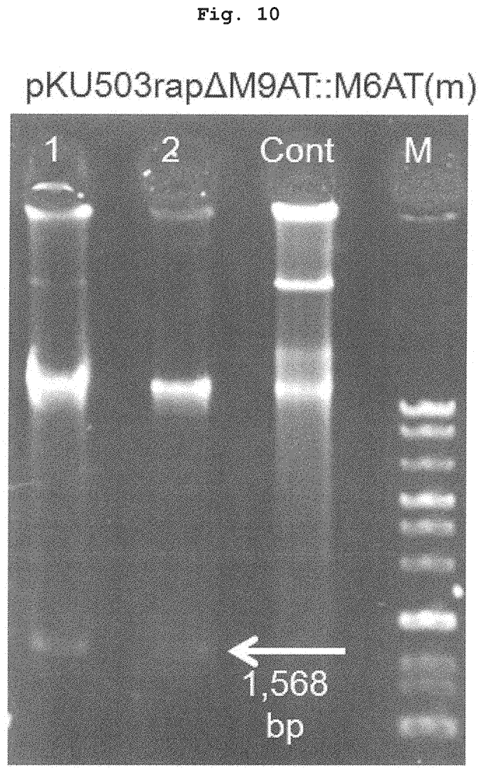

The cleaved 1568 bp fragment shown in FIG. 10 was prepared from BAC cleavage by CROSPR/Cas9 reaction using the sgRNAs.

Preparation of a modified biosynthesis gene by Gibson assembly was also performed according to the method of Example 2-1. The information of primer and the like is as shown below.

### <Primer List>

### (1) Rap_ΔM9mAT->M6mmAT_Left_Fw:

(SEQ ID NO: 34)
GCTGGTGACGGAGAACCAGTCCTGGCCCGAAGCCGGTCGGCCGCGCCGGG
CAGGCGTGTCGTCCTTCGGAGTCAGTGGCACTAATGCCCACGTCATCCTG
GAGAGCGCACCCCCCGCTCAGCCCGCGGAGG

### Rap_ΔM9mAT->M6mmAT_Left_Rv:

(SEQ ID NO: 35)
CACCACCGCACCCAGCAACGGATGCCCACCCGCAGCCGAGCGATCCACAC
CCTCGAC

### (2) Rap_ΔM9mAT->M6mmAT_Right_Fw:

| | |
|---|---|
| GGGCATCCGTTGCTGGGTGCGGTGGTGGCGTTGCCG | (SEQ ID NO: 36) |

### Rap_ΔM9mAT->M6mmAT_Right_Rv:

| | |
|---|---|
| GTGTCCGGACTCGTCAGCCTCACCA | ( SEQ ID NO: 37) |

### <Protocol>

1. pKU503rapP11-B6 was treated with restriction enzyme FspAI, electrophoresis was performed, and a fragment containing module 6 to module 10 was cut out from the gel and purified.
2. The fragment of 1. was ligated with pKU518 treated with restriction enzyme NruI, and introduced into Escherichia coli NEB 10-beta.
3. The obtained transformant was cultured and BAC was extracted.
4. Using BAC purified in 3. as a template, and using primers (1) and (2), polynucleotide for modification is amplified in two divided fragments.
5. The two fragments of 4. which have been cut out from the gel and purified are mixed to give a template, and PCR is performed using the forward primer of (1) and the reverse primer of (2).
6. PCR product cut out from the gel is purified, and used as polynucleotide for modification.

Specific polynucleotide sequence for modification is as follows:

(SEQ ID NO: 38)
GCTGGTGACGGAGAACCAGTCCTGGCCCGAAGCCGGTCGGCCGCGCCGGG
CAGGCGTGTCGTCCTTCGGAGTCAGTGGCACTAATGCCCACGTCATCCTG
GAGAGCGCACCCCCCGCTCAGCCCGCGGAGGAGGCGCAGCCTGTTGAGAC
GCCGGTGGTGGCCTCGGATGTGCTGCCGCTGGTGATATCGGCCAAGACCC
AGCCCGCCCTGACCGAACACGAAGACCGGCTGCGCGCCTACCTGGCGGCG
TCGCCCGGAGTGGATACACGGGCTGTTGCATCAACGCTCGCGGTGACACG
GTCGGTGTTCGAGCACCGCGCCGTACTCCTTGGAGACGACACCGTCACCG
GCACCGCTGTGTCCGATCCCCGGGTGGTGTTTGTTTTCCCGGGGCAGGGG
TGGCAGTGGCTGGGGATGGGCAGTGCGCTGCGCGATTCCTCGATCGTGTT
CGCCGAGCGGATGGCCGAGTGCGCGGCCGCGTTGCGCGAGTTCGTGGACT
GGGACCTGTTCACGGTTCTGGATGATCCGGCGGTGGTGGACCGGGTTGAT
GTGGTCCAGCCCGCTTCCTGGGCGATGATGGTCTCCCTGGCCGCGGTGTG
GCAGGCGGCCGGTGTGCGGCCGGATGCGGTGATCGGCCATTCACAGGGTG
AGATCGCCGCGGCGTGTGTGGCGGGTGCGGTGTCGATGCGGGATGCCGCC
CGGATCGTGACCTTGCGCAGCCAGGCGATCGCCCGGGGCCTGGCGGGCCG
GGGCGCGATGGCATCCGTCGCCCTGCCCGCACAGGATGTCGAGCTGGTCG
ACGGGGCCTGGATCGCCGCCCACAACGGTCCCGCCTCCACCGTGATCGCG
GGCACCCCGGAAGCGGTCGACCATGTCCTCACCGCTCATGAAGCGCGAGG
GGTGCGGGTGCGGCGGATCACCGTCGACTACGCCTCGCACACCCCGCACG
TCGAGCTGATCCGCGACGAACTGCTCGACATCACTAGCGACAGCAGCTCG
CAGGCCCCGGTCGTGCCGTGGCTGTCGACCGTGGACGGCTCCTGGGTCGA
CAGCCCGCTCGATGTGGAGTACTGGTACCGGAACCTCCGTGAGCCGGTCG
GTTTCCACCCCGCCGTCGGCCAGTTGCAGGCCCAGGGCGACACCGTGTTC
GTCGAGGTCAGCGCCAGCCCGGTGCTGTTGCAGGCGATGGACGACGATGT
CGTCACGGTTGCCACGCTGCGTCGTGACGACGGCGACGCCACCCGGATGC
TCACCGCCCTGGCACAGGCCTATGTCCACGGCGTCACCGTCGACTGGCCC
GCCATCCTCGGCACCACCACAACCCGGGTACTGGACCTTCCGACCTACGC
CTTCCAACACCAGCGGTACTGGGTCGAGGGTGTGGATCGCTCGGCTGCGG
GTGGGCATCCGTTGCTGGGTGCGGTGGTGGCGTTGCCGGGTTCGGATGGT
GTGCTGTTGACCGGGCGGGTGTCGTTGGCCACGCATGCGTGGCTGGCTGA
TCACGCGGTGCGGGGCAGTGTGCTGCTGCCCGGTACCGGGTTTGTGGAGC
TGGTTGTCCGCGCGGCTGATGAGGTGGGCTGCGACGTCGTTGACGAGCTG
ATCGTCGAAGCCCCGCTTCTGCTGCCGCAGACCGGCGGTGTGCAGGTGTC
GGTATCGGTTGGTGAGGCTGACGAGTCCGGACAC

Introduction of the constructed mother nucleus modification construct into a host, and heterologous expression production were performed according to the method of Example 1.

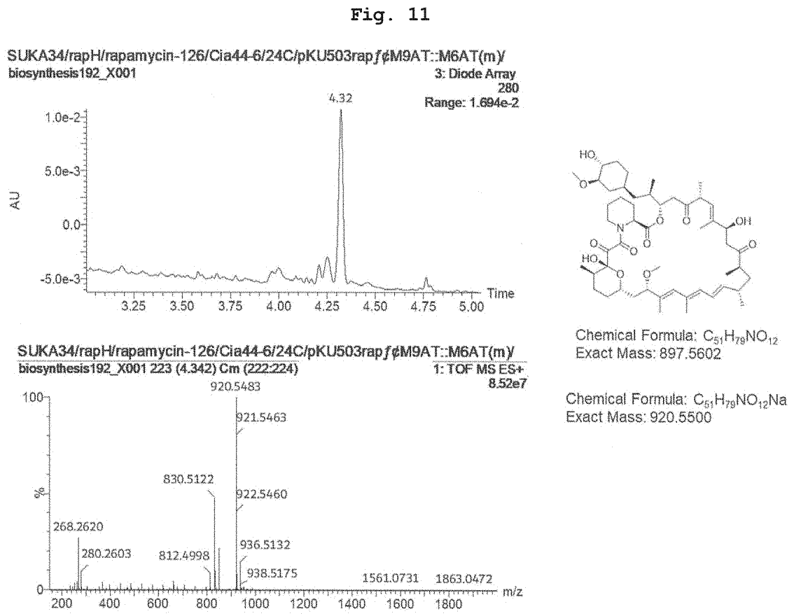

From the above results, novel mother nucleus modified rapamycin was detected as a peak of sodium-added type salt (FIG. 11, C₅₁H₇₉NO₁₂Na, measurement value: 920.5483, calculated value: 920.5500).

### Example 2-3. Production of Modified Rapamycin (Shrunk Macrolide Ring)

A large structural modification of a macrolide compound is a modification of the number of rings due to a lack or addition of a module in the large cyclic structure thereof. This modification involves a larger modification of the biosynthesis gene compared with the modification of the mother nucleus in which the domain of the module is modified, because deletion or addition treatments of the whole module is performed.

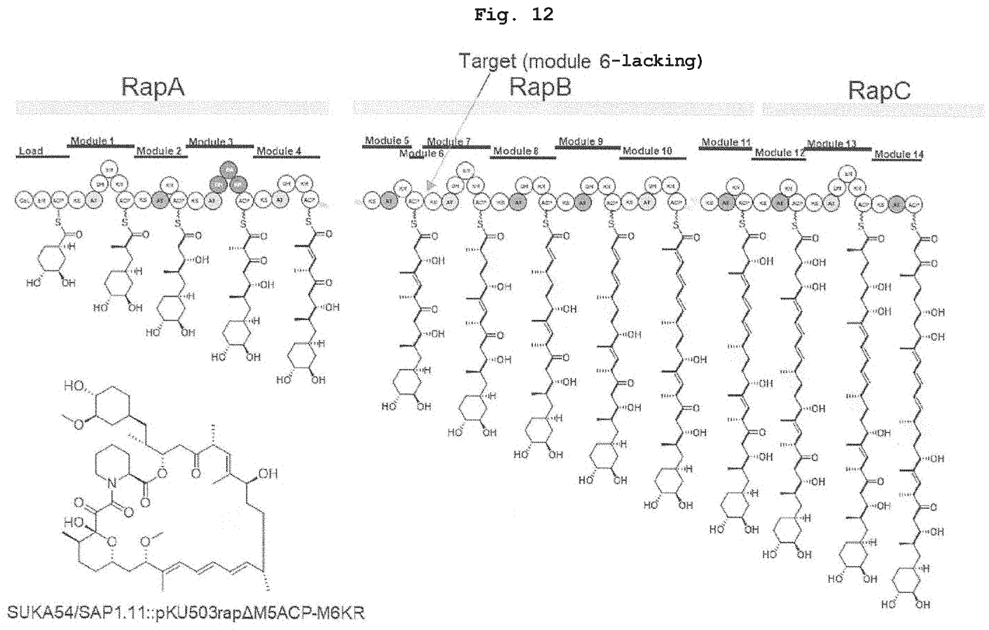

A rapamycin ring-shrunk compound lacking module 6 was produced as Example 2-3 (FIG. 12).

For the cleavage by CRISPR/Cas9 and the module editing by Gibson assembly, methods similar to those of Example 2-1 were used.

In Example 2-3, sgRNAs produced by transcription from the following oligonucleotide by T7RNA polymerase were used for the cleavage by CRISPR/Cas9.

<sgRNA Oligo>
rap_M5_KR_3′_sgRNA:

(SEQ ID NO: 39)
TTCTAATACGACTCACTATAGAGCGGCTGGAGACCGTATTCGTTTTAGAG
CTAGA

rap_M6_KR-ACP_sgRNA:

(SEQ ID NO: 40)
TTCTAATACGACTCACTATAGCAGCAACGCCGGAACCTCCGGTTTTAGAG
CTAGA

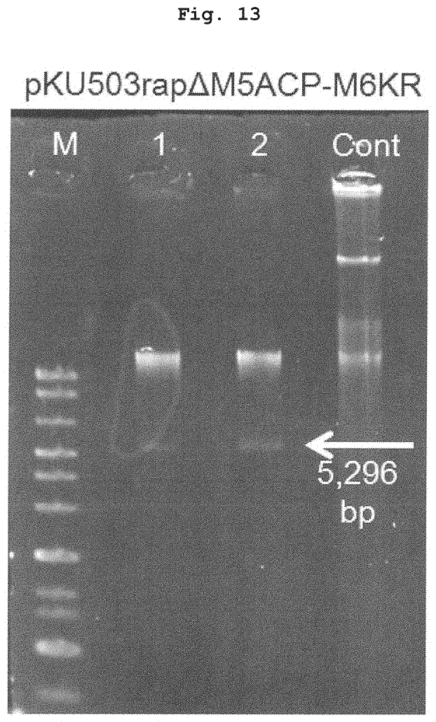

By conducting BAC cleavage by CRISPR/Cas9 reaction using the sgRNAs, the cleaved 5296 bp fragment shown in FIG. 13 was prepared.

Preparation of the modified biosynthesis gene by Gibson assembly was also performed according to the method of Example 2-1. The information of primers and the like is as shown below.

### <Primer List>

See the following protocol for Template.

### Rap_M5KR YF_Left_Fw:

(SEQ ID NO: 41)
TGTCGTTGAGTCCCTGAGCGCGCAGCGGCTGGAGACCGTATTCC

### Rap_ΔM5KR-ACP-ΔM6KR-ACP_Rv:

(SEQ ID NO: 42)
ACCGGGCGACGCAACGAACGCAGCAACGCCGGAACCTCCGCGTC
CCGTACCGGCTCCATCGGCGCGGCCACCAGAACCGGTTCACTGT
GGCGTGACGCGT

### <Protocol>

1. pKU503rapP11-B6 was treated with restriction enzyme FspAI, electrophoresis was performed, and a fragment containing module 1 to module 5 was cut out from the gel and purified.
2. The fragment of 1. was ligated with pKU518 treated with restriction enzyme NruI, and introduced into Escherichia coli NEB 10-beta.
3. The obtained transformant was cultured and BAC was extracted.
4. Using BAC purified in 3. as a template, polynucleotide for modification is amplified by PCR.
5. PCR products were cut out from the gel and purified, and used as polynucleotide for modification.

Specific polynucleotide sequence for modification is as follows:

(SEQ ID NO: 43)
TGTCGTTGAGTCCCTGAGCGCGCAGCGGCTGGAGAC
CGTATTCCGGCCCAAGGCCGATGGTGCTTGGCATT
TGCACGAGCTCACCCGGGACGCCGACCTGGCGGCG
TTCGTCATGTATTCCTCGGCTGCCGGTGTCATGGG
CGGTGCGGGTCAGGGTAACTACGCGGCGGCAAACG
CGTTCCTGGACGCGCTCGCCGAAGAACGCCGAGCC
GAGGGCCTGCCCGCACTCGCGGTGGCCTGGGGCCT
CTGGGAGGACGCCAGCGGCCTGACCGCGCAACTGA
CCGACACGGACCGTGACCGGATCCGGCGCGGTGGC
CTGCGGGCCATCTCCGCCGAGCACGGGATGCGGCT
GTTCGACAACGCGTCACGCCACAGTGAACCGGTTC
TGGTGGCCGCGCCGATGGAGCCGGTACGGGACGCG
GAGGTTCCGGCGTTGCTGCGTTCGTTGCGTCGCCC
GGT

Introduction of the constructed mother nucleus modification construct into a host, and heterologous expression production were performed according to the method of Example 1.

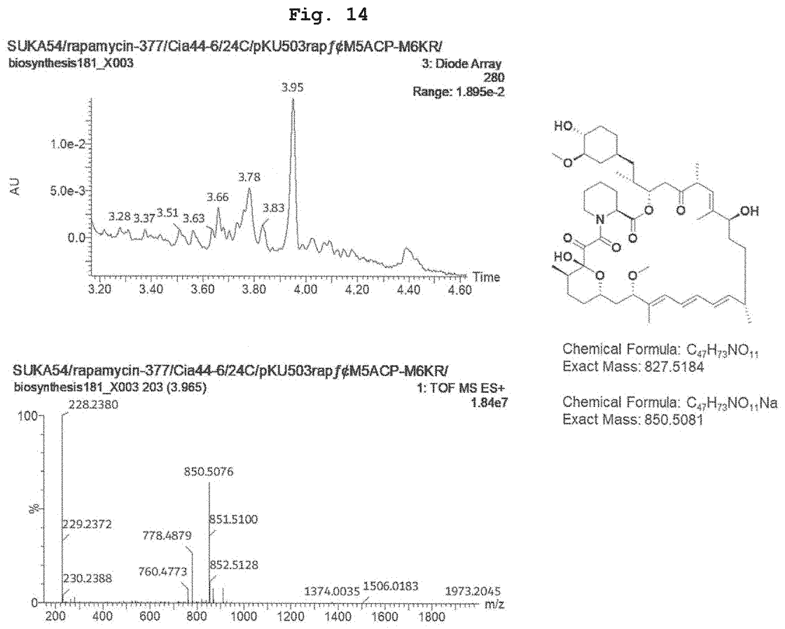

From the above results, novel mother nucleus modified rapamycin was detected as a peak of sodium-added type salt (FIG. 14, C₄₇H₇₃NO₁₁Na, measurement value: 850.5076, calculated value: 850.5081).

### Example 2-4. Production of Modified Rapamycin (Macrolide Ring Expansion)

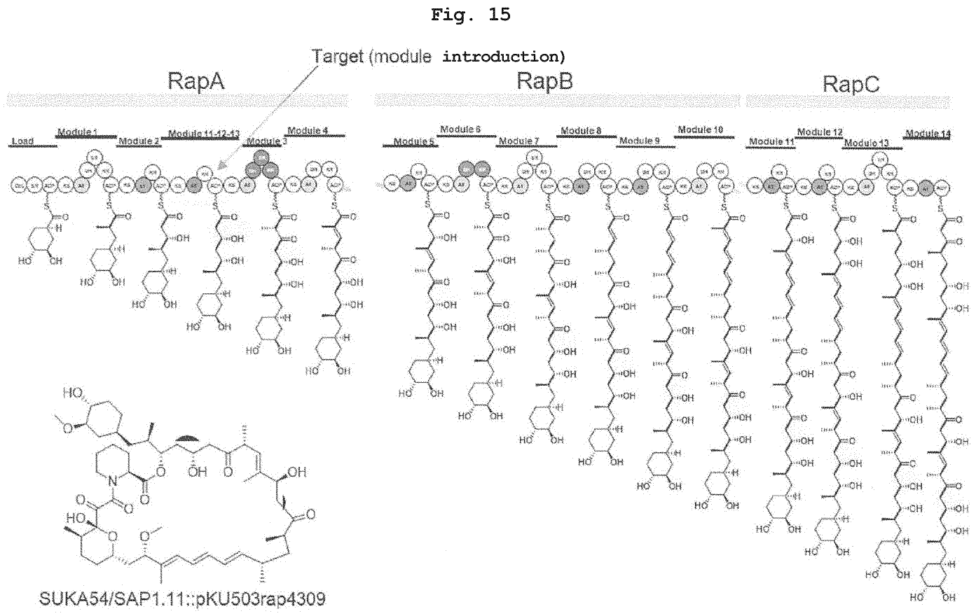

A large structural modification of a macrolide compound is a modification of the number of rings due to a lack or addition of a module in the large cyclic structure thereof. A rapamycin ring expanded compound having module 12 added between module 2 and module 3 was produced as Example 2-4 (FIG. 15).

This compound is the same as the compound described in non-patent document 2, and the compound name is Rap4309, which is the same as that in this paper. Different from the present invention, the compound in this paper was produced by chance by conventional homologous recombination. In the present invention, genome modification and heterologous expression production were performed in accordance with the design.

For the cleavage by CRISPR/Cas9 and the module editing by Gibson assembly, methods similar to those of Example 2-1 were used.

In Example 2-4, sgRNAs produced by transcription from the following oligonucleotide by T7RNA polymerase were used for the cleavage by CRISPR/Cas9.

<sgRNA Oligo>
rap_M2_KS_3′_sgRNA:

(SEQ ID NO: 44)
TTCTAATACGACTCACTATAGGCACTCCCC
ACACAGCCTGCGTTTTAGAGCTAGA

rap_M3_DH_3′_sgRNA:

(SEQ ID NO: 45)
TTCTAATACGACTCACTATAGCGTGGCCAC
CAGCCCAGGCCGTTTTAGAGCTAGA

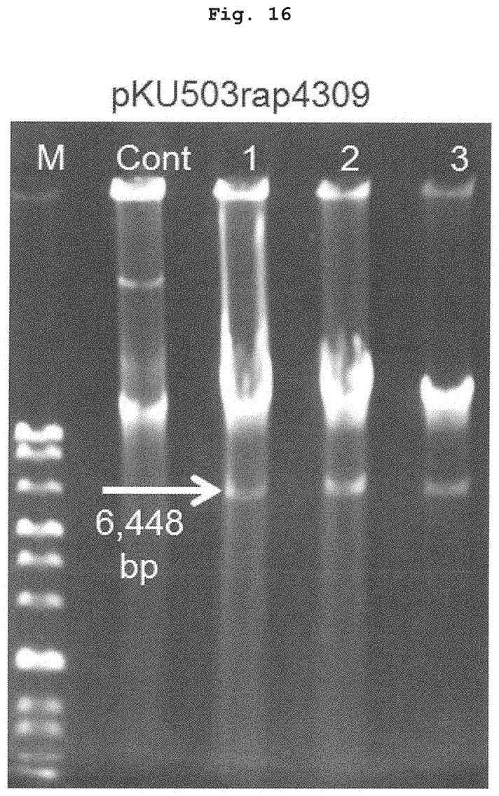

By conducting BAC cleavage by CRISPR/Cas9 reaction using the sgRNAs, the cleaved 6448 bp fragment shown in FIG. 16 was prepared.

Preparation of the modified biosynthesis gene by Gibson assembly was also performed according to the method of Example 2-1. The information of primer and the like is as shown below.

### <Primer List>

Template: pRED vector (document: Proc. Natl. Acad. Sci. USA 107: 2646-2651, 2010)

### (1) Rap4309_fra1-2_pRed_Fw:

(SEQ ID NO: 46)
GGAGTGCGCTTTCCAGGATGACGTGGGCGTtct
agaTGCCAGGAAGATACTTAACAG

### Rap4309_fra1-2_pRed_Rv:

(SEQ ID NO: 47)
CTGTTCGCAATGCAGGTGGCTCTGTTCGGGCtc
tagaCCATTCATCCGCTTATTATC

### (2) Rap4309_fra3-5_pRed_Fw:

(SEQ ID NO: 48)
CCCACGATTCCAGCAGCCCGAACAGAGCCACCT
GCATTtctagaTGCCAGGAAGATACTTAACAG

### Rap4309_fra3-5_pRed_Rv:

(SEQ ID NO: 49)
GTGAGCGTGGCCGACTTCTACGACCGGCTGGtc
tagaCCATTCATCCGCTTATTATC

### (3) Rap4309_fra1_pRed_Fw:

(SEQ ID NO: 50)
GGATGACGTGGGCGTtctagaTGCCAGGAAGATACTTAACAG

Rap4309_fra5 pRed_Rv:

(SEQ ID NO: 51)
TCTACGACCGGCTGGtctagaCCATTCATCCGCTTATTATC

Template: pKU503rapP11-B6 (SEQ ID NO: 22)
(4) 4309_fra1_M2_Fw:

(SEQ ID NO: 52)
ACGCCCACGTCATCCTGGAAAGCGCACTCCCCACA
CAGCCTGCGGGCAACACA

4309_fra1_M2-M11_Rv:

(SEQ ID NO: 53)
CCACCGGCGGCAGCGGCCCGCCGAGCAATC

(5) 4309_fra2_M1_Fw:

(SEQ ID NO: 54)
ATTGCTCGGCGGGCCGCTGCCGCCGGTGGA

4309_fra2_M11_Rv:

(SEQ ID NO: 55)
GCCCGAACAGAGCCACCTGCATT

(6) 4309_fra3_M12_Fw:

(SEQ ID NO: 56)
AATGCAGGTGGCTCTGTTCGGGCTGCTGGAATCGTGGGGTGTACGA

4309_fra3_M12_Rv:

(SEQ ID NO: 57)
TGCGGCGACCAGAATCGGGTTG

(7) 4309_fra4_M13_Fw:

(SEQ ID NO: 58)
CAACCCGATTCTGGTCGCCGCA

4309_fra4_M12-M13_Rv:

(SEQ ID NO: 59)
TGGAAGGCGTAGGTCGGAAGGTCCAGTACCCGGGTTGTGGT

(8) 4309_fra5_M3_Fw:

(SEQ ID NO: 60)
CCACAACCCGGGTACTGGACCTTCCGACCTACGCCTTCCAGCACCAGC
GGTACTGGCTCAG

4309_fra5_M3_Rv:

(SEQ ID NO: 61)
CCAGCCGGTCGTAGAAGTCGGCCACGCT

### <Protocol>

1. Using primers (1)-(3), pRED vector is amplified by PCR.
2. Using primers (4)-(8), polynucleotide for modification of rapamycin is divided into 5 fractions and amplified respectively.
3. PCR products are cut out from the gel, purified, and each PCR fragment is linked in combinations of (1) (4) (5) and (2) (6) (7) (8) by Gibson assembly.
4. Escherichia coli NEB 10-beta is transformed and the plasmid is extracted.
5. Treated with restriction enzyme XbaI, electrophoresed, and fraction 1-2 and fraction 3-5 are cut out from the gel and purified.
6. PCR fragment (3) obtained in 3. and fraction 1-2 and fraction 3-5 purified in 5. are linked by Gibson assembly.
7. Escherichia coli NEB 10-beta is transformed and the plasmid is extracted.
8. DNA fragment after XbaI cutting and purification is used as polynucleotide for modification.

Specific polynucleotide sequence for modification (full-length and each fragment (fraction 1, fraction 2, fraction 3, fraction 4, fraction 5, fraction 1-2, fraction 3-5)) are as follows:

### [Full-Length]

ACGCCCACGTCATCCTGGAAAGCGCACTCCCCACACAGCCTGCGGGCAACACAGTGGTCGAGT
CGGCACCGGAGTGGGTGCCGTTGGTGATTTCGGCGAGGACCCAGTCGGCACTGGCTGAATACG
AGGGCCGGTTGCGTGCGTATCTGGCGGCGTCGCCCGGGGCGGATACGCGGGCTGTGGCATCGA
CGCTGGCGATGACACGGTCGGTGTTCGAGTACCGGGCCGTACTCATTGGAGATGACACCGTCA
CCGGTACCGCGGCGACCGATCCGCGGGTGGTGTTCGTCTTCCCGGGTCAGGGGTCGCAGCGTG
CTGGTATGGGTGAGGAACTGGCCGCCGCGTTCCCCGTCTTCGCGCGGATCCATCAGCAGGTGT
GGGATCTGCTGGATGTGCCCGATCTCGATGTGAATGAGACCGGGTATGCCCAGCCGGCCCTGT
TCGCTTTGCAGGTGGCTCTGTTCGGGTTGCTGGAATCGTGGGGTGTACGGCCGGATGCGGTGG
TCGGTCACTCTGTCGGTGAGCTCGCCGCCGGATACGTCTCCGGGTTGTGGTCGTTGGAGGATG
CCTGCACTTTGGTGTCGGCGCGGGCTCGTCTGATGCAGGCTCTGCCTGCGGGTGGGGTGATGG
TCGCTGTCCCGGTCTCGGAGGATGAGGCTCGGGCCGTGCTGGGTGAGGGTGTGGAGATCGCCG
CGGTCAACGGGCCGTCGTCGGTGGTTCTCTCCGGTGATGAGGCCGCCGTGCTGCAGGCCGCGG
AGGGGCTGGGGAAGTGGACGCGGCTGGCGACCAGTCACGCGTTCCATTCCGCCCGTATGGAAC
CGATGCTGGAGGAGTTCCGGGCGGTCGCTGAAGGCCTGACCTACCGGACGCCGCAGGTCGCCA
TGGCCGCTGGTGATCAGGTGATGACCGCTGAGTACTGGGTGCGGCAGGTCCGGGACACGGTCC
GGTTCGGCGAGCAGGTGGCCTCGTTCGAGGATGCGGTGTTCGTCGAGCTGGGTGCCGACCGGT
CACTGGCCCGCCTGGTCGATGGCATCGCGATGCTGCACGGTGACCATGAGGCGCAGGCCGCTG
TCGGTGCCCTGGCTCACCTGTACGTGAACGGCGTGAGTGTCGAGTGGTCCGCGGTGCTGGGTG
ATGTCCCGGTAACCCGGGTGCTGGATCTTCCGACGTACGCCTTCCAGCACCAGCGGTACTGGC
TTGAGGGCACGGACCGGGCGACTGCGGGTGGTCATCCGTTGCTGGGTTCGGTGGTGCGGCTGG
CCGAGGCCAGTGGGGTGTTGTTCACTGCCCGGGTTTCCCGGAGCGGTGATCTGTGGCTGCGGG
ACCAGACGGTTCTGCCCGCGACGGTGTTCGTGGAGATGGCGCTGGCAGCGGCGGACGAGGTCG
GCTGCGGTCTGGTTGAGGATCTGAGTGTGGAAGCGTTGCTGCTGCTTCCCGATGATGGCGCCG
TCGAGGTACAGACCTGGGTGGGCGAACCGGATGAGGGCGGTCGGCGCCGGCTCAGTGTCCACG
CCCGTTACGGTGACGGCGAGCCCTGGACCTGCTTGGCCACCGCAACCCTGGCCACCACTACGG
GTGTGGCCGCTGCCGCGGTCGGCTGGCAGGCCGGTGGGGTGTGGCCGCCGGCCGGTGCGGTCC
CGGTCGGGACATCGGCACCCTCACTGCGGGCGGTGTGGCGCCTGGGCAGCGACATCTTCGCCG
AGGTGGCCCTGGACGATGCCCATGATGCCACCAGGTTTGTGCTTCATCCCGGCCTGATGGCCG
CCGCGCTCACCACCGTAGGCGAGGAGACTCCCGCCGTGTGGCAGGGCCTGACCCTGCACGCCG
GCAATCCCGGCGAGCTGCGCGTCCGCCTCACCTCACACGATGACGGCACCCTGTCGGCAGAGG
CCACCGACAGCACAGGCCTCCCCGTCCTGACCGCCCGCTCGCTCACCCTGCGCACCGTCCCCG
TATACGAACCGGCCACCAGCACCGACGACCTGCTCACCCTGACCTGGGCAGGAATCCCCACCC
CCCAGCAGACCGGCCTGACGGTGGGTGCGTTTGAAGACCTGGCGGCGGACGGCGATGTGCCGG
TACCCGAGGTGGCGGTCTTCACCGCACTCCCCGACAGCGACGATCCGCTGGAGCAAACACGAA
AGCTGACCGCTCAGGTCCTCCACACACTCCAGGAGTGGCTTGGCGGGGAGCGCTTCAGCGACA
GCACGCTGGTGGTGCGGACCGGCACCGGGTTGGCCGCTGCTGGGGTGTCGGGGTTGATGCGCT
CGGCCCAGTCCGAACACCCCGGCCGGTTCGTCCTGGTCGAAAGCGACGACGCCCTCACCCAGG
ATCAGCTGGCGGCGGCGGTCGGACTGGATGAGCCGCGGCTGCGGGTCAGCGACGGCCGGTACG
AAGTACCACGGCTGACCCGCACACATGCCGAAGAGCCTGAGCCTGAAAGGACGTGGGATCCGG
ATGGCACGGTCCTGATCACGGGCGGTTCAGGTGTGCTGGCGGGGATCGCCGCCCGGCACCTGG
TGACCGAACGCGGCGTGCGTCATCTCCTGCTGCTGTCCCGCAGCGCCCCGGATGAGGCGCTGA
TCGGCGAGCTTGGTGAACTGGGGGCCCGGGTCGAGACAGCGGCCTGTGACGTGTCCGATCCTG
CCGCGCTGACGCAGGTGCTGGCGGGTGTCTCGCCGGAGCATCCCCTGACGGCCGTGATTCACA
CCGCGGGCGTGGTGGATGACGGTGTTGTGGAGTCTTTGACCGTGCAGCGGCTGGAGACGGTAC
TGCGGCCCAAGGCCGACGGTGCGTGGAACCTGCACGAGCTCACCCGGGATGCCGACCTGGCCG
CGTTCGTCATGTATTCCTCCGCCGCCGGTGTGCTCGGTAGTGCGGGGCAGGGCAACTACGCGG
CGGCCAATGCGTTCCTGGACGCGCTGGCTGAGCAGCGTCACGCTGAGGGTCTGCCCGCACTCG
CGGTGGCCTGGGGTCTGTGGGAGGACGCCAGTGGCCTGACCGCGCAACTGACCGACACGGACC
GTGACCGGATCCGGCGCGGTGGCCTGCGGGCCATCTCCGCCGAGCACGGGATGGGGCTGTTCG
ACAGCGCGTCACGCCACAGTGAACCGGTTCTGGTGGCCGCGCCGATGGAGCCGGTACGGGACG
CGGAAGTCCCGGCATTGCTGCGGTCGTTGCACCGCCCGATTGCTCGGCGGGCCGCTGCCGCCG
GTGGAGCGCGGTGGCTGGCCGCCCTGGCACCGGCCGAGCGGGAGAAGGCACTGCTGAAGCTGG
TGTCTGACGGCGCCGCGACGGTTCTGGGACACGCCGACACCAGCACGATTCCGGCAACCACGG
CGTTCAAGGATCTGGGCATCAATTCGCTGACCGCGGTGGAACTGCGCAACAGCCTGGCGAAGG
CCACGGAGCTGCGGCTGCCCGCCACGCTGGTGTTCGACTACCCCACCCCGGCCGCCTTGGCTG
CCCGGTTGGACGAGTTGTTCACCGGCGAGAACCCCGTACCGGTACGCGGGCCGGTGTCGGCGG
TGGCGCAGGACGAGCCGCTGGCGATCGTGGGAATGGCCTGCCGCCTACCCGGTGGAGTCTCGT
CGCCTGAGGATCTGTGGCGTCTCCTGGAGTCGGGTACAGATGCGGTCTCCGGTTTCCCCACCG
ACCGTGGCTGGGACGTCGAGAACCTGTACGACATGGCTGGAAAATCGCACCGTGCTGAGGGTG
GCTTCCTGGATGCCGCGGCTGGCTTTGATGCCGGATTCTTCGGGATCAGTCCGCGTGAGGCGT
TGGCGATGGATCCGCAGCAGCGGCTGGTGCTGGAGGTGTCCTGGGAGGCGTTCGAGCGGGCCG
GGATCGAGCCCGGTTCCGTACGCGGCAGCGATACCGGCGTTTTCATGGGTGCGTACCCCGGTG
GCTACGGCATCGGTGCCGACCTCGGCGGCTTCGGGGCCACCGCCAGTTCGGTCAGTGTCCTGT
CCGGCCGGGTGTCGTACTTCTTCGGCCTCGAGGGTCCCGCGTTCACAGTCGACACGGCCTGCT
CGTCATCGTTGGTGGCGTTGCATCAGGCGGGGTATGCCCTCCGGCAGGGAGAGTGTTCGCTGG
CCCTGGTCGGCGGTGTCACTGTGATGGCCACGCCACAGACTTTCGTGGAGTTCTCCCGCCAGG
GCGGCCTGGCCTCCGACGGCCGCTGCAAAGCGTTCGCCGACGCCGCGGACGGCACGGGATGGG
CTGAAGGTGTCGGTGTCCTGCTCGTAGAGCGACTCTCCGATGCCCGCCGTAACGGTCACCAGG
TGTTGGCGGTGGTGCGTGGATCAGCGGTGAACCAGGACGGTGCGTCGAACGGTCTGACCGCGC
CGAATGGTCCTTCGCAGCAGCGGGTGATCCGGGCCGCTCTCAGCAACGCGGGTCTGAGCACGG
CTGAGGTGGATGTGGTCGAGGCGCACGGCACGGGCACAACGCTGGGTGACCCGATCGAGGCCC
AGGCGCTGATCGCTACCTATGGCCAGGACCGTGACCAGCCTGTGCTGCTGGGTTCGGTGAAGT
CGAACCTGGGTCATACGCAGGCCGCTGCGGGTGTGTCCGGTGTCATCAAGATGGTGATGGCCC
TGCAACACGGTCTGGTGCCGCGCACGTTGCATGTCGATGAGCCGTCACGGCATGTGGACTGGT
CGGCGGGCGCGGTGCAGCTCGTGACGGAGAACCAGCCGTGGCCGGATATGGGCCGAGCGCGCC
GGGCAGGCGTGTCGTCCTTCGGGATCAGTGGCACCAACGCCCACGTCATCCTGGAAAGCGCAC
CCCCCACTCAGCCTGCGGACAACGCGGTGATCGAGCGGGCACCGGAGTGGGTGCCGTTGGTGA
TTTCGGCCAGGACCCAGTCGGCTTTGACTGAGCACGAGGGCCGGTTGCGTGCGTATCTGGCGG
CGTCGCCCGGGGTGGATATGCGGGCTGTGGCATCGACGCTGGCGATGACACGGTCGGTGTTCG
AGCACCGTGCCGTGCTGCTGGGAGATGACACCGTCACCGGCACCGCTGTGTCTGACCCTCGGG
CGGTGTTCGTCTTCCCGGGACAGGGGTCGCAGCGTGCTGGCATGGGTGAGGAACTGGCCGCCG
CGTTCCCCGTCTTCGCGCGGATCCATCAGCAGGTGTGGGACCTGCTCGATGTGCCCGATCTGG
AGGTGAACGAGACCGGTTACGCCCAGCCGGCCCTGTTCGCAATGCAGGTGGCTCTGTTCGGGC
TGCTGGAATCGTGGGGTGTACGACCGGACGCGGTGATCGGCCATTCGGTGGGTGAGCTTGCGG
CTGCGTATGTGTCCGGGGTGTGGTCGTTGGAGGATGCCTGCACTTTGGTGTCGGCGCGGGCTC
GTCTGATGCAGGCTCTGCCCGCGGGTGGGGTGATGGTCGCTGTCCCGGTCTCGGAGGATGAGG
CCCGGGCCGTGCTGGGTGAGGGTGTGGAGATCGCCGCGGTCAACGGCCCGTCGTCGGTGGTTC
TCTCCGGTGATGAGGCCGCCGTGCTGCAGGCCGCGGAGGGGCTGGGGAAGTGGACGCGGCTGG
CGACCAGCCACGCGTTCCATTCCGCCCGTATGGAACCCATGCTGGAGGAGTTCCGGGCGGTCG
CCGAAGGCCTGACCTACCGGACGCCGCAGGTCTCCATGGCCGTTGGTGATCAGGTGACCACCG
CTGAGTACTGGGTGCGGCAGGTCCGGGACACGGTCCGGTTCGGCGAGCAGGTGGCCTCGTACG
AGGACGCCGTGTTCGTCGAGCTGGGTGCCGACCGGTCACTGGCCCGCCTGGTCGACGGTGTCG
CGATGCTGCACGGCGACCACGAAATCCAGGCCGCGATCGGCGCCCTGGCCCACCTGTATGTCA
ACGGCGTCACGGTCGACTGGCCCGCGCTCCTGGGCGATGCTCCGGCAACACGGGTGCTGGACC
TTCCGACATACGCCTTCCAGCACCAGCGCTACTGGCTCGAGGGCACGGACCGGGCGACTGCGG
GTGGCCATCCGTTGCTGGGTTCGGCGGTGCGGCTGGCCGAGGCCAGCGGGGTGTTGTTCACTG
CCCGGGTTTCCCGGAGCGGCGATCTGTGGCTGCGGGACCAGACGGTTCTGCCCGCGACGGTGT
TCGTGGAGATGGCGCTGGCAGCGGCGGACGAGGCCGGCTGCGGTCTGGTTGAGGACCTGAACG
TGGAAGCGTTGCTGCTGCTTCCTGACGATGGCGCCGTCCAGGTACAGACCTGGGTGAGCGAAC
CGGACGAGGCCGGTCGCCACCGGCTCAGTATCCACGCCCGTTACAGCGACAGCGAGCCCTGGA
CACGCTTGGCCACCGCAACCCTCGCCACCAGGGGAACGGTATCCGGCTGGCAGGCCGGGGAGG
CGTGGCCGCCGACCGGTGCGGTCCCGGTCGAGACCGGAGTACCGTCACTGCGCGGGGTGTGGC
GCCGAGGCAACGAAGTGTTCGCCGAGGTCGCCCTGGACAGCACCCACGACGCCACCACATATG
CCCTGCACCCTGCCCTCCTGACCGCCGCCCTCACCACCGCCGGTGAGGAAACCCCCGCCGCGT
GGCAGGCGCTGACCCTGCACGCCCGCAACCCTGCCGAGCTGCGCGTCCGCCTCATCTCACACG
ATGACGGCACCCTGTCCGTGGACGCCACCGACAGCACAGGCCTCCCCGTCCTGACCGTCCGCT
CCCTCACCCTGCGCACCGTCCCCGTCTACGAACCTGCCACCAGCACCGACGACCTGCTCACCC
TGACCTGGGCGGAGATCCCGGCCCCTCAGGAAACCGGCCTGACGGTCGGCCGGTTCGAGGACC
TGGTGTCGGACGCTGATGTGCCGGTACCCGAGGTGGCGGTCTTCACCGCACTCCCCGACAGCA
GCGAGAACCCGCTGGAACAGACCCGCGTACTGACCGCTCAGGTCCTCCAGGCAGTCCAGACCT
GGCTTGGCGGGGAACGTTTCACCGACAGCACGCTGGTCGTGCGGACCGGCACCCGGTTGGCCG
CCGCTGGGGTGTCGGGGTTGATGCGATCGGCTCAATCGGAACACCCCGGCCGGTTCGTCCTGG
TCGAGAGCGACGACGACACGCTCGCCCCGGACCAGTTGGCCGCCACCGTCGGGCTCGACGAGC
CGCGGCTGCGGGTCAGCGGCGACCGGTACGAGGCACCGCGACTGGCTCGTGTGAACGCCAGTG
GGTCTGAGCCTGAAGCGGTTTGGGATCCGGATGGCACGGTTCTGATCACCGGTGGTTCGGGTG
TGCTGGCGGGGATCGCCGCCCGGCACCTGGTGGCCGAACGCGGCGTGCGTCATCTGCTGCTGC
TGTCCCGCAGCGCCCCGGACGAGGCACTGATCAACCAACTCGGCGAACTGGGCGCCCGGGTCG
AGACAGCGGCCTGTGACGTGTCCGATCGTGCCGCGCTGGCCCAGGTGCTGGCGGGTGTGTCAC
CGGAGCACCCCCTGACGGCAGTGATTCACACCGCGGGCGTACTCGATGACGGTGTTGTCGAGT
CCCTGACCGCGCAGCGGCTCGACACGGTACTGCGGCCCAAGGCCGACGGCGCCTGGCATCTGC
ACGAACTCACCCGCAACACCGACCTGGCCGCCTTCGTCATGTACTCCTCCGCCGCCGGTGTCA
TGGGCGGTGGGGGGCAAGGTAACTACGCGGCGGCAAACGCGTTCCTGGACGCGCTCGCCGAAG
AACGCCGCGCCGAGGGCCTGCCCGCACTCGCGGTGGCCTGGGGTCTGTGGGAGGACGCCAGTG
GCCTGACCACGCAACTGACCGACACGGACCGTGACCGGATCCGGCGCGGTGGCCTGCGGACTA
TCACCGCCGAGTACGGGATGCGGCTGTTCGACACCGCATCACGCCATGGCAACCCGATTCTGG
TCGCCGCACCGATGGACCCGGTTTGGGACGCGGAAGTCCCCGCGCTCCTCCGCTCGTTGCATC
GTCCCGTCGCCCGGCGGGCCGCCTCTACCAGCGACTCGTCAGCGCGGTGGCTGGCGGCCCTGG
CACCGGCCGAGCGGGAAGACGCACTGCTGAAGCTGGTGCGTGACAGCGCCGCTCTGGTCCTGG
GACACGCTGACGCCAGCACCATCCCCGCAGCCGCCGCATTCAAGGATCTGGGTATCGATTCGC
TGACCGCGGTGGAACTGCGCAACAGCCTGGCGAAAGCCACAGGGCTGCGGCTGCCCAACACGA
CGGTGTTCGACTACCCCACCCCGGCCATCCTGGCCACCCGGCTGGGTGAGCTGTTCACCGGCG
AGAACCCTGCACCGGTACGCCCGTCGGTGTCGGTGGTGGGGCAGGACGAGCCGCTGGCGGTCG
TGGGTATGGCCTGCCGTCTGCCCGGCGGGGTGTCGTCGCCTGAGGATCTGTGGCGCCTTGTGG
AGTCGGGTACGGATGCGATTTCCGGTTTCCCCGCCGACCGTGGGTGGGACGCGGAGAGCCTGT
TCGATCCGGACCCGGACGCGGTCGGGAAGTCGTACTGCGTAGAGGGCGGCTTCCTCGACAGCG
CAGCCAGCTTCGACGCCGGATTCTTCGGCATCAGCCCACGCGAGGCTCTGGCGATGGACCCGC
AGCAGCGGCTGATCATGGAGGTGTCCTGGGAGGCCTTCGAGCGGGCCGGGATCGAGCCCGGTT
CCGTGCGCGGCAGCGACACCGGCGTCTTCATGGGCGCGTACGCCGGTGGCTACGGTGCCGGTG
CTGACCTCGGCGGCTTCGCGGCCACCGCCAGCGCGACCAGTGTCCTGTCCGGCCGGGTGTCGT
ACTTCTTCGGCCTCGAAGGCCCCGCCATCACAGTCGACACAGCCTGCTCGTCATCACTGGTGG
CACTGCACCAGGCCGGGTATGCCCTCCGGCAGGGAGAGTGTTCCCTGGCCCTGGTCGGCGGCG
TCACCGTGATGGCCACACCACAAAGCTTCGTGGAATTCTCCCGCCAGCGTGGTCTGGCCTCCG
ATGGCCGGTGCAAGGCGTTCGCAGACAGCGCGGACGGCACGGGATGGGCTGAAGGCGTTGGTG
TGCTGCTGGTAGAGCGGCTTTCCGACGCCCAGGCCAAGGGCCATCAGGTGTTGGCGGTGGTCC
GTAGCTCGGCGGTCAACCAGGACGGCGCGTCCAACGGTCTGACCGCGCCGAACGGTCCTTCGC
AGCAGCGGGTGATCCAAGCCGCTCTCAGTAACGCCGGCCTCGCCGCGCACGAGGTGGATGTGG
TCGAGGCCCACGGCACGGGCACGACGCTGGGCGACCCGATCGAGGCCCAGGCGCTGATCGCCA
CTTACGGTCAGGACCGGGAACGGCCCCTGCTGCTGGGTTCGCTGAAGTCGAACATCGGTCATG
CTCAGGCCGCCTCGGGCGTGTCGGGTGTCATCAAGATGGTCATGGCCCTGCAGCACAACACGG
TTCCCCGCACCCTGCACGTGGATGAGCCGTCGCGGCACGTGGACTGGGCGGCGGGTGCGGTTG
AGCTGGTGAGGGAGAACCAGCCCTGGCCCGGCACCGACCGGCCCCGTCGGGCGGGCGTGTCGT
CCTTCGGAGTCAGCGGCACCAACGCCCACGTCATCCTGGAGAGCGCACCCCCCGCTCAGCCCG
CGGAGGAGGCGCAGCCTGTTGAGACGCCGGTGGTGGCCTCGGATGTGCTGCCGCTGGTGATAT
CGGCCAAGACCCAGCCCGCCCTGACCGAACACGAAGACCGGCTGCGCGCCTACCTGGCGGCGT
CGCCCGGGGCGGATATACGGGCTGTGGCATCGACGCTGGCGGTGACACGGTCGGTGTTCGAGC
ACCGCGCCGTACTCCTTGGAGATGACACCGTCACCGGCACCGCGGTGACCGACCCCAGGATCG
TGTTTGTCTTTCCCGGGCAGGGGTGGCAGTGGCTGGGGATGGGCAGTGCACTGCGCGATTCGT
CGGTGGTGTTCGCCGAGCGGATGGCCGAGTGTGCGGCGGCGTTGCGCGAGTTCGTGGACTGGG
ATCTGTTCACGGTTCTGGATGATCCGGCGGTGGTGGACCGGGTTGATGTGGTCCAGCCCGCTT
CCTGGGCGATGATGGTTTCCCTGGCCGCGGTGTGGCAGGCGGCCGGTGTGCGGCCGGATGCGG
TGATCGGCCATTCGCAGGGTGAGATCGCCGCAGCTTGTGTGGCGGGTGCGGTGTCACTACGCG
ATGCCGCCCGGATCGTGACCTTGCGCAGCCAGGCGATCGCCCGGGGCCTGGCGGGCCGGGGCG
CGATGGCATCCGTCGCCCTGCCCGCGCAGGATGT

(continued from the above-mentioned sequence)

(SEQ ID NO: 62)
CGAGCTGGTCGACGGGGCCTGGATCGCCGCCCACAACGGGCCCGCCTCC
ACCGTGATCGCGGGCACCCCGGAAGCGGTCGACCATGTCCTCACCGCTC
ATGAGGCACAAGGGGTGCGGGTGCGGCGGATCACCGTCGACTATGCCTC
GCACACCCCGCACGTCGAGCTGATCCGCGACGAACTACTCGACATCACT
AGCGACAGCAGCTCGCAGACCCCGCTCGTGCCGTGGCTGTCGACCGTGG
ACGGCACCTGGGTCGACAGCCCGCTGGACGGGGAGTACTGGTACCGGAA
CCTGCGTGAACCGGTCGGTTTCCACCCCGCCGTCAGCCAGTTGCAGGCC
CAGGGCGACACCGTGTTCGTCGAGGTCAGCGCCAGCCCGGTGTTGTTGC
AGGCGATGGACGACGATGTCGTCACGGTTGCCACGCTGCGTCGTGACGA
CGGCGACGCCACCCGGATGCTCACCGCCCTGGCACAGGCCTATGTCCAC
GGCGTCACCGTCGACTGGCCCGCCATCCTCGGCACCACCACAACCCGGG
TACTGGACCTTCCGACCTACGCCTTCCAGCACCAGCGGTACTGGCTCAG
GAGCGTGGACCGGGCGGCTGCCGACGGTCATCCACTGCTGGGCACCGTA
GTGGCACTGCCCGGCTCCGACGGTGTGGTGCTCACCGGGCGGGTGTCGC
TGGCCACCCATACATGGCTGGCCGATCACGCGGTCCGGGGCAGTGTCCT
GCTACCCGGGACCGCATTTGTGGAACTGGTCGTCCGCGCCGCCGACGAG
GTCGAGTGCGACGTCGTTGACGAGTTGGTGATCGAAACCCCGCTCCTGC
TGCCGCAGACCGGAGGCGTCCAACTGTCCGTGTCCGTCGGCGGAGCCGA
CGAGTCCGGGCACCGCGCGGTGATGGTCTTCTCCCAGGCGGACAACACC
GATACCTGGACCCGGCACGTCACGGCGACAGTCAGCACCTCTGACTCGA
CGGTCTCGCTGCCGGAGTTTGCCTCGTGGCCACCAGCCCAGGCCCGGCC
GGTGAGCGTGGCCGACTTCTACGACCGGCTGG

### [Fraction1]

(SEQ ID NO: 63)
ACGCCCACGTCATCCTGGAAAGCGCACTCCCCACACAGCCTGCGGGCAA
CACAGTGGTCGAGTCGGCACCGGAGTGGGTGCCGTTGGTGATTTCGGCG
AGGACCCAGTCGGCACTGGCTGAATACGAGGGCCGGTTGCGTGCGTATC
TGGCGGCGTCGCCCGGGGCGGATACGCGGGCTGTGGCATCGACGCTGGC
GATGACACGGTCGGTGTTCGAGTACCGGGCCGTACTCATTGGAGATGAC
ACCGTCACCGGTACCGCGGCGACCGATCCGCGGGTGGTGTTCGTCTTCC
CGGGTCAGGGGTCGCAGCGTGCTGGTATGGGTGAGGAACTGGCCGCCGC
GTTCCCCGTCTTCGCGCGGATCCATCAGCAGGTGTGGGATCTGCTGGAT
GTGCCCGATCTCGATGTGAATGAGACCGGGTATGCCCAGCCGGCCCTGT
TCGCTTTGCAGGTGGCTCTGTTCGGGTTGCTGGAATCGTGGGGTGTACG
GCCGGATGCGGTGGTCGGTCACTCTGTCGGTGAGCTCGCCGCCGGATAC
GTCTCCGGGTTGTGGTCGTTGGAGGATGCCTGCACTTTGGTGTCGGCGC
GGGCTCGTCTGATGCAGGCTCTGCCTGCGGGTGGGGTGATGGTCGCTGT
CCCGGTCTCGGAGGATGAGGCTCGGGCCGTGCTGGGTGAGGGTGTGGAG
ATCGCCGCGGTCAACGGGCCGTCGTCGGTGGTTCTCTCCGGTGATGAGG
CCGCCGTGCTGCAGGCCGCGGAGGGGCTGGGGAAGTGGACGCGGCTGGC
GACCAGTCACGCGTTCCATTCCGCCCGTATGGAACCGATGCTGGAGGAG
TTCCGGGCGGTCGCTGAAGGCCTGACCTACCGGACGCCGCAGGTCGCCA
TGGCCGCTGGTGATCAGGTGATGACCGCTGAGTACTGGGTGCGGCAGGT
CCGGGACACGGTCCGGTTCGGCGAGCAGGTGGCCTCGTTCGAGGATGCG
GTGTTCGTCGAGCTGGGTGCCGACCGGTCACTGGCCCGCCTGGTCGATG
GCATCGCGATGCTGCACGGTGACCATGAGGCGCAGGCCGCTGTCGGTGC
CCTGGCTCACCTGTACGTGAACGGCGTGAGTGTCGAGTGGTCCGCGGTG
CTGGGTGATGTCCCGGTAACCCGGGTGCTGGATCTTCCGACGTACGCCT
TCCAGCACCAGCGGTACTGGCTTGAGGGCACGGACCGGGCGACTGCGGG
TGGTCATCCGTTGCTGGGTTCGGTGGTGCGGCTGGCCGAGGCCAGTGGG
GTGTTGTTCACTGCCCGGGTTTCCCGGAGCGGTGATCTGTGGCTGCGGG
ACCAGACGGTTCTGCCCGCGACGGTGTTCGTGGAGATGGCGCTGGCAGC
GGCGGACGAGGTCGGCTGCGGTCTGGTTGAGGATCTGAGTGTGGAAGCG
TTGCTGCTGCTTCCCGATGATGGCGCCGTCGAGGTACAGACCTGGGTGG
GCGAACCGGATGAGGGCGGTCGGCGCCGGCTCAGTGTCCACGCCCGTTA
CGGTGACGGCGAGCCCTGGACCTGCTTGGCCACCGCAACCCTGGCCACC
ACTACGGGTGTGGCCGCTGCCGCGGTCGGCTGGCAGGCCGGTGGGGTGT
GGCCGCCGGCCGGTGCGGTCCCGGTCGGGACATCGGCACCCTCACTGCG
GGCGGTGTGGCGCCTGGGCAGCGACATCTTCGCCGAGGTGGCCCTGGAC
GATGCCCATGATGCCACCAGGTTTGTGCTTCATCCCGGCCTGATGGCCG
CCGCGCTCACCACCGTAGGCGAGGAGACTCCCGCCGTGTGGCAGGGCCT
GACCCTGCACGCCGGCAATCCCGGCGAGCTGCGCGTCCGCCTCACCTCA
CACGATGACGGCACCCTGTCGGCAGAGGCCACCGACAGCACAGGCCTCC
CCGTCCTGACCGCCCGCTCGCTCACCCTGCGCACCGTCCCCGTATACGA
ACCGGCCACCAGCACCGACGACCTGCTCACCCTGACCTGGGCAGGAATC
CCCACCCCCCAGCAGACCGGCCTGACGGTGGGTGCGTTTGAAGACCTGG
CGGCGGACGGCGATGTGCCGGTACCCGAGGTGGCGGTCTTCACCGCACT
CCCCGACAGCGACGATCCGCTGGAGCAAACACGAAAGCTGACCGCTCAG
GTCCTCCACACACTCCAGGAGTGGCTTGGCGGGGAGCGCTTCAGCGACA
GCACGCTGGTGGTGCGGACCGGCACCGGGTTGGCCGCTGCTGGGGTGTC
GGGGTTGATGCGCTCGGCCCAGTCCGAACACCCCGGCCGGTTCGTCCTG
GTCGAAAGCGACGACGCCCTCACCCAGGATCAGCTGGCGGCGGCGGTCG
GACTGGATGAGCCGCGGCTGCGGGTCAGCGACGGCCGGTACGAAGTACC
ACGGCTGACCCGCACACATGCCGAAGAGCCTGAGCCTGAAAGGACGTGG
GATCCGGATGGCACGGTCCTGATCACGGGCGGTTCAGGTGTGCTGGCGG
GGATCGCCGCCCGGCACCTGGTGACCGAACGCGGCGTGCGTCATCTCCT
GCTGCTGTCCCGCAGCGCCCCGGATGAGGCGCTGATCGGCGAGCTTGGT
GAACTGGGGGCCCGGGTCGAGACAGCGGCCTGTGACGTGTCCGATCCTG
CCGCGCTGACGCAGGTGCTGGCGGGTGTCTCGCCGGAGCATCCCCTGAC
GGCCGTGATTCACACCGCGGGCGTGGTGGATGACGGTGTTGTGGAGTCT
TTGACCGTGCAGCGGCTGGAGACGGTACTGCGGCCCAAGGCCGACGGTG
CGTGGAACCTGCACGAGCTCACCCGGGATGCCGACCTGGCCGCGTTCGT
CATGTATTCCTCCGCCGCCGGTGTGCTCGGTAGTGCGGGGCAGGGCAAC
TACGCGGCGGCCAATGCGTTCCTGGACGCGCTGGCTGAGCAGCGTCACG
CTGAGGGTCTGCCCGCACTCGCGGTGGCCTGGGGTCTGTGGGAGGACGC
CAGTGGCCTGACCGCGCAACTGACCGACACGGACCGTGACCGGATCCGG
CGCGGTGGCCTGCGGGCCATCTCCGCCGAGCACGGGATGGGGCTGTTCG
ACAGCGCGTCACGCCACAGTGAACCGGTTCTGGTGGCCGCGCCGATGGA
GCCGGTACGGGACGCGGAAGTCCCGGCATTGCTGCGGTCGTTGCACCGC
CCGATTGCTCGGCGGGCCGCTGCCGCCGGTGG

### [Fraction2]

(SEQ ID NO: 64)
ATTGCTCGGCGGGCCGCTGCCGCCGGTGGAGCGCGGTGGCTGGCCGCCC
TGGCACCGGCCGAGCGGGAGAAGGCACTGCTGAAGCTGGTGTCTGACGG
CGCCGCGACGGTTCTGGGACACGCCGACACCAGCACGATTCCGGCAACC
ACGGCGTTCAAGGATCTGGGCATCAATTCGCTGACCGCGGTGGAACTGC
GCAACAGCCTGGCGAAGGCCACGGAGCTGCGGCTGCCCGCCACGCTGGT
GTTCGACTACCCCACCCCGGCCGCCTTGGCTGCCCGGTTGGACGAGTTG
TTCACCGGCGAGAACCCCGTACCGGTACGCGGGCCGGTGTCGGCGGTGG
CGCAGGACGAGCCGCTGGCGATCGTGGGAATGGCCTGCCGCCTACCCGG
TGGAGTCTCGTCGCCTGAGGATCTGTGGCGTCTCCTGGAGTCGGGTACA
GATGCGGTCTCCGGTTTCCCCACCGACCGTGGCTGGGACGTCGAGAACC
TGTACGACATGGCTGGAAAATCGCACCGTGCTGAGGGTGGCTTCCTGGA
TGCCGCGGCTGGCTTTGATGCCGGATTCTTCGGGATCAGTCCGCGTGAG
GCGTTGGCGATGGATCCGCAGCAGCGGCTGGTGCTGGAGGTGTCCTGGG
AGGCGTTCGAGCGGGCCGGGATCGAGCCCGGTTCCGTACGCGGCAGCGA
TACCGGCGTTTTCATGGGTGCGTACCCCGGTGGCTACGGCATCGGTGCC
GACCTCGGCGGCTTCGGGGCCACCGCCAGTTCGGTCAGTGTCCTGTCCG
GCCGGGTGTCGTACTTCTTCGGCCTCGAGGGTCCCGCGTTCACAGTCGA
CACGGCCTGCTCGTCATCGTTGGTGGCGTTGCATCAGGCGGGGTATGCC
CTCCGGCAGGGAGAGTGTTCGCTGGCCCTGGTCGGCGGTGTCACTGTGA
TGGCCACGCCACAGACTTTCGTGGAGTTCTCCCGCCAGGGCGGCCTGGC
CTCCGACGGCCGCTGCAAAGCGTTCGCCGACGCCGCGGACGGCACGGGA
TGGGCTGAAGGTGTCGGTGTCCTGCTCGTAGAGCGACTCTCCGATGCCC
GCCGTAACGGTCACCAGGTGTTGGCGGTGGTGCGTGGATCAGCGGTGAA
CCAGGACGGTGCGTCGAACGGTCTGACCGCGCCGAATGGTCCTTCGCAG
CAGCGGGTGATCCGGGCCGCTCTCAGCAACGCGGGTCTGAGCACGGCTG
AGGTGGATGTGGTCGAGGCGCACGGCACGGGCACAACGCTGGGTGACCC
GATCGAGGCCCAGGCGCTGATCGCTACCTATGGCCAGGACCGTGACCAG
CCTGTGCTGCTGGGTTCGGTGAAGTCGAACCTGGGTCATACGCAGGCCG
CTGCGGGTGTGTCCGGTGTCATCAAGATGGTGATGGCCCTGCAACACGG
TCTGGTGCCGCGCACGTTGCATGTCGATGAGCCGTCACGGCATGTGGAC
TGGTCGGCGGGCGCGGTGCAGCTCGTGACGGAGAACCAGCCGTGGCCGG
ATATGGGCCGAGCGCGCCGGGCAGGCGTGTCGTCCTTCGGGATCAGTGG
CACCAACGCCCACGTCATCCTGGAAAGCGCACCCCCCACTCAGCCTGCG
GACAACGCGGTGATCGAGCGGGCACCGGAGTGGGTGCCGTTGGTGATTT
CGGCCAGGACCCAGTCGGCTTTGACTGAGCACGAGGGCCGGTTGCGTGC
GTATCTGGCGGCGTCGCCCGGGGTGGATATGCGGGCTGTGGCATCGACG
CTGGCGATGACACGGTCGGTGTTCGAGCACCGTGCCGTGCTGCTGGGAG
ATGACACCGTCACCGGCACCGCTGTGTCTGACCCTCGGGCGGTGTTCGT
CTTCCCGGGACAGGGGTCGCAGCGTGCTGGCATGGGTGAGGAACTGGCC
GCCGCGTTCCCCGTCTTCGCGCGGATCCATCAGCAGGTGTGGGACCTGC
TCGATGTGCCCGATCTGGAGGTGAACGAGACCGGTTACGCCCAGCCGGC
CCTGTTCGCAATGCAGGTGGCTCTGTTCGGGC

### [Fraction3]

(SEQ ID NO: 65)
AATGCAGGTGGCTCTGTTCGGGCTGCTGGAATCGTGGGGTGTACGACCG
GACGCGGTGATCGGCCATTCGGTGGGTGAGCTTGCGGCTGCGTATGTGT
CCGGGGTGTGGTCGTTGGAGGATGCCTGCACTTTGGTGTCGGCGCGGGC
TCGTCTGATGCAGGCTCTGCCCGCGGGTGGGGTGATGGTCGCTGTCCCG
GTCTCGGAGGATGAGGCCCGGGCCGTGCTGGGTGAGGGTGTGGAGATCG
CCGCGGTCAACGGCCCGTCGTCGGTGGTTCTCTCCGGTGATGAGGCCGC
CGTGCTGCAGGCCGCGGAGGGGCTGGGGAAGTGGACGCGGCTGGCGACC
AGCCACGCGTTCCATTCCGCCCGTATGGAACCCATGCTGGAGGAGTTCC
GGGCGGTCGCCGAAGGCCTGACCTACCGGACGCCGCAGGTCTCCATGGC
CGTTGGTGATCAGGTGACCACCGCTGAGTACTGGGTGCGGCAGGTCCGG
GACACGGTCCGGTTCGGCGAGCAGGTGGCCTCGTACGAGGACGCCGTGT
TCGTCGAGCTGGGTGCCGACCGGTCACTGGCCCGCCTGGTCGACGGTGT
CGCGATGCTGCACGGCGACCACGAAATCCAGGCCGCGATCGGCGCCCTG
GCCCACCTGTATGTCAACGGCGTCACGGTCGACTGGCCCGCGCTCCTGG
GCGATGCTCCGGCAACACGGGTGCTGGACCTTCCGACATACGCCTTCCA
GCACCAGCGCTACTGGCTCGAGGGCACGGACCGGGCGACTGCGGGTGGC
CATCCGTTGCTGGGTTCGGCGGTGCGGCTGGCCGAGGCCAGCGGGGTGT
TGTTCACTGCCCGGGTTTCCCGGAGCGGCGATCTGTGGCTGCGGGACCA
GACGGTTCTGCCCGCGACGGTGTTCGTGGAGATGGCGCTGGCAGCGGCG
GACGAGGCCGGCTGCGGTCTGGTTGAGGACCTGAACGTGGAAGCGTTGC
TGCTGCTTCCTGACGATGGCGCCGTCCAGGTACAGACCTGGGTGAGCGA
ACCGGACGAGGCCGGTCGCCACCGGCTCAGTATCCACGCCCGTTACAGC
GACAGCGAGCCCTGGACACGCTTGGCCACCGCAACCCTCGCCACCAGGG
GAACGGTATCCGGCTGGCAGGCCGGGGAGGCGTGGCCGCCGACCGGTGC
GGTCCCGGTCGAGACCGGAGTACCGTCACTGCGCGGGGTGTGGCGCCGA
GGCAACGAAGTGTTCGCCGAGGTCGCCCTGGACAGCACCCACGACGCCA
CCACATATGCCCTGCACCCTGCCCTCCTGACCGCCGCCCTCACCACCGC
CGGTGAGGAAACCCCCGCCGCGTGGCAGGCGCTGACCCTGCACGCCCGC
AACCCTGCCGAGCTGCGCGTCCGCCTCATCTCACACGATGACGGCACCC
TGTCCGTGGACGCCACCGACAGCACAGGCCTCCCCGTCCTGACCGTCCG
CTCCCTCACCCTGCGCACCGTCCCCGTCTACGAACCTGCCACCAGCACC
GACGACCTGCTCACCCTGACCTGGGCGGAGATCCCGGCCCCTCAGGAAA
CCGGCCTGACGGTCGGCCGGTTCGAGGACCTGGTGTCGGACGCTGATGT
GCCGGTACCCGAGGTGGCGGTCTTCACCGCACTCCCCGACAGCAGCGAG
AACCCGCTGGAACAGACCCGCGTACTGACCGCTCAGGTCCTCCAGGCAG
TCCAGACCTGGCTTGGCGGGGAACGTTTCACCGACAGCACGCTGGTCGT
GCGGACCGGCACCCGGTTGGCCGCCGCTGGGGTGTCGGGGTTGATGCGA
TCGGCTCAATCGGAACACCCCGGCCGGTTCGTCCTGGTCGAGAGCGACG
ACGACACGCTCGCCCCGGACCAGTTGGCCGCCACCGTCGGGCTCGACGA
GCCGCGGCTGCGGGTCAGCGGCGACCGGTACGAGGCACCGCGACTGGCT
CGTGTGAACGCCAGTGGGTCTGAGCCTGAAGCGGTTTGGGATCCGGATG
GCACGGTTCTGATCACCGGTGGTTCGGGTGTGCTGGCGGGGATCGCCGC
CCGGCACCTGGTGGCCGAACGCGGCGTGCGTCATCTGCTGCTGCTGTCC
CGCAGCGCCCCGGACGAGGCACTGATCAACCAACTCGGCGAACTGGGCG
CCCGGGTCGAGACAGCGGCCTGTGACGTGTCCGATCGTGCCGCGCTGGC
CCAGGTGCTGGCGGGTGTGTCACCGGAGCACCCCCTGACGGCAGTGATT
CACACCGCGGGCGTACTCGATGACGGTGTTGTCGAGTCCCTGACCGCGC
AGCGGCTCGACACGGTACTGCGGCCCAAGGCCGACGGCGCCTGGCATCT
GCACGAACTCACCCGCAACACCGACCTGGCCGCCTTCGTCATGTACTCC
TCCGCCGCCGGTGTCATGGGCGGTGGGGGGCAAGGTAACTACGCGGCGG
CAAACGCGTTCCTGGACGCGCTCGCCGAAGAACGCCGCGCCGAGGGCCT
GCCCGCACTCGCGGTGGCCTGGGGTCTGTGGGAGGACGCCAGTGGCCTG
ACCACGCAACTGACCGACACGGACCGTGACCGGATCCGGCGCGGTGGCC
TGCGGACTATCACCGCCGAGTACGGGATGCGGCTGTTCGACACCGCATC
ACGCCATGGCAACCCGATTCTGGTCGCCGCA

### [Fraction4]

(SEQ ID NO: 66)
CAACCCGATTCTGGTCGCCGCACCGATGGACCCGGTTTGGGACGCGGAA
GTCCCCGCGCTCCTCCGCTCGTTGCATCGTCCCGTCGCCCGGCGGGCCG
CCTCTACCAGCGACTCGTCAGCGCGGTGGCTGGCGGCCCTGGCACCGGC
CGAGCGGGAAGACGCACTGCTGAAGCTGGTGCGTGACAGCGCCGCTCTG
GTCCTGGGACACGCTGACGCCAGCACCATCCCCGCAGCCGCCGCATTCA
AGGATCTGGGTATCGATTCGCTGACCGCGGTGGAACTGCGCAACAGCCT
GGCGAAAGCCACAGGGCTGCGGCTGCCCAACACGACGGTGTTCGACTAC
CCCACCCCGGCCATCCTGGCCACCCGGCTGGGTGAGCTGTTCACCGGCG
AGAACCCTGCACCGGTACGCCCGTCGGTGTCGGTGGTGGGGCAGGACGA
GCCGCTGGCGGTCGTGGGTATGGCCTGCCGTCTGCCCGGCGGGGTGTCG
TCGCCTGAGGATCTGTGGCGCCTTGTGGAGTCGGGTACGGATGCGATTT
CCGGTTTCCCCGCCGACCGTGGGTGGGACGCGGAGAGCCTGTTCGATCC
GGACCCGGACGCGGTCGGGAAGTCGTACTGCGTAGAGGGCGGCTTCCTC
GACAGCGCAGCCAGCTTCGACGCCGGATTCTTCGGCATCAGCCCACGCG
AGGCTCTGGCGATGGACCCGCAGCAGCGGCTGATCATGGAGGTGTCCTG
GGAGGCCTTCGAGCGGGCCGGGATCGAGCCCGGTTCCGTGCGCGGCAGC
GACACCGGCGTCTTCATGGGCGCGTACGCCGGTGGCTACGGTGCCGGTG
CTGACCTCGGCGGCTTCGCGGCCACCGCCAGCGCGACCAGTGTCCTGTC
CGGCCGGGTGTCGTACTTCTTCGGCCTCGAAGGCCCCGCCATCACAGTC
GACACAGCCTGCTCGTCATCACTGGTGGCACTGCACCAGGCCGGGTATG
CCCTCCGGCAGGGAGAGTGTTCCCTGGCCCTGGTCGGCGGCGTCACCGT
GATGGCCACACCACAAAGCTTCGTGGAATTCTCCCGCCAGCGTGGTCTG
GCCTCCGATGGCCGGTGCAAGGCGTTCGCAGACAGCGCGGACGGCACGG
GATGGGCTGAAGGCGTTGGTGTGCTGCTGGTAGAGCGGCTTTCCGACGC
CCAGGCCAAGGGCCATCAGGTGTTGGCGGTGGTCCGTAGCTCGGCGGTC
AACCAGGACGGCGCGTCCAACGGTCTGACCGCGCCGAACGGTCCTTCGC
AGCAGCGGGTGATCCAAGCCGCTCTCAGTAACGCCGGCCTCGCCGCGCA
CGAGGTGGATGTGGTCGAGGCCCACGGCACGGGCACGACGCTGGGCGAC
CCGATCGAGGCCCAGGCGCTGATCGCCACTTACGGTCAGGACCGGGAAC
GGCCCCTGCTGCTGGGTTCGCTGAAGTCGAACATCGGTCATGCTCAGGC
CGCCTCGGGCGTGTCGGGTGTCATCAAGATGGTCATGGCCCTGCAGCAC
AACACGGTTCCCCGCACCCTGCACGTGGATGAGCCGTCGCGGCACGTGG
ACTGGGCGGCGGGTGCGGTTGAGCTGGTGAGGGAGAACCAGCCCTGGCC
CGGCACCGACCGGCCCCGTCGGGCGGGCGTGTCGTCCTTCGGAGTCAGC
GGCACCAACGCCCACGTCATCCTGGAGAGCGCACCCCCCGCTCAGCCCG
CGGAGGAGGCGCAGCCTGTTGAGACGCCGGTGGTGGCCTCGGATGTGCT
GCCGCTGGTGATATCGGCCAAGACCCAGCCCGCCCTGACCGAACACGAA
GACCGGCTGCGCGCCTACCTGGCGGCGTCGCCCGGGGCGGATATACGGG
CTGTGGCATCGACGCTGGCGGTGACACGGTCGGTGTTCGAGCACCGCGC
CGTACTCCTTGGAGATGACACCGTCACCGGCACCGCGGTGACCGACCCC
AGGATCGTGTTTGTCTTTCCCGGGCAGGGGTGGCAGTGGCTGGGGATGG
GCAGTGCACTGCGCGATTCGTCGGTGGTGTTCGCCGAGCGGATGGCCGA
GTGTGCGGCGGCGTTGCGCGAGTTCGTGGACTGGGATCTGTTCACGGTT
CTGGATGATCCGGCGGTGGTGGACCGGGTTGATGTGGTCCAGCCCGCTT
CCTGGGCGATGATGGTTTCCCTGGCCGCGGTGTGGCAGGCGGCCGGTGT
GCGGCCGGATGCGGTGATCGGCCATTCGCAGGGTGAGATCGCCGCAGCT
TGTGTGGCGGGTGCGGTGTCACTACGCGATGCCGCCCGGATCGTGACCT
TGCGCAGCCAGGCGATCGCCCGGGGCCTGGCGGGCCGGGGCGCGATGGC
ATCCGTCGCCCTGCCCGCGCAGGATGTCGAGCTGGTCGACGGGGCCTGG
ATCGCCGCCCACAACGGGCCCGCCTCCACCGTGATCGCGGGCACCCCGG
AAGCGGTCGACCATGTCCTCACCGCTCATGAGGCACAAGGGGTGCGGGT
GCGGCGGATCACCGTCGACTATGCCTCGCACACCCCGCACGTCGAGCTG
ATCCGCGACGAACTACTCGACATCACTAGCGACAGCAGCTCGCAGACCC
CGCTCGTGCCGTGGCTGTCGACCGTGGACGGCACCTGGGTCGACAGCCC
GCTGGACGGGGAGTACTGGTACCGGAACCTGCGTGAACCGGTCGGTTTC
CACCCCGCCGTCAGCCAGTTGCAGGCCCAGGGCGACACCGTGTTCGTCG
AGGTCAGCGCCAGCCCGGTGTTGTTGCAGGCGATGGACGACGATGTCGT
CACGGTTGCCACGCTGCGTCGTGACGACGGCGACGCCACCCGGATGCTC
ACCGCCCTGGCACAGGCCTATGTCCACGGCGTCACCGTCGACTGGCCCG
CCATCCTCGGCACCACCACAACCCGGGTACTGGACCTTCCGACCTACGC
CTTCCA

### [Fraction5]

(SEQ ID NO: 67)
CCACAACCCGGGTACTGGACCTTCCGACCTACGCCTTCCAGCACCAGCG
GTACTGGCTCAGGAGCGTGGACCGGGCGGCTGCCGACGGTCATCCACTG
CTGGGCACCGTAGTGGCACTGCCCGGCTCCGACGGTGTGGTGCTCACCG
GGCGGGTGTCGCTGGCCACCCATACATGGCTGGCCGATCACGCGGTCCG
GGGCAGTGTCCTGCTACCCGGGACCGCATTTGTGGAACTGGTCGTCCGC
GCCGCCGACGAGGTCGAGTGCGACGTCGTTGACGAGTTGGTGATCGAAA
CCCCGCTCCTGCTGCCGCAGACCGGAGGCGTCCAACTGTCCGTGTCCGT
CGGCGGAGCCGACGAGTCCGGGCACCGCGCGGTGATGGTCTTCTCCCAG
GCGGACAACACCGATACCTGGACCCGGCACGTCACGGCGACAGTCAGCA
CCTCTGACTCGACGGTCTCGCTGCCGGAGTTTGCCTCGTGGCCACCAGC
CCAGGCCCGGCCGGTGAGCGTGGCCGACTTCTACGACCGGCTGG

### [Fraction1-2]

(SEQ ID NO: 68)
ACGCCCACGTCATCCTGGAAAGCGCACTCCCCACACAGCCTGCGGGCAA
CACAGTGGTCGAGTCGGCACCGGAGTGGGTGCCGTTGGTGATTTCGGCG
AGGACCCAGTCGGCACTGGCTGAATACGAGGGCCGGTTGCGTGCGTATC
TGGCGGCGTCGCCCGGGGCGGATACGCGGGCTGTGGCATCGACGCTGGC
GATGACACGGTCGGTGTTCGAGTACCGGGCCGTACTCATTGGAGATGAC
ACCGTCACCGGTACCGCGGCGACCGATCCGCGGGTGGTGTTCGTCTTCC
CGGGTCAGGGGTCGCAGCGTGCTGGTATGGGTGAGGAACTGGCCGCCGC
GTTCCCCGTCTTCGCGCGGATCCATCAGCAGGTGTGGGATCTGCTGGAT
GTGCCCGATCTCGATGTGAATGAGACCGGGTATGCCCAGCCGGCCCTGT
TCGCTTTGCAGGTGGCTCTGTTCGGGTTGCTGGAATCGTGGGGTGTACG
GCCGGATGCGGTGGTCGGTCACTCTGTCGGTGAGCTCGCCGCCGGATAC
GTCTCCGGGTTGTGGTCGTTGGAGGATGCCTGCACTTTGGTGTCGGCGC
GGGCTCGTCTGATGCAGGCTCTGCCTGCGGGTGGGGTGATGGTCGCTGT
CCCGGTCTCGGAGGATGAGGCTCGGGCCGTGCTGGGTGAGGGTGTGGAG
ATCGCCGCGGTCAACGGGCCGTCGTCGGTGGTTCTCTCCGGTGATGAGG
CCGCCGTGCTGCAGGCCGCGGAGGGGCTGGGGAAGTGGACGCGGCTGGC
GACCAGTCACGCGTTCCATTCCGCCCGTATGGAACCGATGCTGGAGGAG
TTCCGGGCGGTCGCTGAAGGCCTGACCTACCGGACGCCGCAGGTCGCCA
TGGCCGCTGGTGATCAGGTGATGACCGCTGAGTACTGGGTGCGGCAGGT
CCGGGACACGGTCCGGTTCGGCGAGCAGGTGGCCTCGTTCGAGGATGCG
GTGTTCGTCGAGCTGGGTGCCGACCGGTCACTGGCCCGCCTGGTCGATG
GCATCGCGATGCTGCACGGTGACCATGAGGCGCAGGCCGCTGTCGGTGC
CCTGGCTCACCTGTACGTGAACGGCGTGAGTGTCGAGTGGTCCGCGGTG
CTGGGTGATGTCCCGGTAACCCGGGTGCTGGATCTTCCGACGTACGCCT
TCCAGCACCAGCGGTACTGGCTTGAGGGCACGGACCGGGCGACTGCGGG
TGGTCATCCGTTGCTGGGTTCGGTGGTGCGGCTGGCCGAGGCCAGTGGG
GTGTTGTTCACTGCCCGGGTTTCCCGGAGCGGTGATCTGTGGCTGCGGG
ACCAGACGGTTCTGCCCGCGACGGTGTTCGTGGAGATGGCGCTGGCAGC
GGCGGACGAGGTCGGCTGCGGTCTGGTTGAGGATCTGAGTGTGGAAGCG
TTGCTGCTGCTTCCCGATGATGGCGCCGTCGAGGTACAGACCTGGGTGG
GCGAACCGGATGAGGGCGGTCGGCGCCGGCTCAGTGTCCACGCCCGTTA
CGGTGACGGCGAGCCCTGGACCTGCTTGGCCACCGCAACCCTGGCCACC
ACTACGGGTGTGGCCGCTGCCGCGGTCGGCTGGCAGGCCGGTGGGGTGT
GGCCGCCGGCCGGTGCGGTCCCGGTCGGGACATCGGCACCCTCACTGCG
GGCGGTGTGGCGCCTGGGCAGCGACATCTTCGCCGAGGTGGCCCTGGAC
GATGCCCATGATGCCACCAGGTTTGTGCTTCATCCCGGCCTGATGGCCG
CCGCGCTCACCACCGTAGGCGAGGAGACTCCCGCCGTGTGGCAGGGCCT
GACCCTGCACGCCGGCAATCCCGGCGAGCTGCGCGTCCGCCTCACCTCA
CACGATGACGGCACCCTGTCGGCAGAGGCCACCGACAGCACAGGCCTCC
CCGTCCTGACCGCCCGCTCGCTCACCCTGCGCACCGTCCCCGTATACGA
ACCGGCCACCAGCACCGACGACCTGCTCACCCTGACCTGGGCAGGAATC
CCCACCCCCCAGCAGACCGGCCTGACGGTGGGTGCGTTTGAAGACCTGG
CGGCGGACGGCGATGTGCCGGTACCCGAGGTGGCGGTCTTCACCGCACT
CCCCGACAGCGACGATCCGCTGGAGCAAACACGAAAGCTGACCGCTCAG
GTCCTCCACACACTCCAGGAGTGGCTTGGCGGGGAGCGCTTCAGCGACA
GCACGCTGGTGGTGCGGACCGGCACCGGGTTGGCCGCTGCTGGGGTGTC
GGGGTTGATGCGCTCGGCCCAGTCCGAACACCCCGGCCGGTTCGTCCTG
GTCGAAAGCGACGACGCCCTCACCCAGGATCAGCTGGCGGCGGCGGTCG
GACTGGATGAGCCGCGGCTGCGGGTCAGCGACGGCCGGTACGAAGTACC
ACGGCTGACCCGCACACATGCCGAAGAGCCTGAGCCTGAAAGGACGTGG
GATCCGGATGGCACGGTCCTGATCACGGGCGGTTCAGGTGTGCTGGCGG
GGATCGCCGCCCGGCACCTGGTGACCGAACGCGGCGTGCGTCATCTCCT
GCTGCTGTCCCGCAGCGCCCCGGATGAGGCGCTGATCGGCGAGCTTGGT
GAACTGGGGGCCCGGGTCGAGACAGCGGCCTGTGACGTGTCCGATCCTG
CCGCGCTGACGCAGGTGCTGGCGGGTGTCTCGCCGGAGCATCCCCTGAC
GGCCGTGATTCACACCGCGGGCGTGGTGGATGACGGTGTTGTGGAGTCT
TTGACCGTGCAGCGGCTGGAGACGGTACTGCGGCCCAAGGCCGACGGTG
CGTGGAACCTGCACGAGCTCACCCGGGATGCCGACCTGGCCGCGTTCGT
CATGTATTCCTCCGCCGCCGGTGTGCTCGGTAGTGCGGGGCAGGGCAAC
TACGCGGCGGCCAATGCGTTCCTGGACGCGCTGGCTGAGCAGCGTCACG
CTGAGGGTCTGCCCGCACTCGCGGTGGCCTGGGGTCTGTGGGAGGACGC
CAGTGGCCTGACCGCGCAACTGACCGACACGGACCGTGACCGGATCCGG
CGCGGTGGCCTGCGGGCCATCTCCGCCGAGCACGGGATGGGGCTGTTCG
ACAGCGCGTCACGCCACAGTGAACCGGTTCTGGTGGCCGCGCCGATGGA
GCCGGTACGGGACGCGGAAGTCCCGGCATTGCTGCGGTCGTTGCACCGC
CCGATTGCTCGGCGGGCCGCTGCCGCCGGTGGAGCGCGGTGGCTGGCCG
CCCTGGCACCGGCCGAGCGGGAGAAGGCACTGCTGAAGCTGGTGTCTGA
CGGCGCCGCGACGGTTCTGGGACACGCCGACACCAGCACGATTCCGGCA
ACCACGGCGTTCAAGGATCTGGGCATCAATTCGCTGACCGCGGTGGAAC
TGCGCAACAGCCTGGCGAAGGCCACGGAGCTGCGGCTGCCCGCCACGCT
GGTGTTCGACTACCCCACCCCGGCCGCCTTGGCTGCCCGGTTGGACGAG
TTGTTCACCGGCGAGAACCCCGTACCGGTACGCGGGCCGGTGTCGGCGG
TGGCGCAGGACGAGCCGCTGGCGATCGTGGGAATGGCCTGCCGCCTACC
CGGTGGAGTCTCGTCGCCTGAGGATCTGTGGCGTCTCCTGGAGTCGGGT
ACAGATGCGGTCTCCGGTTTCCCCACCGACCGTGGCTGGGACGTCGAGA
ACCTGTACGACATGGCTGGAAAATCGCACCGTGCTGAGGGTGGCTTCCT
GGATGCCGCGGCTGGCTTTGATGCCGGATTCTTCGGGATCAGTCCGCGT
GAGGCGTTGGCGATGGATCCGCAGCAGCGGCTGGTGCTGGAGGTGTCCT
GGGAGGCGTTCGAGCGGGCCGGGATCGAGCCCGGTTCCGTACGCGGCAG
CGATACCGGCGTTTTCATGGGTGCGTACCCCGGTGGCTACGGCATCGGT
GCCGACCTCGGCGGCTTCGGGGCCACCGCCAGTTCGGTCAGTGTCCTGT
CCGGCCGGGTGTCGTACTTCTTCGGCCTCGAGGGTCCCGCGTTCACAGT
CGACACGGCCTGCTCGTCATCGTTGGTGGCGTTGCATCAGGCGGGGTAT
GCCCTCCGGCAGGGAGAGTGTTCGCTGGCCCTGGTCGGCGGTGTCACTG
TGATGGCCACGCCACAGACTTTCGTGGAGTTCTCCCGCCAGGGCGGCCT
GGCCTCCGACGGCCGCTGCAAAGCGTTCGCCGACGCCGCGGACGGCACG
GGATGGGCTGAAGGTGTCGGTGTCCTGCTCGTAGAGCGACTCTCCGATG
CCCGCCGTAACGGTCACCAGGTGTTGGCGGTGGTGCGTGGATCAGCGGT
GAACCAGGACGGTGCGTCGAACGGTCTGACCGCGCCGAATGGTCCTTCG
CAGCAGCGGGTGATCCGGGCCGCTCTCAGCAACGCGGGTCTGAGCACGG
CTGAGGTGGATGTGGTCGAGGCGCACGGCACGGGCACAACGCTGGGTGA
CCCGATCGAGGCCCAGGCGCTGATCGCTACCTATGGCCAGGACCGTGAC
CAGCCTGTGCTGCTGGGTTCGGTGAAGTCGAACCTGGGTCATACGCAGG
CCGCTGCGGGTGTGTCCGGTGTCATCAAGATGGTGATGGCCCTGCAACA
CGGTCTGGTGCCGCGCACGTTGCATGTCGATGAGCCGTCACGGCATGTG
GACTGGTCGGCGGGCGCGGTGCAGCTCGTGACGGAGAACCAGCCGTGGC
CGGATATGGGCCGAGCGCGCCGGGCAGGCGTGTCGTCCTTCGGGATCAG
TGGCACCAACGCCCACGTCATCCTGGAAAGCGCACCCCCCACTCAGCCT
GCGGACAACGCGGTGATCGAGCGGGCACCGGAGTGGGTGCCGTTGGTGA
TTTCGGCCAGGACCCAGTCGGCTTTGACTGAGCACGAGGGCCGGTTGCG
TGCGTATCTGGCGGCGTCGCCCGGGGTGGATATGCGGGCTGTGGCATCG
ACGCTGGCGATGACACGGTCGGTGTTCGAGCACCGTGCCGTGCTGCTGG
GAGATGACACCGTCACCGGCACCGCTGTGTCTGACCCTCGGGCGGTGTT
CGTCTTCCCGGGACAGGGGTCGCAGCGTGCTGGCATGGGTGAGGAACTG
GCCGCCGCGTTCCCCGTCTTCGCGCGGATCCATCAGCAGGTGTGGGACC
TGCTCGATGTGCCCGATCTGGAGGTGAACGAGACCGGTTACGCCCAGCC
GGCCCTGTTCGCAATGCAGGTGGCTCTGTTCGGGC

### [Fraction 3-5]

(SEQ ID NO: 69)
AATGCAGGTGGCTCTGTTCGGGCTGCTGGAATCGTGGGGTGTACGACCGGACGCGGTGATCGG
CCATTCGGTGGGTGAGCTTGCGGCTGCGTATGTGTCCGGGGTGTGGTCGTTGGAGGATGCCTG
CACTTTGGTGTCGGCGCGGGCTCGTCTGATGCAGGCTCTGCCCGCGGGTGGGGTGATGGTCGC
TGTCCCGGTCTCGGAGGATGAGGCCCGGGCCGTGCTGGGTGAGGGTGTGGAGATCGCCGCGGT
CAACGGCCCGTCGTCGGTGGTTCTCTCCGGTGATGAGGCCGCCGTGCTGCAGGCCGCGGAGGG
GCTGGGGAAGTGGACGCGGCTGGCGACCAGCCACGCGTTCCATTCCGCCCGTATGGAACCCAT
GCTGGAGGAGTTCCGGGCGGTCGCCGAAGGCCTGACCTACCGGACGCCGCAGGTCTCCATGGC
CGTTGGTGATCAGGTGACCACCGCTGAGTACTGGGTGCGGCAGGTCCGGGACACGGTCCGGTT
CGGCGAGCAGGTGGCCTCGTACGAGGACGCCGTGTTCGTCGAGCTGGGTGCCGACCGGTCACT
GGCCCGCCTGGTCGACGGTGTCGCGATGCTGCACGGCGACCACGAAATCCAGGCCGCGATCGG
CGCCCTGGCCCACCTGTATGTCAACGGCGTCACGGTCGACTGGCCCGCGCTCCTGGGCGATGC
TCCGGCAACACGGGTGCTGGACCTTCCGACATACGCCTTCCAGCACCAGCGCTACTGGCTCGA
GGGCACGGACCGGGCGACTGCGGGTGGCCATCCGTTGCTGGGTTCGGCGGTGCGGCTGGCCGA
GGCCAGCGGGGTGTTGTTCACTGCCCGGGTTTCCCGGAGCGGCGATCTGTGGCTGCGGGACCA
GACGGTTCTGCCCGCGACGGTGTTCGTGGAGATGGCGCTGGCAGCGGCGGACGAGGCCGGCTG
CGGTCTGGTTGAGGACCTGAACGTGGAAGCGTTGCTGCTGCTTCCTGACGATGGCGCCGTCCA
GGTACAGACCTGGGTGAGCGAACCGGACGAGGCCGGTCGCCACCGGCTCAGTATCCACGCCCG
TTACAGCGACAGCGAGCCCTGGACACGCTTGGCCACCGCAACCCTCGCCACCAGGGGAACGGT
ATCCGGCTGGCAGGCCGGGGAGGCGTGGCCGCCGACCGGTGCGGTCCCGGTCGAGACCGGAGT
ACCGTCACTGCGCGGGGTGTGGCGCCGAGGCAACGAAGTGTTCGCCGAGGTCGCCCTGGACAG
CACCCACGACGCCACCACATATGCCCTGCACCCTGCCCTCCTGACCGCCGCCCTCACCACCGC
CGGTGAGGAAACCCCCGCCGCGTGGCAGGCGCTGACCCTGCACGCCCGCAACCCTGCCGAGCT
GCGCGTCCGCCTCATCTCACACGATGACGGCACCCTGTCCGTGGACGCCACCGACAGCACAGG
CCTCCCCGTCCTGACCGTCCGCTCCCTCACCCTGCGCACCGTCCCCGTCTACGAACCTGCCAC
CAGCACCGACGACCTGCTCACCCTGACCTGGGCGGAGATCCCGGCCCCTCAGGAAACCGGCCT
GACGGTCGGCCGGTTCGAGGACCTGGTGTCGGACGCTGATGTGCCGGTACCCGAGGTGGCGGT
CTTCACCGCACTCCCCGACAGCAGCGAGAACCCGCTGGAACAGACCCGCGTACTGACCGCTCA
GGTCCTCCAGGCAGTCCAGACCTGGCTTGGCGGGGAACGTTTCACCGACAGCACGCTGGTCGT
GCGGACCGGCACCCGGTTGGCCGCCGCTGGGGTGTCGGGGTTGATGCGATCGGCTCAATCGGA
ACACCCCGGCCGGTTCGTCCTGGTCGAGAGCGACGACGACACGCTCGCCCCGGACCAGTTGGC
CGCCACCGTCGGGCTCGACGAGCCGCGGCTGCGGGTCAGCGGCGACCGGTACGAGGCACCGCG
ACTGGCTCGTGTGAACGCCAGTGGGTCTGAGCCTGAAGCGGTTTGGGATCCGGATGGCACGGT
TCTGATCACCGGTGGTTCGGGTGTGCTGGCGGGGATCGCCGCCCGGCACCTGGTGGCCGAACG
CGGCGTGCGTCATCTGCTGCTGCTGTCCCGCAGCGCCCCGGACGAGGCACTGATCAACCAACT
CGGCGAACTGGGCGCCCGGGTCGAGACAGCGGCCTGTGACGTGTCCGATCGTGCCGCGCTGGC
CCAGGTGCTGGCGGGTGTGTCACCGGAGCACCCCCTGACGGCAGTGATTCACACCGCGGGCGT
ACTCGATGACGGTGTTGTCGAGTCCCTGACCGCGCAGCGGCTCGACACGGTACTGCGGCCCAA
GGCCGACGGCGCCTGGCATCTGCACGAACTCACCCGCAACACCGACCTGGCCGCCTTCGTCAT
GTACTCCTCCGCCGCCGGTGTCATGGGCGGTGGGGGGCAAGGTAACTACGCGGCGGCAAACGC
GTTCCTGGACGCGCTCGCCGAAGAACGCCGCGCCGAGGGCCTGCCCGCACTCGCGGTGGCCTG
GGGTCTGTGGGAGGACGCCAGTGGCCTGACCACGCAACTGACCGACACGGACCGTGACCGGAT
CCGGCGCGGTGGCCTGCGGACTATCACCGCCGAGTACGGGATGCGGCTGTTCGACACCGCATC
ACGCCATGGCAACCCGATTCTGGTCGCCGCACCGATGGACCCGGTTTGGGACGCGGAAGTCCC
CGCGCTCCTCCGCTCGTTGCATCGTCCCGTCGCCCGGCGGGCCGCCTCTACCAGCGACTCGTC
AGCGCGGTGGCTGGCGGCCCTGGCACCGGCCGAGCGGGAAGACGCACTGCTGAAGCTGGTGCG
TGACAGCGCCGCTCTGGTCCTGGGACACGCTGACGCCAGCACCATCCCCGCAGCCGCCGCATT
CAAGGATCTGGGTATCGATTCGCTGACCGCGGTGGAACTGCGCAACAGCCTGGCGAAAGCCAC
AGGGCTGCGGCTGCCCAACACGACGGTGTTCGACTACCCCACCCCGGCCATCCTGGCCACCCG
GCTGGGTGAGCTGTTCACCGGCGAGAACCCTGCACCGGTACGCCCGTCGGTGTCGGTGGTGGG
GCAGGACGAGCCGCTGGCGGTCGTGGGTATGGCCTGCCGTCTGCCCGGCGGGGTGTCGTCGCC
TGAGGATCTGTGGCGCCTTGTGGAGTCGGGTACGGATGCGATTTCCGGTTTCCCCGCCGACCG
TGGGTGGGACGCGGAGAGCCTGTTCGATCCGGACCCGGACGCGGTCGGGAAGTCGTACTGCGT
AGAGGGCGGCTTCCTCGACAGCGCAGCCAGCTTCGACGCCGGATTCTTCGGCATCAGCCCACG
CGAGGCTCTGGCGATGGACCCGCAGCAGCGGCTGATCATGGAGGTGTCCTGGGAGGCCTTCGA
GCGGGCCGGGATCGAGCCCGGTTCCGTGCGCGGCAGCGACACCGGCGTCTTCATGGGCGCGTA
CGCCGGTGGCTACGGTGCCGGTGCTGACCTCGGCGGCTTCGCGGCCACCGCCAGCGCGACCAG
TGTCCTGTCCGGCCGGGTGTCGTACTTCTTCGGCCTCGAAGGCCCCGCCATCACAGTCGACAC
AGCCTGCTCGTCATCACTGGTGGCACTGCACCAGGCCGGGTATGCCCTCCGGCAGGGAGAGTG
TTCCCTGGCCCTGGTCGGCGGCGTCACCGTGATGGCCACACCACAAAGCTTCGTGGAATTCTC
CCGCCAGCGTGGTCTGGCCTCCGATGGCCGGTGCAAGGCGTTCGCAGACAGCGCGGACGGCAC
GGGATGGGCTGAAGGCGTTGGTGTGCTGCTGGTAGAGCGGCTTTCCGACGCCCAGGCCAAGGG
CCATCAGGTGTTGGCGGTGGTCCGTAGCTCGGCGGTCAACCAGGACGGCGCGTCCAACGGTCT
GACCGCGCCGAACGGTCCTTCGCAGCAGCGGGTGATCCAAGCCGCTCTCAGTAACGCCGGCCT
CGCCGCGCACGAGGTGGATGTGGTCGAGGCCCACGGCACGGGCACGACGCTGGGCGACCCGAT
CGAGGCCCAGGCGCTGATCGCCACTTACGGTCAGGACCGGGAACGGCCCCTGCTGCTGGGTTC
GCTGAAGTCGAACATCGGTCATGCTCAGGCCGCCTCGGGCGTGTCGGGTGTCATCAAGATGGT
CATGGCCCTGCAGCACAACACGGTTCCCCGCACCCTGCACGTGGATGAGCCGTCGCGGCACGT
GGACTGGGCGGCGGGTGCGGTTGAGCTGGTGAGGGAGAACCAGCCCTGGCCCGGCACCGACCG
GCCCCGTCGGGCGGGCGTGTCGTCCTTCGGAGTCAGCGGCACCAACGCCCACGTCATCCTGGA
GAGCGCACCCCCCGCTCAGCCCGCGGAGGAGGCGCAGCCTGTTGAGACGCCGGTGGTGGCCTC
GGATGTGCTGCCGCTGGTGATATCGGCCAAGACCCAGCCCGCCCTGACCGAACACGAAGACCG
GCTGCGCGCCTACCTGGCGGCGTCGCCCGGGGCGGATATACGGGCTGTGGCATCGACGCTGGC
GGTGACACGGTCGGTGTTCGAGCACCGCGCCGTACTCCTTGGAGATGACACCGTCACCGGCAC
CGCGGTGACCGACCCCAGGATCGTGTTTGTCTTTCCCGGGCAGGGGTGGCAGTGGCTGGGGAT
GGGCAGTGCACTGCGCGATTCGTCGGTGGTGTTCGCCGAGCGGATGGCCGAGTGTGCGGCGGC
GTTGCGCGAGTTCGTGGACTGGGATCTGTTCACGGTTCTGGATGATCCGGCGGTGGTGGACCG
GGTTGATGTGGTCCAGCCCGCTTCCTGGGCGATGATGGTTTCCCTGGCCGCGGTGTGGCAGGC
GGCCGGTGTGCGGCCGGATGCGGTGATCGGCCATTCGCAGGGTGAGATCGCCGCAGCTTGTGT
GGCGGGTGCGGTGTCACTACGCGATGCCGCCCGGATCGTGACCTTGCGCAGCCAGGCGATCGC
CCGGGGCCTGGCGGGCCGGGGCGCGATGGCATCCGTCGCCCTGCCCGCGCAGGATGTCGAGCT
GGTCGACGGGGCCTGGATCGCCGCCCACAACGGGCCCGCCTCCACCGTGATCGCGGGCACCCC
GGAAGCGGTCGACCATGTCCTCACCGCTCATGAGGCACAAGGGGTGCGGGTGCGGCGGATCAC
CGTCGACTATGCCTCGCACACCCCGCACGTCGAGCTGATCCGCGACGAACTACTCGACATCAC
TAGCGACAGCAGCTCGCAGACCCCGCTCGTGCCGTGGCTGTCGACCGTGGACGGCACCTGGGT
CGACAGCCCGCTGGACGGGGAGTACTGGTACCGGAACCTGCGTGAACCGGTCGGTTTCCACCC
CGCCGTCAGCCAGTTGCAGGCCCAGGGCGACACCGTGTTCGTCGAGGTCAGCGCCAGCCCGGT
GTTGTTGCAGGCGATGGACGACGATGTCGTCACGGTTGCCACGCTGCGTCGTGACGACGGCGA
CGCCACCCGGATGCTCACCGCCCTGGCACAGGCCTATGTCCACGGCGTCACCGTCGACTGGCC
CGCCATCCTCGGCACCACCACAACCCGGGTACTGGACCTTCCGACCTACGCCTTCCAGCACCA
GCGGTACTGGCTCAGGAGCGTGGACCGGGCGGCTGCCGACGGTCATCCACTGCTGGGCACCGT
AGTGGCACTGCCCGGCTCCGACGGTGTGGTGCTCACCGGGCGGGTGTCGCTGGCCACCCATAC
ATGGCTGGCCGATCACGCGGTCCGGGGCAGTGTCCTGCTACCCGGGACCGCATTTGTGGAACT
GGTCGTCCGCGCCGCCGACGAGGTCGAGTGCGACGTCGTTGACGAGTTGGTGATCGAAACCCC
GCTCCTGCTGCCGCAGACCGGAGGCGTCCAACTGTCCGTGTCCGTCGGCGGAGCCGACGAGTC
CGGGCACCGCGCGGTGATGGTCTTCTCCCAGGCGGACAACACCGATACCTGGACCCGGCACGT
CACGGCGACAGTCAGCACCTCTGACTCGACGGTCTCGCTGCCGGAGTTTGCCTCGTGGCCACC
AGCCCAGGCCCGGCCGGTGAGCGTGGCCGACTTCTACGACCGGCTGG

Introduction of the constructed mother nucleus modification construct into a host, and heterologous expression production were performed according to the method of Example 1.

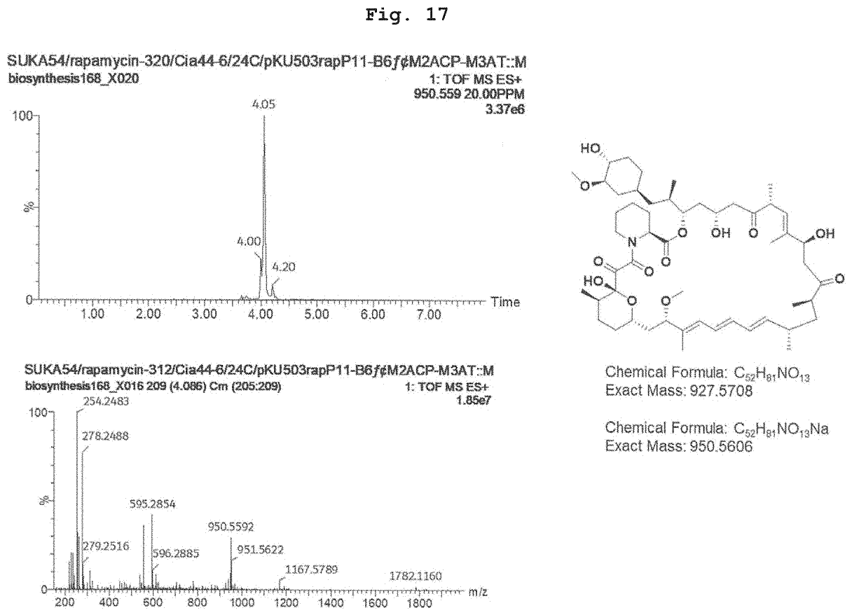

As the above results, novel mother nucleus modified rapamycin was detected as a peak of sodium-added type salt (FIG. 17, C₅₂H₈₁NO₁₃Na, measurement value: 950.5592, calculated value: 950.5606).

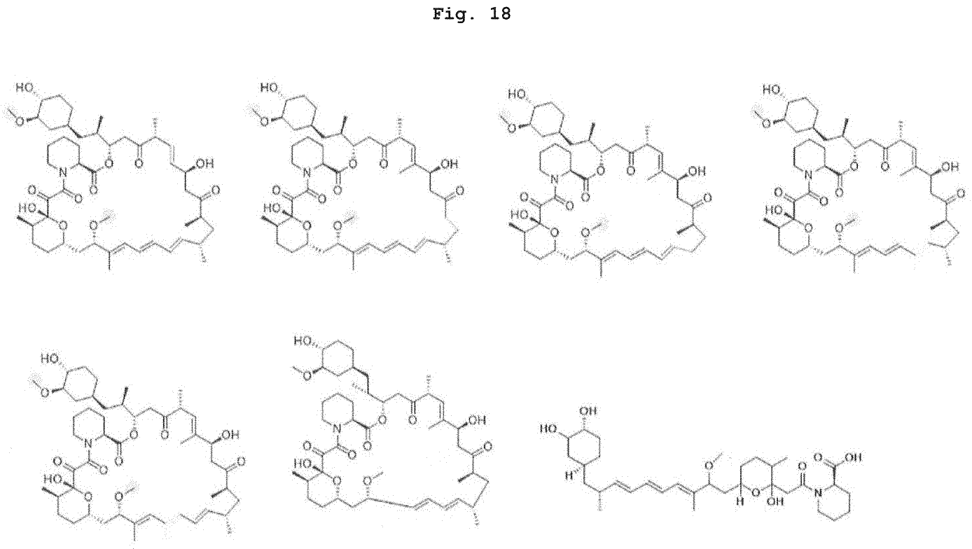

As described above, the present invention is an epoch-making technique that enables even an additional modification of a huge module. Examples of the compound created by the present invention are shown below (FIG. 18).

### INDUSTRIAL APPLICABILITY

According to the present invention, a compound having a desired mother nucleus modification can be prepared extremely highly efficiently. Therefore, the present invention is extremely useful, for example, in the field of drug discovery.

This application is based on a patent application No. 2019-016531 filed in Japan (filing date: Jan. 31, 2019), the contents of which are incorporated in full herein.

1. A method for producing a modified compound, comprising the following steps:
(1) a step of cleaving in vitro using CRISPR/Cas9 system, a target site in a gene cluster involved in the biosynthesis of a compound,
(2) a step of linking in vitro using Gibson assembly, the gene cluster cleaved in step (1) and a polynucleotide for modification, and
(3) a step of expressing the modified gene cluster obtained in step (2) in a microorganism expression system.

2. The method according to claim 1, further comprising the following step (A) before step (1):
(A) a step of inserting a gene cluster involved in the biosynthesis of a compound into an expression vector.

3. The method according to claim 2, wherein the expression vector is a chromosome-integrated expression vector.

4. The method according to claim 3, wherein the expression vector is selected from the group consisting of a Cosmid vector, a BAC vector, and a YAC vector.

5. The method according to claim 1, wherein the microorganism expression system is a heterologous expression system.

6. The method according to claim 1, wherein a Streptomyces lividans or SUKA strain is used in the microorganism expression system.

7. The method according to claim 4, wherein the microorganism expression system is a heterologous expression system.

8. The method according to claim 7, wherein a Streptomyces lividans or SUKA strain is used in the microorganism expression system.
